(19) United States
(12) Patent Application Publication
(10) Pub. No.: US 2004/0092552 A1
(43) Pub. Date: May 13, 2004

(54) 2-Fluorobenzenesulfonyl compounds for the treatment of inflammation

(76) Inventors: David L. Brown, Chesterfield, MO (US); Matthew J. Graneto, Chesterfield, MO (US); Cindy L. Ludwig, St. Louis, MO (US); John J. Talley, Cambridge, MA (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/258,493

(22) PCT Filed: Apr. 20, 2001
(86) PCT No.: PCT/US01/12983

(51) Int. Cl. A61K 31/4545; A61K 31/445; A61K 31/4439; A61K 31/40; A61K 31/277

(52) U.S. Cl. 514/317; 514/318; 514/343; 514/408; 514/520; 514/562; 514/602; 546/194; 546/229; 546/276.4; 548/577; 558/413; 562/430

(57) ABSTRACT

Methods of treating cyclooxygenase-2 mediated disorders comprising administering to a subject a therapeutically effective amount of one or more 2-fluorobenzenesulfonyl compounds corresponding to Formula (I) wherein A, R¹, R², and R³ are as described in the specification, and novel 2-fluorobenzenesulfonyl compounds within Formula (I).

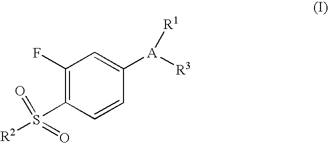

### FIELD OF THE INVENTION

This invention is in the field of anti-inflammatory pharmaceutical agents and generally relates to compounds, compositions and methods for treating cyclooxygenase-2 mediated disorders, such as inflammation and inflammation-related disorders. The invention particularly relates to 2-fluorobenzenesulfonyl compounds, compositions and methods for treating cyclooxygenase-2 mediated disorders.

### BACKGROUND OF THE INVENTION

Prostaglandins play a major role in the inflammation process and the inhibition of prostaglandin production, especially production of PGG₂, PGH₂ and PGE₂, has been a common target of antiinflammatory drug discovery. Common non-steroidal antiinflammatory drugs (“NSAIDs”) that are active in reducing the prostaglandin-induced pain and swelling associated with the inflammation process are, however, also active in affecting other prostaglandin-regulated processes not associated with the inflammation process. Thus, use of high doses of most common NSAIDs can produce severe side effects, including life threatening ulcers, that limit their therapeutic potential. An alternative to NSAIDs is the use of corticosteroids, which have even more drastic side effects, especially when long term therapy is involved.

Previous NSAIDs have been found to prevent the production of prostaglandins by inhibiting enzymes in the human arachidonic acid/prostaglandin pathway, including the enzyme cyclooxygenase (COX). The recent discovery of an inducible enzyme associated with inflammation (named “cyclooxygenase-2 (COX-2)” or “prostaglandin G/H synthase II”) provides a viable target of inhibition which more effectively reduces inflammation and produces fewer and less drastic side effects.

Recently, there has been significant research into some of the roles of cyclooxygenase-2. It has been found that COX-2 is upregulated in benign and malignant tumors (K. Subbararnaiah et al., Proc. Soc. Exp. Biol. Med., 216, 201 (1997)) including lung cancer (T. Hida et al., Anticancer Res., 18, 775-82 (1998)), Barrett's esophagus (K. Wilson, Cancer Res., 58, 2929-34 (1998)) and skin cancer (S. Buckman et al., Carcinogenesis, 19, 723-29 (1998)). It is expressed in airway cells with implication in asthma (P. Barnes et al., Lung Biol. Health Dis., 114, 111-27 (1998)). Cox-2 also has a role in pre-term labor, angiogenesis (M. Tsujii et al. Cell, 93, 705-16 (1998)), vascular rejection (M. Bustos, J. Clin. Invest., 100, 1150-58 (1997)), HIV induced apoptosis (G. Bagetta et al., Biochem. Biophys. Res. Comnmun., 244, 819-24 (1998)), neurodegeneration (T. Sandhya et al., Brain Res., 788, 223-31 (1998)), inflammatory bowel disease, colitis, (I. Singer et al., Gastroenterology, 115, 297-306 (1998)), cerebral ischemia (S. Nogawa et al., Proc. Natl. Acad. Sci., 95, 10966-71 (1998)), and hypertension (A. Nasjletti, Hypertension, 31, 194-200 (1997)), among others.

Drugs that inhibit cyclooxygenase affect colon cancer (T. Kawamori et al., Cancer Res., 58, 409-12 (1998)), allergic neuritis (K. Miyamoto et al., Neuro Report, 9, 2331-4 (1998)), dementia, burn infections (M. Shoup, J. Trauma: Inj., Infec., Crit care, 45, 215-21 (1998)), cytomegalovirus infectivity (E. Speir et al., Circ. Res., 83, 210-16 (1998)), and lumbago (H. Bosch, Curr. Med. Res. Opin., 14, 29-38 (1997)), among others.

The references below disclose compounds having antiinflammatory activity and show that efforts are continuing to find a safe and effective antiinflammatory agent. WO96/19463 describes oxazoles substituted with a [(2- or 3)-halo-4-(alkylsulfonyl, aminosulfonyl or alkylaminosulfonyl)]phenyl group that selectively inhibit cyclooxygenase-2. U.S. Pat. No. 5,380,738 describes 4-fluoro-phenyl and 4-methylsulfonyl substituted oxazoles that selectively inhibit cyclooxygenase-2. U.S. Pat. No. 5,719,163 describes substituted oxazoles that selectively inhibit cyclooxygenase-2. WO96/36617 describes oxazoles that selectively inhibit cyclooxygenase-2. WO96/19462 describes oxazoles that selectively inhibit cyclooxygenase-2. WO98/11080 describes 3,4-diaryl-oxazolones that selectively inhibit cyclooxygenase-2.

EP 799,823 A1 describes 1- or 2-[3-halo-4-(methylsulfonyl, aminosulfonyl or substituted aminosulfonyl)phenyl]-pyrroles that selectively inhibit cyclooxygenase-2. U.S. Pat. No. 5,935,990 describes substituted pyrroles that selectively inhibit cyclooxygenase-2.

WO99/64415 describes 1-(4-bromophenyl)-2-[3-fluoro-4-(methylsulfonyl)phenyl]-5-methyl-1H-pyrrole; 5-(4-bromophenyl)-1-[3-fluoro-4-(methylsulfonyl)phenyl]-3-(hydrogen, cyano, nitro, trifluoromethyl or ethoxycarbonyl)-1H-pyrazole; 2-[(4-bromophenyl) or (3-methyl-4-bromophenyl)]-1-[3-fluoro-4-(methylsulfonyl)phenyl]-4-trifluoromethyl-1H-imidazole; 4-[2-(4-bromophenyl)-4-hydroxy-4-trifluoromethyl-1H-imidazol-1-yl]-2-fluorobenzene sulfonamide; 3-(4-bromophenyl)-4-[3-fluoro-4-(methylsulfonyl)phenyl]-2(5H)-furanone; 3-(4-bromophenyl)-4-[3-fluoro-4-(methylsulfonyl or aminosulfonyl)phenyl]-5-methylisoxazole; 4-(4-bromophenyl)-5-[3-fluoro-4-(methylsulfonyl or aminosulfonyl)phenyl]-2-methyl-1,3-oxazole; and 4-(3-methyl-4-bromophenyl)-5-[3-fluoro-4-(methylsulfonyl)phenyl]-2-methyl-1,3-oxazole as intermediates used in the preparation of sulfonylbenzene compounds comprising an aryl or heteroaryl substituted phenyl moiety. WO99/64415 states that the disclosed sulfonylbenzene compounds are useful in the treatment of cyclooxygenase mediated diseases.

U.S. Pat. Nos. 5,466,823, 5,504,215, 5,508,426, 5,510,496, 5,516,907, 5,521,207 and 5,760,068 describe substituted pyrazolyl benzenesulfonamides that selectively inhibit cyclooxygenase-2. U.S. Pat. No. 5,475,018 describes 1,5-diphenyl pyrazoles that selectively inhibit cyclooxygenase-2. U.S. Pat. Nos. 5,486,534 and 5,756,529 describe 3,4-substituted pyrazoles that selectively inhibit cyclooxygenase-2. U.S. Pat. Nos. 5,401,765 and 5,639,777 describe 1,4,5-trisubstituted pyrazoles that selectively inhibit cyclooxygenase-2. U.S. Pat. Nos. 5,434,178 and 5,908,852 describe 1,3,5-trisubstituted pyrazoles that selectively inhibit cyclooxygenase-2.

WO94/15932 describes thiophene and furan derivatives that selectively inhibit cyclooxygenase-2. WO94/26731 describes thiophene compounds that selectively inhibit cyclooxygenase-2. WO97/16435 describes 3,4-diaryl-2-hydroxy-2,5-dihydrofurans as prodrugs of compounds that are cyclooxygenase-2 inhibitors. GB 2,294,879 describes substituted furanones as cyclooxygenase-2 inhibitors.

U.S. Pat. No. 5,859,257 describes substituted isooxazoles that selectively inhibit cyclooxygenase-2. EP 745596 describes substituted isooxazoles that selectively inhibit cyclooxygenase-2.

U.S. Pat. Nos. 5,344,991, 5,420,287 and 5,663,180 describe substituted cyclopentenes that selectively inhibit cyclooxygenase-2.

U.S. Pat. No. 5,418,254 describes substituted cyclopentadienyls that selectively inhibit cyclooxygenase-2.

U.S. Pat. No. 5,916,905 describes 2,3-substituted pyridines that selectively inhibit cyclooxygenase-2. U.S. Pat. No. 5,596,008 describes 3,4-diaryl substituted pyridines that selectively inhibit cyclooxygenase-2. WO98/03484 describes substituted pyridines that selectively inhibit cyclooxygenase-2.

U.S. Pat. Nos. 5,393,790 and 5,736,579 describe substituted spiro compounds that selectively inhibit cyclooxygenase-2.

U.S. Pat. Nos. 5,670,510, 5,672,626 and 5,672,627 describe spirodienes that selectively inhibit cyclooxygenase-2.

U.S. Pat. No. 5,668,161 describes substituted thiazoles that selectively inhibit cyclooxygenase-2.

U.S. Pat. No. 5,616,601 describes 1,2-substituted imidazoles that selectively inhibit cyclooxygenase-2. WO96/03387 describes 4,5-substituted imidazoles that selectively inhibit cyclooxygenase-2. WO96/03388 describes 1,2-substituted imidazoles that selectively inhibit cyclooxygenase-2. U.S. Pat. Nos. 5,521,193 and 5,534,521 describe benzimidazoles that selectively inhibit cyclooxygenase-2.

WO94/13635 describes 5-methanesulfonamido-1-indanones that selectively inhibit cyclooxygenase-2. WO94/20480 describes alkanesulfonamido-1-indanones that selectively inhibit cyclooxygenase-2.

WO96/09293 describes benz[g]indazolyls that selectively inhibit cyclooxygenase-2.

WO98/47890 describes benzopyran derivatives that selectively inhibit cyclooxygenase-2. U.S. Pat. No. 5,886,016 describes benzopyranopyrazolyls that selectively inhibit cyclooxygenase-2. U.S. Pat. No. 5,565,482 describes heteroarylpyranopyrazolyls that selectively inhibit cyclooxygenase-2.

U.S. Pat. No. 5,739,166 describes substituted terphenyls that selectively inhibit cyclooxygenase-2.

Finally, various additional substituted sulfonamides have been described. Pyrazolyl-sulfonylureas have been described as having possible hypoglycemic activity [H. Faid-Allah and H. Mokhtar, Ind. J. Chem, 27, 245 (1988)]. JP 1,045,374 describes water soluble tetrazolium compounds useful in assays for determining reducing substances. D. Mukerjee et al [Acta. Pharma. Jugosl., 31, 151 (1981)] describe tetrazolium sulfonamides as antiviral agents. JP 4,277,724 describes triphenyl pyrazolines as nonlinear optical material. JP 5,323,522 describes the use of heterocyclic compounds in black and white photographic material. U.S. Pat. No. 5,389,635 describes substituted imidazoles as angiotensin II antagonists. U.S. Pat. No. 5,387,592 describes substituted benzimidazole derivatives as angiotensin II antagonists. G. Dorofeenko et al [Khim. Farm. Zh., 16, 920 (1982)] describe pyridinium salts as antiviral agents. U.S. Pat. No. 5,338,749 describes diaryl-substituted heterocyclyl compounds as antiarthritis agents.

Compounds of the current invention, however, have not been described as antiinflammatory cyclooxygenase inhibitors.

### DESCRIPTION OF THE INVENTION

The present invention comprises methods of treating cyclooxygenase-2 mediated disorders, such as inflammation, in a subject having or susceptible to such disorders by administering to the subject a therapeutically-effective amount of one or more compounds of Formulae I-VII as described below. The methods of the present invention also include prophylatic treatment of a subject. The compounds of Formulae I-VII comprise a class of 2-fluorobenzene sulfonyl compounds that are safe and effective anti-inflammatory agents. These compounds generally exhibit improved selectivity and/or potency in inhibiting cyclooxygenase-2 over cyclooxygenase-1 relative to the corresponding sulfonamides or methylsulfones lacking the orthofluoro substituent. The present invention further comprises those novel 2-fluorobenzene sulfonyl compounds within the class of compounds of Formulae I-VII.

More specifically, the present method of treating cyclooxygenase-2 mediated disorders comprises administering to the subject a therapeutically-effective amount of one or more compounds selected from the class of compounds defined by Formula I:

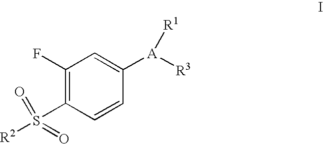

wherein:

A is a 5- or 6-member ring substituent selected from partially saturated or unsaturated heterocyclic and carbocyclic rings;

R¹ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;

R² is alkyl (particularly methyl) or amino; and

R³ represents one or more radicals selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-dalkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-aryl amino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl;

or a pharmaceutically-acceptable salt, tautomer or prodrug thereof;

provided that, (a) A is not pyrrolyl, and (b) A is not oxazolyl other than oxazolonyl.

Within the above-described group of compounds, as well as for the compounds disclosed in the various embodiments of the invention set forth throughout the instant application, one or more of the following conditions preferably is satisfied:

(1) when R¹ is 4-bromophenyl: (a) A is not pyrazolyl when R² is methyl and R³ is hydrogen, cyano, trifluoromethyl or ethoxycarbonyl; (b) A is not imidazolyl when R³ is trifluoromethyl; (c) A is not isoxazolyl when R³ is methyl; and (d) A is not 2-furanonyl when R³ is hydrogen;

(2) when R¹ is 3-methyl-4-bromophenyl, R² is methyl and R³ is trifluoromethyl, A is not imidazolyl;

(3) R¹ is other than 4-bromophenyl;

(4) R¹ is other than 4-bromophenyl or 3-methyl-4-bromophenyl; and/or

(5) R¹ is other than bromophenyl.

In another embodiment, the present method of treating cyclooxygenase-2 mediated disorders comprises administering to the subject a therapeutically-effective amount of one or more compounds selected from a preferred class of compounds consisting of those compounds of Formula I wherein:

A is a 5- or 6-member ring substituent selected from partially saturated or unsaturated heterocyclic and carbocyclic rings;

R¹ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;

R² is methyl or amino; and

R³ represents one or more radicals selected from hydrido, halo, C₁₋₂-alkyl, C₂₋₃-alkenyl, C₂₋₃-alkynyl, oxo, cyano, carboxyl, cyano-C₁₋₃-alkyl, (5- or 6-member ring heterocyclyl)oxy, C₁₋₃-alkoxy, C₁₋₃-alkylthio, C₁₋₃-alkylcarbonyl, C₃₋₆-cycloalkyl, phenyl, C₁₋₃-haloalkyl, 5- or 6-member ring heterocyclyl, C₃₋₆-cycloalkenyl, phenyl-C₁₋₃-alkyl, (5- or 6-member ring heterocyclyl)-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxy-C₁₋₃-alkyl, C₁₋₃-alkoxyphenyl-C₁₋₃-alkoxy -C₁₋₃-alkyl, C₁₋₃-alkoxycarbonyl-C₁₋₃-alkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-phenylamino, N-(phenyl-C₁₋₃-alkyl)amino, N-(C₁₋₃-alkyl)-N-(phenyl-C₁₋₃-alkyl)amino, N-(C₁₋₃-alkyl)-N-phenylamino, amino-C₁₋₃-alkyl, C₁₋₃-alkylamino-C₁₋₃-alkyl, N-phenylamino-C₁₋₃-alkyl, N-phenyl-C₁₋₃-alkylamino-C₁₋₃-alkyl, N-(C₁₋₃-alkyl)-N-phenyl-C₁₋₃-alkylamino-C₁₋₃-alkyl, N-(C₁₋₃-alkyl)-N-phenylamino-C₁₋₃-alkyl, phenyloxy, phenyl-C₁₋₃-alkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl;

or a pharmaceutically-acceptable salt, tautomer or prodrug thereof.

Within this preferred class of compounds, A preferably is a radical selected from thienyl, furyl, furanone, thiazolyl, oxothiazolyl, thioxothiazolyl, imidazolyl, benzofuryl, indenyl, benzothienyl, isoxazolyl, oxooxazolyl, pyrazolyl, cyclopentenyl, cyclopentadienyl, benzindazolyl, benzopyranopyrazolyl, phenyl, and pyridyl. More preferably, A is a radical selected from thienyl, furyl, furanone, thiazolyl, oxothiazolyl, thioxothiazolyl, imidazolyl, benzofuryl, indenyl, benzothienyl, isoxazolyl, pyrazolyl, cyclopentenyl, cyclopentadienyl, benzindazolyl, benzopyranopyrazolyl, phenyl, and pyridyl. Still more preferably, A is a radical selected from thienyl, furanone, isoxazolyl, pyrazolyl, cyclopentenyl and pyridinyl. Still more preferably, A is a radical selected from furanone, isoxazolyl, and pyrazolyl.

In another embodiment, the present method of treating cyclooxygenase-2 mediated disorders comprises administering to the subject a therapeutically-effective amount of one or more compounds selected from a more preferred class of compounds consisting of compounds of Formula I wherein one or both of R¹ and R³ are defined as follows:

R¹ is cyclohexyl, pyridinyl, or phenyl, wherein said cyclohexyl, pyridinyl, and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio; and/or

R³ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

In another embodiment, the present method of treating cyclooxygenase-2 mediated disorders comprises administering to the subject a therapeutically-effective amount of one or more compounds selected from a still more preferred class of compounds consisting of those compounds of Formula I wherein:

R¹ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and

R³ is a radical selected from hydrido, C₁₋₂-alkyl, C₁₋₃-alkoxy, C₁₋₃-alkylcarbonyl, C₁₋₃-haloalkyl, and C₁₋₃-alkoxycarbonyl.

In another embodiment, the present method of treating cyclooxygenase-2 mediated disorders comprises administering to the subject a therapeutically-effective amount of one or more compounds selected from a still more preferred group of compounds consisting of those compounds of Formula I wherein:

R¹ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, and methoxy; and

R³ is a radical selected from hydrido, methyl, methoxy, methylcarbonyl, trifluoromethyl, difluoromethyl, and methoxycarbonyl.

In another embodiment, the present method of treating cyclooxygenase-2 mediated disorders comprises administering to the subject a therapeutically-effective amount of one or more compounds selected from the group consisting of those compounds having a structure identical to compounds of Formula I except that the fluoro radical is in the meta position of the phenyl ring relative to the sulfonyl group rather than in the ortho position.

Utility of Methods and Compounds

The methods and compounds of the present invention would be useful for, but not limited to, the treatment of inflammation in a subject, and for treatment of other cyclooxygenase-2 mediated disorders, such as, as an analgesic in the treatment of pain and headaches, or as an antipyretic for the treatment of fever. For example, the methods and compounds of the invention would be useful to treat arthritis, including but not limited to rheumatoid arthritis, spondyloarthropathies, gouty arthritis, osteoarthritis, systemic lupus erythematosus and juvenile arthritis. Such methods and compounds of the invention would be useful in the treatment of asthma, bronchitis, menstrual cramps, preterm labor, tendinitis, bursitis, allergic neuritis, cytomegalovirus infectivity, apoptosis including HIV induced apoptosis, lumbago, liver disease including hepatitis, skin-related conditions such as psoriasis, eczema, acne, UV damage, burns and dermatitis, and from post-operative inflammation including from ophthalmic surgery such as cataract surgery and refractive surgery.

The methods and compounds of the invention also would be useful to treat gastrointestinal conditions such as inflammatory bowel disease, Crohn's disease, gastritis, irritable bowel syndrome and ulcerative colitis.

The methods and compounds of the invention would be useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

The methods and compounds would also be useful in the treatment of ophthalmic diseases, such as retinitis, conjunctivitis, retinopathies, uveitis, ocular photophobia, and of acute injury to the eye tissue.

The methods and compounds would also be useful in the treatment of pulmonary inflammation, such as that associated with viral infections and cystic fibrosis, and in bone resorption such as associated with osteoporosis.

The methods and compounds would also be useful for the treatment of certain central nervous system disorders, such as cortical dementias including Alzheimer's disease, neurodegeneration, and central nervous system damage resulting from stroke, ischemia and trauma. The term “treatment” includes partial or total inhibition of the dementia, including Alzheimer's disease, vascular dementia, multi-infarct dementia, pre-senile dementia, alcoholic dementia, and senile dementia.

The methods and compounds of the invention are useful as anti-inflammatory agents, such as for the treatment of arthritis, with the additional benefit of having significantly less harmful side effects. These methods and compounds would also be useful in the treatment of allergic rhinitis, respiratory distress syndrome, endotoxin shock syndrome, and liver disease. The methods and compounds would also be useful in the treatment of pain, but not limited to postoperative pain, dental pain, muscular pain, and pain resulting from cancer.

The methods and compounds above would be useful for, but not limited to, treating and preventing inflammation-related cardiovascular disorders in a subject. The methods and compounds would be useful for treatment and prevention of vascular diseases, coronary artery disease, aneurysm, vascular rejection, arteriosclerosis, atherosclerosis including cardiac transplant atherosclerosis, myocardial infarction, embolism, stroke, thrombosis, including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

The methods and compounds would be useful for, but not limited to, the treatment of angiogenesis-related disorders in a subject. According to the present invention, the methods and compounds can be used in the treatment of a subject in need of angiogenesis inhibition. The methods and compounds would be useful for treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but nonmalignant, conditions such as hemangiomas, including infantile hemaginomas, angiofibroma of the nasopharynx and avascular necrosis of bone; and disorders of the female reproductive system such as endometriosis.

The methods and compounds of the invention would be useful for the prevention or treatment of benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that affect epithelial cells throughout the body. Preferably, neoplasia is selected from gastrointestinal cancer, Barrett's esophagus, liver cancer, bladder cancer, pancreas cancer, ovary cancer, prostate cancer, cervical cancer, lung cancer, breast cancer and skin cancer, such as squamus cell and basal cell cancers. The methods and compounds can also be used to treat the fibrosis which occurs with radiation therapy. The methods and compounds can be used to treat subjects having adenomatous polyps, including those with familial adenomatous polyposis (FAP). Additionally, the methods and compounds can be used to prevent polyps from forming in patients at risk of FAP.

The methods and compounds of the present invention may be used alone or in conjunction with additional therapies and/or compounds known to those skilled in the art in the prevention or treatment of neoplasia. Alternatively, the methods and compounds described herein may be used in conjunctive therapy. By way of example, the compounds may be administered alone or in conjunction with other antineoplastic agents or other growth inhibiting agents or other drugs or nutrients.

The present methods and compounds may also be used in co-therapies, partially or completely, in addition to other antiinflammatories, such as together with steroids, NSAIDs, iNOS inhibitors, p38 inhibitors, MMP inhibitors, TNF inhibitors, 5-lipoxygenase inhibitors, LTB₄ receptor antagonists and LTA₄ hydrolase inhibitors.

The present methods and compounds may also be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. More preferred would be combinations with compounds selected from morphine, meperidine, codeine, pentazocine, buprenorphine, butorphanol, dezocine, meptazinol, hydrocodone, oxycodone, methadone, Tramadol [(+) enantiomer], DuP 747, Dynorphine A, Enadoline, RP-60180, HN-11608, E-2078, ICI-204448, acetominophen (paracetamol), propoxyphene, nalbuphine, E-4018, filenadol, mirfentanil, amitriptyline, DuP631, Tramadol [(−) enantiomer], GP-531, acadesine, AKI-1, AKI-2, GP-1683, GP-3269, 4030W92, tramadol racemate, Dynorphine A, E-2078, AXC3742, SNX-111, ADL2-1294, ICI-204448, CT-3, CP-99,994, and CP-99,994.

The methods and compounds can be used in co-therapies, in place of other conventional antiinflammatories, in combination with one or more antihistamines, decongestants, diuretics, antitussive agents or with other agents previously known to be effective in combination with antiinflammatory agents.

Subjects of Treatment

Besides being useful for human treatment, these methods and compounds are also useful for veterinary treatment of companion animals, exotic animals and farm animals, including mammals, rodents, and the like. More preferred animals include horses, dogs, and cats.

Selectivity of Compounds

The present novel methods preferably employ compounds which selectively inhibit cyclooxygenase-2 over cyclooxygenase-1. Preferably, the compounds have a cyclooxygenase-2 IC₅₀ of less than about 0.5 μM, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 5 μM. Such preferred selectivity may indicate an ability to reduce the incidence of common NSAID-induced side effects.

Pyrazoles

In still another embodiment, the present method of treating cyclooxygenase-2 mediated disorders comprises administering to the subject a therapeutically-effective amount of one or more compounds selected from a subclass of compounds of Formula I corresponding to Formula II:

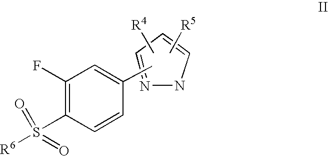

wherein substituents R⁴, R⁵ and R⁶ have the same definitions and sub-definitions as substituents R¹, R³ and R², respectively, set forth above for the compounds of Formula I, and the pharmaceutically-acceptable salts, tautomers and prodrugs thereof. Preferably, R⁴ is not 4-bromophenyl when R⁶ is methyl and R⁵ is hydrogen, cyano, trifluoromethyl or ethoxycarbonyl.

Within this subclass of compounds, a preferred group of compounds consists of those compounds of Formula IIA or Formula IIB:

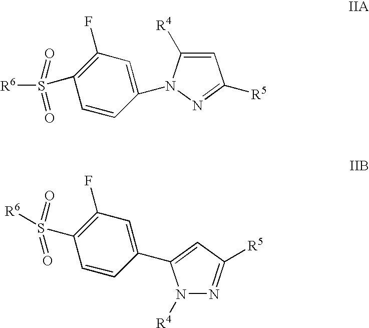

wherein R⁴, R⁵ and R⁶ are as defined above, and the pharmaceutically-acceptable salts, tautomers and prodrugs thereof.

Preferred species within this subclass include, but are not limited to:

5-phenyl-1-[3-fluoro-4-(methylsulfonyl)phenyl]-3-(trifluoromethyl)-1H-pyrazole;

5-(3-chlorophenyl)-1-[3-fluoro-4-(methylsuffonyl)phenyl]-3-(trifluoromethyl)-1H-pyrazole;

5-(4-chlorophenyl)-1-[3-fluoro-4-(methylsulfonyl)phenyl]-3-(trifluoromethyl)-1H-pyrazole;

5-(3-bromophenyl)-1-[3-fluoro-4-(methylsulfonyl)phenyl]-3-(trifluoromethyl)-1H-pyrazole;

5-(4-bromophenyl)-1-[3-fluoro-4-(methylsulfonyl)phenyl]-3-(trifluoromethyl)-1H-pyrazole;

5-(3-fluorophenyl)-1-[3-fluoro-4-(methylsulfonyl)phenyl]-3-(trifluoromethyl)-1H-pyrazole;

5-(4-fluorophenyl)-1-[3-fluoro-4-(methylsulfonyl)phenyl]-3-(trifluoromethyl)-1H-pyrazole;

5-(3-methylphenyl)-1-[3-fluoro-4-(methylsulfonyl)phenyl]-3-(trifluoromethyl)-1H-pyrazole;

5-(4-methylphenyl)-1-[3-fluoro-4-(methylsulfonyl)phenyl]-3-(trifluoromethyl)-1H-pyrazole;

5-(3-cyanophenyl)-1-[3-fluoro-4-(methylsulfonyl)phenyl]-3-(trifluoromethyl)-1H-pyrazole;

5-(4-cyanophenyl)-1-[3-fluoro-4-(methylsulfonyl)phenyl]-3-(trifluoromethyl)-1H-pyrazole;

5-(3-trifluoromethylphenyl)-1-[3-fluoro-4-(methylsulfonyl)phenyl]-3-(trifluoromethyl)-1H-pyrazole;

5-(4-trifluoromethylphenyl)-1-[3-fluoro-4-(methylsulfonyl)phenyl]-3-(trifluoromethyl)-1H-pyrazole;

5-(3-trifluoromethoxyphenyl)-1-[3-fluoro-4-(methylsulfonyl)phenyl]-3-(trifluoromethyl)-1H-pyrazole;

5-(4-trifluoromethoxyphenyl)-1-[3-fluoro-4-(methylsulfonyl)phenyl]-3-(trifluoromethyl)-1H-pyrazole;

5-(3,4-dichlorophenyl)-1-[3-fluoro-4-(methylsulfonyl)phenyl]-3-(trifluoromethyl)-1H-pyrazole;

5-(3-dibromophenyl)-1-[3-fluoro-4-(methylsulfonyl)phenyl]-3-(trifluoromethyl)-1H-pyrazole;

5-(3,4-difluorophenyl)-1-[3-fluoro-4-(methylsulfonyl)phenyl]-3-(trifluoromethyl)-1H-pyrazole;

5-(3,5-dichlorophenyl)-1-[3-fluoro-4-(methylsulfonyl)phenyl]-3-(trifluoromethyl)-1H-pyrazole;

5-(3,5-dibromophenyl)-1-[3-fluoro-4-(methylsulfonyl)phenyl]-3-(trifluoromethyl)-1H-pyrazole;

5-(3,5-difluorophenyl)-1-[3-fluoro-4-(methylsulfonyl)phenyl]-3-(trifluoromethyl)-1H-pyrazole;

5-(3,4-dimethylphenyl)-1-[3-fluoro-4-(methylsulfonyl)phenyl]-3-(trifluoromethyl)-1H-pyrazole;

5-(3,5-dimethylphenyl)-[3-fluoro-4-(methylsulfonyl)phenyl]-3-(trifluoromethyl)-1H-pyrazole;

5-(3-methyl-4-chlorophenyl)-1-[3-fluoro-4 (methylsulfonyl)phenyl]-3-(trifluoromethyl)-1H-pyrazole;

5-(4-methyl-3-chlorophenyl)-1-[3-fluoro-4-(methylsulfonyl)phenyl]-3-(trifluoromethyl)-1H-pyrazole;

5-(3-methyl-4-fluorophenyl)-1-[3-fluoro-4-(methylsulfonyl)phenyl]-3-(trifluoromethyl)-1H-pyrazole;

5-(4-methyl-3-fluorophenyl)-1-[3-fluoro-4-(methylsulfonyl)phenyl]-3-(trifluoromethyl)-1H-pyrazole;

5-(3-methyl-4-bromophenyl)-1-[3-fluoro-4-(methylsulfonyl)phenyl]-3-(trifluoromethyl)-1H-pyrazole;

5-(4-methyl-3-bromophenyl)-1-[3-fluoro-4-(methylsulfonyl)phenyl]-3-(trifluoromethyl)-1H-pyrazole;

5-(3-methyl-4-trifluoromethylphenyl)-1-[3-fluoro-4-(methylsulfonyl)phenyl]-3-(trifluoromethyl)-1H-pyrazole;

5-(4-methyl-3-trifluoromethylphenyl)-1-[3-fluoro-4-(methylsulfonyl)phenyl]-3-(trifluoromethyl)-1H-pyrazole;

5-(3-methyl-4-trifluoromethoxyphenyl)-1-[3-fluoro-4-(methylsulfonyl)phenyl]-3-(trifluoromethyl)-1H-pyrazole;

5-(4-methyl-3-trifluoromethoxyphenyl)-1-[3-fluoro-4-(methylsulfonyl)phenyl]-3-(trifluoromethyl)-1H-pyrazole;

5-(3-cyano-4-methylphenyl)-1-[3-fluoro-4-(methylsulfonyl)phenyl]-3-(trifluoromethyl)-1H-pyrazole;

5-(4-cyano-3-methylphenyl)-1-[3-fluoro-4-(methylsulfonyl)phenyl]-3-(trifluoromethyl)-1H-pyrazole;

5-(3-chloro-4-methoxyphenyl)-1-[3-fluoro-4-(methylsulfonyl)phenyl]-3-(trifluoromethyl)-1H-pyrazole;

5-(4-chloro-3-methoxyphenyl)-1-[3-fluoro-4-(methylsulfonyl)phenyl]-3-(trifluoromethyl)-1H-pyrazole;

5-(2-methylpyridin-6-yl)-1-[3-fluoro-4-(methylsulfonyl)phenyl]-3-(trifluoromethyl)-1H-pyrazole;

5-(2-methylthiazol-4-yl)-1-[3-fluoro-4 (methylsulfonyl)phenyl]-3-(trifluoromethyl)-1H-pyrazole;

5-(4-methylthiazol-2-yl)-1-[3-fluoro-4-(methylsulfonyl)phenyl]-3-(trifluoromethyl)-1H-pyrazole;

5-(2-methylpyridin-3-yl)-1-[3-fluoro-4 (methylsulfonyl)phenyl]-3-(trifluoromethyl)-1H-pyrazole;

5-(2-methylpyridin-3-yl)-1-[3-fluoro-4-(methylsulfonyl)phenyl]-3-(trifluoromethyl)-1H-pyrazole;

5-(3-pyridinyl)-1-[3-fluoro-4-(methylsulfonyl)phenyl]-3-(trifluoromethyl)-1H-pyrazole;

5-(5-methylpyridin-3-yl)-1-[3-fluoro-4-(methylsulfonyl)phenyl]-3-(trifluoromethyl)-1H-pyrazole;

5-(2-methylpyridin-3-yl)-1-[3-fluoro-4-(methylsulfonyl)phenyl]-3-(trifluoromethyl)-1H-pyrazole;

5-cyclohexyl-1-[3-fluoro-4-(methylsulfonyl)phenyl]-3-(trifluoromethyl)-1H-pyrazole;

5-cyclopentyl-1-[3-fluoro-4-(methylsulfonyl)phenyl]-3-(trifluoromethyl)-1H-pyrazole;

5-phenyl-1-[3-fluoro-4-(methylsulfonyl)phenyl]-3-(difluoromethyl)-1H-pyrazole;

5-(3-chlorophenyl)-1-[3-fluoro-4-(methylsulfonyl)phenyl]-3(difluoromethyl)-1H-pyrazole;

5-(4-chlorophenyl)-1-[3-fluoro-4-(methylsulfonyl)phenyl]-3-(difluoromethyl)-1H-pyrazole;

5-(3-bromophenyl)-1-[3-fluoro-4-(methylsulfonyl)phenyl]-3-(difluoromethyl)-1H-pyrazole;

5-(4-bromophenyl)-1-[3-fluoro-4-(methylsulfonyl)phenyl]-3-(difluoromethyl)-1H-pyrazole;

5-(3-fluorophenyl)-1-[3-fluoro-4-(methylsulfonyl)phenyl]-3-(difluoromethyl)-1H-pyrazole;

5-(4-fluorophenyl)-1-[3-fluoro-4-(methylsulfonyl)phenyl]-3-(difluoromethyl)-1H-pyrazole;

5-(3-methylphenyl)-1-[3-fluoro-4-(methylsulfonyl)phenyl]-3-(difluoromethyl)-1H-pyrazole;

5-(4-methylphenyl)-1-[3-fluoro-4-(methylsulfonyl)phenyl]-3-(difluoromethyl)-1H-pyrazole;

5-(3-cyanophenyl)-1-[3-fluoro-4-(methylsulfonyl)phenyl]-3-(difluoromethyl)-1H-pyrazole;

5-(4-cyanophenyl)-1-[3-fluoro-4-(methylsulfonyl)phenyl]-3-(difluoromethyl)-1H-pyrazole;

5-(3-trifluoromethylphenyl)-1-[3-fluoro-4-(methylsulfonyl)phenyl]-3-(difluoromethyl)-1H-pyrazole;

5-(4-trifluoromethylphenyl)-1-[3-fluoro-4-(methylsulfonyl)phenyl]-3-(difluoromethyl)-1H-pyrazole;

5-(3-trifluoromethoxyphenyl)-1-[3-fluoro-4-(methylsulfonyl)phenyl]-3-(difluoromethyl)-1H-pyrazole;

5-(4-trifluoromethoxyphenyl)-1-[3-fluoro-4-(methylsulfonyl)phenyl]-3-(difluoromethyl)-1H-pyrazole;

5-(3,4-dichlorophenyl)-1-[3-fluoro-4-(methylsulfonyl)phenyl]-3-(difluoromethyl)-1H-pyrazole;

5-(3,4-dibromophenyl)-1-[3-fluoro-4-(methylsulfonyl)phenyl]-3-(difluoromethyl)-1H-pyrazole;

5-(3,4-difluorophenyl)-1-[3-fluoro-4-(methylsulfonyl)phenyl]-3-(difluoromethyl)-1H-pyrazole;

5-(3,5-dichlorophenyl)-1-[3-fluoro-4-(methylsulfonyl)phenyl]-3-(difluoromethyl)-1H-pyrazole;

5-(3,5-dibromophenyl)-1-[3-fluoro-4-(methylsulfonyl)phenyl]-3-(difluoromethyl)-1H-pyrazole;

5-(3,5-difluorophenyl)-1-[3-fluoro-4-(methylsulfonyl)phenyl]-3-(difluoromethyl)-1H-pyrazole;

5-(3,4-dimethylphenyl)-1-[3-fluoro-4-(methylsulfonyl)phenyl]-3-(difluoromethyl)-1H-pyrazole;

5-(3,5-dimethylphenyl)-1-[3-fluoro-4-(methylsulfonyl)phenyl]-3-(difluoromethyl)-1H-pyrazole;

5-(3-methyl-4-chlorophenyl)-1-[3-fluoro-4-(methylsulfonyl)phenyl]-3-(difluoromethyl)-1H-pyrazole;

5-(4-methyl-3-chlorophenyl)-1-[3-fluoro-4-(methylsulfonyl)phenyl]-3-(difluoromethyl)-1H-pyrazole;

5-(3-methyl-4-fluorophenyl)-1-[3-fluoro-4-(methylsulfonyl)phenyl]-3-(difluoromethyl)-1H-pyrazole;

5-(4-methyl-3-fluorophenyl)-1-[3-fluoro-4-(methylsulfonyl)phenyl]-3-(difluoromethyl)-1H-pyrazole;

5-(3-methyl-4-bromophenyl)-1-[3-fluoro-4-(methylsulfonyl)phenyl]-3-(difluoromethyl)-1H-pyrazole;

5-(4-methyl-3-bromophenyl)-1-[3-fluoro-4-(methylsulfonyl)phenyl]-3-(difluoromethyl)-1H-pyrazole;

5-(3-methyl-4-trifluoromethylphenyl)-1-[3-fluoro-4-(methylsulfonyl)phenyl]-3-(difluoromethyl)-1H-pyrazole;

5-(4-methyl-3-trifluoromethylphenyl)-1-[3-fluoro-4-(methylsulfonyl)phenyl]-3-(difluoromethyl)-1H-pyrazole;

5-(3-methyl-4-trifluoromethoxyphenyl)-1-[3-fluoro-4-(methylsulfonyl)phenyl]-3-(difluoromethyl)-1H-pyrazole;

5-(4-methyl-3-trifluoromethoxyphenyl)-1-[3-fluoro-4-(methylsulfonyl)phenyl]-3-(difluoromethyl)-1H-pyrazole;

5-(3-cyano-4-methylphenyl)-1-[3-fluoro-4-(methylsulfonyl)phenyl]-3-(difluoromethyl)-1H-pyrazole;

5-(4-cyano-3-methylphenyl)-1-[3-fluoro-4-(methylsulfonyl)phenyl]-3-(difluoromethyl)-1H-pyrazole;

5-(3-chloro-4-methoxyphenyl)-1-[3-fluoro-4-(methylsulfonyl)phenyl]-3-(difluoromethyl)-1H-pyrazole;

5-(4-chloro-3-methoxyphenyl)-1-[3-fluoro-4-(methylsulfonyl)phenyl]-3-(difluoromethyl)-1H-pyrazole;

5-(2-methylpyridin-6-yl)-1-[3-fluoro-4-(methylsulfonyl)phenyl]-3-(difluoromethyl)-1H-pyrazole;

5-(2-methylthiazol-4-yl)-1-[3-fluoro-4-(methylsulfonyl)phenyl]-3-(difluoromethyl)-1H-pyrazole;

5-(4-methylthiazol-2-yl)-1-[3-fluoro-4-(methylsulfonyl)phenyl]-3-(difluoromethyl)-1H-pyrazole;

5-(2-methylpyridin-3-yl)-1-[3-fluoro-4-(methylsulfonyl)phenyl]-3-(difluoromethyl)-1H-pyrazole;

5-(2-methylpyridin-3-yl)-1-[3-fluoro-4-(methylsulfonyl)phenyl]-3-(difluoromethyl)-1H-pyrazole;

5-(3-pyridinyl)-1-[3-fluoro-4-(methylsulfonyl)phenyl]-3-(difluoromethyl)-1H-pyrazole;

5-(5-methylpyridin-3-yl)-1-[3-fluoro-4-(methylsulfonyl)phenyl]-3-(difluoromethyl)-1H-pyrazole;

5-(2-methylpyridin-3-yl)-1-[3-fluoro-4-(methylsulfonyl)phenyl]-3-(difluoromethyl)-1H-pyrazole;

5-cyclohexyl-1-[3-fluoro-4-(methylsulfonyl)phenyl]-3-(difluoromethyl)-1H-pyrazole;

5-cyclopentyl-1-[3-fluoro-4-(methylsulfonyl)phenyl]-3-(difluoromethyl)-1H-pyrazole;

1-phenyl-5-[3-fluoro-4-(methylsulfonyl)phenyl]-3-(trifluoromethyl)-1H-pyrazole;

1-(3-chlorophenyl)-5-[3-fluoro-4-(methylsulfonyl)phenyl]-3-(trifluoromethyl)-1H-pyrazole;

1-(4-chlorophenyl)-5-[3-fluoro-4-(methylsulfonyl)phenyl]-3-(trifluoromethyl)-1H-pyrazole;

1-(3-bromophenyl)-5-[3-fluoro-4-(methylsulfonyl)phenyl]-3-(trifluoromethyl)-1H-pyrazole;

1-(4-bromophenyl)-5-[3-fluoro-4-(methylsulfonyl)phenyl]-3-(trifluoromethyl)-1H-pyrazole;

1-(3-fluorophenyl)-5-[3-fluoro-4-(methylsulfonyl)phenyl]-3-(trifluoromethyl)-1H-pyrazole;

1-(4-fluorophenyl)-5-[3-fluoro-4-(methylsulfonyl)phenyl]-3-(trifluoromethyl)-1H-pyrazole;

1-(3-methylphenyl)-5-[3-fluoro-4-(methylsulfonyl)phenyl]-3-(trifluoromethyl)-1H-pyrazole;

1-(4-methylphenyl)-5-[3-fluoro-4-(methylsulfonyl)phenyl]-3-(trifluoromethyl)-1H-pyrazole;

1-(3-cyanophenyl)-5-[3-fluoro-4-(methylsulfonyl)phenyl]-3-(trifluoromethyl)-1H-pyrazole;

1-(4-cyanophenyl)-5-[3-fluoro-4-(methylsulfonyl)phenyl]-3-(trifluoromethyl)-1H-pyrazole;

1-(3-trifluoromethylphenyl)-5-[3-fluoro-4-(methylsulfonyl)phenyl]-3-(trifluoromethyl)-1H-pyrazole;

1-(4-trifluoromethylphenyl)-5-[3-fluoro-4-(methylsulfonyl)phenyl]-3-(trifluoromethyl)-1H-pyrazole;

1-(3-trifluoromethoxyphenyl)-5-[3-fluoro-4-(methylsulfonyl)phenyl]-3-(trifluoromethyl)-1H-pyrazole;

1-(4-trifluoromethoxyphenyl)-5-[3-fluoro-4-(methylsulfonyl)phenyl]-3-(trifluoromethyl)-1H-pyrazole;

1-(3,4-dichlorophenyl)-5-[3-fluoro-4-(methylsulfonyl)phenyl]-3-(trifluoromethyl)-1H-pyrazole;

1-(3,4-dibromophenyl)-5-[3-fluoro-4-(methylsulfonyl)phenyl]-3-(trifluoromethyl)-1H-pyrazole;

1-(3,4-difluorophenyl)-5-[3-fluoro-4-(methylsulfonyl)phenyl]-3-(trifluoromethyl)-1H-pyrazole;

1-(3,5-dichlorophenyl)-5-[3-fluoro-4-(methylsulfonyl)phenyl]-3-(trifluoromethyl)-1H-pyrazole;

1-(3,5-dibromophenyl)-5-[3-fluoro-4-(methylsulfonyl)phenyl]-3-(trifluoromethyl)-1H-pyrazole;

1-(3,5-difluorophenyl)-5-[3-fluoro-4-(methylsulfonyl)phenyl]-3-(trifluoromethyl)-1H-pyrazole;

1-(3,4-dimethylphenyl)-5-[3-fluoro-4-(methylsulfonyl)phenyl]-3-(trifluoromethyl)-1H-pyrazole;

1-(3,5-dimethylphenyl)-5-[3-fluoro-4-(methylsulfonyl)phenyl]-3-(trifluoromethyl)-1H-pyrazole;

1-(3-methyl-4-chlorophenyl)-5-[3-fluoro-4-(methylsulfonyl)phenyl]-3-(trifluoromethyl)-1H-pyrazole;

1-(4-methyl-3-chlorophenyl)-5-[3-fluoro-4-(methylsulfonyl)phenyl]-3-(trifluoromethyl)-1H-pyrazole;

1-(3-methyl-4-fluorophenyl)-5-[3-fluoro-4-(methylsulfonyl)phenyl]-3-(trifluoromethyl)-1H-pyrazole;

1-(4-methyl-3-fluorophenyl)-5-[3-fluoro-4-(methylsulfonyl)phenyl]-3-(trifluoromethyl)-1H-pyrazole;

1-(3-methyl-4-bromophenyl)-5-[3-fluoro-4-(methylsulfonyl)phenyl]-3-(trifluoromethyl)-1H-pyrazole;

1-(4-methyl-3-bromophenyl)-5-[3-fluoro-4-(methylsulfonyl)phenyl]-3-(trifluoromethyl)-1H-pyrazole;

1-(3-methyl-4-trifluoromethylphenyl)-5-[3-fluoro-4-(methylsulfonyl)phenyl]-3-(trifluoromethyl)-1H-pyrazole;

1-(4-methyl-3-trifluoromethylphenyl)-5-[3-fluoro-4-(methylsulfonyl)phenyl]-3-(trifluoromethyl)-1H-pyrazole;

1-(3-methyl-4-trifluoromethoxyphenyl)-5-[3-fluoro-4-(methylsulfonyl)phenyl]-3-(trifluoromethyl)-1H-pyrazole;

1-(4-methyl-3-trifluoromethoxyphenyl)-5-[3-fluoro-4-(methylsulfonyl)phenyl]-3-(trifluoromethyl)-1H-pyrazole;

1-(3-cyano-4-methylphenyl)-5-[3-fluoro-4-(methylsulfonyl)phenyl]-3-(trifluoromethyl)-1H-pyrazole;

1-(4-cyano-3-methylphenyl)-5-[3-fluoro-4-(methylsulfonyl)phenyl]-3-(trifluoromethyl)-1H-pyrazole;

1-(3-chloro-4-methoxyphenyl)-5-[3-fluoro-4-(methylsulfonyl)phenyl]-3-(trifluoromethyl)-1H-pyrazole;

1-(4-chloro-3-methoxyphenyl)-5-[3-fluoro-4-(methylsulfonyl)phenyl]-3-(trifluoromethyl)-1H-pyrazole;

1-(2-methylpyridin-6-yl)-5-[3-fluoro-4-(methylsulfonyl)phenyl]-3-(trifluoromethyl)-1H-pyrazole;

1-(2-methylthiazol-4-yl)-5-[3-fluoro-4-(methylsulfonyl)phenyl]-3-(trifluoromethyl)-1H-pyrazole;

1-(4-methylthiazol-2-yl)-5-[3-fluoro-4-(methylsulfonyl)phenyl]-3-(trifluoromethyl)-1H-pyrazole;

1-(2-methylpyridin-3-yl)-5-[3-fluoro-4-(methylsulfonyl)phenyl]-3-(trifluoromethyl)-1H-pyrazole;

1-(2-methylpyridin-3-yl)-5-[3-fluoro-4-(methylsulfonyl)phenyl]-3-(trifluoromethyl)-1H-pyrazole;

1-(3-pyridinyl)-5-[3-fluoro-4-(methylsulfonyl)phenyl]-3-(trifluoromethyl)-1H-pyrazole;

1-(5-methylpyridin-3-yl)-5-[3-fluoro-4-(methylsulfonyl)phenyl]-3-(trifluoromethyl)-1H-pyrazole;

1-(2-methylpyridin-3-yl)-5-[3-fluoro-4-(methylsulfonyl)phenyl]-3-(trifluoromethyl)-1H-pyrazole;

1-cyclohexyl-5-[3-fluoro-4-(methylsulfonyl)phenyl]-3-(trifluoromethyl)-1H-pyrazole;

1-cyclopentyl-5-[3-fluoro-4-(methylsulfonyl)phenyl]-3-(trifluoromethyl)-1H-pyrazole;

1-phenyl-5-[3-fluoro-4-(methylsulfonyl)phenyl]-3-(difluoromethyl)-1H-pyrazole;

1-(3-chlorophenyl)-5-[3-fluoro-4-(methylsulfonyl)phenyl]-3-(difluoromethyl)-1H-pyrazole;

1-(4-chlorophenyl)-5-[3-fluoro-4-(methylsulfonyl)phenyl]-3-(difluoromethyl)-1H-pyrazole;

1-(3-bromophenyl)-5-[3-fluoro-4-(methylsulfonyl)phenyl]-3-(difluoromethyl)-1H-pyrazole;

1-(4-bromophenyl)-5-[3-fluoro-4-(methylsulfonyl)phenyl]-3-(difluoromethyl)-1H-pyrazole;

1-(3-fluorophenyl)-5-[3-fluoro-4-(methylsulfonyl)phenyl]-3-(difluoromethyl)-1H-pyrazole;

1-(4-fluorophenyl)-5-[3-fluoro-4-(methylsulfonyl)phenyl]-3-(difluoromethyl)-1H-pyrazole;

1-(3-methylphenyl)-5-[3-fluoro-4-(methylsulfonyl)phenyl]-3-(difluoromethyl)-1H-pyrazole;

1-(4-methylphenyl)-5-[3-fluoro-4-(methylsulfonyl)phenyl]-3-(difluoromethyl)-1H-pyrazole;

1-(3-cyanophenyl)-5-[3-fluoro-4-(methylsulfonyl)phenyl]-3-(difluoromethyl)-1H-pyrazole;

1-(4-cyanophenyl)-5-[3-fluoro-4-(methylsulfonyl)phenyl]-3-(difluoromethyl)-1H-pyrazole;

1-(3-trifluoromethylphenyl)-5-[3-fluoro-4-(methylsulfonyl)phenyl]-3-(difluoromethyl)-1H-pyrazole;

1-(4-trifluoromethylphenyl)-5-[3-fluoro-4-(methylsulfonyl)phenyl]-3-(difluoromethyl)-1H-pyrazole;

1-(3-trifluoromethoxyphenyl)-5-[3-fluoro-4-(methylsulfonyl)phenyl]-3-(difluoromethyl)-1H-pyrazole;

1-(4-trifluoromethoxyphenyl)-5-[3-fluoro-4-(methylsulfonyl)phenyl]-3-(difluoromethyl)-1H-pyrazole;

1-(3,4-dichlorophenyl)-5-[3-fluoro-4-(methylsulfonyl)phenyl]-3-(difluoromethyl)-1H-pyrazole;

1-(3,4-dibromophenyl)-5-[3-fluoro-4-(methylsulfonyl)phenyl]-3-(difluoromethyl)-1H-pyrazole;

1-(3,4-difluorophenyl)-5-[3-fluoro-4-(methylsulfonyl)phenyl]-3-(difluoromethyl)-1H-pyrazole;

1-(3,5-dichlorophenyl)-5-[3-fluoro-4-(methylsulfonyl)phenyl]-3-(difluoromethyl)-1H-pyrazole;

1-(3,5-dibromophenyl)-5-[3-fluoro-4-(methylsulfonyl)phenyl]-3-(difluoromethyl)-1H-pyrazole;

1-(3,5-difluorophenyl)-5-[3-fluoro-4-(methylsulfonyl)phenyl]-3-(difluoromethyl)-1H-pyrazole;

1-(3,4-dimethylphenyl)-5-[3-fluoro-4-(methylsulfonyl)phenyl]-3-(difluoromethyl)-1H-pyrazole;

1-(3,5-dimethylphenyl)-5-[3-fluoro-4-(methylsulfonyl)phenyl]-3-(difluoromethyl)-1H-pyrazole;

1-(3-methyl-4-chlorophenyl)-5-[3-fluoro-4-(methylsulfonyl)phenyl]-3-(difluoromethyl)-1H-pyrazole;

1-(4-methyl-3-chlorophenyl)-5-[3-fluoro-4-(methylsulfonyl)phenyl]-3-(difluoromethyl)-1H-pyrazole;

1-(3-methyl-4-fluorophenyl)-5-[3-fluoro-4-(methylsulfonyl)phenyl]-3-(difluoromethyl)-1H-pyrazole;

1-(4-methyl-3-fluorophenyl)-5-[3-fluoro-4-(methylsulfonyl)phenyl]-3-(difluoromethyl)-1H-pyrazole;

1-(3-methyl-4-bromophenyl)-5-[3-fluoro-4-(methylsulfonyl)phenyl]-3-(difluoromethyl)-1H-pyrazole;

1-(4-methyl-3-bromophenyl)-5-[3-fluoro-4-(methylsulfonyl)phenyl]-3-(difluoromethyl)-1H-pyrazole;

1-(3-methyl-4-trifluoromethylphenyl)-5-[3-fluoro-4-(methylsulfonyl)phenyl]-3-(difluoromethyl)-1H-pyrazole;

1-(4-methyl-3-trifluoromethylphenyl)-5-[3-fluoro-4-(methylsulfonyl)phenyl]-3-(difluoromethyl)-1H-pyrazole;

1-(3-methyl-4-trifluoromethoxyphenyl)-5-[3-fluoro-4-(methylsulfonyl)phenyl]-3-(difluoromethyl)-1H-pyrazole;

1-(4-methyl-3-trifluoromethoxyphenyl)-5-[3-fluoro-4-(methylsulfonyl)phenyl]-3-(difluoromethyl)-1H-pyrazole;

1-(3-cyano-4-methylphenyl)-5-[3-fluoro-4-(methylsulfonyl)phenyl]-3-(difluoromethyl)-1H-pyrazole;

1-(4-cyano-3-methylphenyl)-5-[3-fluoro-4-(methylsulfonyl)phenyl]-3-(difluoromethyl)-1H-pyrazole;

-(3-chloro-4-methoxyphenyl)-5-[3-fluoro-4-(methylsulfonyl)phenyl]-3-(difluoromethyl)-1H-pyrazole;

1-(4-chloro-3-methoxyphenyl)-5-[3-fluoro-4-(methylsulfonyl)phenyl]-3-(difluoromethyl)-1H-pyrazole;

1-(2-methylpyridin-6-yl)-5-[3-fluoro-4-(methylsulfonyl)phenyl]-3-(difluoromethyl)-1H-pyrazole;

1-(2-methylthiazol-4-yl)-5-[3-fluoro-4-(methylsulfonyl)phenyl]-3-(difluoromethyl)-1H-pyrazole;

1-(4-methylthiazol-2-yl)-5-[3-fluoro-4-(methylsulfonyl)phenyl]-3-(difluoromethyl)-1H-pyrazole;

1-(2-methylpyridin-3-yl)-5-(3-fluoro-4-(methylsulfonyl)phenyl]-3-(difluoromethyl)-1H-pyrazole;

1-(2-methylpyridin-3-yl)-5-[3-fluoro-4-(methylsulfonyl)phenyl]-3-(difluoromethyl)-1H-pyrazole;

1-(3-pyridinyl)-5-[3-fluoro-4-(methylsulfonyl)phenyl]-3-(difluoromethyl)-1H-pyrazole; 1-(5-methylpyridin-3-yl)-5-[3-fluoro-4-(methylsulfonyl)phenyl]-3-(difluoromethyl)-1H-pyrazole;

1-(2-methylpyridin-3-yl)-5-[3-fluoro-4-(methylsulfonyl)phenyl]-3-(difluoromethyl)-1H-pyrazole;

1-cyclohexyl-5-[3-fluoro-4-(methylsulfonyl)phenyl]-3-(difluoromethyl)-1H-pyrazole;

1-cyclopentyl-5-[3-fluoro-4-(methylsulfonyl)phenyl]-3-(difluoromethyl)-1H-pyrazole;

2-fluoro-4-[1-phenyl-3-(difluoromethyl)-1H-pyrazol-5-yl]benezenesulfonamide;

2-fluoro-4-[1-(3-chlorophenyl)-3-(difluoromethyl)-1H-pyrazol-5-yl]benezenesulfonamide;

2-fluoro-4-[1-(4-chlorophenyl)-3-(difluoromethyl)-1H-pyrazol-5-yl]benezenesulfonamide;

2-fluoro-4-[1-(3-bromophenyl)-3-(difluoromethyl)-1H-pyrazol-5-yl]benezenesulfonamide;

2-fluoro-4-[1-(4-bromophenyl)-3-(difluoromethyl)-1H-pyrazol-5-yl]benezenesulfonamide;

2-fluoro-4-[1-(3-fluorophenyl)-3-(difluoromethyl)-1H-pyrazol-5-yl]benezenesulfonamide;

2-fluoro-4-[1-(4-fluorophenyl)-3-(difluoromethyl)-1H-pyrazol-5-yl]benezenesulfonamide;

2-fluoro-4-[1-(3-methylphenyl)-3-(difluoromethyl)-1H-pyrazol-5-yl]benezenesulfonamide;

2-fluoro-4-[1-(4-methylphenyl)-3-(difluoromethyl)-1H-pyrazol-5-yl]benezenesulfonamide;

2-fluoro-4-[1-(3-cyanophenyl)-3-(difluoromethyl)-1H-pyrazol-5-yl]benezenesulfonamide;

2-fluoro-4-[1-(4-cyanophenyl)-3-(difluoromethyl)-1H-pyrazol-5-yl]benezenesulfonamide;

2-fluoro-4-[1-(3-trifluoromethylphenyl)-3-(difluoromethyl)-1H-pyrazol-5-yl]benezenesulfonamide;

2-fluoro-4-[1-(4-trifluoromethylphenyl)-3-(difluoromethyl)-1H-pyrazol-5-yl]benezenesulfonamide;

2-fluoro-4-[1-(3-trifluoromethoxyphenyl)-3-(difluoromethyl)-1H-pyrazol-5-yl]benezenesulfonamide;

2-fluoro-4-[1-(4-trifluoromethoxyphenyl)-3-(difluoromethyl)-1H-pyrazol-5-yl]benezenesulfonamide;

2-fluoro-4-[1-(3,4-dichlorophenyl)-3-(difluoromethyl)-1H-pyrazol-5-yl]benezenesulfonamide;

2-fluoro-4-[1-(3,4-dibromophenyl)-3-(difluoromethyl)-1H-pyrazol-5-yl]benezenesulfonamide

2-fluoro-4-[1-(3,4-difluorophenyl)-3-(difluoromethyl)-1H-pyrazol-5-yl]benezenesulfonamide;

2-fluoro-4-[1-(3,5-dichlorophenyl)-3-(difluoromethyl)-1H-pyrazol-5-yl]benezenesulfonamide;

2-fluoro-4-[1-(3,5-dibromophenyl)-3-(difluoromethyl)-1H-pyrazol-5-yl]benezenesulfonamide;

2-fluoro-4-[1-(3,5-difluorophenyl)-3-(difluoromethyl)-1H-pyrazol-5-yl]benezenesulfonamide;

2-fluoro-4-[1-(3,4-dimethylphenyl)-3-(difluoromethyl)-1H-pyrazol-5-yl]benezenesulfonamide;

2-fluoro-4-[1-(3,5-dimethylphenyl)-3-(difluoromethyl)-1H-pyrazol-5-yl]benezenesulfonamide;

2-fluoro-4-[1-(3-methyl-4-chlorophenyl)-3-(difluoromethyl)-1H-pyrazol-5-yl]benezenesulfonamide;

2-fluoro-4-[1-(4-methyl-3-chlorophenyl)-3-(difluoromethyl)-1H-pyrazol-5-yl]benezenesulfonamide;

2-fluoro-4-[1-(3-methyl-4-fluorophenyl)-3-(difluoromethyl)-1H-pyrazol-5-yl]benezenesulfonamide;

2-fluoro-4-[1-(4-methyl-3-fluorophenyl)-3-(difluoromethyl)-1H-pyrazol-5-yl]benezenesulfonamide;

2-fluoro-4-[1-(3-methyl-4-bromophenyl)-3-(difluoromethyl)-1H-pyrazol-5-yl]benezenesulfonamide;

2-fluoro-4-[1-(4-methyl-3-bromophenyl)-3-(difluoromethyl)-1H-pyrazol-5-yl]benezenesulfonamide;

2-fluoro-4-[1-(3-methyl-4-trifluoromethylphenyl)-3-(difluoromethyl)-1H-pyrazol-5-yl]benezenesulfonamide;

2-fluoro-4-[1-(4-methyl-3-trifluoromethylphenyl)-3-(difluoromethyl)-1H-pyrazol-5-yl]benezenesulfonamide;

2-fluoro-4-[1-(3-methyl-4-trifluoromethoxyphenyl)-3-(difluoromethyl)-1H-pyrazol-5-yl]benezenesulfonamide;

2-fluoro-4-[1-(4-methyl-3-trifluoromethoxyphenyl)-3-(difluoromethyl)-1H-pyrazol-5-yl]benezenesulfonamide;

2-fluoro-4-[1-(3-cyano-4-methylphenyl)-3-(difluoromethyl)-1H-pyrazol-5-yl]benezenesulfonamide;

2-fluoro-4-[1-(4-cyano-3-methylphenyl)-3-(difluoromethyl)-1H-pyrazol-5-yl]benezenesulfonamide;

2-fluoro-4-[1-(3-chloro-4-methoxyphenyl)-3-(difluoromethyl)-1H-pyrazol-5-yl]benezenesulfonamide;

2-fluoro-4-[1-(4-chloro-3-methoxyphenyl)-3-(difluoromethyl)-1H-pyrazol-5-yl]benezenesulfonamide;

2-fluoro-4-[1-(2-methylpyridin-6-yl)-3-(difluoromethyl)-1H-pyrazol-5-yl]benezenesulfonamide;

2-fluoro-4-[1-(2-methylthiazol-4-yl)-3-(difluoromethyl)-1H-pyrazol-5-yl]benezenesulfonamide;

2-fluoro-4-[1-(4-methylthiazol-2-yl)-3-(difluoromethyl)-1H-pyrazol-5-yl]benezenesulfonamide;

2-fluoro-4-[1-(2-methylpyridin-3-yl)-3-(difluoromethyl)-1H-pyrazol-5-yl]benezenesulfonamide;

2-fluoro-4-[1-(2-methylpyridin-3-yl)-3-(difluoromethyl)-1H-pyrazol-5r -yl]benezenesulfonamide;

2-fluoro-4-[1-(3-pyridinyl)-3-(difluoromethyl)-1H-pyrazol-5-yl]benezenesulfonamide;

2-fluoro-4-[1-(5-methylpyridin-3-yl)-3-(difluoromethyl)-1H-pyrazol-5-yl]benezenesulfonamide;

2-fluoro-4-[1-(2-methylpyridin-3-yl)-3-(difluoromethyl)-1H-pyrazol-5-yl]benezenesulfonamide;

2-fluoro-4-[1-cyclohexyl-3-(difluoromethyl)-1H-pyrazol-5-yl]benezenesulfonamide;

2-fluoro-4-[1-cyclopentyl-3-(difluoromethyl)-1H-pyrazol-5-yl]benezenesulfonamide;

2-fluoro-4-[1-phenyl-3-(trifluoromethyl)-1H-pyrazol-5-yl]benezenesulfonamide;

2-fluoro-4-[1-(3-chlorophenyl)-3-(trifluoromethyl)-1H-pyrazol-5-yl]benezenesulfonamide;

2-fluoro-4-[1-(4-chlorophenyl)-3-(trifluoromethyl)-1H-pyrazol-5-yl]benezenesulfonamide;

2-fluoro-4-[1-(3-bromophenyl)-3-(trifluoromethyl)-1H-pyrazol-5-yl]benezenesulfonamide;

2-fluoro-4-[1-(4-bromophenyl)-3-(trifluoromethyl)-1H-pyrazol-5-yl]benezenesulfonamide;

2-fluoro-4-[1-(3-fluorophenyl)-3-(trifluoromethyl)-1H-pyrazol-5-yl]benezenesulfonamide;

2-fluoro-4-[1-(4-fluorophenyl)-3-(trifluoromethyl)-1H-pyrazol-5-yl]benezenesulfonamide;

2-fluoro-4-[1-(3-methylphenyl)-3-(trifluoromethyl)-1H-pyrazol-5-yl]benezenesulfonamide;

2-fluoro-4-[1-(4-methylphenyl)-3-(trifluoromethyl)-1H-pyrazol-5-yl]benezenesulfonamide;

2-fluoro-4-[1-(3-cyanophenyl)-3-(trifluoromethyl)-1H-pyrazol-5-yl]benezenesulfonamide;

2-fluoro-4-[1-(4-cyanophenyl)-3-(trifluoromethyl)-1H-pyrazol-5-yl]benezenesulfonamide;

2-fluoro-4-[1-(3-trifluoromethylphenyl)-3-(trifluoromethyl)-1H-pyrazol-5-yl]benezenesulfonamide;

2-fluoro-4-[1-(4-trifluoromethylphenyl)-3-(trifluoromethyl)-1H-pyrazol-5-yl]benezenesulfonamide;

2-fluoro-4-[1-(3-trifluoromethoxyphenyl)-3-(trifluoromethyl)-1H-pyrazol-5-yl]benezenesulfonamide;

2-fluoro-4-[1-(4-trifluoromethoxyphenyl)-3-(trifluoromethyl)-1H-pyrazol-5-yl]benezenesulfonamide;

2-fluoro-4-[1-(3,4-dichlorophenyl)-3-(trifluoromethyl)-1H-pyrazol-5-yl]benezenesulfonamide;

2-fluoro-4-[1-(3,4-dibromophenyl)-3-(trifluoromethyl)-1H-pyrazol-5-yl]benezenesulfonamide

2-fluoro-4-[1-(3,4-difluorophenyl)-3-(trifluoromethyl)-1H-pyrazol-5-yl]benezenesulfonamide;

2-fluoro-4-[1-(3,5-dichlorophenyl)-3-(trifluoromethyl)-1H-pyrazol-5-yl]benezenesulfonamide;

2-fluoro-4-[1-(3,5-dibromophenyl)-3-(trifluoromethyl)-1H-pyrazol-5-yl]benezenesulfonamide;

2-fluoro-4-[1-(3,5-difluorophenyl)-3-(trifluoromethyl)-1H-pyrazol-5-yl]benezenesulfonamide;

2-fluoro-4-[1-(3,4-dimethylphenyl)-3-(trifluoromethyl)-1H-pyrazol-5-yl]benezenesulfonamide;

2-fluoro-4-[1-(3,5-dimethylphenyl)-3-(trifluoromethyl)-1H-pyrazol-5-yl]benezenesulfonamide;

2-fluoro-4-[1-(3-methyl-4-chlorophenyl)-3-(trifluoromethyl)-1H-pyrazol-5-yl]benezenesulfonamide;

2-fluoro-4-[1-(4-methyl-3-chlorophenyl)-3-(trifluoromethyl)-1H-pyrazol-5-yl]benezenesulfonamide;

2-fluoro-4-[1-(3-methyl-4-fluorophenyl)-3-(trifluoromethyl)-1H-pyrazol-5-yl]benezenesulfonamide;

2-fluoro-4-[1-(4-methyl-3-fluorophenyl)-3-(trifluoromethyl)-1H-pyrazol-5-yl]benezenesulfonamide;

2-fluoro-4-[1-(3-methyl-4-bromophenyl)-3-(trifluoromethyl)-1H-pyrazol-5-yl]benezenesulfonamide;

2-fluoro-4-[1-(4-methyl-3-bromophenyl)-3-(trifluoromethyl)-1H-pyrazol-5-yl]benezenesulfonamide;

2-fluoro-4-[1-(3-methyl-4-trifluoromethylphenyl)-3-(trifluoromethyl)-1H-pyrazol-5-yl]benezenesulfonamide;

2-fluoro-4-[1-(4-methyl-3-trifluoromethylphenyl)-3-(trifluoromethyl)-1H-pyrazol-5-yl]benezenesulfonamide;

2-fluoro-4-[1-(3-methyl-4-trifluoromethoxyphenyl)-3-(trifluoromethyl)-1H-pyrazol-5-yl]benezenesulfonamide;

2-fluoro-4-[1-(4-methyl-3-trifluoromethoxyphenyl)-3-(trifluoromethyl)-1H-pyrazol-5-yl]benezenesulfonamide;

2-fluoro-4-[1-(3-cyano-4-methylphenyl)-3-(trifluoromethyl)-1H-pyrazol-5-yl]benezenesulfonamide;

2-fluoro-4-[1-(4-cyano-3-methylphenyl)-3-(trifluoromethyl)-1H-pyrazol-5-yl]benezenesulfonamide;

2-fluoro-4-[1-(3-chloro-4-methoxyphenyl)-3-(trifluoromethyl)-1H-pyrazol-5-yl]benezenesulfonamide;

2-fluoro-4-[1-(4-chloro-3-methoxyphenyl)-3-(trifluoromethyl)-1H-pyrazol-5-yl]benezenesulfonamide;

2-fluoro-4-[1-(2-methylpyridin-6-yl)-3-(trifluoromethyl)-1H-pyrazol-5-yl]benezenesulfonamide;

2-fluoro-4-[1-(2-methylthiazol-4-yl)-3-(trifluoromethyl)-1H-pyrazol-5-yl]benezenesulfonamide;

2-fluoro-4-[1-(4-methylthiazol-2-yl)-3-(trifluoromethyl)-1H-pyrazol-5-yl]benezenesulfonamide;

2-fluoro-4-[1-(2-methylpyridin-3-yl)-3-(trifluoromethyl)-1H-pyrazol-5-yl]benezenesulfonamide;

2-fluoro-4-[1-(2-methylpyridin-3-yl)-3-(trifluoromethyl)-1H-pyrazol-5-yl]benezenesulfonamide;

2-fluoro-4-[1-(3-pyridinyl)-3-(trifluoromethyl)-1H-pyrazol-5-yl]benezenesulfonamide;

2-fluoro-4-[1-(5-methylpyridin-3-yl)-3-(trifluoromethyl)-1H-pyrazol-5-yl]benezenesulfonamide;

2-fluoro-4-[1-(2-methylpyridin-3-yl)-3-(trifluoromethyl)-1H-pyrazol-5-yl]benezenesulfonamide;

2-fluoro-4-[1-cyclohexyl-3-(trifluoromethyl)-1H-pyrazol-5-yl]benezenesulfonamide;

2-fluoro-4-[1-cyclopentyl-3-(trifluoromethyl)-1H-pyrazol-5-yl]benezenesulfonamide;

4-[5-(4-methylphenyl)-3-(trifluoromethyl)-1H-pyrazol-1-yl]-2-fluorobenzenesulfonamide;

4-[3-(difluoromethyl)-5-(3-fluoro-4-methoxyphenyl)-1H-pyrazole-1-yl]-2-fluorobenzenesulfonamide;

and the pharmaceutically-acceptable salts, tautomers and prodrugs thereof.

In one embodiment, the species is:

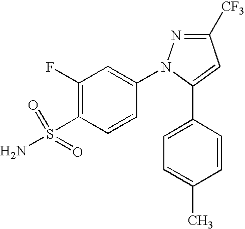

and the pharmaceutically-acceptable salts, tautomers and prodrugs thereof. In another embodiment, the fluoro radical is at the meta position relative to the sulfonyl group.

In another embodiment, the species is:

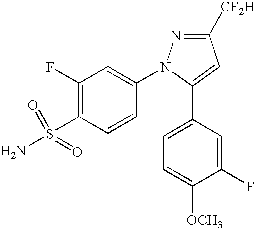

and the pharmaceutically-acceptable salts, tautomers and prodrugs thereof. In another embodiment, the fluoro radical is at the meta position relative to the sulfonyl group.

Thiophenes

In still another embodiment, the present method of treating cyclooxygenase-2 mediated disorders comprises administering to the subject a therapeutically-effective amount of one or more compounds selected from a subclass of compounds of Formula I corresponding to Formula III:

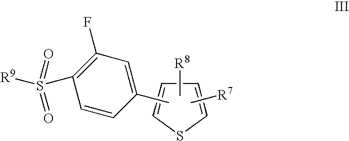

wherein substituents R⁷, R⁸ and R⁹ have the same definitions and sub-definitions as substituents R¹, R³ and R², respectively, set forth above for the compounds of Formula I, and the pharmaceutically-acceptable salts, tautomers and prodrugs thereof.

Within this subclass of compounds, a preferred group of compounds consists of those compounds of Formula IIIA:

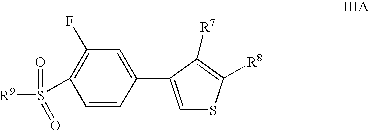

wherein R⁷, R⁸ and R⁹ are as defined above, and the pharmaceutically-acceptable salts, tautomers and prodrugs thereof.

Within this subclass of compounds, another preferred group of compounds, in addition to those embodiments previously described with respect to compounds of Formula I, consists of those compounds of Formula III wherein:

R⁷ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and

R⁸ is a radical selected from hydrido, halogen, C₁₋₂-alkyl, C₁₋₃-alkoxy, C₁₋₃-alkylcarbonyl, C₁₋₃-haloalkyl, and C₁₋₃-alkoxycarbonyl.

Within this subclass of compounds, another preferred group of compounds, in addition to those embodiments previously described with respect to compounds of Formula I, consists of those compounds of Formula III wherein:

R⁷ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, and methoxy; and

R⁸ is a radical selected from hydrido, chloro, fluoro, bromo, iodo, cyano, methyl, methoxy, methylcarbonyl, trifluoromethyl, difluoromethyl, and methoxycarbonyl.

Preferred species within this subclass include, but are not limited to:

3-phenyl-4-[3-fluoro-4-(methylsulfonyl)phenyl]thiophene;

3-(3-chlorophenyl)-4-[3-fluoro-4-(methylsulfonyl)phenyl]thiophene;

3-(4-chlorophenyl)-4-[3-fluoro-4-(methylsulfonyl)phenyl]thiophene;

3-(3-bromophenyl)-4-[3-fluoro-4-(methylsulfonyl)phenyl]thiophene;

3-(4-bromophenyl)-4-[3-fluoro-4-(methylsulfonyl)phenyl]thiophene;

3-(3-fluorophenyl)-4-[3-fluoro-4-(methylsulfonyl)phenyl]thiophene;

3-(4-fluorophenyl)-4-[3-fluoro-4-(methylsulfonyl)phenyl]thiophene;

3-(3-methylphenyl)-4-[3-fluoro-4-(methylsulfonyl)phenyl]thiophene;

3-(4-methylphenyl)-4-[3-fluoro-4-(methylsulfonyl)phenyl]thiophene;

3-(3-cyanophenyl)-4-[3-fluoro-4-(methylsulfonyl)phenyl]thiophene;

3-(4-cyanophenyl)-4-[3-fluoro-4-(methylsulfonyl)phenyl]thiophene;

3-(3-trifluoromethylphenyl)-4-[3-fluoro-4-(methylsulfonyl)phenyl]thiophene;

3-(4-trifluoromethylphenyl)-4-[3-fluoro-4-(methylsulfonyl)phenyl]thiophene;

3-(3-trifluoromethoxyphenyl)-4-[3-fluoro-4-(methylsulfonyl)phenyl]thiophene;

3-(4-trifluoromethoxyphenyl)-4-[3-fluoro-4-(methylsulfonyl)phenyl]thiophene;

3-(3,4-dichlorophenyl)-4-[3-fluoro-4-(methylsulfonyl)phenyl]thiophene;

3-(3,4-dibromophenyl)-4-[3-fluoro-4-(methylsulfonyl)phenyl]thiophene;

3-(3,4-difluorophenyl)-4-[3-fluoro-4-(methylsulfonyl)phenyl]thiophene;

3-(3,5-dichlorophenyl)-4-[3-fluoro-4-(methylsulfonyl)phenyl]thiophene;

3-(3,5-dibromophenyl)-4-[3-fluoro-4-(methylsulfonyl)phenyl]thiophene;

3-(3,5-difluorophenyl)-4-[3-fluoro-4-(methylsulfonyl)phenyl]thiophene;

3-(3,4-dimethylphenyl)-4-[3-fluoro-4-(methylsulfonyl)phenyl]thiophene;

3-(3,5-dimethylphenyl)-4-[3-fluoro-4-(methylsulfonyl)phenyl]thiophene;

3-(3-methyl-4-chlorophenyl)-4-[3-fluoro-4-(methylsulfonyl)phenyl]thiophene;

3-(4-methyl-3-chlorophenyl)-4-[3-fluoro-4-(methylsulfonyl)phenyl]thiophene;

3-(3-methyl-4-fluorophenyl)-4-[3-fluoro-4-(methylsulfonyl)phenyl]thiophene;

3-(4-methyl-3-fluorophenyl)-4-[3-fluoro-4-(methylsulfonyl)phenyl]thiophene;

3-(3-methyl-4-bromophenyl)-4-[3-fluoro-4-(methylsulfonyl)phenyl]thiophene;

3-(4-methyl-3-bromophenyl)-4-p-fluoro-4-(methylsulfonyl)phenyl]thiophene;

3-(3-methyl-4-trifluoromethylphenyl)-4-[3-fluoro-4-(methylsulfonyl)phenyl]-thiophene;

3-(4-methyl-3-trifluoromethylphenyl)-4-[3-fluoro-4-(methylsulfonyl)phenyl]-thiophene;

3-(3-methyl-4-trifluoromethoxyphenyl)-4-[3-fluoro-4-(methylsulfonyl)phenyl]-thiophene;

3-(4-methyl-3-trifluoromethoxyphenyl)-4-[3-fluoro-4-(methylsulfonyl)phenyl]-thiophene;

3-(3-cyano-4-methylphenyl)-4-[3-fluoro-4-(methylsulfonyl)phenyl]thiophene;

3-(4-cyano-3-methylphenyl)-4-[3-fluoro-4-(methylsulfonyl)phenyl]thiophene;

3-(3-chloro-4-methoxyphenyl)-4-[3-fluoro-4-(methylsulfonyl)phenyl]thiophene;

3-(4-chloro-3-methoxyphenyl)-4-[3-fluoro-4-(methylsulfonyl)phenyl]thiophene;

3-(2-methylpyridin-6-yl)-4-[3-fluoro-4-(methylsulfonyl)phenyl]thiophene;

3-(2-methylthiazol-4-yl)-4-[3-fluoro-4-(methylsulfonyl)phenyl]thiophene;

3-(4-methylthiazol-2-yl)-4-[3-fluoro-4-(methylsulfonyl)phenyl]thiophene;

3-(2-methylpyridin-3-yl)-4-[3-fluoro-4-(methylsulfonyl)phenyl]thiophene;

3-(2-methylpyridin-3-yl)-4-[3-fluoro-4-(methylsulfonyl)phenyl]thiophene;

3-(3-pyridinyl)-4-[3-fluoro-4-(methylsulfonyl)phenyl]thiophene;

3-(5-methylpyridin-3-yl)-4-[3-fluoro-4-(methylsulfonyl)phenyl]thiophene;

3-(2-methylpyridin-3-yl)-4-[3-fluoro-4-(methylsulfonyl)phenyl]thiophene;

3-cyclohexyl-4-[3-fluoro-4-(methylsulfonyl)phenyl]thiophene;

3-cyclopentyl-4-[3-fluoro-4-(methylsulfonyl)phenyl]thiophene;

2-fluoro-4-[4-phenyl-3-thiophenyl]benezenesulfonamide;

2-fluoro-4-[4-(3-chlorophenyl)-3-thiophenyl]benezenesulfonamide;

2-fluoro-4-[4-(4-chlorophenyl)-3-thiophenyl]benezenesulfonamide;

2-fluoro-4-[4-(3-bromophenyl)-3-thiophenyl]benezenesulfonamide;

2-fluoro-4-[4-(4-bromophenyl)-3-thiophenyl]benezenesulfonamide;

2-fluoro-4-[4-(3-fluorophenyl)-3-thiophenyl]benezenesulfonamide;

2-fluoro-4-[4-(4-fluorophenyl)-3-thiophenyl]benezenesulfonamide;

2-fluoro-4-[4-(3-methylphenyl)-3-thiophenyl]benezenesulfonamide;

2-fluoro-4-[4-(4-methylphenyl)-3-thiophenyl]benezenesulfonamide;

2-fluoro-4-[4-(3-cyanophenyl)-3-thiophenyl]benezenesulfonamide;

2-fluoro-4-[4-(4-cyanophenyl)-3-thiophenyl]benezenesulfonamide;

2-fluoro-4-[4-(3-trifluoromethylphenyl)-3-thiophenyl]benezenesulfonamide;

2-fluoro-4-[4-(4-trifluoromethylphenyl)-3-thiophenyl]benezenesulfonamide;

2-fluoro-4-[4-(3-trifluoromethoxyphenyl)-3-thiophenyl]benezenesulfonamide;

2-fluoro-4-[4-(4-trifluoromethoxyphenyl)-3-thiophenyl]benezenesulfonamide;

2-fluoro-4-[4-(3,4-dichlorophenyl)-3-thiophenyl]benezenesulfonamide;

2-fluoro-4-[4-(3,4-dibromophenyl)-3-thiophenyl]benezenesulfonamide

2-fluoro-4-[4-(3,4-difluorophenyl)-3-thiophenyl]benezenesulfonamide;

2-fluoro-4-[4-(3,5-dichlorophenyl)-3-thiophenyl]benezenesulfonamide;

2-fluoro-4-[4-(3,5-dibromophenyl)-3-thiophenyl]benezenesulfonamide;

2-fluoro-4-[4-(3,5-difluorophenyl)-3-thiophenyl]benezenesulfonamide;

2-fluoro-4-[4-(3,4-dimethylphenyl)-3-thiophenyl]benezenesulfonamide;

2-fluoro-4-[4-(3,5-dimethylphenyl)-3-thiophenyl]benezenesulfonamide;

2-fluoro-4-[4-(3-methyl-4-chlorophenyl)-3-thiophenyl]benezenesulfonamide;

2-fluoro-4-[4-(4-methyl-3-chlorophenyl)-3-thiophenyl]benezenesulfonamide;

2-fluoro-4-[4-(3-methyl-4-fluorophenyl)-3-thiophenyl]benezenesulfonamide;

2-fluoro-4-[4-(4-methyl-3-fluorophenyl)-3-thiophenyl]benezenesulfonamide;

2-fluoro-4-[4-(3-methyl-4-bromophenyl)-3-thiophenyl]benezenesulfonamide;

2-fluoro-4-[4-(4-methyl-3-bromophenyl)-3-thiophenyl]benezenesulfonamide;

2-fluoro-4-[4-(3-methyl-4-trifluoromethylphenyl)-3-thiophenyl]benezenesulfonamide;

2-fluoro-4-[4-(4-methyl-3-trifluoromethylphenyl)-3-thiophenyl]benezenesulfonamide;

2-fluoro-4-[4-(3-methyl-4-trifluoromethoxyphenyl)-3-thiophenyl]benezenesulfonamide;

2-fluoro-4-[4-(4-methyl-3-trifluoromethoxyphenyl)-3-thiophenyl]benezenesulfonamide;

2-fluoro-4-[4-(3-cyano-4-methylphenyl)-3-thiophenyl]benezenesulfonamide;

2-fluoro-4-[4-(4-cyano-3-methylphenyl)-3-thiophenyl]benezenesulfonamide;

2-fluoro-4-[4-(3-chloro-4-methoxyphenyl)-3-thiophenyl]benezenesulfonamide;

2-fluoro-4-[4-(4-chloro-3-methoxyphenyl)-3-thiophenyl]benezenesulfonamide;

2-fluoro-4-[4-(2-methylpyridin-6-yl)-3-thiophenyl]benezenesulfonamide;

2-fluoro-4-[4-(2-methylthiazol-4-yl)-3-thiophenyl]benezenesulfonamide;

2-fluoro-4-[4-(4-methylthiazol-2-yl)-3-thiophenyl]benezenesulfonamide;

2-fluoro-4-[4-(2-methylpyridin-3-yl)-3-thiophenyl]benezenesulfonamide;

2-fluoro-4-[4-(2-methylpyridin-3-yl)-3-thiophenyl]benezenesulfonamide;

2-fluoro-4-[4-(3-pyridinyl)-3-thiophenyl]benezenesulfonamide;

2-fluoro-4-[4-(5-methylpyridin-3-yl)-3-thiophenyl]benezenesulfonamide;

2-fluoro-4-[4-(2-methylpyridin-3-yl)-3-thiophenyl]benezenesulfonamide;

2-fluoro-4-[4-cyclohexyl-3-thiophenyl]benezenesulfonamide;

2-fluoro-4-[4-cyclopentyl-3-thiophenyl]benezenesulfonamide;

and the pharmaceutically-acceptable salts, tautomers and prodrugs thereof.

Isoxazoles

In still another embodiment, the present method of treating cyclooxygenase-2 mediated disorders comprises administering to the subject a therapeutically-effective amount of one or more compounds selected from a subclass of compounds of Formula I corresponding to Formula IV:

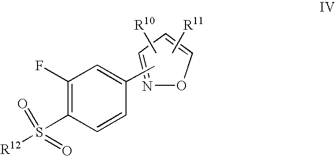

wherein substituents R¹⁰, R¹¹ and R¹² have the same definitions and sub-definitions as substituents R¹, R³ and R², respectively, set forth above for the compounds of Formula I, and the pharmaceutically-acceptable salts, tautomers and prodrugs thereof. Preferably, R¹⁰ is not 4-bromophenyl when R¹¹ is methyl.

Within this subclass of compounds, a preferred group of compounds consists of those compounds of Formula IVA and IVB:

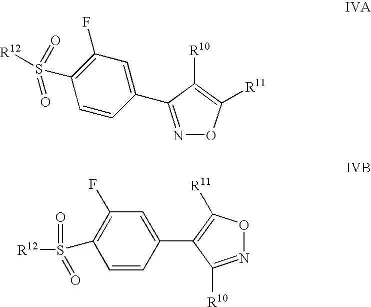

wherein R¹⁰, R¹¹ and R¹² are as defined above, and the pharmaceutically-acceptable salts, tautomers and prodrugs thereof.

Preferred species within this subclass include, but are not limited to:

3-phenyl-4-[3-fluoro-4-(methylsulfonyl)phenyl]-5-trifluoromethylisoxazole;

3-(3-chlorophenyl)-4-[3-fluoro-4-(methylsulfonyl)phenyl]-5-trifluoromethylisoxazole;

3-(4-chlorophenyl)-4-[3-fluoro-4-(methylsulfonyl)phenyl]-5-trifluoromethylisoxazole;

3-(3-bromophenyl)-4-[3-fluoro-4-(methylsulfonyl)phenyl]-5-trifluoromethylisoxazole;

3-(4-bromophenyl)-4-[3-fluoro-4-(methylsulfonyl)phenyl]-5-trifluoromethylisoxazole;

3-(3-fluorophenyl)-4-[3-fluoro-4-(methylsulfonyl)phenyl]-5-trifluoromethylisoxazole;

3-(4-fluorophenyl)-4-[3-fluoro-4-(methylsulfonyl)phenyl]-5-trifluoromethylisoxazole;

3-(3-methylphenyl)-4-[3-fluoro-4-(methylsulfonyl)phenyl]-5-trifluoromethylisoxazole;

3-(4-methylphenyl)-4-[3-fluoro-4-(methylsulfonyl)phenyl]-5-trifluoromethylisoxazole;

3-(3-cyanophenyl)-4-[3-fluoro-4-(methylsulfonyl)phenyl]-5-trifluoromethylisoxazole;

3-(4-cyanophenyl)-4-[3-fluoro-4-(methylsulfonyl)phenyl]-5-trifluoromethylisoxazole;

3-(3-trifluoromethylphenyl)-4-[3-fluoro-4-(methylsulfonyl)phenyl]-5-trifluoromethylisoxazole;

3-(4-trifluoromethylphenyl)-4-[3-fluoro-4-(methylsulfonyl)phenyl]-5-trifluoromethylisoxazole;

3-(3-trifluoromethoxyphenyl)-4-[3-fluoro-4-(methylsulfonyl)phenyl]-5-trifluoromethylisoxazole;

3-(4-trifluoromethoxyphenyl)-4-[3-fluoro-4-(methylsulfonyl)phenyl]-5-trifluoromethylisoxazole;

3-(3,4-dichlorophenyl)-4-[3-fluoro-4-(methylsulfonyl)phenyl]-5-trifluoromethylisoxazole;

3-(3,4-dibromophenyl)-4-[3-fluoro-4-(methylsulfonyl)phenyl]-5-trifluoromethylisoxazole;

3-(3,4-difluorophenyl)-4-[3-fluoro-4-(methylsulfonyl)phenyl]-5-trifluoromethylisoxazole;

3-(3,5-dichlorophenyl)-4-[3-fluoro-4-(methylsulfonyl)phenyl]-5-trifluoromethylisoxazole;

3-(3,5-dibromophenyl)-4-[3-fluoro-4-(methylsulfonyl)phenyl]-5-trifluoromethylisoxazole;

3-(3,5-difluorophenyl)-4-[3-fluoro-4-(methylsulfonyl)phenyl]-5-trifluoromethylisoxazole;

3-(3,4-dimethylphenyl)-4-[3-fluoro-4-(methylsulfonyl)phenyl)-5-trifluoromethylisoxazole;

3-(3,5-dimethylphenyl)-4-[3-fluoro-4-(methylsulfonyl)phenyl]-5-trifluoromethylisoxazole;

3-(3-methyl-4-chlorophenyl)-4-[3-fluoro-4-(methylsulfonyl)phenyl]-5-trifluoromethylisoxazole;

3-(4-methyl-3-chlorophenyl)-4-[3-fluoro-4-(methylsulfonyl)phenyl]-5-trifluoromethylisoxazole;

3-(3-methyl-4-fluorophenyl)-4-[3-fluoro-4-(methylsulfonyl)phenyl]-5-trifluoromethylisoxazole;

3-(4-methyl-3-fluorophenyl)-4-[3-fluoro-4-(methylsulfonyl)phenyl]-5-trifluoromethylisoxazole;

3-(3-methyl-4-bromophenyl)-4-[3-fluoro-4-(methylsulfonyl)phenyl]-5-trifluoromethylisoxazole;

3-(4-methyl-3-bromophenyl)-4-[3-fluoro-4-(methylsulfonyl)phenyl]-5-trifluoromethylisoxazole;

3-(3-methyl-4-trifluoromethylphenyl)-4-[3-fluoro-4-(methylsulfonyl)phenyl]-5-trifluoromethylisoxazole;

3-(4-methyl-3-trifluoromethylphenyl)-4-[3-fluoro-4-(methylsulfonyl)phenyl]-5-trifluoromethylisoxazole;

3-(3-methyl-4-trifluoromethoxyphenyl)-4-[3-fluoro-4-(methylsulfonyl)phenyl]-5-trifluoromethylisoxazole;

3-(4-methyl-3-trifluoromethoxyphenyl)-4-[3-fluoro-4-(methylsulfonyl)phenyl]-5-trifluoromethylisoxazole;

3-(3-cyano-4-methylphenyl)-4-[3-fluoro-4-(methylsulfonyl)phenyl]-5-trifluoromethylisoxazole;

3-(4-cyano-3-methylphenyl)-4-[3-fluoro-4-(methylsulfonyl)phenyl]-5-trifluoromethylisoxazole;

3-(3-chloro-4-methoxyphenyl)-4-[3-fluoro-4-(methylsulfonyl)phenyl]-5-trifluoromethylisoxazole;

3-(4-chloro-3-methoxyphenyl)-4-[3-fluoro-4-(methylsulfonyl)phenyl]-5-trifluoromethylisoxazole;

3-(2-methylpyridin-6-yl)-4-[3-fluoro-4-(methylsulfonyl)phenyl]-5-trifluoromethylisoxazole;

3-(2-methylthiazol-4-yl)-4-[3-fluoro-4-(methylsulfonyl)phenyl]-5-trifluoromethylisoxazole;

3-(4-methylthiazol-2-yl)-4-[3-fluoro-4-(methylsulfonyl)phenyl]-5-trifluoromethylisoxazole;

3-(2-methylpyridin-3-yl)-4-[3-fluoro-4-(methylsulfonyl)phenyl]-5-trifluoromethylisoxazole;

3-(2-methylpyridin-3-yl)-4-[3-fluoro-4-(methylsulfonyl)phenyl]-5-trifluoromethylisoxazole;

3-(3-pyridinyl)-4-[3-fluoro-4-(methylsulfonyl)phenyl]-5-trifluoromethylisoxazole;

3-(5-methylpyridin-3-yl)-4-[3-fluoro-4-(methylsulfonyl)phenyl]-5-trifluoromethylisoxazole;

3-(2-methylpyridin-3-yl)-4-[3-fluoro-4-(methylsulfonyl)phenyl]-5-trifluoromethylisoxazole;

3-cyclohexyl-4-[3-fluoro-4-(methylsulfonyl)phenyl]-5-trifluoromethylisoxazole;

3-cyclopentyl-4-(3-fluoro-4-(methylsulfonyl)phenyl]-5-trifluoromethylisoxazole;

2-fluoro-4-[3-phenyl-5-fluoromethylisoxazol-4-yl]benezenesulfonamide;

2-fluoro-4-[3-(3-chlorophenyl)-5-fluoromethylisoxazol-4-yl]benezenesulfonamide;

2-fluoro-4-[3-(4-chlorophenyl)-5-fluoromethylisoxazol-4-yl]benezenesulfonamide;

2-fluoro-4-[3-(3-bromophenyl)-5-fluoromethylisoxazol-4-yl]benezenesulfonamide;

2-fluoro-4-[3-(4-bromophenyl)-5-fluoromethylisoxazol-4-yl]benezenesulfonamide;

2-fluoro-4-[3-(3-fluorophenyl)-5-fluoromethylisoxazol-4-yl]benezenesulfonamide;

2-fluoro-4-[3-(4-fluorophenyl)-5-fluoromethylisoxazol-4-yl]benezenesulfonamide;

2-fluoro-4-[3-(3-methylphenyl)-5-fluoromethylisoxazol-4-yl]benezenesulfonamide;

2-fluoro-4-[3-(4-methylphenyl)-5-fluoromethylisoxazol-4-yl]benezenesulfonamide;

2-fluoro-4-[3-(3-cyanophenyl)-5-fluoromethylisoxazol-4-yl]benezenesulfonamide;

2-fluoro-4-[3-(4-cyanophenyl)-5-fluoromethylisoxazol-4-yl]benezenesulfonamide;

2-fluoro-4-[3-(3-trifluoromethylphenyl)-5-fluoromethyl-isoxazol-4-yl]benezenesulfonamide;

2-fluoro-4-[3-(4-trifluoromethylphenyl)-5-fluoromethyl-isoxazol-4-yl]benezenesulfonamide;

2-fluoro-4-[3-(3-trifluoromethoxyphenyl)-5-fluoromethylisoxazol-4-yl]benezene-sulfonamide;

2-fluoro-4-[3-(4-trifluoromethoxyphenyl)-5-fluoromethylisoxazol-4-yl]benezene-sulfonamide;

2-fluoro-4-[3-(3,4-dichlorophenyl)-5-fluoromethyl-soxazol-4-yl]benezenesulfonamide;

2-fluoro-4-[3-(3,4-dibromophenyl)-5-fluoromethyl-isoxazol-4-yl]benezenesulfonamide

2-fluoro-4-[3-(3,4-difluorophenyl)-5-fluoromethyl-isoxazol-4-yl]benezenesulfonamide;

2-fluoro-4-[3-(3,5-dichlorophenyl)-5-fluoromethyl-isoxazol-4-yl]benezenesulfonamide;

2-fluoro-4-[3-(3,5-dibromophenyl)-5-fluoromethyl-isoxazol-4-yl]benezenesulfonamide;

2-fluoro-4-[3-(3,5-difluorophenyl)-5-fluoromethyl-isoxazol-4-yl]benezenesulfonamide;

2-fluoro-4-[3-(3,4-dimethylphenyl)-5-fluoromethyl-isoxazol-4-yl]benezenesulfonamide;

2-fluoro-4-[3-(3,5-dimethylphenyl)-5-fluoromethyl-isoxazol-4-yl]benezenesulfonamide;

2-fluoro-4-[3-(3-methyl-4-chlorophenyl)-5-fluoromethyl-isoxazol-4-yl]benezene-sulfonamide;

2-fluoro-4-[3-(4-methyl-3-chlorophenyl)-5-fluoromethyl-isoxazol-4-yl]benezene-sulfonamide;

2-fluoro-4-[3-(3-methyl-4-fluorophenyl)-5-fluoromethyl-isoxazol-4-yl]benezenesulfonamide;

2-fluoro-4-[3-(4-methyl-3-fluorophenyl)-5-fluoromethyl-isoxazol-4-yl]benezenesulfonamide;

2-fluoro-4-[3-(3-methyl-4-bromophenyl)-5-fluoromethyl-isoxazol-4-yl]benezene-sulfonamide;

2-fluoro-4-[3-(4-methyl-3-bromophenyl)-5-fluoromethyl-isoxazol-4-yl]benezene-sulfonamide;

2-fluoro-4-[3-(3-methyl-4-trifluoromethylphenyl)-5-fluoromethylisoxazol-4-yl]benezenesulfonamide;

2-fluoro-4-[3-(4-methyl-3-trifluoromethylphenyl)-5-fluoromethylisoxazol-4-yl]benezenesulfonamide;

2-fluoro-4-[3-(3-methyl-4-trifluoromethoxyphenyl)-5-fluoromethylisoxazol-4-yl]benezenesulfonamide;

2-fluoro-4-[3-(4-methyl-3-trifluoromethoxyphenyl)-5-fluoromethylisoxazol-4-yl]benezenesulfonamide;

2-fluoro-4-[3-(3-cyano-4-methylphenyl)-5-fluoromethylisoxazol-4-yl]benezenesulfonamide;

52-fluoro-4-[3-(4-cyano-3-methylphenyl)-5-fluoromethyl-isoxazol-4-yl]benezenesulfonamide;

2-fluoro-4-[3-(3-chloro-4-methoxyphenyl)-5-fluoromethylisoxazol-4-yl]benezene-sulfonamide;

2-fluoro-4-[3-(4-chloro-3-methoxyphenyl)-5-fluoromethylisoxazol-4-yl]benezene-sulfonamide;

2-fluoro-4-[3-(2-methylpyridin-6-yl)-5-fluoromethyl-isoxazol-4-yl]benezenesulfonamide;

2-fluoro-4-[3-(2-methylthiazol-4-yl)-5-fluoromethyl-isoxazol-4-yl]benezenesulfonamide;

2-fluoro-4-[3-(4-methylthiazol-2-yl)-5-fluoromethyl-isoxazol-4-yl]benezenesulfonamide;

2-fluoro-4-[3-(2-methylpyridin-3-yl)-5-fluoromethyl-isoxazol-4-yl]benezenesulfonamide;

2-fluoro-4-[3-(2-methylpyridin-3-yl)-5-fluoromethyl-isoxazol-4-yl]benezenesulfonamide;

2-fluoro-4-[3-(3-pyridinyl)-5-fluoromethylisoxazol-4-yl]benezenesulfonamide;

2-fluoro-4-[3-(5-methylpyridin-3-yl)-5-fluoromethyl-isoxazol-4-yl]benezenesulfonamide;

2-fluoro-4-[3-(2-methylpyridin-3-yl)-5-fluoromethyl-isoxazol-4-yl]benezenesulfonamide;

2-fluoro-4-[3-cyclohexyl-5-fluoromethylisoxazol-4-yl]benezenesulfonamide;

2-fluoro-4-[3-cyclopentyl-5-fluoromethylisoxazol-4-yl]benezenesulfonamide;

2-fluoro-4-[3-phenyl-5-difluoromethylisoxazol-4-yl]benezenesulfonamide;

2-fluoro-4-[3-(3-chlorophenyl)-5-difluoromethyl-isoxazol-4-yl]benezenesulfonamide;

2-fluoro-4-[3-(4-chlorophenyl)-5-difluoromethyl-isoxazol-4-yl]benezenesulfonamide;

2-fluoro-4-[3-(3-bromophenyl)-5-difluoromethylisoxazol-4-yl]benezenesulfonamide;

2-fluoro-4-[3-(4-bromophenyl)-5-difluoromethylisoxazol-4-yl]benezenesulfonamide;

2-fluoro-4-[3-(3-fluorophenyl)-5-difluoromethyl-isoxazol-4-yl]benezenesulfonamide;

2-fluoro-4-[3-(4-fluorophenyl)-5-difluoromethyl-isoxazol-4-yl]benezenesulfonamide;

2-fluoro-4-[3-(3-methylphenyl)-5-difluoromethyl-isoxazol-4-yl]benezenesulfonamide;

2-fluoro-4-[3-(4-methylphenyl)-5-difluoromethyl-isoxazol-4-yl]benezenesulfonamide;

2-fluoro-4-[3-(3-cyanophenyl)-5-difluoromethylisoxazol-4-yl]benezenesulfonamide;

2-fluoro-4-[3-(4-cyanophenyl)-5-difluoromethylisoxazol-4-yl]benezenesulfonamide;

2-fluoro-4-[3-(3-trifluoromethylphenyl)-5-difluoro-methylisoxazol-4-yl]benezene-sulfonamide;

2-fluoro-4-[3-(4-trifluoromethylphenyl)-5-difluoro-methylisoxazol-4-yl]benezene-sulfonamide;

2-fluoro-4-[3-(3-trifluoromethoxyphenyl)-5-difluoro-methylisoxazol-4-yl]benezenesulfonamide;

2-fluoro-4-[3-(4-trifluoromethoxyphenyl)-5-difluoro-methylisoxazol-4-yl]benezenesulfonamide;

2-fluoro-4-[3-(3,4-dichlorophenyl)-5-difluoro-methylisoxazol-4-yl]benezenesulfonamide;

2-fluoro-4-[3-(3,4-dibromophenyl)-5-difluoro-methylisoxazol-4-yl]benezenesulfonamide;

2-fluoro-4-[3-(3,4-difluorophenyl)-5-difluoro-methylisoxazol-4-yl]benezenesulfonamide;

2-fluoro-4-[3-(3,5-dichlorophenyl)-5-difluoro-methylisoxazol-4-yl]benezenesulfonamide;

2-fluoro-4-[3-(3,5-dibromophenyl)-5-difluoro-methylisoxazol-4-yl]benezenesulfonamide;

2-fluoro-4-[3-(3,5-difluorophenyl)-5-difluoro-methylisoxazol-4-yl]benezenesulfonamide;

2-fluoro-4-[3-(3,4-dimethylphenyl)-5-difluoro-methylisoxazol-4-yl]benezenesulfonamide;

2-fluoro-4-[3-(3,5-dimethylphenyl)-5-difluoro-methylisoxazol-4-yl]benezenesulfonamide;

2-fluoro-4-[3-(3-methyl-4-chlorophenyl)-5-difluoro-methylisoxazol-4-yl]benezenesulfonamide;

2-fluoro-4-[3-(4-methyl-3-chlorophenyl)-5-difluoro-methylisoxazol-4-yl]benezenesulfonamide;

2-fluoro-4-[3-(3-methyl-4-fluorophenyl)-5-difluoro-methylisoxazol-4-yl]benezenesulfonamide;

2-fluoro-4-[3-(4-methyl-3-fluorophenyl)-5-difluoro-methylisoxazol-4-yl]benezenesulfonamide;

2-fluoro-4-[3-(3-methyl-4-bromophenyl)-5-difluoro-methylisoxazol-4-yl]benezenesulfonamide;

2-fluoro-4-[3-(4-methyl-3-bromophenyl)-5-difluoro-methylisoxazol-4-yl]benezenesulfonamide;

2-fluoro-4-[3-(3-methyl-4-trifluoromethylphenyl)-5-difluoromethylisoxazol-4-yl]benezenesulfonamide;

2-fluoro-4-[3-(4-methyl-3-trifluoromethylphenyl)-5-difluoromethylisoxazol-4-yl]benezenesulfonamide;

2-fluoro-4-[3-(3-methyl-4-trifluoromethoxyphenyl)-5-difluoromethylisoxazol-4-yl]benezenesulfonamide;

2-fluoro-4-[3-(4-methyl-3-trifluoromethoxyphenyl)-5-difluoromethylisoxazol-4-yl]benezenesulfonamide;

2-fluoro-4-[3-(3-cyano-4-methylphenyl)-5-difluoro-methylisoxazol-4-yl]benezenesulfonamide;

2-fluoro-4-[3-(4-cyano-3-methylphenyl)-5-difluoro-methylisoxazol-4-yl]benezenesulfonamide;

2-fluoro-4-[3-(3-chloro-4-methoxyphenyl)-5-difluoro-methylisoxazol-4-yl]benezenesulfonamide;

2-fluoro-4-[3-(4-chloro-3-methoxyphenyl)-5-difluoro-methylisoxazol-4-yl]benezenesulfonamide;

2-fluoro-4-[3-(2-methylpyridin-6-yl)-5-difluoro-methylisoxazol-4-yl]benezenesulfonamide;

2-fluoro-4-[3-(2-methylthiazol-4-yl)-5-difluoro-methylisoxazol-4-yl]benezenesulfonamide;

2-fluoro-4-[3-(4-methylthiazol-2-yl)-5-difluoro-methylisoxazol-4-yl]benezenesulfonamide;

2-fluoro-4-[3-(2-methylpyridin-3-yl)-5-difluoro-methylisoxazol-4-yl]benezenesulfonamide;

2-fluoro-4-[3-(2-methylpyridin-3-yl)-5-difluoro-methylisoxazol-4-yl]benezenesulfonamide;

2-fluoro-4-[3-(3-pyridinyl)-5-difluoromethylisoxazol-4-yl]benezenesulfonamide;

2-fluoro-4-[3-(5-methylpyridin-3-yl)-5-difluoro-methylisoxazol-4-yl]benezenesulfonamide;

2-fluoro-4-[3-(2-methylpyridin-3-yl)-5-difluoro-methylisoxazol-4-yl]benezenesulfonamide;

2-fluoro-4-[3-cyclohexyl-5-difluoromethylisoxazol-4-yl]benezenesulfonamide;

2-fluoro-4-[3-cyclopentyl-5-difluoromethylisoxazol-4-yl]benezenesulfonamide;

3-phenyl-4-[3-fluoro-4-(methylsulfonyl)phenyl]-5-hydroxymethylisoxazole;

3-(3-chlorophenyl)-4-[3-fluoro-4-(methylsulfonyl)phenyl]-5-hydroxymethylisoxazole;

3-(4-chlorophenyl)-4-[3-fluoro-4-(methylsulfonyl)phenyl]-5-hydroxymethylisoxazole;

3-(3-bromophenyl)-4-[3-fluoro-4-(methylsulfonyl)phenyl]-5-hydroxymethylisoxazole;

3-(4-bromophenyl)-4-[3-fluoro-4-(methylsulfonyl)phenyl]-5-hydroxymethylisoxazole;

3-(3-fluorophenyl)-4-[3-fluoro-4-(methylsulfonyl)phenyl]-5-hydroxymethylisoxazole;

3-(4-fluorophenyl)-4-[3-fluoro-4-(methylsulfonyl)phenyl]-5-hydroxymethylisoxazole;

3-(3-methylphenyl)-4-[3-fluoro-4-(methylsulfonyl)phenyl]-5-hydroxymethylisoxazole;

3-(4-methylphenyl)-4-[3-fluoro-4-(methylsulfonyl)phenyl]-5-hydroxymethylisoxazole;

3-(3-cyanophenyl)-4-[3-fluoro-4-(methylsulfonyl)phenyl]-5-hydroxymethylisoxazole;

3-(4-cyanophenyl)-4-[3-fluoro-4-(methylsulfonyl)phenyl]-5-hydroxymethylisoxazole;

3-(3-trifluoromethylphenyl)-4-[3-fluoro-4-(methylsulfonyl)phenyl]-5-hydroxymethylisoxazole;

3-(4-trifluoromethylphenyl)-4-[3-fluoro-4-(methylsulfonyl)phenyl]-5-hydroxymethylisoxazole;

3-(3-trifluoromethoxyphenyl)-4-[3-fluoro-4-(methylsulfonyl)phenyl]-5-hydroxymethylisoxazole;

3-(4-trifluoromethoxyphenyl)-4-[3-fluoro-4-(methylsulfonyl)phenyl]-5-hydroxymethylisoxazole;

3-(3,4-dichlorophenyl)-4-[3-fluoro-4-(methylsulfonyl)phenyl]-5-hydroxymethylisoxazole;

3-(3,4-dibromophenyl)-4-[3-fluoro-4-(methylsulfonyl)phenyl]-5-hydroxymethylisoxazole;

3-(3,4-difluorophenyl)-4-[3-fluoro-4-(methylsulfonyl)phenyl]-5-hydroxymethylisoxazole;

3-(3,5-dichlorophenyl)-4-[3-fluoro-4-(methylsulfonyl)phenyl]-5-hydroxymethylisoxazole;

3-(3,5-dibromophenyl)-4-[3-fluoro-4-(methylsulfonyl)phenyl]-5-hydroxymethylisoxazole;

3-(3,5-difluorophenyl)-4-[3-fluoro-4-(methylsulfonyl)phenyl]-5-hydroxymethylisoxazole;

3-(3,4-dimethylphenyl)-4-[3-fluoro-4-(methylsulfonyl)phenyl]-5-hydroxymethylisoxazole;

3-(3,5-dimethylphenyl)-4-[3-fluoro-4-(methylsulfonyl)phenyl]-5-hydroxymethylisoxazole;

3-(3-methyl-4-chlorophenyl)-4-[3-fluoro-4-(methylsulfonyl)phenyl]-5-hydroxymethylisoxazole;

3-(4-methyl-3-chlorophenyl)-4-[3-fluoro-4-(methylsulfonyl)phenyl]-5-hydroxymethylisoxazole;

3-(3-methyl-4-fluorophenyl)-4-[3-fluoro-4-(methylsulfonyl)phenyl]-5-hydroxymethylisoxazole;

3-(4-methyl-3-fluorophenyl)-4-[3-fluoro-4-(methylsulfonyl)phenyl]-5-hydroxymethylisoxazole;

3-(3-methyl-4-bromophenyl)-4-[3-fluoro-4-(methylsulfonyl)phenyl]-5-hydroxymethylisoxazole;

3-(4-methyl-3-bromophenyl)-4-[3-fluoro-4-(methylsulfonyl)phenyl]-5-hydroxymethylisoxazole;

3-(3-methyl-4-trifluoromethylphenyl)-4-[3-fluoro-4-(methylsulfonyl)phenyl]-5-hydroxymethylisoxazole;

3-(4-methyl-3-trifluoromethylphenyl)-4-[3-fluoro-4-(methylsulfonyl)phenyl]-5-hydroxymethylisoxazole;

3-(3-methyl-4-trifluoromethoxyphenyl)-4-[3-fluoro-4-(methylsulfonyl)phenyl]-5-hydroxymethylisoxazole;

3-(4-methyl-3-trifluoromethoxyphenyl)-4-[3-fluoro-4-(methylsulfonyl)phenyl]-5-hydroxymethylisoxazole;

3-(3-cyano-4-methylphenyl)-4-[3-fluoro-4-(methylsulfonyl)phenyl]-5-hydroxymethylisoxazole;

3-(4-cyano-3-methylphenyl)-4-[3-fluoro-4-(methylsulfonyl)phenyl]-5-hydroxymethylisoxazole;

3-(3-chloro-4-methoxyphenyl)-4-[3-fluoro-4-(methylsulfonyl)phenyl]-5-hydroxymethylisoxazole;

3-(4-chloro-3-methoxyphenyl)-4-[3-fluoro-4-(methylsulfonyl)phenyl]-5-hydroxymethylisoxazole;

3-(2-methylpyridin-6-yl)-4-[3-fluoro-4-(methylsulfonyl)phenyl]-5-hydroxymethylisoxazole;

3-(2-methylthiazol-4-yl)-4-[3-fluoro-4-(methylsulfonyl)phenyl]-5-hydroxymethylisoxazole;

3-(4-methylthiazol-2-yl)-4-[3-fluoro-4-(methylsulfonyl)phenyl]-5-hydroxymethylisoxazole;

3-(2-methylpyridin-3-yl)-4-[3-fluoro-4-(methylsulfonyl)phenyl]-5-hydroxymethylisoxazole;

3-(2-methylpyridin-3-yl)-4-[3-fluoro-4-(methylsulfonyl)phenyl]-5-hydroxymethylisoxazole;

3-(3-pyridinyl)-4-[3-fluoro-4-(methylsulfonyl)phenyl]-5-hydroxymethylisoxazole;

3-(5-methylpyridin-3-yl)-4-[3-fluoro-4-(methylsulfonyl)phenyl]-5-hydroxymethylisoxazole;

3-(2-methylpyridin-3-yl)-4-[3-fluoro-4-(methylsulfonyl)phenyl]-5-hydroxymethylisoxazole;

3-cyclohexyl-4-[3-fluoro-4-(methylsulfonyl)phenyl]-5-hydroxymethylisoxazole;

3-cyclopentyl-4-[3-fluoro-4-(methylsulfonyl)phenyl]-5-hydroxymethylisoxazole;

3-phenyl-4-[3-fluoro-4-(methylsulfonyl)phenyl]-5-fluoromethylisoxazole;

3-(3-chlorophenyl)-4-[3-fluoro-4-(methylsulfonyl)phenyl]-5-fluoromethylisoxazole;

3-(4-chlorophenyl)-4-[3-fluoro-4-(methylsulfonyl)phenyl]-5-fluoromethylisoxazole;

3-(3-bromophenyl)-4-[3-fluoro-4-(methylsulfonyl)phenyl]-5-fluoromethylisoxazole;

3-(4-bromophenyl)-4-[3-fluoro-4-(methylsulfonyl)phenyl]-5-fluoromethylisoxazole;

3-(3-fluorophenyl)-4-[3-fluoro-4-(methylsulfonyl)phenyl]-5-fluoromethylisoxazole;

3-(4-fluorophenyl)-4-[3-fluoro-4-(methylsulfonyl)phenyl]-5-fluoromethylisoxazole;

3-(3-methylphenyl)-4-[3-fluoro-4-(methylsulfonyl)phenyl]-5-fluoromethylisoxazole;

3-(4-methylphenyl)-4-[3-fluoro-4-(methylsulfonyl)phenyl]-5-fluoromethylisoxazole;

3-(3-cyanophenyl)-4-[3-fluoro-4-(methylsulfonyl)phenyl]-5-fluoromethylisoxazole;

3-(4-cyanophenyl)-4-[3-fluoro-4-(methylsulfonyl)phenyl]-5-fluoromethylisoxazole;

3-(3-trifluoromethylphenyl)-4-[3-fluoro-4-(methylsulfonyl)phenyl]-5-fluoromethylisoxazole;

3-(4-trifluoromethylphenyl)-4-[3-fluoro-4-(methylsulfonyl)phenyl]-5-fluoromethylisoxazole;

3-(3-trifluoromethoxyphenyl)-4-[3-fluoro-4-(methylsulfonyl)phenyl]-5-fluoromethylisoxazole;

3-(4-trifluoromethoxyphenyl)-4-[3-fluoro-4-(methylsulfonyl)phenyl]-5-fluoromethylisoxazole;

3-(3,4-dichlorophenyl)-4-[3-fluoro-4-(methylsulfonyl)phenyl]-5-fluoromethylisoxazole;

3-(3,4-dibromophenyl)-4-[3-fluoro-4-(methylsulfonyl)phenyl]-5-fluoromethylisoxazole;

3-(3,4-difluorophenyl)-4-[3-fluoro-4-(methylsulfonyl)phenyl]-5-fluoromethylisoxazole;

3-(3,5-dichlorophenyl)-4-[3-fluoro-4-(methylsulfonyl)phenyl]-5-fluoromethylisoxazole;

3-(3,5-dibromophenyl)-4-[3-fluoro-4-(methylsulfonyl)phenyl]-5-fluoromethylisoxazole;

3-(3,5-difluorophenyl)-4-[3-fluoro-4-(methylsulfonyl)phenyl]-5-fluoromethylisoxazole;

3-(3,4-dimethylphenyl)-4-[3-fluoro-4-(methylsulfonyl)phenyl]-5-fluoromethylisoxazole;

3-(3,5-dimethylphenyl)-4-[3-fluoro-4-(methylsulfonyl)phenyl]-5-fluoromethylisoxazole;

3-(3-methyl-4-chlorophenyl)-4-[3-fluoro-4-(methylsulfonyl)phenyl]-5-fluoromethylisoxazole;

3-(4-methyl-3-chlorophenyl)-4-[3-fluoro-4-(methylsulfonyl)phenyl]-5-fluoromethylisoxazole;

3-(3-methyl-4-fluorophenyl)-4-[3-fluoro-4-(methylsulfonyl)phenyl]-5-fluoromethylisoxazole;

3-(4-methyl-3-fluorophenyl)-4-[3-fluoro-4-(methylsulfonyl)phenyl]-5-fluoromethylisoxazole;

3-(3-methyl-4-bromophenyl)-4-[3-fluoro-4-(methylsulfonyl)phenyl]-5-fluoromethylisoxazole;

3-(4-methyl-3-bromophenyl)-4-[3-fluoro-4-(methylsulfonyl)phenyl]-5-fluoromethylisoxazole;

3-(3-methyl-4-trifluoromethylphenyl)-4-[3-fluoro-4-(methylsulfonyl)phenyl]-5-fluoromethylisoxazole;

3-(4-methyl-3-trifluoromethylphenyl)-4-[3-fluoro-4-(methylsulfonyl)phenyl]-5-fluoromethylisoxazole;

3-(3-methyl-4-trifluoromethoxyphenyl)-4-[3-fluoro-4-(methylsulfonyl)phenyl]-5-fluoromethylisoxazole;

3-(4-methyl-3-trifluoromethoxyphenyl)-4-[3-fluoro-4-(methylsulfonyl)phenyl]-5-fluoromethylisoxazole;

3-(3-cyano-4-methylphenyl)-4-[3-fluoro-4-(methylsulfonyl)phenyl]-5-fluoromethylisoxazole;

3-(4-cyano-3-methylphenyl)-4-[3-fluoro-4-(methylsulfonyl)phenyl]-5-fluoromethylisoxazole;

3-(3-chloro-4-methoxyphenyl)-4-[3-fluoro-4-(methylsulfonyl)phenyl]-5-fluoromethylisoxazole;

3-(4-chloro-3-methoxyphenyl)-4-[3-fluoro-4-(methylsulfonyl)phenyl]-5-fluoromethylisoxazole;

3-(2-methylpyridin-6-yl)-4-[3-fluoro-4-(methylsulfonyl)phenyl]-5-fluoromethylisoxazole;

3-(2-methylthiazol-4-yl)-4-[3-fluoro-4-(methylsulfonyl)phenyl]-5-fluoromethylisoxazole;

3-(4-methylthiazol-2-yl)-4-[3-fluoro-4-(methylsulfonyl)phenyl]-5-fluoromethylisoxazole;

3-(2-methylpyridin-3-yl)-4-[3-fluoro-4-(methylsulfonyl)phenyl]-5-fluoromethylisoxazole;

3-(2-methylpyridin-3-yl)-4-[3-fluoro-4-(methylsulfonyl)phenyl]-5-fluoromethylisoxazole;

3-(3-pyridinyl)-4-[3-fluoro-4-(methylsulfonyl)phenyl]-5-fluoromethylisoxazole;

3-(5-methylpyridin-3-yl)-4-[3-fluoro-4-(methylsulfonyl)phenyl]-5-fluoromethylisoxazole;

3-(2-methylpyridin-3-yl)-4-[3-fluoro-4-(methylsulfonyl)phenyl]-5-fluoromethylisoxazole;

3-cyclohexyl-4-[3-fluoro-4-(methylsulfonyl)phenyl]-5-fluoromethylisoxazole;

3-cyclopentyl-4-[3-fluoro-4-(methylsulfonyl)phenyl]-5-fluoromethylisoxazole;

3-phenyl-4-[3-fluoro-4-(methylsulfonyl)phenyl]-5-difluoromethylisoxazole;

3-(3-chlorophenyl)-4-[3-fluoro-4-(methylsulfonyl)phenyl]-5-difluoromethylisoxazole;

3-(4-chlorophenyl)-4-[3-fluoro-4-(methylsulfonyl)phenyl]-5-difluoromethylisoxazole;

3-(3-bromophenyl)-4-[3-fluoro-4-(methylsulfonyl)phenyl]-5-difluoromethylisoxazole;

3-(4-bromophenyl)-4-[3-fluoro-4-(methylsulfonyl)phenyl]-5-difluoromethylisoxazole;

3-(3-fluorophenyl)-4-[3-fluoro-4-(methylsulfonyl)phenyl]-5-difluoromethylisoxazole;

3-(4-fluorophenyl)-4-[3-fluoro-4-(methylsulfonyl)phenyl]-5-difluoromethylisoxazole;

3-(3-methylphenyl)-4-[3-fluoro-4-(methylsulfonyl)phenyl]-5-difluoromethylisoxazole;

3-(4-methylphenyl)-4-[3-fluoro-4-(methylsulfonyl)phenyl]-5-difluoromethylisoxazole;

3-(3-cyanophenyl)-4-[3-fluoro-4-(methylsulfonyl)phenyl]-5-difluoromethylisoxazole;

3-(4-cyanophenyl)-4-[3-fluoro-4-(methylsulfonyl)phenyl]-5-difluoromethylisoxazole;

3-(3-trifluoromethylphenyl)-4-[3-fluoro-4-(methylsulfonyl)phenyl]-5-difluoromethylisoxazole;

3-(4-trifluoromethylphenyl)-4-[3-fluoro-4-(methylsulfonyl)phenyl]-5-difluoromethylisoxazole;

3-(3-trifluoromethoxyphenyl)-4-[3-fluoro-4-(methylsulfonyl)phenyl]-5-difluoromethylisoxazole;

3-(4-trifluoromethoxyphenyl)-4-[3-fluoro-4-(methylsulfonyl)phenyl]-5-difluoromethylisoxazole;

3-(3,4-dichlorophenyl)-4-[3-fluoro-4-(methylsulfonyl)phenyl]-5-difluoromethylisoxazole;

3-(3,4-dibromophenyl)-4-[3-fluoro-4-(methylsulfonyl)phenyl]-5-difluoromethylisoxazole;

3-(3,4-difluorophenyl)-4-[3-fluoro-4-(methylsulfonyl)phenyl]-5-difluoromethylisoxazole;

3-(3,5-dichlorophenyl)-4-[3-fluoro-4-(methylsulfonyl)phenyl]-5-difluoromethylisoxazole;

3-(3,5-dibromophenyl)-4-[3-fluoro-4-(methylsulfonyl)phenyl]-5-difluoromethylisoxazole;

3-(3,5-difluorophenyl)-4-[3-fluoro-4-(methylsulfonyl)phenyl]-5-difluoromethylisoxazole;

3-(3,4-dimethylphenyl)-4-[3-fluoro-4-(methylsulfonyl)phenyl]-5-difluoromethylisoxazole;

3-(3,5-dimethylphenyl)-4-[3-fluoro-4-(methylsulfonyl)phenyl]-5-difluoromethylisoxazole;

3-(3-methyl-4-chlorophenyl)-4-[3-fluoro-4-(methylsulfonyl)phenyl]-5-difluoromethylisoxazole;

3-(4-methyl-3-chlorophenyl)-4-[3-fluoro-4-(methylsulfonyl)phenyl])-5 difluoromethylisoxazole;

3-(3-methyl-4-fluorophenyl)-4-[3-fluoro-4-(methylsulfonyl)phenyl]-5-difluoromethylisoxazole;

3-(4-methyl-3-fluorophenyl)-4-[3-fluoro-4-(methylsulfonyl)phenyl]-5-difluoromethylisoxazole;

3-(3-methyl-4-bromophenyl)-4-[3-fluoro-4-(methylsulfonyl)phenyl]-5-difluoromethylisoxazole;

3-(4-methyl-3-bromophenyl)-4-[3-fluoro-4-(methylsulfonyl)phenyl]-5-difluoromethylisoxazole;

3-(3-methyl-4-trifluoromethylphenyl)-4-[3-fluoro-4-(methylsulfonyl)phenyl]-5-difluoromethylisoxazole;

3-(4-methyl-3-trifluoromethylphenyl)-4-[3-fluoro-4-(methylsulfonyl)phenyl]-5-difluoromethylisoxazole;

3-(3-methyl-4-trifluoromethoxyphenyl)-4-[3-fluoro-4-(methylsulfonyl)phenyl]-5-difluoromethylisoxazole;

3-(4-methyl-3-trifluoromethoxyphenyl)-4-[3-fluoro-4-(methylsulfonyl)phenyl]-5-difluoromethylisoxazole;

3-(3-cyano-4-methylphenyl)-4-[3-fluoro-4-(methylsulfonyl)phenyl]-5-difluoromethylisoxazole;

3-(4-cyano-3-methylphenyl)-4-[3-fluoro-4-(methylsulfonyl)phenyl]-5-difluoromethylisoxazole;

3-(3-chloro-4-methoxyphenyl)-4-[3-fluoro-4-(methylsulfonyl)phenyl]-5-difluoromethylisoxazole;

3-(4-chloro-3-methoxyphenyl)-4-[3-fluoro-4-(methylsulfonyl)phenyl]-5-difluoromethylisoxazole;

3-(2-methylpyridin-6-yl)-4-[3-fluoro-4-(methylsulfonyl)phenyl]-5-difluoromethylisoxazole;

3-(2-methylthiazol-4-yl)-4-[3-fluoro-4-(methylsulfonyl)phenyl]-5-difluoromethylisoxazole;

3-(4-methylthiazol-2-yl)-4-[3-fluoro-4-(methylsulfonyl)phenyl]-5-difluoromethylisoxazole;

3-(2-methylpyridin-3-yl)-4-[3-fluoro-4-(methylsulfonyl)phenyl]-5-difluoromethylisoxazole;

3-(2-methylpyridin-3-yl)-4-[3-fluoro-4-(methylsulfonyl)phenyl]-5-difluoromethylisoxazole;

3-(3-pyridinyl)-4-[3-fluoro-4-(methylsulfonyl)phenyl]-5-difluoromethylisoxazole;

3-(5-methylpyridin-3-yl)-4-[3-fluoro-4-(methylsulfonyl)phenyl]-5-difluoromethylisoxazole;

3-(2-methylpyridin-3-yl)-4-[3-fluoro-4-(methylsulfonyl)phenyl]-5-difluoromethylisoxazole;

3-cyclohexyl-4-[3-fluoro-4-(methylsulfonyl)phenyl]-5-difluoromethylisoxazole;

3-cyclopentyl-4-[3-fluoro-4-(methylsulfonyl)phenyl]-5-difluoromethylisoxazole;

3-phenyl-4-[3-fluoro-4-(methylsulfonyl)phenyl]-5-methylisoxazole;

3-(3-chlorophenyl)-4-[3-fluoro-4-(methylsulfonyl)phenyl]-5-methylisoxazole;

3-(4-chlorophenyl)-4-[3-fluoro-4-(methylsulfonyl)phenyl]-5-methylisoxazole;

3-(3-bromophenyl)-4-[3-fluoro-4-(methylsulfonyl)phenyl]-5-methylisoxazole;

3-(4-bromophenyl)-4-[3-fluoro-4-(methylsulfonyl)phenyl]-5-methylisoxazole;

3-(3-fluorophenyl)-4-[3-fluoro-4-(methylsulfonyl)phenyl]-5-methylisoxazole;

3-(4-fluorophenyl)-4-[3-fluoro-4-(methylsulfonyl)phenyl]-5-methylisoxazole;

3-(3-methylphenyl)-4-[3-fluoro-4-(methylsulfonyl)phenyl]-5-methylisoxazole;

3-(4-methylphenyl)-4-[3-fluoro-4-(methylsulfonyl)phenyl]-5-methylisoxazole;

3-(3-cyanophenyl)-4-[3-fluoro-4-(methylsulfonyl)phenyl]-5-methylisoxazole;

3-(4-cyanophenyl)-4-[3-fluoro-4-(methylsulfonyl)phenyl]-5-methylisoxazole;

3-(3-trifluoromethylphenyl)-4-[3-fluoro-4-(methylsulfonyl)phenyl]-5-methylisoxazole;

3-(4-trifluoromethylphenyl)-4-[3-fluoro-4-(methylsulfonyl)phenyl]-5-methylisoxazole;

3-(3-trifluoromethoxyphenyl)-4-[3-fluoro-4-(methylsulfonyl)phenyl]-5-methylisoxazole;

3-(4-trifluoromethoxyphenyl)-4-[3-fluoro-4-(methylsulfonyl)phenyl]-5-methylisoxazole;

3-(3,4-dichlorophenyl)-4-[3-fluoro-4-(methylsulfonyl)phenyl]-5-methylisoxazole;

3-(3,4-dibromophenyl)-4-[3-fluoro-4-(methylsulfonyl)phenyl]-5-methylisoxazole;

3-(3,4-difluorophenyl)-4-[3-fluoro-4-(methylsuffonyl)phenyl]-5-methylisoxazole;

3-(3,5-dichlorophenyl)-4-[3-fluoro-4-(methylsulfonyl)phenyl]-5-methylisoxazole;

3-(3,5-dibromophenyl)-4-[3-fluoro-4-(methylsulfonyl)phenyl]-5-methylisoxazole;

3-(3,5-difluorophenyl)-4-[3-fluoro-4-(methylsulfonyl)phenyl]-5-methylisoxazole;

3-(3,4-dimethylphenyl)-4-[3-fluoro-4-(methylsulfonyl)phenyl]-5-methylisoxazole;

3-(3,5-dimethylphenyl)-4-[3-fluoro-4-(methylsulfonyl)phenyl]-5-methylisoxazole;

3-(3-methyl-4-chlorophenyl)-4-[3-fluoro-4-(methylsulfonyl)phenyl]-5-methylisoxazole;

3-(4-methyl-3-chlorophenyl)-4-[3-fluoro-4-(methylsulfonyl)phenyl]-5-methylisoxazole;

3-(3-methyl-4-fluorophenyl)-4-[3-fluoro-4-(methylsulfonyl)phenyl]-5-methylisoxazole;

3-(4-methyl-3-fluorophenyl)-4-[3-fluoro-4-(methylsulfonyl)phenyl]-5-methylisoxazole;

3-(3-methyl-4-bromophenyl)-4-[3-fluoro-4-(methylsulfonyl)phenyl]-5-methylisoxazole;

3-(4-methyl-3-bromophenyl)-4-[3-fluoro-4-(methylsulfonyl)phenyl]-5-methylisoxazole;

3-(3-methyl-4-trifluoromethylphenyl)-4-[3-fluoro-4-(methylsulfonyl)phenyl]-5-methylisoxazole;

3-(4-methyl-3-trifluoromethylphenyl)-4-[3-fluoro-4-(methylsulfonyl)phenyl]-5-methylisoxazole;

3-(3-methyl-4-trifluoromethoxyphenyl)-4-[3-fluoro-4-(methylsulfonyl)phenyl]-5-methylisoxazole;

3-(4-methyl-3-trifluoromethoxyphenyl)-4-[3-fluoro-4-(methylsulfonyl)phenyl]-5-methylisoxazole;

3-(3-cyano-4-methylphenyl)-4-[3-fluoro-4-(methylsulfonyl)phenyl]-5-methylisoxazole;

3-(4-cyano-3-methylphenyl)-4-[3-fluoro-4-(methylsulfonyl)phenyl]-5-methylisoxazole;

3-(3-chloro-4-methoxyphenyl)-4-[3-fluoro-4-(methylsulfonyl)phenyl]-5-methylisoxazole;

3-(4-chloro-3-methoxyphenyl)-4-[3-fluoro-4-(methylsulfonyl)phenyl]-5-methylisoxazole;

3-(2-methylpyridin-6-yl)-4-[3-fluoro-4-methylsulfonyl)phenyl]-5-methylisoxazole;

3-(2-methylthiazol-4-yl)-4-[3-fluoro-4-(methylsulfonyl)phenyl]-5-methylisoxazole;

3-(4-methylthiazol-2-yl)-4-[3-fluoro-4-(methylsulfonyl)phenyl]-5-methylisoxazole;

3-(2-methylpyridin-3-yl)-4-[3-fluoro-4-(methylsulfonyl)phenyl]-5-methylisoxazole;

3-(2-methylpyridin-3-yl)-4-[3-fluoro-4-(methylsulfonyl)phenyl]-5-methylisoxazole;

3-(3-pyridinyl)-4-[3-fluoro-4-(methylsulfonyl)phenyl]-5-methylisoxazole;

3-(5-methylpyridin-3-yl)-4-[3-fluoro-4-(methylsulfonyl)phenyl]-5-methylisoxazole;

3-(2-methylpyridin-3-yl)-4-[3-fluoro-4-(methylsulfonyl)phenyl]-5-methylisoxazole;

3-cyclohexyl-4-[3-fluoro-4-(methylsulfonyl)phenyl]-5-methylisoxazole;

3-cyclopentyl-4-[3-fluoro-4-(methylsulfonyl)phenyl]-5-methylisoxazole;

2-fluoro-4-[3-phenyl-5-hydroxymethylisoxazol-4-yl]benezenesulfonamide;

2-fluoro-4-[3-(3-chlorophenyl)-5-hydroxymethylisoxazol-4-yl]benezenesulfonamide;

2-fluoro-4-[3-(4-chlorophenyl)-5-hydroxymethylisoxazol-4-yl]benezenesulfonamide;

2-fluoro-4-[3-(3-bromophenyl)-5-hydroxymethylisoxazol-4-yl]benezenesulfonamide;

2-fluoro-4-[3-(4-bromophenyl)-5-hydroxymethylisoxazol-4-yl]benezenesulfonamide;

2-fluoro-4-[3-(3-fluorophenyl)-5-hydroxymethylisoxazol-4-yl]benezenesulfonamide;

2-fluoro-4-[3-(4-fluorophenyl)-5-hydroxymethylisoxazol-4-yl]benezenesulfonamide;

2-fluoro-4-[3-(3-methylphenyl)-5-hydroxymethylisoxazol-4-yl]benezenesulfonamide;

2-fluoro-4-[3-(4-methylphenyl)-5-hydroxymethylisoxazol-4-yl]benezenesulfonamide;

2-fluoro-4-[3-(3-cyanophenyl)-5-hydroxymethylisoxazol-4-yl]benezenesulfonamide;

2-fluoro-4-[3-(4-cyanophenyl)-5-hydroxymethylisoxazol-4-yl]benezenesulfonamide;

2-fluoro-4-[3-(3-trifluoromethylphenyl)-5-hydroxymethylisoxazol-4-yl]benezenesulfonamide;

2-fluoro-4-[3-(4-trifluoromethylphenyl)-5-hydroxymethylisoxazol-4-yl]benezenesulfonamide;

2-fluoro-4-[3-(3-trifluoromethoxyphenyl)-5-hydroxymethylisoxazol-4-yl]benezenesulfonamide;

2-fluoro-4-[3-(4-trifluoromethoxyphenyl)-5-hydroxymethylisoxazol-4-yl]benezenesulfonamide;

2-fluoro-4-[3-(3,4-dichlorophenyl)-5-hydroxymethylisoxazol-4-yl]benezenesulfonamide;

2-fluoro-4-[3-(3,4-dibromophenyl)-5-hydroxymethyl-isoxazol-4-yl]benezenesulfonamide

2-fluoro-4-[3-(3,4-difluorophenyl)-5-hydroxymethyl-isoxazol-4-yl]benezenesulfonamide;

2-fluoro-4-[3-(3,5-dichlorophenyl)-5-hydroxymethyl-isoxazol-4-yl]benezenesulfonamide;

2-fluoro-4-[3-(3,5-dibromophenyl)-5-hydroxymethyl-isoxazol-4-yl]benezenesulfonamide;

2-fluoro-4-[3-(3,5-difluorophenyl)-5-hydroxymethyl-isoxazol-4-yl]benezenesulfonamide;

2-fluoro-4-[3-(3,4-dimethylphenyl)-5-hydroxymethyl-isoxazol-4-yl]benezenesulfonamide; %

2-fluoro-4-[3-(3,5-dimethylphenyl)-5-hydroxymethyl-isoxazol-4-yl]benezenesulfonamide;

2-fluoro-4-[3-(3-methyl-4-chlorophenyl)-5-hydroxy-methylisoxazol-4-yl]benezenesulfonamide;

2-fluoro-4-[3-(4-methyl-3-chlorophenyl)-5-hydroxy-methylisoxazol-4-yl]benezenesulfonamide;

2-fluoro-4-[3-(3-methyl-4-fluorophenyl)-5-hydroxy-methylisoxazol-4-yl]benezenesulfonamide;

2-fluoro-4-[3-(4-methyl-3-fluorophenyl)-5-hydroxy-methylisoxazol-4-yl]benezenesulfonamide;

2-fluoro-4-[3-(3-methyl-4-bromophenyl)-5-hydroxy-methylisoxazol-4-yl]benezenesulfonamide;

2-fluoro-4-[3-(4-methyl-3-bromophenyl)-5-hydroxy-methylisoxazol-4-yl]benezenesulfonamide;

2-fluoro-4-[3-(3-methyl-4-trifluoromethylphenyl)-5-hydroxymethylisoxazol-4-yl]benezenesulfonamide;

2-fluoro-4-[3-(4-methyl-3-trifluoromethylphenyl)-5-hydroxymethylisoxazol-4-yl]benezenesulfonamide;

2-fluoro-4-[3-(3-methyl-4-trifluoromethoxyphenyl)-5-hydroxymethylisoxazol-4-yl]benezenesulfonamide;

2-fluoro-4-[3-(4-methyl-3-trifluoromethoxyphenyl)-5-hydroxymethylisoxazol-4-yl]benezenesulfonamide;

2-fluoro-4-[3-(3-cyano-4-methylphenyl)-5-hydroxy-methylisoxazol-4-yl]benezenesulfonamide;

2-fluoro-4-[3-(4-cyano-3-methylphenyl)-5-hydroxy-methylisoxazol-4-yl]benezenesulfonamide;

2-fluoro-4-[3-(3-chloro-4-methoxyphenyl)-5-hydroxy-methylisoxazol-4-yl]benezenesulfonamide;

2-fluoro-4-[3-(4-chloro-3-methoxyphenyl)-5-hydroxy-methylisoxazol-4-yl]benezenesulfonamide;

2-fluoro-4-[3-(2-methylpyridin-6-yl)-5-hydroxymethyl-isoxazol-4-yl]benezenesulfonamide;

2-fluoro-4-[3-(2-methylthiazol-4-yl)-5-hydroxymethylisoxazol-4-yl]benezenesulfonamide;

2-fluoro-4-[3-(4-methylthiazol-2-yl)-5-hydroxy-methylisoxazol-4-yl]benezenesulfonamide;

2-fluoro-4-[3-(2-methylpyridin-3-yl)-5-hydroxymethy-lisoxazol-4-yl]benezenesulfonamide;

2-fluoro-4-[3-(2-methylpyridin-3-yl)-5-hydroxymethyl-isoxazol-4-yl]benezenesulfonamide;

2-fluoro-4-[3-(3-pyridinyl)-5-hydroxymethylisoxazol-4-yl]benezenesulfonamide;

2-fluoro-4-[3-(5-methylpyridin-3-yl)-5-hydroxymethyl-isoxazol-4-yl]benezenesulfonamide;

2-fluoro-4-[3-(2-methylpyridin-3-yl)-5-hydroxymethyl-isoxazol-4-yl]benezenesulfonamide;

2-fluoro-4-[3-cyclohexyl-5-hydroxymethylisoxazol-4-yl]benezenesulfonamide;

2-fluoro-4-[3-cyclopentyl-5-hydroxymethylisoxazol-4-yl]benezenesulfonamide;

2-fluoro-4-[3-phenyl-5-methylisoxazol-4-yl]benezenesulfonamide;

2-fluoro-4-[3-(3-chlorophenyl)-5-methylisoxazol-4-yl]benezenesulfonamide;

2-fluoro-4-[3-(4-chlorophenyl)-5-methylisoxazol-4-yl]benezenesulfonamide;

2-fluoro-4-[3-(3-bromophenyl)-5-methylisoxazol-4-yl]benezenesulfonamide;

2-fluoro-4-[3-(4-bromophenyl)-5-methylisoxazol-4-yl]benezenesulfonamide;

2-fluoro-4-[3-(3-fluorophenyl)-5-methylisoxazol-4-yl]benezenesulfonamide;

2-fluoro-4-[3-(4-fluorophenyl)-5-methylisoxazol-4-yl]benezenesulfonamide;

2-fluoro-4-[3-(3-methylphenyl)-5-methylisoxazol-4-yl]benezenesulfonamide;

2-fluoro-4-[3-(4-methylphenyl)-5-methylisoxazol-4-yl]benezenesulfonamide;

2-fluoro-4-[3-(3-cyanophenyl)-5-methylisoxazol-4-yl]benezenesulfonamide;

2-fluoro-4-[3-(4-cyanophenyl)-5-methylisoxazol-4-yl]benezenesulfonamide;

2-fluoro-4-[3-(3-trifluoromethylphenyl)-5-methylisoxazol-4-yl]benezenesulfonamide;

2-fluoro-4-[3-(4-trifluoromethylphenyl)-5-methylisoxazol-4-yl]benezenesulfonamide;

2-fluoro-4-[3-(3-trifluoromethoxyphenyl)-5-methylisoxazol-4-yl]benezenesulfonamide;

2-fluoro-4-[3-(4-trifluoromethoxyphenyl)-5-methylisoxazol-4-yl]benezenesulfonamide;

2-fluoro-4-[3-(3,4-dichlorophenyl)-5-methylisoxazol-4-yl]benezenesulfonamide;

2-fluoro-4-[3-(3,4-dibromophenyl)-5-methylisoxazol-4-yl]benezenesulfonamide

2-fluoro-4-[3-(3,4-difluorophenyl)-5-methylisoxazol-4-yl]benezenesulfonamide;

2-fluoro-4-[3-(3,5-dichlorophenyl)-5-methylisoxazol-4-yl]benezenesulfonamide;

2-fluoro-4-[3-(3,5-dibromophenyl)-5-methylisoxazol-4-yl]benezenesulfonamide;

2-fluoro-4-[3-(3,5-difluorophenyl)-5-methylisoxazol-4-yl]benezenesulfonamide;

2-fluoro-4-[3-(3,4-dimethylphenyl)-5-methylisoxazol-4-yl]benezenesulfonamide;

2-fluoro-4-[3-(3,5-dimethylphenyl)-5-methylisoxazol-4-yl]benezenesulfonamide;

2-fluoro-4-[3-(3-methyl-4-chlorophenyl)-5-methyl-isoxazol-4-yl]benezenesulfonamide;

2-fluoro-4-[3-(4-methyl-3-chlorophenyl)-5-methyl-isoxazol-4-yl]benezenesulfonamide;

2-fluoro-4-[3-(3-methyl-4-fluorophenyl)-5-methyl-isoxazol-4-yl]benezenesulfonamide;

2-fluoro-4-[3-(4-methyl-3-fluorophenyl)-5-methylisoxazol-4-yl]benezenesulfonamide;

2-fluoro-4-[3-(3-methyl-4-bromophenyl)-5-methyl-isoxazol-4-yl]benezenesulfonamide;

2-fluoro-4-[3-(4-methyl-3-bromophenyl)-5-methyl-isoxazol-4-yl]benezenesulfonamide;

2-fluoro-4-[3-(3-methyl-4-trifluoromethylphenyl)-5-methylisoxazol-4-yl]benezenesulfonamide;

2-fluoro-4-[3-(4-methyl-3-trifluoromethylphenyl)-5-methylisoxazol-4-yl]benezenesulfonamide;

2-fluoro-4-[3-(3-methyl-4-trifluoromethoxyphenyl)-5-methylisoxazol-4-yl]benezenesulfonamide;

2-fluoro-4-[3-(4-methyl-3-trifluoromethoxyphenyl)-5-methylisoxazol-4-yl]benezenesulfonamide;

2-fluoro-4-[3-(3-cyano-4-methylphenyl)-5-methylisoxazol 4-yl]benezenesulfonamide;

2-fluoro-4-[3-(4-cyano-3-methylphenyl)-5-methylisoxazol-4-yl]benezenesulfonamide;

2-fluoro-4-[3-(3-chloro-4-methoxyphenyl)-5-methyl-isoxazol-4-yl]benezenesulfonamide;

2-fluoro-4-[3-(4-chloro-3-methoxyphenyl)-5-methyl-isoxazol-4-yl]benezenesulfonamide;

2-fluoro-4-[3-(2-methylpyridin-6-yl)-5-methylisoxazol-4-yl]benezenesulfonamide;

2-fluoro-4-[3-(2-methylthiazol-4-yl)-5-methylisoxazol-4-yl]benezenesulfonamide;

2-fluoro-4-[3-(4-methylthiazol-2-yl)-5-methylisoxazol-4-yl]benezenesulfonamide;

2-fluoro-4-[3-(2-methylpyridin-3-yl)-5-methylisoxazol-4-yl]benezenesulfonamide;

2-fluoro-4-[3-(2-methylpyridin-3-yl)-5-methylisoxazol-4-yl]benezenesulfonamide;

2-fluoro-4-[3-(3-pyridinyl)-5-methylisoxazol-4-yl]benezenesulfonamide;

2-fluoro-4-[3-(5-methylpyridin-3-yl)-5-methylisoxazol-4-yl]benezenesulfonamide;

2-fluoro-4-[3-(2-methylpyridin-3-yl)-5-methylisoxazol-4-yl]benezenesulfonamide;

2-fluoro-4-[3-cyclohexyl-5-methylisoxazol-4-yl]benezenesulfonamide;

2-fluoro-4-[3-cyclopentyl-5-methylisoxazol-4-yl]benezenesulfonamide;

2-fluoro-4-[3-phenyl-5-trifluoromethylisoxazol-4-yl]benezenesulfonamide;

2-fluoro-4-[3-(3-chlorophenyl)-5 trifluoromethyl-isoxazol-4-yl]benezenesulfonamide;

2-fluoro-4-[3-(4-chlorophenyl)-5-trifluoromethyl-isoxazol-4-yl]benezenesulfonamide;

2-fluoro-4-[3-(3-bromophenyl)-5-trifluoromethyl-isoxazol-4-yl]benezenesulfonamide;

2-fluoro-4-[3-(4-bromophenyl)-5-trifluoromethyl-isoxazol-4-yl]benezenesulfonamide;

2-fluoro-4-[3-(3-fluorophenyl)-5-trifluoromethyl-isoxazol-4-yl]benezenesulfonamide;

2-fluoro-4-[3-(4-fluorophenyl)-5-trifluoromethyl-isoxazol-4-yl]benezenesulfonamide;

2-fluoro-4-[3-(3-methylphenyl)-5-trifluoromethyl-isoxazol-4-yl]benezenesulfonamide;

2-fluoro-4-[3-(4-methylphenyl)-5-trifluoromethyl-isoxazol-4-yl]benezenesulfonamide;

2-fluoro-4-[3-(3-cyanophenyl)-5-trifluoromethyl-isoxazol-4-yl]benezenesulfonamide;

2-fluoro-4-[3-(4-cyanophenyl)-5-trifluoromethyl-isoxazol-4-yl]benezenesulfonamide;

2-fluoro-4-[3-(3-trifluoromethylphenyl)-5-trifluoro-methylisoxazol-4-yl]benezenesulfonamide;

2-fluoro-4-[3-(4-trifluoromethylphenyl)-5-trifluoro-methylisoxazol-4-yl]benezenesulfonamide;

2-fluoro-4-[3-(3-trifluoromethoxyphenyl)-5-trifluoro-methylisoxazol-4-yl]benezenesulfonamide;

2-fluoro-4-[3-(4-trifluoromethoxyphenyl)-5-trifluoro-methylisoxazol-4-yl]benezenesulfonamide;

2-fluoro-4-[3-(3,4-dichlorophenyl)-5-trifluoro-methylisoxazol-4-yl]benezenesulfonamide;

2-fluoro-4-[3-(3,4-dibromophenyl)-5-trifluoro-methylisoxazol-4-yl]benezenesulfonamide

2-fluoro-4-[3-(3,4-difluorophenyl)-5-trifluoro-methylisoxazol-4-yl]benezenesulfonamide;

2-fluoro-4-[3-(3,5-dichlorophenyl)-5-trifluoromethyl-isoxazol-4 yl]benezenesulfonamide;

2-fluoro-4-[3-(3,5-dibromophenyl)-5-trifluoromethyl-isoxazol-4-yl]benezenesulfonamide;

2-fluoro-4-[3-(3,5-difluorophenyl)-5-trifluoromethyl-isoxazol-4-yl]benezenesulfonamide;

2-fluoro-4-[3-(3,4-dimethylphenyl)-5-trifluoromethyl-isoxazol-4-yl]benezenesulfonamide;

2-fluoro-4-[3-(3,5-dimethylphenyl)-5-trifluoromethyl-isoxazol-4-yl]benezenesulfonamide;

2-fluoro-4-[3-(3-methyl-4-chlorophenyl)-5-trifluoro-methylisoxazol-4-yl]benezenesulfonamide;

2-fluoro-4-[3-(4-methyl-3-chlorophenyl)-5-trifluoro-methylisoxazol-4-yl]benezenesulfonamide;

2-fluoro-4-[3-(3-methyl-4-fluorophenyl)-5-trifluoro-methylisoxazol-4-yl]benezenesulfonamide;

2-fluoro-4-[3-(4-methyl-3-fluorophenyl)-5-trifluoro-methylisoxazol-4-yl]benezenesulfonamide;

2-fluoro-4-[3-(3-methyl-4-bromophenyl)-5-trifluoro-methylisoxazol-4-yl]benezenesulfonamide;

2-fluoro-4-[3-(4-methyl-3-bromophenyl)-5-trifluoro-methylisoxazol-4-yl]benezenesulfonamide;

2-fluoro-4-[3-(3-methyl-4-trifluoromethylphenyl)-5-trifluoromethylisoxazol-4-yl]benezenesulfonamide;

2-fluoro-4-[3-(4-methyl-3-trifluoromethylphenyl)-5-trifluoromethylisoxazol-4-yl]benezenesulfonamide;

2-fluoro-4-[3-(3-methyl-4-trifluoromethoxyphenyl)-5-trifluoromethylisoxazol-4-yl]benezenesulfonamide;

2-fluoro-4-[3-(4-methyl-3-trifluoromethoxyphenyl)-5-trifluoromethylisoxazol-4-yl]benezenesulfonamide;

2-fluoro-4-[3-(3-cyano-4-methylphenyl)-5-trifluoromethylisoxazol-4-yl]benezenesulfonamide;

2-fluoro-4-[3-(4-cyano-3-methylphenyl)-5-trifluoro-methylisoxazol-4-yl]benezenesulfonamide;

2-fluoro-4-[3-(3-chloro-4-methoxyphenyl)-5-trifluoro-methylisoxazol-4-yl]benezenesulfonamide;

2-fluoro-4-[3-(4-chloro-3-methoxyphenyl)-5-trifluoro-methylisoxazol-4-yl]benezenesulfonamide;

2-fluoro-4-[3-(2-methylpyridin-6-yl)-5-trifluoromethyl-isoxazol-4-yl]benezenesulfonamide;

2-fluoro-4-[3-(2-methylthiazol-4-yl)-5-trifluoromethyl-isoxazol-4-yl]benezenesulfonamide;

2-fluoro-4-[3-(4-methylthiazol-2-yl)-5-trifluoromethyl-isoxazol-4-yl]benezenesulfonamide;

2-fluoro-4-[3-(2-methylpyridin-3-yl)-5-trifluoromethyl-isoxazol-4-yl]benezenesulfonamide;

2-fluoro-4-[3-(2-methylpyridin-3-yl)-5-trifluoromethyl-isoxazol-4-yl]benezenesulfonamide;

2-fluoro-4-[3-(3-pyridinyl)-5-trifluoromethylisoxazol-4-yl]benezenesulfonamide;

2-fluoro-4-[3-(5-methylpyridin-3-yl)-5-trifluoromethyl-isoxazol-4-yl]benezenesulfonamide;

2-fluoro-4-[3-(2-methylpyridin-3-yl)-5-trifluoromethyl-isoxazol-4-yl]benezenesulfonamide;

2-fluoro-4-[3-cyclohexyl-5-trifluoromethylisoxazol-4-yl]benezenesulfonamide;

2-fluoro-4-[3-cyclopentyl-5-trifluoromethylisoxazol-4-yl]benezenesulfonamide;

2-fluoro-4-[3-phenyl-5-methyl-4-isoxazolyl]-benzenesulfonamide;

and the pharmaceutically-acceptable salts, tautomers and prodrugs thereof.

In one embodiment, the species is:

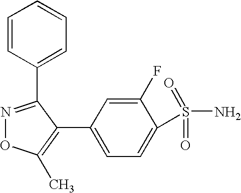

and the pharmaceutically-acceptable salts, tautomers and prodrugs thereof. In another embodiment, the fluoro radical is at the meta position relative to the sulfonyl group.

In another embodiment, the species is:

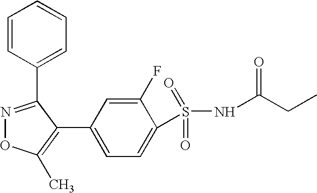

and the pharmaceutically-acceptable salts, tautomers and prodrugs thereof. In another embodiment, the fluoro radical is at the meta position relative to the sulfonyl group.

Furanones

In still another embodiment, the present method of treating cyclooxygenase-2 mediated disorders comprises administering to the subject a therapeutically-effective amount of one or more compounds selected from a subclass of compounds of Formula I corresponding to Formula V:

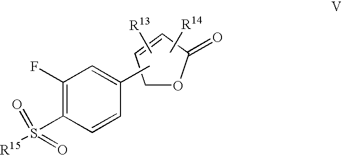

wherein substituents R¹³, R¹⁴ and R¹⁵ have the same definitions and sub-definitions as substituents R¹, R³ and R², respectively, set forth above for the compounds of Formula I, and the pharmaceutically-acceptable salts, tautomers and prodrugs thereof. Preferably, R¹³ is not 4-bromophenyl when R¹⁴ is hydrogen.

Within this subclass of compounds, a preferred group of compounds consists of those compounds of Formula VA and VB:

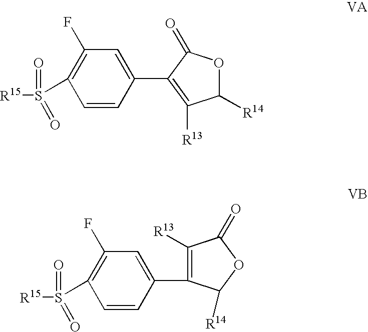

wherein R¹³, R¹⁴ and R¹⁵ are as defined above, and the pharmaceutically-acceptable salts, tautomers and prodrugs thereof.

Preferred species within this subclass include, but are not limited to:

2-fluoro-4-[4-5-oxo-2,5-dihydro-3-furanyl]benzenesulfonamide;

2-fluoro-4-[4-(3-chlorophenyl)-5-oxo-2,5-dihydro-3-furanyl]benzenesulfonamide;

2-fluoro-4-[4-(4-chlorophenyl)-5-oxo-2,5-dihydro-3-furanyl]benzenesulfonamide;

2-fluoro-4-[4-(3-bromophenyl)-5-oxo-2,5-dihydro-3-furanyl]benzenesulfonamide;

2-fluoro-4-[4-(4-bromophenyl)-5-oxo-2,5-dihydro-3-furanyl]benzenesulfonamide;

2-fluoro-4-[4-(3-fluorophenyl)-5-oxo-2,5-dihydro-3-furanyl]benzenesulfonamide;

2-fluoro-4-[4-(4-fluorophenyl)-5-oxo-2,5-dihydro-3-furanyl]benzenesulfonamide;

2-fluoro-4-[4-(3-methylphenyl)-5-oxo-2,5-dihydro-3-furanyl]benzenesulfonamide;

2-fluoro-4-[4-(4-methylphenyl)-5-oxo-2,5-dihydro-3-furanyl]benzenesulfonamide;

2-fluoro-4-[4-(3-cyanophenyl)-5-oxo-2,5-dihydro-3-furanyl]benzenesulfonamide;

2-fluoro-4-[4-(4-cyanophenyl)-5-oxo-2,5-dihydro-3-furanyl]benzenesulfonamide;

2-fluoro-4-[4-(3-trifluoromethylphenyl)-5-oxo-2,5-dihydro-3-furanyl]benzenesulfonamide;

2-fluoro-4-[4-(4-trifluoromethylphenyl)-5-oxo-2,5-dihydro-3-furanyl]benzenesulfonamide;

2-fluoro-4-[4-(3-trifluoromethoxyphenyl)-5-oxo-2,5-dihydro-3-furanyl]benzenesulfonamide;

2-fluoro-4-[4-(4-trifluoromethoxyphenyl)-5-oxo-2,5-dihydro-3-furanyl]benzenesulfonamide;

2-fluoro-4-[4-(3,4-dichlorophenyl)-5-oxo-2,5-dihydro-3-furanyl]benzenesulfonamide;

2-fluoro-4-[4-(3,4-dibromophenyl)-5-oxo-2,5-dihydro-3-furanyl]benzenesulfonamide

2-fluoro-4-[4-(3,4-difluorophenyl)-5-oxo-2,5-dihydro-3-furanyl]benzenesulfonamide;

2-fluoro-4-[4-(3,5-dichlorophenyl)-5-oxo-2,5-dihydro-3-furanyl]benzenesulfonamide;

2-fluoro-4-[4-(3,5-dibromophenyl)-5-oxo-2,5-dihydro-3-furanyl]benzenesulfonamide;

2-fluoro-4-[4-(3,5-difluorophenyl)-5-oxo-2,5-dihydro-3-furanyl]benzenesulfonamide;

2-fluoro-4-[4-(3,4-dimethylphenyl)-5-oxo-2,5-dihydro-3-furanyl]benzenesulfonamide;

2-fluoro-4-[4-(3,5-dimethylphenyl)-5-oxo-2,5-dihydro-3-furanyl]benzenesulfonamide;

2-fluoro-4-[4-(3-methyl-4-chlorophenyl)-5-oxo-2,5-dihydro-3-furanyl]benzenesulfonamide;

2-fluoro-4-[4-(4-methyl-3-chlorophenyl)-5-oxo-2,5-dihydro-3-furanyl]benzenesulfonamide;

2-fluoro-4-[4-(3-methyl-4-fluorophenyl)-5-oxo-2,5-dihydro-3-furanyl]benzenesulfonamide;

2-fluoro-4-[4-(4-methyl-3-fluorophenyl)-5-oxo-2,5-dihydro-3-furanyl]benzenesulfonamide;

2-fluoro-4-[4-(3-methyl-4-bromophenyl)-5-oxo-2,5-dihydro-3-furanyl]benzenesulfonamide;

2-fluoro-4-[4-(4-methyl-3-bromophenyl)-5-oxo-2,5-dihydro-3-furanyl]benzenesulfonamide;

2-fluoro-4-[4-(3-methyl-4-trifluoromethylphenyl)-5-oxo-2,5-dihydro-3-furanyl]benzenesulfonamide;

2-fluoro-4-[4-(4-methyl-3-trifluoromethylphenyl)-5-oxo-2,5-dihydro-3-furanyl]benzenesulfonamide;

2-fluoro-4-[4-(3-methyl-4-trifluoromethoxyphenyl)-5-oxo-2,5-dihydro-3-furanyl]benzenesulfonamide;

2-fluoro-4-[4-(4-methyl-3-trifluoromethoxyphenyl)-5-oxo-2,5-dihydro-3-furanyl]benzenesulfonamide;

2-fluoro-4-[4-(3-cyano-4-methylphenyl)-5-oxo-2,5-dihydro-3-furanyl]benzenesulfonamide;

2-fluoro-4-[4-(4-cyano-3-methylphenyl)-5-oxo-2,5-dihydro-3-furanyl]benzenesulfonamide;

2-fluoro-4-[4-(3-chloro-4-methoxyphenyl)-5-oxo-2,5-dihydro-3-furanyl]benzenesulfonamide;

2-fluoro-4-[4-(4-chloro-3-methoxyphenyl)-5-oxo-2,5-dihydro-3-furanyl]benzenesulfonamide;

2-fluoro-4-[4-(2-methylpyridin-6-yl)-5-oxo-2,5-dihydro-3-furanyl]benzenesulfonamide;

2-fluoro-4-[4-(2-methylthiazol-4-yl)-5-oxo-2,5-dihydro-3-furanyl]benzenesulfonamide;

2-fluoro-4-[4-(4-methylthiazol-2-yl)-5-oxo-2,5-dihydro-3-furanyl]benzenesulfonamide;

2-fluoro-4-[4-(2-methylpyridin-3-yl)-5-oxo-2,5-dihydro-3-furanyl]benzenesulfonamide;

2-fluoro-4-[4-(2-methylpyridin-3-yl)-5-oxo-2,5-dihydro-3-furanyl]benzenesulfonamide;

2-fluoro-4-[4-(3-pyridinyl)-5-oxo-2,5-dihydro-3-furanyl]benzenesulfonamide;

2-fluoro-4-[4-(5-methylpyridin-3-yl)-5-oxo-2,5-dihydro-3-furanyl]benzenesulfonamide;

2-fluoro-4-[4-(2-methylpyridin-3-yl)-5-oxo-2,5-dihydro-3-furanyl]benzenesulfonamide;

2-fluoro-4-[4-cyclohexyl-5-oxo-2,5-dihydro-3-furanyl]benzenesulfonamide;

2-fluoro-4-[4-cyclopentyl-5-oxo-2,5-dihydro-3-furanyl]benzenesulfonamide;

3-phenyl-4-[3-fluoro-4-(methylsulfonyl)phenyl]-2(5H)-furanone;

3-(3-chlorophenyl)-4-[3-fluoro-4-(methylsulfonyl)phenyl]-2(5H)-furanone;

3-(4-chlorophenyl)-4-[3-fluoro-4-(methylsulfonyl)phenyl]-2(5H)-furanone;

3-(3-bromophenyl)-4-[3-fluoro-4-(methylsulfonyl)phenyl]-2(5H)-furanone;

3-(4-bromophenyl)-4-[3-fluoro-4-(methylsulfonyl)phenyl]-2(5H)-furanone;

3-(3-fluorophenyl)-4-[3-fluoro-4-(methylsulfonyl)phenyl]-2(5H)-furanone;

3-(4-fluorophenyl)-4-[3-fluoro-4-(methylsulfonyl)phenyl]-2(5H)-furanone;

3-(3-methylphenyl)-4-[3-fluoro-4-(methylsulfonyl)phenyl]-2(5H)-furanone;

3-(4-methylphenyl)-4-[3-fluoro-4-(methylsulfonyl)phenyl]-2(5H)-furanone;

3-(3-cyanophenyl)-4-[3-fluoro-4-(methylsulfonyl)phenyl]-2(5H)-furanone;

3-(4-cyanophenyl)-4-[3-fluoro-4-(methylsulfonyl)phenyl]-2(5H)-furanone;

3-(3-trifluoromethylphenyl)-4-[3-fluoro-4-(methyl-sulfonyl)phenyl]-2(5H)-furanone;

3-(4-trifluoromethylphenyl)-4-[3-fluoro-4-(methyl-sulfonyl)phenyl]-2(5H)-furanone;

3-(3-trifluoromethoxyphenyl)-4-[3-fluoro-4-(methyl-sulfonyl)phenyl]-2(5H)-furanone;

3-(4-trifluoromethoxyphenyl)-4-[3-fluoro-4-(methyl-sulfonyl)phenyl]-2(5H)-furanone;

3-(3,4-dichlorophenyl)-4-[3-fluoro-4-(methylsulfonyl)phenyl]-2(5H)-furanone;

3-(3,4-dibromophenyl)-4-[3-fluoro-4-(methylsulfonyl)phenyl]-2(5H)-furanone

3-(3,4-difluorophenyl)-4-[3-fluoro-4-(methylsulfonyl)phenyl]-2(5H)-furanone;

3-(3,5-dichlorophenyl)-4-[3-fluoro-4-(methylsulfonyl)phenyl]-2(5H)-furanone;

3-(3,5-dibromophenyl)-4-[3-fluoro-4-(methylsulfonyl)phenyl]-2(5H)-furanone;

3-(3,5-difluorophenyl)-4-[3-fluoro-4-(methylsulfonyl)phenyl]-2(5H)-furanone;

3-(3,4-dimethylphenyl)-4-[3-fluoro-4-(methylsulfonyl)phenyl]-2(5H)-furanone;

3-(3,5-dimethylphenyl)-4-[3-fluoro-4-(methylsulfonyl)phenyl]-2(5H)-furanone;

3-(3-methyl-4-chlorophenyl)-4-[3-fluoro-4-(methyl-sulfonyl)phenyl]-2(5H)-furanone;

3-(4-methyl-3-chlorophenyl)-4-[3-fluoro-4-(methyl-sulfonyl)phenyl]-2(5H)-furanone;

3-(3-methyl-4-fluorophenyl)-4-[3-fluoro-4-(methyl-sulfonyl)phenyl]-2(5H)-furanone;

3-(4-methyl-3-fluorophenyl)-4-[3-fluoro-4-(methyl-sulfonyl)phenyl]-2(5H)-furanone;

3-(3-methyl-4-bromophenyl)-4-[3-fluoro-4-(methyl-sulfonyl)phenyl]-2(5H)-furanone;

3-(4-methyl-3-bromophenyl)-4-[3-fluoro-4-(methyl-sulfonyl)phenyl]-2(5H)-furanone;

3-(3-methyl-4-trifluoromethylphenyl)-4-[3-fluoro-4-(methylsulfonyl)phenyl]-2(5H)-furanone;

3-(4-methyl-3-trifluoromethylphenyl)-4-[3-fluoro-4-(methylsulfonyl)phenyl]-2(5H)-furanone;

3-(3-methyl-4-trifluoromethoxyphenyl)-4-[3-fluoro-4-(methylsulfonyl)phenyl]-2(5H)-furanone;

3-(4-methyl-3-trifluoromethoxyphenyl)-4-[3-fluoro-4-(methylsulfonyl)phenyl]-2(5H)-furanone;

3-(3-cyano-4-methylphenyl)-4-[3-fluoro-4-(methyl-sulfonyl)phenyl]-2(5H)-furanone;

3-(4-cyano-3-methylphenyl)-4-[3-fluoro-4-(methyl-sulfonyl)phenyl]-2(5H)-furanone;

3-(3-chloro-4-methoxyphenyl)-4-[3-fluoro-4-(methyl-sulfonyl)phenyl]-2(5H)-furanone;

3-(4-chloro-3-methoxyphenyl)-4-[3-fluoro-4-(methyl-sulfonyl)phenyl]-2(5H)-furanone;

3-(2-methylpyridin-6-yl)-4-[3-fluoro-4-(methylsulfonyl)phenyl]-2(5H)-furanone;

3-(2-methylthiazol-4-yl)-4-[3-fluoro-4-(methylsulfonyl)phenyl]-2(5H)-furanone;

3-(4-methylthiazol-2-yl)-4-[3-fluoro-4-(methylsulfonyl)phenyl]-2(5H)-furanone;

3-(2-methylpyridin-3-yl)-4-[3-fluoro-4-(methylsulfonyl)phenyl]-2(5H)-furanone;

3-2-methylpyridin-3-yl)-4-[3-fluoro-4-(methylsulfonyl)phenyl]-2(5H)-furanone;

3-(3-pyridinyl)-4-[3-fluoro-4-(methylsulfonyl)phenyl]-2(5H)-furanone;

3-(5-methylpyridin-3-yl)-4-[3-fluoro-4-(methylsulfonyl)phenyl]-2(5H)-furanone;

3-(2-methylpyridin-3-yl)-4-[3-fluoro-4-(methylsulfonyl)phenyl]-2(5H)-furanone;

3-cyclohexyl-4-[3-fluoro-4-(methylsulfonyl)phenyl]-2(5H)-furanone;

3-cyclopentyl-4-[3-fluoro-4-(methylsulfonyl)phenyl]-2(5H)-furanone;

4-[3-fluoro-4-(methylsulfonyl)phenyl]-3-phenyl-2(5H)-furanone;

and the pharmaceutically-acceptable salts, tautomers and prodrugs thereof.

In one embodiment, the species is:

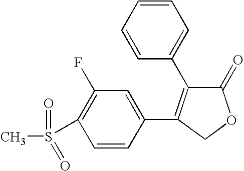

and the pharmaceutically-acceptable salts, tautomers and prodrugs thereof. In another embodiment, the fluoro radical is at the meta position relative to the sulfonyl group.

Cyclopentenes

In still another embodiment, the present method of treating cyclooxygenase-2 mediated disorders comprises administering to the subject a therapeutically-effective amount of one or more compounds selected from a subclass of compounds of Formula I corresponding to Formula VI:

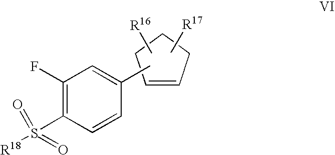

wherein substituents R¹⁶, R¹⁷ and R¹⁸ have the same definitions and sub-definitions as substituents R¹, R³ and R², respectively, set forth above for the compounds of Formula I, and the pharmaceutically-acceptable salts, tautomers and prodrugs thereof.

Within this subclass of compounds, a preferred group of compounds consists of those compounds of Formula VIA:

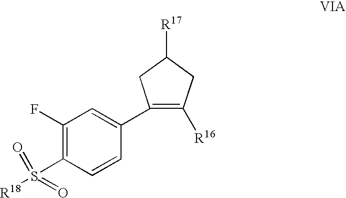

wherein R¹⁶, R¹⁷ and R¹⁸ are as defined above, and the pharmaceutically-acceptable salts, tautomers and prodrugs thereof.

Preferred species within this subclass include, but are not limited to:

2-fluoro-4-[2-phenylcyclopenten-1-yl]benezenesulfonamide;

2-fluoro-4-[2-(3-chlorophenyl)cyclopenten-1-yl]benezenesulfonamide;

2-fluoro-4-[2-(4-chlorophenyl)cyclopenten-1-yl]benezenesulfonamide;

2-fluoro-4-[2-(3-bromophenyl)cyclopenten-1-yl]benezenesulfonamide;

2-fluoro-4-[2-(4-bromophenyl)cyclopenten-1-yl]benezenesulfonamide;

2-fluoro-4-[2-(3-fluorophenyl)cyclopenten-1-yl]benezenesulfonamide;

2-fluoro-4-[2-(4-fluorophenyl)cyclopenten-1-yl]benezenesulfonamide;

2-fluoro-4-[2-(3-methylphenyl)cyclopenten-1-yl]benezenesulfonamide;

2-fluoro-4-[2-(4-methylphenyl)cyclopenten-1-yl]benezenesulfonamide;

2-fluoro-4-[2-(3-cyanophenyl)cyclopenten-1-yl]benezenesulfonamide;

2-fluoro-4-[2-(4-cyanophenyl)cyclopenten-1-yl]benezenesulfonamide;

2-fluoro-4-[2-(3-trifluoromethylphenyl)cyclopenten-1-yl]benezenesulfonamide;

2-fluoro-4-[2-(4-trifluoromethylphenyl)cyclopenten-1-yl]benezenesulfonamide;

2-fluoro-4-[2-(3-trifluoromethoxyphenyl)cyclopenten-1-yl]benezenesulfonamide;

2-fluoro-4-[2-(4-trifluoromethoxyphenyl)cyclopenten-1-yl]benezenesulfonamide;

2-fluoro-4-[2-(3,4-dichlorophenyl)cyclopenten-1-yl]benezenesulfonamide;

2-fluoro-4-[2-(3,4-dibromophenyl)cyclopenten-1-yl]benezenesulfonamide;

2-fluoro-4-[2-(3,4-difluorophenyl)cyclopenten-1-yl]benezenesulfonamide;

2-fluoro-4-[2-(3,5-dichlorophenyl)cyclopenten-1-yl]benezenesulfonamide;

2-fluoro-4-[2-(3,5-dibromophenyl)cyclopenten-1-yl]benezenesulfonamide;

2-fluoro-4-[2-(3,5-difluorophenyl)cyclopenten-1-yl]benezenesulfonamide;

2-fluoro-4-[2-(3,4-dimethylphenyl)cyclopenten-1-yl]benezenesulfonamide;

2-fluoro-4-[2-(3,5-dimethylphenyl)cyclopenten-1-yl]benezenesulfonamide;

2-fluoro-4-[2-(3-methyl-4-chlorophenyl)cyclopenten-1-yl]benezenesulfonamide;

2-fluoro-4-[2-(4-methyl-3-chlorophenyl)cyclopenten-1-yl]benezenesulfonamide;

2-fluoro-4-[2-(3-methyl-4-fluorophenyl)cyclopenten-1-yl]benezenesulfonamide;

2-fluoro-4-[2-(4-methyl-3-fluorophenyl)cyclopenten-1-yl]benezenesulfonamide;

2-fluoro-4-[2-(3-methyl-4-bromophenyl)cyclopenten-1-yl]benezenesulfonamide;

2-fluoro-4-[2-(4-methyl-3-bromophenyl)cyclopenten-1-yl]benezenesulfonamide;

2-fluoro-4-[2-(3-methyl-4-trifluoromethylphenyl)cyclopenten-1-yl]benezenesulfonamide;

2-fluoro-4-[2-(4-methyl-3-trifluoromethylphenyl)cyclopenten-1-yl]benezenesulfonamide;

2-fluoro-4-[2-(3-methyl-4-trifluoromethoxyphenyl)cyclopenten-1-yl]benezenesulfonamide;

2-fluoro-4-[2-(4-methyl-3-trifluoromethoxyphenyl)cyclopenten-1-yl]benezenesulfonamide;

2-fluoro-4-[2-(3-cyano-4-methylphenyl)cyclopenten-1-yl]benezenesulfonamide;

2-fluoro-4-[2-(4-cyano-3-methylphenyl)cyclopenten-1-yl]benezenesulfonamide;

2-fluoro-4-[2-(3-chloro-4-methoxyphenyl)cyclopenten-1-yl]benezenesulfonamide;

2-fluoro-4-[2-(4-chloro-3-methoxyphenyl)cyclopenten-1-yl]benezenesulfonamide;

2-fluoro-4-[2-(2-methylpyridin-6-yl)cyclopenten-1-yl]benezenesulfonamide;

2-fluoro-4-[2-(2-methylthiazol-4-yl)cyclopenten-1-yl]benezenesulfonamide;

2-fluoro-4-[2-(4-methylthiazol-2-yl)cyclopenten-1-yl]benezenesulfonamide;

2-fluoro-4-[2-(2-methylpyridin-3-yl)cyclopenten-1-yl]benezenesulfonamide;

2-fluoro-4-[2-(2-methylpyridin-3-yl)cyclopenten-1-yl]benezenesulfonamide;

2-fluoro-4-[2-(3-pyridinyl)cyclopenten-1-yl]benezenesulfonamide;

2-fluoro-4-[2-(5-methylpyridin-3-yl)cyclopenten-1-yl]benezenesulfonamide;

2-fluoro-4-[2-(2-methylpyridin-3-yl)cyclopenten-1-yl]benezenesulfonamide;

2-fluoro-4-[2-cyclohexylcyclopenten-1-yl]benezenesulfonamide;

2-fluoro-4-[2-cyclopentylcyclopenten-1-yl]benezenesulfonamide;

4-[2-phenylcyclopenten-1-yl]-2-fluorophenylmethylsulfone;

4-[2-(3-chlorophenyl)cyclopenten-1-yl]-2-fluorophenylmethylsulfone;

4-[2-(4-chlorophenyl)cyclopenten-1-yl]-2-fluorophenylmethylsulfone;

4-[2-(3-bromophenyl)cyclopenten-1-yl]-2-fluorophenylmethylsulfone;

4-[2-(4-bromophenyl)cyclopenten-1-yl]-2-fluorophenylmethylsulfone;

4-[2-(3-fluorophenyl)cyclopenten-1-yl]-2-fluorophenylmethylsulfone;

4-[2-(4-fluorophenyl)cyclopenten-1-yl]-2-fluorophenylmethylsulfone;

4-[2-(3-methylphenyl)cyclopenten-1-yl]-2-fluorophenylmethylsulfone;

4-[2-(4-methylphenyl)cyclopenten-1-yl]-2-fluorophenylmethylsulfone;

4-[2-(3-cyanophenyl)cyclopenten-1-yl]-2-fluorophenylmethylsulfone;

4-[2-(4-cyanophenyl)cyclopenten-1-yl]-2-fluorophenylmethylsulfone;

4-[2-(3-trifluoromethylphenyl)cyclopenten-1-yl]-2-fluorophenylmethylsulfone;

4-[2-(4-trifluoromethylphenyl)cyclopenten-1-yl]-2-fluorophenylmethylsulfone;

4-[2-(3-trifluoromethoxyphenyl)cyclopenten-1-yl]-2-fluorophenylmethyl sulfone;

4-[2-(4-trifluoromethoxyphenyl)cyclopenten-1-yl]-2-fluorophenylmethyl sulfone;

4-[2-(3,4-dichlorophenyl)cyclopenten-1-yl]-2-fluorophenylmethylsulfone;

4-[2-(3,4-dibromophenyl)cyclopenten-1-yl]-2-fluorophenylmethylsulfone;

4-[2-(3,4-difluorophenyl)cyclopenten-1-yl]-2-fluorophenylmethylsulfone;

4-[2-(3,5-dichlorophenyl)cyclopenten-1-yl]-2-fluorophenylmethylsulfone;

4-[2-(3,5-dibromophenyl)cyclopenten-1-yl]-2-fluorophenylmethylsulfone;

4-[2-(3,5-difluorophenyl)cyclopenten-1-yl]-2-fluorophenylmethylsulfone;

4-[2-(3,4-dimethylphenyl)cyclopenten-1-yl]-2-fluorophenylmethylsulfone;

4-[2-(3,5-dimethylphenyl)cyclopenten-1-yl]-2-fluorophenylmethylsulfone;

4-[2-(3-methyl-4-chlorophenyl)cyclopenten-1-yl]-2-fluorophenylmethylsulfone;

4-[2-(4-methyl-3-chlorophenyl)cyclopenten-1-yl]-2-fluorophenylmethylsulfone;

4-[2-(3-methyl-4-fluorophenyl)cyclopenten-1-yl]-2-fluorophenylmethylsulfone;

4-[2-(4-methyl-3-fluorophenyl)cyclopenten-1-yl]-2-fluorophenylmethylsulfone;

4-[2-(3-methyl-4-bromophenyl)cyclopenten-1-yl]-2-fluorophenylmethylsulfone;

4-[2-(4-methyl-3-bromophenyl)cyclopenten-1-yl]-2-fluorophenylmethylsulfone;

4-[2-(3-methyl-4-trifluoromethylphenyl)cyclopenten-1-yl]-2-fluorophenylmethyl sulfone;

4-[2-(4-methyl-3-trifluoromethylphenyl)cyclopenten-1-yl]-2-fluorophenylmethyl sulfone;

4-[2-(3-methyl-4-trifluoromethoxyphenyl)cyclopenten-1-yl]-2-fluorophenyl methyl sulfone;

4-[2-(4-methyl-3-trifluoromethoxyphenyl)cyclopenten-1-yl]-2-fluorophenyl methyl sulfone;

4-[2-(3-cyano-4-methylphenyl)cyclopenten-1-yl]-2-fluorophenylmethylsulfone;

4-[2-(4-cyano-3-methylphenyl)cyclopenten-1-yl]-2-fluorophenylmethylsulfone;

4-[2-(3-chloro-4-methoxyphenyl)cyclopenten-1-yl]-2-fluorophenylmethyl sulfone;

4-[2-(4-chloro-3-methoxyphenyl)cyclopenten-1-yl]-2-fluorophenylmethyl sulfone;

4-[2-(2-methylpyridin-6-yl)cyclopenten-1-yl]-2-fluorophenylmethylsulfone;

4-[2-(2-methylthiazol-4-yl)cyclopenten-1-yl]-2-fluorophenylmethylsulfone;

4-[2-(4-methylthiazol-2-yl)cyclopenten-1-yl]-2-fluorophenylmethylsulfone;

4-[2-(2-methylpyridin-3-yl)cyclopenten-1-yl]-2-fluorophenylmethylsulfone;

4-[2-(2-methylpyridin-3-yl)cyclopenten-1-yl]-2-fluorophenylmethylsulfone;

4-[2-(3-pyridinyl)cyclopenten-1-yl]-2-fluorophenylmethylsulfone;

4-[2-(5-methylpyridin-3-yl)cyclopenten-1-yl]-2-fluorophenylmethylsulfone;

4-[2-(2-methylpyridin-3-yl)cyclopenten-1-yl]-2-fluorophenylmethylsulfone;

4-[2-cyclohexylcyclopenten-1-yl]-2-fluorophenylmethylsulfone;

4-[2-cyclopentylcyclopenten-1-yl]-2-fluorophenylmethylsulfone;

2-(3,5-difluorophenyl)-3-(3-fluoro-4-(methylsulfonyl)phenyl))-2-cyclopenten-1-one;

and the pharmaceutically-acceptable salts, tautomers and prodrugs thereof.

In one embodiment, the species is:

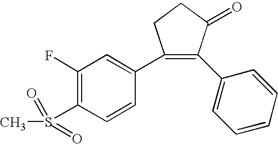

and the pharmaceutically-acceptable salts, tautomers and prodrugs thereof. In another embodiment, the fluoro radical is at the meta position relative to the sulfonyl group.

Pyridines

In still another embodiment, the present method of treating cyclooxygenase-2 mediated disorders comprises administering to the subject a therapeutically-effective amount of one or more compounds selected from a subclass of compounds of Formula I corresponding to Formula VII:

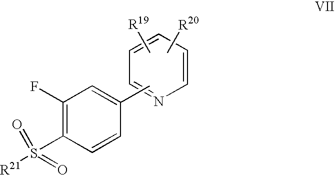

wherein substituents R¹⁹, R²⁰ and R²¹ have the same definitions and sub-definitions as substituents R¹, R³ and R², respectively, set forth above for the compounds of Formula I, and the pharmaceutically-acceptable salts, tautomers and prodrugs thereof.

Within this subclass of compounds, a preferred group of compounds consists of those compounds of Formula VIIA:

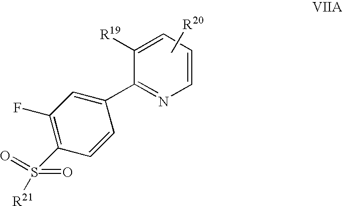

wherein R¹⁹, R²⁰ and R²¹ are as defined above, and the pharmaceutically-acceptable salts, tautomers and prodrugs thereof.

Preferred species within this subclass include, but are not limited to:

2-fluoro-4-[3-phenyl-3-pyridinyl]-benezenesulfonamide;

2-fluoro-4-[3-(3-chlorophenyl)-3-pyridinyl]-benezene-sulfonamide;

2-fluoro-4-[3-(4-chlorophenyl)-3-pyridinyl]-benezene-sulfonamide;

2-fluoro-4-[3-(3-bromophenyl)-3-pyridinyl]-benezene-sulfonamide;

2-fluoro-4-[3-(4-bromophenyl)-3-pyridinyl]-benezene-sulfonamide;

2-fluoro-4-[3-(3-fluorophenyl)-3-pyridinyl]-benezene-sulfonamide;

2-fluoro-4-[3-(4-fluorophenyl)-3-pyridinyl]-benezene-sulfonamide;

2-fluoro-4-[3-(3-methylphenyl)-3-pyridinyl]-benezene-sulfonamide;

2-fluoro-4-[3-(4-methylphenyl)-3-pyridinyl]-benezene-sulfonamide;

2-fluoro-4-[3-(3-cyanophenyl)-3-pyridinyl]-benezene-sulfonamide;

2-fluoro-4-[3-(4-cyanophenyl)-3-pyridinyl]-benezene-sulfonamide;

2-fluoro-4-[3-(3-trifluoromethylphenyl)-3-pyridinyl]-benezenesulfonamide;

2-fluoro-4-[3-(4-trifluoromethylphenyl)-3-pyridinyl]-benezenesulfonamide;

2-fluoro-4-[3-(3-trifluoromethoxyphenyl)-3-pyridinyl]-benezenesulfonamide;

2-fluoro-4-[3-(4-trifluoromethoxyphenyl)-3-pyridinyl]-benezenesulfonamide;

2-fluoro-4-[3-(3,4-dichlorophenyl)-3-pyridinyl]-benezenesulfonamide;

2-fluoro-4-[3-(3,4-dibromophenyl)-3-pyridinyl]-benezenesulfonamide;

2-fluoro-4-[3-(3,4-difluorophenyl)-3-pyridinyl]-benezenesulfonamide;

2-fluoro-4-[3-(3,5-dichlorophenyl)-3-pyridinyl]-benezenesulfonamide;

2-fluoro-4-[3-(3,5-dibromophenyl)-3-pyridinyl]-benezenesulfonamide;

2-fluoro-4-[3-(3,5-difluorophenyl)-3-pyridinyl]-benezenesulfonamide;

2-fluoro-4-[3-(3,4-dimethylphenyl)-3-pyridinyl]-benezenesulfonamide;

2-fluoro-4-[3-(3,5-dimethylphenyl)-3-pyridinyl]-benezenesulfonamide;

2-fluoro-4-[3-(3-methyl-4-chlorophenyl)-3-pyridinyl]-benezenesulfonamide;

2-fluoro-4-[3-(4-methyl-3-chlorophenyl)-3-pyridinyl]-benezenesulfonamide;

2-fluoro-4-[3-(3-methyl-4-fluorophenyl)-3-pyridinyl]-benezenesulfonamide;

2-fluoro-4-[3-(4-methyl-3-fluorophenyl)-3-pyridinyl]-benezenesulfonamide;

2-fluoro-4-[3-(3-methyl-4-bromophenyl)-3-pyridinyl]-benezenesulfonamide;

2-fluoro-4-[3-(4-methyl-3-bromophenyl)-3-pyridinyl]-benezenesulfonamide;

2-fluoro-4-[3-(3-methyl-4-trifluoromethylphenyl)-3-pyridinyl]-benezenesulfonamide;

2-fluoro-4-[3-(4-methyl-3-trifluoromethylphenyl)-3-pyridinyl]-benezenesulfonamide;

2-fluoro-4-[3-(3-methyl-4-trifluoromethoxyphenyl)-3-pyridinyl]-benezenesulfonamide;

2-fluoro-4-[3-(4-methyl-3-trifluoromethoxyphenyl)-3-pyridinyl]-benezenesulfonamide;

2-fluoro-4-[3-(3-cyano-4-methylphenyl)-3-pyridinyl]-benezenesulfonamide;

2-fluoro-4-[3-(4-cyano-3-methylphenyl)-3-pyridinyl]-benezenesulfonamide;

2-fluoro-4-[3-(3-chloro-4-methoxyphenyl)-3-pyridinyl]-benezenesulfonamide;

2-fluoro-4-[3-(4-chloro-3-methoxyphenyl)-3-pyridinyl]-benezenesulfonamide;

2-fluoro-4-[3-(2-methylpyridin-6-yl)-3-pyridinyl]-benezenesulfonamide;

2-fluoro-4-[3-(2-methylthiazol-4-yl)-3-pyridinyl]-benezenesulfonamide;

2-fluoro-4-[3-(4-methylthiazol-2-yl)-3-pyridinyl]-benezenesulfonamide;

2-fluoro-4-[3-(2-methylpyridin-3-yl)-3-pyridinyl]-benezenesulfonamide;

2-fluoro-4-[3-(2-methylpyridin-3-yl)-3-pyridinyl]-benezenesulfonamide;

2-fluoro-4-[3-(3-pyridinyl)-3-pyridinyl]-benezene-sulfonamide;

2-fluoro-4-[3-(5-methylpyridin-3-yl)-3-pyridinyl]-benezenesulfonamide;

2-fluoro-4-[3-(2-methylpyridin-3-yl)-3-pyridinyl]-benezenesulfonamide;

2-fluoro-4-[3-cyclohexyl-3-pyridinyl]-benezene-sulfonamide;

2-fluoro-4-[3-cyclopentyl-3-pyridinyl]-benezene-sulfonamide;

2-fluoro-4-[3-phenyl-2-pyridinyl]-benezenesulfonamide;

2-fluoro-4-[3-(3-chlorophenyl)-2-pyridinyl]-benezene-sulfonamide;

2-fluoro-4-[3-(4-chlorophenyl)-2-pyridinyl]-benezenesulfonamide;

2-fluoro-4-[3-(3-bromophenyl)-2-pyridinyl]-benezenesulfonamide;

2-fluoro-4-[3-(4-bromophenyl)-2-pyridinyl]-benezenesulfonamide;

2-fluoro-4-[3-(3-fluorophenyl)-2-pyridinyl]-benezenesulfonamide;

2-fluoro-4-[3-(4-fluorophenyl)-2-pyridinyl]-benezenesulfonamide;

2-fluoro-4-[3-(3-methylphenyl)-2-pyridinyl]-benezenesulfonamide;

2-fluoro-4-[3-(4-methylphenyl)-2-pyridinyl]-benezenesulfonamide;

2-fluoro-4-[3-(3-cyanophenyl)-2-pyridinyl]-benezenesulfonamide;

2-fluoro-4-[3-(4-cyanophenyl)-2-pyridinyl]-benezenesulfonamide;

2-fluoro-4-[3-(3-trifluoromethylphenyl)-2-pyridinyl]-benezenesulfonamide;

2-fluoro-4-[3-(4-trifluoromethylphenyl)-2-pyridinyl]-benezenesulfonamide;

2-fluoro-4-[3-(3-trifluoromethoxyphenyl)-2-pyridinyl]-benezenesulfonamide;

2-fluoro-4-[3-(4-trifluoromethoxyphenyl)-2-pyridinyl]-benezenesulfonamide;

2-fluoro-4-[3-(3,4-dichlorophenyl)-2-pyridinyl]-benezenesulfonamide;

2-fluoro-4-[3-(3,4-dibromophenyl)-2-pyridinyl]-benezenesulfonamide;

2-fluoro-4-[3-(3,4-difluorophenyl)-2-pyridinyl]-benezenesulfonamide;

2-fluoro-4-[3-(3,5-dichlorophenyl)-2-pyridinyl]-benezenesulfonamide;

2-fluoro-4-[3-(3,5-dibromophenyl)-2-pyridinyl]-benezenesulfonamide;

2-fluoro-4-[3-(3,5-difluorophenyl)-2-pyridinyl]-benezenesulfonamide;

2-fluoro-4-[3-(3,4-dimethylphenyl)-2-pyridinyl]-benezenesulfonamide;

2-fluoro-4-[3-(3,5-dimethylphenyl)-2-pyridinyl]-benezenesulfonamide;

2-fluoro-4-[3-(3-methyl-4-chlorophenyl)-2-pyridinyl]-benezenesulfonamide;

2-fluoro-1-[3-(4-methyl-3-chlorophenyl)-2-pyridinyl]-benezenesulfonamide;

2-fluoro-4-[3-(3-methyl-4-fluorophenyl)-2-pyridinyl]-benezenesulfonamide;

2-fluoro-4-[3-(4-methyl-3-fluorophenyl)-2-pyridinyl]-benezenesulfonamide;

2-fluoro-4-[3-(3-methyl-4-bromophenyl)-2-pyridinyl]-benezenesulfonamide;

2-fluoro-4-[3-(4-methyl-3-bromophenyl)-2-pyridinyl]-benezenesulfonamide;

2-fluoro-4-[3-(3-methyl-4-trifluoromethylphenyl)-2-pyridinyl]-benezenesulfonamide;

2-fluoro-4-[3-(4-methyl-3-trifluoromethylphenyl)-2-pyridinyl]-benezenesulfonamide;

2-fluoro-[3-(3-methyl-4-trifluoromethoxyphenyl)-2-pyridinyl]-benezenesulfonamide;

2-fluoro-4-[3-(4-methyl-3-trifluoromethoxyphenyl)-2-pyridinyl]-benezenesulfonamide;

2-fluoro-4-[3-(3-cyano-4-methylphenyl)-2-pyridinyl]-benezenesulfonamide;

2-fluoro-4-[3-(4-cyano-3-methylphenyl)-2-pyridinyl]-benezenesulfonamide;

2-fluoro-4-[3-(3-chloro-4-methoxyphenyl)-2-pyridinyl]-benezenesulfonamide;

2-fluoro-4-[3-(4-chloro-3-methoxyphenyl)-2-pyridinyl]-benezenesulfonamide;

2-fluoro-4-[3-(2-methylpyridin-6-yl)-2-pyridinyl]-benezenesulfonamide;

2-fluoro-4-[3-(2-methylthiazol-4-yl)-2-pyridinyl]-benezenesulfonamide;

2-fluoro-4-[3-(4-methylthiazol-2-yl)-2-pyridinyl]-benezenesulfonamide;

2-fluoro-4-[3-(2-methylpyridin-3-yl)-2-pyridinyl]-benezenesulfonamide;

2-fluoro-4-[3-(2-methylpyridin-3-yl)-2-pyridinyl]-benezenesulfonamide;

2-fluoro-4-[3-(3-pyridinyl)-2-pyridinyl]-benezenesulfonamide;

2-fluoro-4-[3-(5-methylpyridin-3-yl)-2-pyridinyl]-benezenesulfonamide;

2-fluoro-4-[3-(2-methylpyridin-3-yl)-2-pyridinyl]-benezenesulfonamide;

2-fluoro-4-[3-cyclohexyl-2-pyridinyl]-benezenesulfonamide;

2-fluoro-4-[3-cyclopentyl-2-pyridinyl]-benezenesulfonamide;

4-[3-phenyl-2-pyridinyl]-2-fluorophenylmethylsulfone;

4-[3-(3-chlorophenyl)-2-pyridinyl]-2-fluorophenylmethylsulfone;

4-[3-(4-chlorophenyl)-2-pyridinyl]-2-fluorophenylmethylsulfone;

4-[3-(3-bromophenyl)-2-pyridinyl]-2-fluorophenylmethylsulfone;

4-[3-(4-bromophenyl)-2-pyridinyl]-2-fluorophenylmethylsulfone;

4-[3-(3-fluorophenyl)-2-pyridinyl]-2-fluorophenylmethylsulfone;

4-[3-(4-fluorophenyl)-2-pyridinyl]-2-fluorophenylmethylsulfone;

4-[3-(3-methylphenyl)-2-pyridinyl]-2-fluorophenylmethylsulfone;

4-[3-(4-methylphenyl)-2-pyridinyl]-2-fluorophenylmethylsulfone;

4-[3-(3-cyanophenyl)-2-pyridinyl]-2-fluorophenylmethylsulfone;

4-[3-(4-cyanophenyl)-2-pyridinyl]-2-fluorophenylmethylsulfone;

4-[3-(3-trifluoromethylphenyl)-2-pyridinyl]-2-fluorophenylmethylsulfone;

4-[3-(4-trifluoromethylphenyl)-2-pyridinyl]-2-fluorophenylmethylsulfone;

4-[3-(3-trifluoromethoxyphenyl)-2-pyridinyl]-2-fluorophenylmethylsulfone;

4-[3-(4-trifluoromethoxyphenyl)-2-pyridinyl]-2-fluorophenylmethylsulfone;

4-[3-(3,4-dichlorophenyl)-2-pyridinyl]-2-fluorophenylmethylsulfone;

4-[3-(3,4-dibromophenyl)-2-pyridinyl]-2-fluorophenylmethylsulfone;

4-[3-(3,4-difluorophenyl)-2-pyridinyl]-2-fluorophenylmethylsulfone;

4-[3-(3,5-dichlorophenyl)-2-pyridinyl]-2-fluorophenylmethylsulfone;

4-[3-(3,5-dibromophenyl)-2-pyridinyl]-2-fluorophenylmethylsulfone;

4-[3-(3,5-difluorophenyl)-2-pyridinyl]-2-fluorophenylmethylsulfone;

4-[3-(3,4-methylphenyl)-2-pyridinyl]-2-fluorophenylmethylsulfone;

4-[3-(3,5-dimethylphenyl)-2-pyridinyl]-2-fluorophenylmethylsulfone;

4-[3-(3-methyl-4-chlorophenyl)-2-pyridinyl]-2-fluorophenylmethylsulfone;

4-[3-(4-methyl-3-chlorophenyl)-2-pyridinyl]-2-fluorophenylmethylsulfone;

4-[3-(3-methyl-4-fluorophenyl)-2-pyridinyl]-2-fluorophenylmethylsulfone;

4-[3-(4-methyl-3-fluorophenyl)-2-pyridinyl]-2-fluorophenylmethylsulfone;

4-[3-(3-methyl-4-bromophenyl)-2-pyridinyl]-2-fluorophenylmethylsulfone;

4-[3-(4-methyl-3-bromophenyl)-2-pyridinyl]-2-fluorophenylmethylsulfone;

4-[3-(3-methyl-4-trifluoromethylphenyl)-2-pyridinyl]-2-fluorophenylmethyl sulfone;

4-[3-(4-methyl-3-trifluoromethylphenyl)-2-pyridinyl]-2-fluorophenylmethyl sulfone;

4-[3-(3-methyl-4-trifluoromethoxyphenyl)-2-pyridinyl]-2-fluorophenylmethyl sulfone;

4-[3-(4-methyl-3-trifluoromethoxyphenyl)-2-pyridinyl]-2-fluorophenylmethyl sulfone;

4-[3-(3-cyano-4-methylphenyl)-2-pyridinyl]-2-fluorophenylmethylsulfone;

4-[3-(4-cyano-3-methylphenyl)-2-pyridinyl]-2-fluorophenylmethylsulfone;

4-[3-(3-chloro-4-methoxyphenyl)-2-pyridinyl]-2-fluorophenylmethylsulfone;

4-[3-(4-chloro-3-methoxyphenyl)-2-pyridinyl]-2-fluorophenylmethylsulfone;

4-[3-(2-methylpyridin-6-yl)-2-pyridinyl]-2-fluorophenylmethylsulfone;

4-[3-(2-methylthiazol-4-yl)-2-pyridinyl]-2-fluorophenylmethylsulfone;

4-[3-(4-methylthiazol-2-yl)-2-pyridinyl]-2-fluorophenylmethylsulfone;

4-[3-(2-methylpyridin-3-yl)-2-pyridinyl]-2-fluorophenylmethylsulfone;

4-[3-(2-methylpyridin-3-yl)-2-pyridinyl]-2-fluorophenylmethylsulfone;

4-[3-(3-pyridinyl)-2-pyridinyl]-2-fluorophenylmethylsulfone;

4-[3-(5-methylpyridin-3-yl)-2-pyridinyl]-2-fluorophenylmethylsulfone;

4-[3-(2-methylpyridin-3-yl)-2-pyridinyl]-2-fluorophenylmethylsulfone;

4-[3-cyclohexyl-2-pyridinyl]-2-fluorophenylmethylsulfone;

4-[3-cyclopentyl-2-pyridinyl]-2-fluorophenylmethylsulfone;

4-[4-phenyl-3-pyridinyl]-2-fluorophenylmethylsulfone;

4-[4-(3-chlorophenyl)-3-pyridinyl]-2-fluorophenylmethylsulfone;

4-[4-(4-chlorophenyl)-3-pyridinyl]-2-fluorophenylmethylsulfone;

4-[4-(3-bromophenyl)-3-pyridinyl]-2-fluorophenylmethylsulfone;

4-[4-(4-bromophenyl)-3-pyridinyl]-2-fluorophenylmethylsulfone;

4-[4-(3-fluorophenyl)-3-pyridinyl]-2-fluorophenylmethylsulfone;

4-[4-(4-fluorophenyl)-3-pyridinyl]-2-fluorophenylmethylsulfone;

4-[4-(3-methylphenyl)-3-pyridinyl]-2-fluorophenylmethylsulfone;

4-[4-(4-methylphenyl)-3-pyridinyl]-2-fluorophenylmethylsulfone;

4-[4-(3-cyanophenyl)-3-pyridinyl]-2-fluorophenylmethylsulfone;

4-[4-(4-cyanophenyl)-3-pyridinyl]-2-fluorophenylmethylsulfone;

4-[4-(3-trifluoromethylphenyl)-3-pyridinyl]-2-fluorophenylmethylsulfone;

4-[4-(4-trifluoromethylphenyl)-3-pyridinyl]-2-fluorophenylmethylsulfone;

4-[4-(3-trifluoromethoxyphenyl)-3-pyridinyl]-2-fluorophenylmethylsulfone;

4-[4-(4-trifluoromethoxyphenyl)-3-pyridinyl]-2-fluorophenylmethylsulfone;

4-[4-(3,4-dichlorophenyl)-3-pyridinyl]-2-fluorophenylmethylsulfone;

4-[4-(3,4-dibromophenyl)-3-pyridinyl]-2-fluorophenylmethylsulfone;

4-[4-(3,4-difluorophenyl)-3-pyridinyl]-2-fluorophenylmethylsulfone;

4-[4-(3,5-dichlorophenyl)-3-pyridinyl]-2-fluorophenylmethylsulfone;

4-[4-(3,5-dibromophenyl)-3-pyridinyl]-2-fluorophenylmethylsulfone;

4-[4-(3,5-difluorophenyl)-3-pyridinyl]-2-fluorophenylmethylsulfone;

4-[4-(3,4-dimethylphenyl)-3-pyridinyl]-2-fluorophenylmethylsulfone;

4-[4-(3,5-dimethylphenyl)-3-pyridinyl]-2-fluorophenylmethylsulfone;

4-[4-(3-methyl-4-chlorophenyl)-3-pyridinyl]-2-fluorophenylmethylsulfone;

4-[4-(4-methyl-3-chlorophenyl)-3-pyridinyl]-2-fluorophenylmethylsulfone;

4-[4-(3-methyl-4-fluorophenyl)-3-pyridinyl]-2-fluorophenylmethylsulfone;

4-[4-(4-methyl-3-fluorophenyl)-3-pyridinyl]-2-fluorophenylmethylsulfone;

4-[4-(3-methyl-4-bromophenyl)-3-pyridinyl]-2-fluorophenylmethylsulfone;

4-[4-(4-methyl-3-bromophenyl)-3-pyridinyl]-2-fluorophenylmethylsulfone;

4-[4-(3-methyl-4-trifluoromethylphenyl)-3-pyridinyl]-2-fluorophenylmethyl sulfone;

4-[4-(4-methyl-3-trifluoromethylphenyl)-3-pyridinyl]-2-fluorophenylmethyl sulfone;

4-[4-(3-methyl-4-trifluoromethoxyphenyl)-3-pyridinyl]-2-fluorophenylmethyl sulfone;

4-[4-(4-methyl-3-trifluoromethoxyphenyl)-3-pyridinyl]-2-fluorophenylmethyl sulfone;

4-[4-(3-cyano-4-methylphenyl)-3-pyridinyl]-2-fluorophenylmethylsulfone;

4-[4-(4-cyano-3-methylphenyl)-3-pyridinyl]-2-fluorophenylmethylsulfone;

4-[4-(3-chloro-4-methoxyphenyl)-3-pyridinyl]-2-fluorophenylmethylsulfone;

4-[4-(4-chloro-3-methoxyphenyl)-3-pyridinyl]-2-fluorophenylmethylsulfone;

4-[4-(2-methylpyridin-6-yl)-3-pyridinyl]-2-fluorophenylmethylsulfone;

4-[4-(2-methylthiazol-4-yl)-3-pyridinyl]-2-fluorophenylmethylsulfone;

4-[4-(4-methylthiazol-2-yl)-3-pyridinyl]-2-fluorophenylmethylsulfone;

4-[4-(2-methylpyridin-3-yl)-3-pyridinyl]-2-fluorophenylmethylsulfone;

4-[4-(2-methylpyridin-3-yl)-3-pyridinyl]-2-fluorophenylmethylsulfone;

4-[4-(3-pyridinyl)-3-pyridinyl]-2-fluorophenylmethylsulfone;

4-[4-(5-methylpyridin-3-yl)-3-pyridinyl]-2-fluorophenylmethylsulfone;

4-[4-(2-methylpyridin-3-yl)-3-pyridinyl]-2-fluorophenylmethylsulfone;

4-[4-cyclohexyl-3-pyridinyl]-2-fluorophenylmethylsulfone;

4-[4-cyclopentyl-3-pyridinyl]-2-fluorophenylmethylsulfone;

5-chloro-3-(3-fluoro-4-(methylsulfonyl)phenyl]-2-(methyl-5-pyridinyl)pyridine;

and the pharmaceutically-acceptable salts, tautomers and prodrugs thereof.

In one embodiment, the species is:

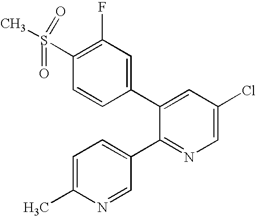

and the pharmaceutically-acceptable salts, tautomers and prodrugs thereof. In another embodiment, the fluoro radical is at the meta position relative to the sulfonyl group.

In another embodiment, the species is:

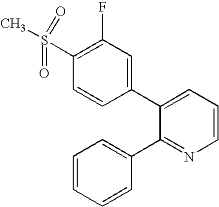

and the pharmaceutically-acceptable salts, tautomers and prodrugs thereof. In another embodiment, the fluoro radical is at the meta position relative to the sulfonyl group.

Additional Compounds

Within Formula I there is another subclass of compounds of interest that includes, but is not limited to:

2-fluoro-4-[3-2-oxo-2,3-dihydro-1,3-thiazol-4-yl]benzenesulfonamide;

2-fluoro-4-[3-(3-chlorophenyl)-2-oxo-2,3-dihydro-1,3-thiazol-4-yl]benzenesulfonamide;

2-fluoro-4-[3-(4-chlorophenyl)-2-oxo-2,3-dihydro-1,3-thiazol-4-yl]benzenesulfonamide;

2-fluoro-4-[3-(3-bromophenyl)-2-oxo-2,3-dihydro-1,3-thiazol-4-yl]benzenesulfonamide;

2-fluoro-4-[3-(4-bromophenyl)-2-oxo-2,3-dihydro-1,3-thiazol-4-yl]benzenesulfonamide;

2-fluoro-4-[3-(3-fluorophenyl)-2-oxo-2,3-dihydro-1,3-thiazol-4-yl]benzenesulfonamide;

2-fluoro-44-[3-(4-fluorophenyl)-2-oxo-2,3-dihydro-1,3-thiazol-4-yl]benzenesulfonamide;

2-fluoro-4-[3-(3-methylphenyl)-4-[3-fluoro-4-(methylsulfonyl)phenyl]-1,3-thiazol-2(3H)-one;

2-fluoro-4-[3-(4-methylphenyl)-2-oxo-2,3-dihydro-1,3-thiazol-4-yl]benzenesulfonamide;

2-fluoro-4-[3-(3-cyanophenyl)-2-oxo-2,3-dihydro-1,3-thiazol-4-yl]benzenesulfonamide;

2-fluoro-4-[3-(4-cyanophenyl)-2-oxo-2,3-dihydro-1,3-thiazol-4-yl]benzenesulfonamide;

2-fluoro-4-[3-(3-trifluoromethylphenyl)-2-oxo-2,3-dihydro-1,3-thiazol-4-yl]benzenesulfonamide;

2-fluoro-4-[3-(4-trifluoromethylphenyl)-2-oxo-2,3-dihydro-1,3-thiazol-4-yl]benzenesulfonamide;

2-fluoro-4-[3-(3-trifluoromethoxyphenyl)-2-oxo-2,3-dihydro-1,3-thiazol-4-yl]benzenesulfonamide;

2-fluoro-4-[3-(4-trifluoromethoxyphenyl)-2-oxo-2,3-dihydro-1,3-thiazol-4-yl]benzenesulfonamide;

2-fluoro-4-[3-(3,4-dichlorophenyl)-2-oxo-2,3-dihydro-1,3-thiazol-4-yl]benzenesulfonamide;

2-fluoro-4-[3-(3,4-dibromophenyl)-2-oxo-2,3-dihydro-1,3-thiazol-4-yl]benzenesulfonamide;

2-fluoro-4-[3-(3,4-difluorophenyl)-2-oxo-2,3-dihydro-1,3-thiazol-4-yl]benzenesulfonamide;

2-fluoro-4-[3-(3,5-dichlorophenyl)-2-oxo-2,3-dihydro-1,3-thiazol-4-yl]benzenesulfonamide;

2-fluoro-4-[3-(3,5-dibromophenyl)-2-oxo-2,3-dihydro-1,3-thiazol-4-yl]benzenesulfonamide;

2-fluoro-4-[3-(3,5-difluorophenyl)-2-oxo-2,3-dihydro-1,3-thiazol-4-yl]benzenesulfonamide;

2-fluoro-4-[3-(3,4-dimethylphenyl)-2-oxo-2,3-dihydro-1,3-thiazol-4-yl]benzenesulfonamide;

2-fluoro-4-[3-(3,5-dimethylphenyl)-2-oxo-2,3-dihydro-1,3-thiazol-4-yl]benzenesulfonamide;

2-fluoro-4-[3-(3-methyl-4-chlorophenyl)-2-oxo-2,3-dihydro-1,3-thiazol-4-yl]benzenesulfonamide;

2-fluoro-4-[3-(4-methyl-3-chlorophenyl)-2-oxo-2,3-dihydro-1,3-thiazol-4-yl]benzenesulfonamide;

2-fluoro-4-[3-(3-methyl-4-fluorophenyl)-2-oxo-2,3-dihydro-1,3-thiazol-4-yl]benzenesulfonamide;

2-fluoro-4-[3-(4-methyl-3-fluorophenyl)-2-oxo-2,3-dihydro-1,3-thiazol-4-yl]benzenesulfonamide;

2-fluoro-4-[3-(3-methyl-4-bromophenyl)-2-oxo-2,3-dihydro-1,3-thiazol-4-yl]benzenesulfonamide;

2-fluoro-4-[3-(4-methyl-3-bromophenyl)-2-oxo-2,3-dihydro-1,3-thiazol-4-yl]benzenesulfonamide;

2-fluoro-4-[3-(3-methyl-4-trifluoromethylphenyl)-2-oxo-2,3-dihydro-1,3-thiazol-4-yl]benzenesulfonamide;

2-fluoro-4-[3-(4-methyl-3-trifluoromethylphenyl)-2-oxo-2,3-dihydro-1,3-thiazol-4-yl]benzenesulfonamide;

2-fluoro-4-[3-(3-methyl-4-trifluoromethoxyphenyl)-2-oxo-2,3-dihydro-1,3-thiazol-4-yl]benzenesulfonamide;

2-fluoro-4-[3-(4-methyl-3-trifluoromethoxyphenyl)-2-oxo-2,3-dihydro-1,3-thiazol-4-yl]benzenesulfonamide;

2-fluoro-4-[3-(3-cyano-4-methylphenyl)-2-oxo-2,3-dihydro-1,3-thiazol-4-yl]benzenesulfonamide;

2-fluoro-4-[3-(4-cyano-3-methylphenyl)-2-oxo-2,3-dihydro-1,3-thiazol-4-yl]benzenesulfonamide;

2-fluoro-4-[3-(3-chloro-4-methoxyphenyl)-2-oxo-2,3-dihydro-1,3-thiazol-4-yl]benzenesulfonamide;

2-fluoro-4-[3-(4-chloro-3-methoxyphenyl)-2-oxo-2,3-dihydro-1,3-thiazol-4-yl]benzenesulfonamide;

2-fluoro-4-[3-(2-methylpyridin-6-yl)-2-oxo-2,3-dihydro-1,3-thiazol-4-yl]benzenesulfonamide;

2-fluoro-4-[3-(2-methylthiazol-4-yl)-2-oxo-2,3-dihydro-1,3-thiazol-4-yl]benzenesulfonamide;

2-fluoro-4-[3-(4-methylthiazol-2-yl)-2-oxo-2,3-dihydro-1,3-thiazol-4-yl]benzenesulfonamide;

2-fluoro-4-[3-(2-methylpyridin-3-yl)-2-oxo-2,3-dihydro-1,3-thiazol-4-yl]benzenesulfonamide;

2-fluoro-4-[3-(2-methylpyridin-3-yl)-2-oxo-2,3-dihydro-1,3-thiazol-4-yl]benzenesulfonamide;

2-fluoro-4-[3-(3-pyridinyl)-2-oxo-2,3-dihydro-1,3-thiazol-4-yl]benzenesulfonamide;

2-fluoro-4-[3-(5-methylpyridin-3-yl)-2-oxo-2,3-dihydro-1,3-thiazol-4-yl]benzenesulfonamide;

2-fluoro-4-[3-(2-methylpyridin-3-yl)-2-oxo-2,3-dihydro-1,3-thiazol-4-yl]benzenesulfonamide;

3-cyclohexyl-2-oxo-2,3-dihydro-1,3-thiazol-4-yl]benzenesulfonamide;

3-cyclopentyl-2-oxo-2,3-dihydro-1,3-thiazol-4-yl]benzenesulfonamide;

3-phenyl-4-[3-fluoro-4-(methylsulfonyl)phenyl]-1,3-thiazol-2(3H)-one;

3-(3-chlorophenyl)-4-[3-fluoro-4-(methylsulfonyl)phenyl]-1,3-thiazol-2(3H)-one;

3-(4-chlorophenyl)-4-[3-fluoro-4-(methylsulfonyl)phenyl]-1,3-thiazol-2(3H)-one;

3-(3-bromophenyl)-4-[3-fluoro-4-(methylsulfonyl)phenyl]-1,3-thiazol-2(3H)-one;

3-(4-bromophenyl)-4-[3-fluoro-4-(methylsulfonyl)phenyl]-1,3-thiazol-2(3H)-one;

3-(3-fluorophenyl)-4-[3-fluoro-4-(methylsulfonyl)phenyl]-1,3-thiazol-2(3H)-one;

3-(4-fluorophenyl)-4-[3-fluoro-4-(methylsulfonyl)phenyl]-1,3-thiazol-2(3H)-one;

3-(3-methylphenyl)-4-[3-fluoro-4-(methylsulfonyl)phenyl]-1,3-thiazol-2(3H)-one;

3-(4-methylphenyl)-4-[3-fluoro-4-(methylsulfonyl)phenyl]-1,3-thiazol-2(3H)-one;

3-(3-cyanophenyl)-4-[3-fluoro-4-(methylsulfonyl)phenyl]-1,3-thiazol-2(3H)-one;

3-(4-cyanophenyl)-4-[3-fluoro-4-(methylsulfonyl)phenyl])-1,3-thiazol-2(3H)-one;

3-(3-trifluoromethylphenyl)-4-[3-fluoro-4-(methyl-sulfonyl)phenyl]-1,3-thiazol-2(3H)-one;

3-(4-trifluoromethylphenyl)-4-[3-fluoro-4-(methyl-sulfonyl)phenyl]-1,3-thiazol-2(3H)-one;

3-(3-trifluoromethoxyphenyl)-4-[3-fluoro-4-(methyl-sulfonyl)phenyl]-1,3-thiazol-2(3H)-one;

3-(4-trifluoromethoxyphenyl)-4-[3-fluoro-4-(methyl-sulfonyl)phenyl]-1,3-thiazol-2(3H)-one;

3-(3,4-dichlorophenyl)-4-[3-fluoro-4-(methylsulfonyl)phenyl]-1,3-thiazol-2(3H)-one;

3-(3,4-dibromophenyl)-4-[3-fluoro-4-(methylsulfonyl)phenyl]-1,3-thiazol-2(3H)-one;

3-(3,4-difluorophenyl)-4-[3-fluoro-4-(methylsulfonyl)phenyl]-1,3-thiazol-2(3H)-one;

3-(3,5-dichlorophenyl)-4-[3-fluoro-4-(methylsulfonyl)phenyl]-1,3-thiazol-2(3H)-one;

3-(3,5-dibromophenyl)-4-[3-fluoro-4-(methylsulfonyl)phenyl]-1,3-thiazol-2(3H)-one;

3-(3,5-difluorophenyl)-4-[3-fluoro-4-(methylsulfonyl)phenyl]-1,3-thiazol-2(3H)-one;

3-(3,4-dimethylphenyl)-4-[3-fluoro-4-(methylsulfonyl)phenyl]-1,3-thiazol-2(3H)-one;

3-(3,5-dimethylphenyl)-4-[3-fluoro-4-(methylsulfonyl)phenyl]-1,3-thiazol-2(3H)-one;

3-(3-methyl-4-chlorophenyl)-4-[3-fluoro-4-(methyl-sulfonyl)phenyl]-1,3-thiazol-2(3H)-one;

3-(4-methyl-3-chlorophenyl)-4-[3-fluoro-4-(methyl-sulfonyl)phenyl]-1,3-thiazol-2(3H)-one;

3-(3-methyl-4-fluorophenyl)-4-[3-fluoro-4-(methyl-sulfonyl)phenyl]-1,3-thiazol-2(3H)-one;

3-(4-methyl-3-fluorophenyl)-4-[3-fluoro-4-(methyl-sulfonyl)phenyl]-1,3-thiazol-2(3H)-one;

3-(3-methyl-4-bromophenyl)-4-[3-fluoro-4-(methyl-sulfonyl)phenyl]-1,3-thiazol-2(3H)-one;

3-(4-methyl-3-bromophenyl)-4-[3-fluoro-4-(methyl-sulfonyl)phenyl]-1,3-thiazol-2(3H)-one;

3-(3-methyl-4-trifluoromethylphenyl)-4-[3-fluoro-4-(methyl-sulfonyl)phenyl]-1,3-thiazol-2(3H)-one;

3-(4-methyl-3-trifluoromethylphenyl)-4-[3-fluoro-4-(methylsulfonyl)phenyl]-1,3-thiazol-2(3H)-one;

3-(3-methyl-4-trifluoromethoxyphenyl)-4-[3-fluoro-4-(methylsulfonyl)phenyl]-1,3-thiazol-2(3H)-one;

3-(4-methyl-3-trifluoromethoxyphenyl)-4-[3-fluoro-4-(methylsulfonyl)phenyl]-1,3-thiazol-2(3H)-one;

3-(3-cyano-4-methylphenyl)-4-[3-fluoro-4-(methyl-sulfonyl)phenyl]-1,3-thiazol-2(3H)-one;

3-(4-cyano-3-methylphenyl)-4-[3-fluoro-4-(methyl-sulfonyl)phenyl]-1,3-thiazol-2(3H)-one;

3-(3-chloro-4-methoxyphenyl)-4-[3-fluoro-4-(methyl-sulfonyl)phenyl]-1,3-thiazol-2(3H)-one;

3-(4-chloro-3-methoxyphenyl)-4-[3-fluoro-4-(methyl-sulfonyl)phenyl]-1,3-thiazol-2(3H)-one;

3-(2-methylpyridin-6-yl)-4-[3-fluoro-4-(methyl-sulfonyl)phenyl]-1,3-thiazol-2(3H)-one;

3-(2-methylthiazol-4-yl)-4-[3-fluoro-4-(methyl-sulfonyl)phenyl]-1,3-thiazol-2(3H)-one;

3-(4-methylthiazol-2-yl)-4-[3-fluoro-4-(methyl-sulfonyl)phenyl]-1,3-thiazol-2(3H)-one;

3-(2-methylpyridin-3-yl)-4-[3-fluoro-4-(methyl-sulfonyl)phenyl]-1,3-thiazol-2(3H)-one;

3-(2-methylpyridin-3-yl)-4-[3-fluoro-4-(methyl-sulfonyl)phenyl]-1,3-thiazol-2(3H)-one;

3-(3-pyridinyl)-4-[3-fluoro-4-(methylsulfonyl)phenyl]-1,3-thiazol-2(3H)-one;

3-(5-methylpyridin-3-yl)-4-[3-fluoro-4-(methylsulfonyl)phenyl]-1,3-thiazol-2(3H)-one;

3-(2-methylpyridin-3-yl)-4-[3-fluoro-4-(methylsulfonyl)phenyl]-1,3-thiazol-2(3H)-one;

3-cyclohexyl-4-[3-fluoro-4-(methylsulfonyl)phenyl]-1,3-thiazol-2(3H)-one; and

3-cyclopentyl-4-[3-fluoro-4-(methylsulfonyl)phenyl]-1,3-thiazol-2(3H)-one;

and the pharmaceutically-acceptable salts, tautomers and prodrugs thereof.

Within Formula I there is another subclass of compounds of interest that includes, but is not limited to:

2-fluoro-4-[3-2-oxo-2,3-dihydro-1,3-oxazol-4-yl]benzenesulfonamide;

2-fluoro-4-[3-(3-chlorophenyl)-2-oxo-2,3-dihydro-1,3-oxazol-4-yl)benzenesulfonamide;

2-fluoro-4-[3-(4-chlorophenyl)-2-oxo-2,3-dihydro-1,3-oxazol-4-yl]benzenesulfonamide;

2-fluoro-4-[3-(3-bromophenyl)-2-oxo-2,3-dihydro-1,3-oxazol-4-yl]benzenesulfonamide;

2-fluoro-4-[3-(4-bromophenyl)-2-oxo-2,3-dihydro-1,3-oxazol-4-yl]benzenesulfonamide;

2-fluoro-4-[3-(3-fluorophenyl)-2-oxo-2,3-dihydro-1,3-oxazol-4-yl]benzenesulfonamide;

2-fluoro-4-[3-(4-fluorophenyl)-2-oxo-2,3-dihydro-1,3-oxazol-4-yl]benzenesulfonamide;

2-fluoro-4-[3-(3-methylphenyl)-4-[3-fluoro-4-(methylsulfonyl)phenyl]-1,3-oxazol-2(3H)-one;

2-fluoro-4-[3-(4-methylphenyl)-2-oxo-2,3-dihydro-1,3-oxazol-4-yl]benzenesulfonamide;

2-fluoro-4-[3-(3-cyanophenyl)-2-oxo-2,3-dihydro-1,3-oxazol-4-yl]benzenesulfonamide;

2-fluoro-4-[3-(4-cyanophenyl)-2-oxo-2,3-dihydro-1,3-oxazol-4-yl]benzenesulfonamide;

2-fluoro-4-[3-(3-trifluoromethylphenyl)-2-oxo-2,3-dihydro-1,3-oxazol-4-yl]benzenesulfonamide;

2-fluoro-4-[3-(4-trifluoromethylphenyl)-2-oxo-2,3-dihydro-1,3-oxazol-4-yl]benzenesulfonamide;

2-fluoro-4-[3-(3-trifluoromethoxyphenyl)-2-oxo-2,3-dihydro-1,3-oxazol-4-yl]benzenesulfonamide;

2-fluoro-4-[3-(4-trifluoromethoxyphenyl)-2-oxo-2,3-dihydro-1,3-oxazol-4-yl]benzenesulfonamide;

2-fluoro-4-[3-(3,4-dichlorophenyl)-2-oxo-2,3-dihydro-1,3-oxazol-4-yl]benzenesulfonamide;

2-fluoro-4-[3-(3,4-dibromophenyl)-2-oxo-2,3-dihydro-1,3-oxazol-4-yl]benzenesulfonamide;

2-fluoro-4-[3-(3,4-difluorophenyl)-2-oxo-2,3-dihydro-1,3-oxazol-4-yl]benzenesulfonamide;

2-fluoro-4-[3-(3,5-dichlorophenyl)-2-oxo-2,3-dihydro-1,3-oxazol-4-yl]benzenesulfonamide;

2-fluoro-4-[3-(3,5-dibromophenyl)-2-oxo-2,3-dihydro-1,3-oxazol-4-yl]benzenesulfonamide;

2-fluoro-4-[3-(3,5-difluorophenyl)-2-oxo-2,3-dihydro-1,3-oxazol-4-yl]benzenesulfonamide;

2-fluoro-4-[3-(3,4-dimethylphenyl)-2-oxo-2,3-dihydro-1,3-oxazol-4-yl]benzenesulfonamide;

2-fluoro-4-[3-(3,5-dimethylphenyl)-2-oxo-2,3-dihydro-1,3-oxazol-4-yl]benzenesulfonamide;

2-fluoro-4-[3-(3-methyl-4-chlorophenyl)-2-oxo-2,3-dihydro-1,3-oxazol-4-yl]benzenesulfonamide;

2-fluoro-4-[3-(4-methyl-3-chlorophenyl)-2-oxo-2,3-dihydro-1,3-oxazol-4-yl]benzenesulfonamide;

2-fluoro-4-[3-(3-methyl-4-fluorophenyl)-2-oxo-2,3-dihydro-1,3-oxazol-4-yl]benzenesulfonamide;

2-fluoro-4-[3-(4-methyl-3-fluorophenyl)-2-oxo-2,3-dihydro-1,3-oxazol-4-yl]benzenesulfonamide;

52-fluoro-4-[3-(3-methyl-4-bromophenyl)-2-oxo-2,3-dihydro-1,3-oxazol-4-yl]benzenesulfonamide;

2-fluoro-4-[3-(4-methyl-3-bromophenyl)-2-oxo-2,3-dihydro-1,3-oxazol-4-yl]benzenesulfonamide;

2-fluoro-4-[3-(3-methyl-4-trifluoromethylphenyl)-2-oxo-2,3-dihydro-1,3-oxazol-4-yl]benzenesulfonamide;

2-fluoro-4-[3-(4-methyl-3-trifluoromethylphenyl)-2-oxo-2,3-dihydro-1,3-oxazol-4-yl]benzenesulfonamide;

2-fluoro-4-[3-(3-methyl-4-trifluoromethoxyphenyl)-2-oxo-2,3-dihydro-1,3-oxazol-4-yl]benzenesulfonamide;

2-fluoro-4-[3-(4-methyl-3-trifluoromethoxyphenyl)-2-oxo-2,3-dihydro-1,3-oxazol-4-yl]benzenesulfonamide;

2-fluoro-4-[3-(3-cyano-4-methylphenyl)-2-oxo-2,3-dihydro-1,3-oxazol-4-yl]benzenesulfonamide;

2-fluoro-4-[3-(4-cyano-3-methylphenyl)-2-oxo-2,3-dihydro-1,3-oxazol-4-yl]benzenesulfonamide;

2-fluoro-4-[3-(3-chloro-4-methoxyphenyl)-2-oxo-2,3-dihydro-1,3-oxazol-4-yl]benzenesulfonamide;

2-fluoro-4-[3-(4-chloro-3-methoxyphenyl)-2-oxo-2,3-dihydro-1,3-oxazol-4-yl]benzenesulfonamide;

2-fluoro-4-[3-(2-methylpyridin-6-yl)-2-oxo-2,3-dihydro-1,3-oxazol-4-yl]benzenesulfonamide;

2-fluoro-4-[3-(2-methylthiazol-4-yl)-2-oxo-2,3-dihydro-1,3-oxazol-4-yl]benzenesulfonamide;

2-fluoro-4-[3-(4-methylthiazol-2-yl)-2-oxo-2,3-dihydro-1,3-oxazol-4-yl]benzenesulfonamide;

2-fluoro-4-[3-(2-methylpyridin-3-yl)-2-oxo-2,3-dihydro-1,3-oxazol-4-yl]benzenesulfonamide;

2-fluoro-4-[3-(2-methylpyridin-3-yl)-2-oxo-2,3-dihydro-1,3-oxazol-4-yl]benzenesulfonamide;

2-fluoro-4-[3-(3-pyridinyl)-2-oxo-2,3-dihydro-1,3-oxazol-4-yl]benzenesulfonamide;

2-fluoro-4-[3-(5-methylpyridin-3-yl)-2-oxo-2,3-dihydro-1,3-oxazol-4-yl]benzenesulfonamide;

2-fluoro-4-[3-(2-methylpyridin-3-yl)-2-oxo-2,3-dihydro-1,3-oxazol-4-yl]benzenesulfonamide;

3-cyclohexyl-2-oxo-2,3-dihydro-1,3-oxazol-4-yl]benzene-sulfonamide;

3-cyclopentyl-2-oxo-2,3-dihydro-1,3-oxazol-4-yl]benzene-sulfonamide;

3-phenyl-4-[3-fluoro-4-(methylsulfonyl)phenyl]-1,3-oxazol-2(3H)-one;

3-(3-chlorophenyl)-4-[3-fluoro-4-(methylsulfonyl)phenyl]-1,3-oxazol-2(3H)-one;

3-(4-chlorophenyl)-4-[3-fluoro-4-(methylsulfonyl)phenyl]-1,3-oxazol-2(3H)-one;

3-(3-bromophenyl)-4-[3-fluoro-4-(methylsulfonyl)phenyl]-1,3-oxazol-2(3H)-one;

3-(4-bromophenyl)-4-[3-fluoro-4-(methylsulfonyl)phenyl]-1,3-oxazol-2(3H)-one;

3-(3-fluorophenyl)-4-[3-fluoro-4-(methylsulfonyl)phenyl]-1,3-oxazol-2(3H)-one;

3-(4-fluorophenyl)-4-[3-fluoro-4-(methylsulfonyl)phenyl]-1,3-oxazol-2(3H)-one;

3-(3-methylphenyl)-4-[3-fluoro-4-(methylsulfonyl)phenyl]-1,3-oxazol-2(3H)-one;

3-(4-methylphenyl)-4-[3-fluoro-4-(methylsulfonyl)phenyl]-1,3-oxazol-2(3H)-one;

3-(3-cyanophenyl)-4-[3-fluoro-4-(methylsulfonyl)phenyl]-1,3-oxazol-2(3H)-one;

3-(4-cyanophenyl)-4-[3-fluoro-4-(methylsulfonyl)phenyl]-1,3-oxazol-2(3H)-one;

3-(3-trifluoromethylphenyl)-4-[3-fluoro-4-(methyl-sulfonyl)phenyl]-1,3-oxazol-2(3H)-one;

3-(4-trifluoromethylphenyl)-4-[3-fluoro-4-(methyl-sulfonyl)phenyl]-1,3-oxazol-2(3H)-one;

3-(3-trifluoromethoxyphenyl)-4-[3-fluoro-4-(methyl-sulfonyl)phenyl]-1,3-oxazol-2(3H)-one;

3-(4-trifluoromethoxyphenyl)-4-[3-fluoro-4-(methyl-sulfonyl)phenyl]-1,3-oxazol-2(3H)-one;

3-(3,4-dichlorophenyl)-4-[3-fluoro-4-(methyl-sulfonyl)phenyl]-1,3-oxazol-2(3H)-one;

3-(3,4-dibromophenyl)-4-[3-fluoro-4-(methyl-sulfonyl)phenyl]-1,3-oxazol-2(3H)-one;

3-(3,4-difluorophenyl)-4-[3-fluoro-4-(methyl-sulfonyl)phenyl]-1,3-oxazol-2(3H)-one;

3-(3,5-dichlorophenyl)-4-[3-fluoro-4-(methyl-sulfonyl)phenyl]-1,3-oxazol-2(3H)-one;

3-(3,5-dibromophenyl)-4-[3-fluoro-4-(methyl-sulfonyl)phenyl]-1,3-oxazol-2(3H)-one;

3-(3,5-difluorophenyl)-4-[3-fluoro-4-(methyl-sulfonyl)phenyl]-1,3-oxazol-2(3H)-one;

3-(3,4-dimethylphenyl)-4-[3-fluoro-4-(methyl-sulfonyl)phenyl]-1,3-oxazol-2(3H)-one;

3-(3,5-dimethylphenyl)-4-[3-fluoro-4-(methyl-sulfonyl)phenyl]-1,3-oxazol-2(3H)-one;

3-(3-methyl-4-chlorophenyl)-4-[3-fluoro-4-(methyl-sulfonyl)phenyl]-1,3-oxazol-2(3H)-one;

3-(4-methyl-3-chlorophenyl)-4-[3-fluoro-4-(methyl-sulfonyl)phenyl]-1,3-oxazol-2(3H)-one;

3-(3-methyl-4-fluorophenyl)-4-[3-fluoro-4-(methyl-sulfonyl)phenyl]-1,3-oxazol-2(3H)-one;

3-(4-methyl-3-fluorophenyl)-4-[3-fluoro-4-(methyl-sulfonyl)phenyl]-1,3-oxazol-2(3H)-one;

3-(3-methyl-4-bromophenyl)-4-[3-fluoro-4-(methyl-sulfonyl)phenyl]-1,3-oxazol-2(3H)-one;

3-(4-methyl-3-bromophenyl)-4-[3-fluoro-4-(methyl-sulfonyl)phenyl]-1,3-oxazol-2(3H)-one;

3-(3-methyl-4-trifluoromethylphenyl)-4-[3-fluoro-4-(methylsulfonyl)phenyl]-1,3-oxazol-2(3H)-one;

3-(4-methyl-3-trifluoromethylphenyl)-4-[3-fluoro-4-(methylsulfonyl)phenyl]-1,3-oxazol-2(3H)-one;

3-(3-methyl-4-trifluoromethoxyphenyl)-4-[3-fluoro-4-(methylsulfonyl)phenyl]-1,3-oxazol-2(3H)-one;

3-(4-methyl-3-trifluoromethoxyphenyl)-4-[3-fluoro-4-(methylsulfonyl)phenyl]-1,3-oxazol-2(3H)-one;

3-(3-cyano-4-methylphenyl)-4-[3-fluoro-4-(methyl-sulfonyl)phenyl]-1,3-oxazol-2(3H)-one;

3-(4-cyano-3-methylphenyl)-4-[3-fluoro-4-(methyl-sulfonyl)phenyl]-1,3-oxazol-2(3H)-one;

3-(3-chloro-4-methoxyphenyl)-4-[3-fluoro-4-(methyl-sulfonyl)phenyl]-1,3-oxazol-2(3H)-one;

3-(4-chloro-3-methoxyphenyl)-4-[3-fluoro-4-(methyl-sulfonyl)phenyl]-1,3-oxazol-2(3H)-one;

3-(2-methylpyridin-6-yl)-4-[3-fluoro-4-(methyl-sulfonyl)phenyl]-1,3-oxazol-2(3H)-one;

3-(2-methylthiazol-4-yl)-4-[3-fluoro-4-(methyl-sulfonyl)phenyl]-1,3-oxazol-2(3H)-one;

3-(4-methylthiazol-2-yl)-4-[3-fluoro-4-(methyl-sulfonyl)phenyl]-1,3-oxazol-2(3H)-one;

3-(2-methylpyridin-3-yl)-4-[3-fluoro-4-(methyl-sulfonyl)phenyl]-1,3-oxazol-2(3H)-one;

3-(2-methylpyridin-3-yl)-4-[3-fluoro-4-(methyl-sulfonyl)phenyl]-1,3-oxazol-2(3H)-one;

3-(3-pyridinyl)-4-[3-fluoro-4-(methylsulfonyl)phenyl]-1,3-oxazol-2(3H)-one;

3-(5-methylpyridin-3-yl)-4-[3-fluoro-4-(methyl-sulfonyl)phenyl]-1,3-oxazol-2(3H)-one;

3-(2-methylpyridin-3-yl)-4-[3-fluoro-4-(methyl-sulfonyl)phenyl]-1,3-oxazol-2(3H)-one;

3-cyclohexyl-4-[3-fluoro-4-(methylsulfonyl)phenyl]-1,3-oxazol-2(3H)-one; and

3-cyclopentyl-4-[3-fluoro-4-(methylsulfonyl)phenyl]-1,3-oxazol-2(3H)-one;

and the pharmaceutically-acceptable salts, tautomers and prodrugs thereof.

Within Formula I there is another subclass of compounds of interest that includes, but is not limited to:

5-phenyl-4-[3-fluoro-4-(methylsulfonyl)phenyl]-2-(difluoromethyl)-1H-imidazole;

5-(3-chlorophenyl)-4-[3-fluoro-4-(methylsulfonyl)phenyl]-2-(difluoromethyl)-1H-imidazole;

5-(4-chlorophenyl)-4-[3-fluoro-4-(methylsulfonyl)phenyl]-2-(difluoromethyl)-1H-imidazole;

5-(3-bromophenyl)-4-[3-fluoro-4-(methylsulfonyl)phenyl]-2-(difluoromethyl)-1H-imidazole;

5-(4-bromophenyl)-4-[3-fluoro-4-(methylsulfonyl)phenyl]-2-(difluoromethyl)-1H-imidazole;

5-(3-fluorophenyl)-4-[3-fluoro-4-(methylsulfonyl)phenyl]-2-(difluoromethyl)-1H-imidazole;

5-(4-fluorophenyl)-4-[3-fluoro-4-(methylsulfonyl)phenyl]-2-(difluoromethyl)-1H-imidazole;

5-(3-methylphenyl)-4-[3-fluoro-4-(methylsulfonyl)phenyl]-2-(difluoromethyl)-1H-imidazole;

5-(4-methylphenyl)-4-[3-fluoro-4-(methylsulfonyl)phenyl]-2-(difluoromethyl)-1H-imidazole;

5-(3-cyanophenyl)-4-[3-fluoro-4-(methylsulfonyl)phenyl]-2-(difluoromethyl)-1H-imidazole;

5-(4-cyanophenyl)-4-[3-fluoro-4-(methylsulfonyl)phenyl]-2-(difluoromethyl)-1H-imidazole;

5-(3-trifluoromethylphenyl)-4-[3-fluoro-4-(methylsulfonyl)phenyl]-2-(difluoromethyl)-1H-imidazole;

5-(4-trifluoromethylphenyl)-4-[3-fluoro-4-(methylsulfonyl)phenyl]-2-(difluoromethyl)-1H-imidazole;

5-(3-trifluoromethoxyphenyl)-4-[3-fluoro-4-(methylsulfonyl)phenyl]-2-(difluoromethyl)-1H-imidazole;

5-(4-trifluoromethoxyphenyl)-4-[3-fluoro-4-(methylsulfonyl)phenyl]-2-(difluoromethyl)-1H-imidazole;

5-(3,4-dichlorophenyl)-4-[3-fluoro-4-(methylsulfonyl)phenyl]-2-(difluoromethyl)-1H-imidazole;

5-(3,4-dibromophenyl)-4-[3-fluoro-4-(methylsulfonyl)phenyl]-2-(difluoromethyl)-1H-imidazole;

5-(3,4-difluorophenyl)-4-[3-fluoro-4-(methylsulfonyl)phenyl]-2-(difluoromethyl)-1H-imidazole;

5-(3,5-dichlorophenyl)-4-[3-fluoro-4-(methylsulfonyl)phenyl]-2-(difluoromethyl)-1H-imidazole;

5-(3,5-dibromophenyl)-4-[3-fluoro-4-(methylsulfonyl)phenyl]-2-(difluoromethyl)-1H-imidazole;

5-(3,5-difluorophenyl)-4-[3-fluoro-4-(methylsulfonyl)phenyl]-2-(difluoromethyl)-1H-imidazole;

5-(3,4-dimethylphenyl)-4-[3-fluoro-4-(methylsulfonyl)phenyl]-2-(difluoromethyl)-1H-imidazole;

5-(3,5-dimethylphenyl)-4-[3-fluoro-4-(methylsulfonyl)phenyl]-2-(difluoromethyl)-1H-imidazole;

5-(3-methyl-4-chlorophenyl)-4-[3-fluoro-4-(methylsulfonyl)phenyl]-2-(difluoromethyl)-1H-imidazole;

5-(4-methyl-3-chlorophenyl)-4-[3-fluoro-4-(methylsulfonyl)phenyl]-2-(difluoromethyl)-1H-imidazole;

5-(3-methyl-4-fluorophenyl)-4-[3-fluoro-4-(methylsulfonyl)phenyl]-2-(difluoromethyl)-1H-imidazole;

5-(4-methyl-3-fluorophenyl)-4-[3-fluoro-4-(methylsulfonyl)phenyl]-2-(difluoromethyl)-1H-imidazole;

5-(3-methyl-4-bromophenyl)-4-[3-fluoro-4-(methylsulfonyl)phenyl]-2-(difluoromethyl)-1H-imidazole;

5-(4-methyl-3-bromophenyl)-4-[3-fluoro-4-(methylsulfonyl)phenyl]-2-(difluoromethyl)-1H-imidazole;

5-(3-methyl-4-trifluoromethylphenyl)-4-[3-fluoro-4-(methylsulfonyl)phenyl]-2-(difluoromethyl)-1H-imidazole;

5-(4-methyl-3-trifluoromethylphenyl)-4-[3-fluoro-4-(methylsulfonyl)phenyl]-2-(difluoromethyl)-1H-imidazole;

5-(3-methyl-4-trifluoromethoxyphenyl)-4-[3-fluoro-4-(methylsulfonyl)phenyl]-2-(difluoromethyl)-1H-imidazole;

5-(4-methyl-3-trifluoromethoxyphenyl)-4-[3-fluoro-4-(methylsulfonyl)phenyl]-2-(difluoromethyl)-1H-imidazole;

5-(3-cyano-4-methylphenyl)-4-[3-fluoro-4-(methyl-sulfonyl)phenyl]-2-(difluoromethyl)-1H-imidazole;

5-(4-cyano-3-methylphenyl)-4-[3-fluoro-4-(methyl-sulfonyl)phenyl]-2-(difluoromethyl)-1H-imidazole;

5-(3-chloro-4-methoxyphenyl)-4-[3-fluoro-4-(methyl-sulfonyl)phenyl]-2-(difluoromethyl)-1H-imidazole;

5-(4-chloro-3-methoxyphenyl)-4-[3-fluoro-4-(methyl-sulfonyl)phenyl]-2-(difluoromethyl)-1H-imidazole;

5-(2-methylpyridin-6-yl)-4-[3-fluoro-4-(methyl-sulfonyl)phenyl]-2-(difluoromethyl)-1H-imidazole;

5-(2-methylthiazol-4-yl)-4-[3-fluoro-4-(methyl-sulfonyl)phenyl]-2-(difluoromethyl)-1H-imidazole;

5-(4-methylthiazol-2-yl)-4-[3-fluoro-4-(methyl-sulfonyl)phenyl]-2-(difluoromethyl)-1H-imidazole;

5-(2-methylpyridin-3-yl)-4-[3-fluoro-4-(methyl-sulfonyl)phenyl]-2-(difluoromethyl)-1H-imidazole;

5-(2-methylpyridin-3-yl)-4-[3-fluoro-4-(methyl-sulfonyl)phenyl]-2-(difluoromethyl)-1H-imidazole;

5-(3-pyridinyl)-4-[3-fluoro-4-(methylsulfonyl)phenyl]-2-(difluoromethyl)-1H-imidazole;

5-(5-methylpyridin-3-yl)-4-[3-fluoro-4-(methylsulfonyl)phenyl]-2-(difluoromethyl)-1H-imidazole;

5-(2-methylpyridin-3-yl)-4-[3-fluoro-4-(methyl-sulfonyl)phenyl]-2-(difluoromethyl)-1H-imidazole;

5-cyclohexyl-4-[3-fluoro-4-(methylsulfonyl)phenyl]-2-(difluoromethyl)-1H-imidazole;

5-cyclopentyl-4-[3-fluoro-4-(methylsulfonyl)phenyl]-2-(difluoromethyl)-1H-imidazole;

5-phenyl-4-[3-fluoro-4-(methylsulfonyl)phenyl]-2-(trifluoromethyl)-1H-imidazole;

5-(3-chlorophenyl)-4-[3-fluoro-4-(methylsulfonyl)phenyl]-2-(trifluoromethyl)-1H-imidazole;

5-(4-chlorophenyl)-4-[3-fluoro-4-(methylsulfonyl)phenyl]-2-(trifluoromethyl)-1H-imidazole;

5-(3-bromophenyl)-4-[3-fluoro-4-(methylsulfonyl)phenyl]-2-(trifluoromethyl)-1H-imidazole;

5-(4-bromophenyl)-4-[3-fluoro-4-(methylsulfonyl)phenyl]-2-(trifluoromethyl)-1H-imidazole;

5-(3-fluorophenyl)-4-[3-fluoro-4-(methylsulfonyl)phenyl]-2-(trifluoromethyl)-1H-imidazole;

5-(4-fluorophenyl)-4-[3-fluoro-4-(methylsulfonyl)phenyl]-2-(trifluoromethyl)-1H-imidazole;

5-(3-methylphenyl)-4-[3-fluoro-4-(methylsulfonyl)phenyl]-2-(trifluoromethyl)-1H-imidazole;

5-(4-methylphenyl)+[3-fluoro-4-(methylsulfonyl)phenyl]-2-(trifluoromethyl)-1H-imidazole;

5-(3-cyanophenyl)-4-[3-fluoro-4-(methylsulfonyl)phenyl]-2-(trifluoromethyl)-1H-imidazole;

5-(4-cyanophenyl)-4-[3-fluoro-4-(methylsulfonyl)phenyl]-2-(trifluoromethyl)-1H-imidazole;

5-(3-trifluoromethylphenyl)-4-[3-fluoro-4-(methylsulfonyl)phenyl]-2-(trifluoromethyl)-1H-imidazole;

5-(4-trifluoromethylphenyl)-4-[3-fluoro-4-(methylsulfonyl)phenyl]-2-(trifluoromethyl)-1H-imidazole;

5-(3-trifluoromethoxyphenyl)-4-[3-fluoro-4-(methylsulfonyl)phenyl]-2-(trifluoromethyl)-1H-imidazole;

5-(4-trifluoromethoxyphenyl)-4-[3-fluoro-4-(methylsulfonyl)phenyl]-2-(trifluoromethyl)-1H-imidazole;

5-(3,4-dichlorophenyl)-4-[3-fluoro-4-(methylsulfonyl)phenyl]-2-(trifluoromethyl)-1H-imidazole;

5-(3,4-dibromophenyl)-4-[3-fluoro-4-(methylsulfonyl)phenyl]-2-(trifluoromethyl)-1H-imidazole;

5-(3,4-difluorophenyl)-4-[3-fluoro-4-(methylsulfonyl)phenyl]-2-(trifluoromethyl)-1H-imidazole;

5-(3,5-dichlorophenyl)-4-[3-fluoro-4-(methylsulfonyl)phenyl]-2-(trifluoromethyl)-1H-imidazole;

5-(3,5-dibromophenyl)-4-[3-fluoro-4-(methylsulfonyl)phenyl]-2-(trifluoromethyl)-1H-imidazole;

5-(3,5-difluorophenyl)-4-[3-fluoro-4-(methylsulfonyl)phenyl]-2-(trifluoromethyl)-1H-imidazole;

5-(3,4-dimethylphenyl)-4-[3-fluoro-4-(methylsulfonyl)phenyl]-2-(trifluoromethyl)-1H-imidazole;

5-(3,5-dimethylphenyl)-4-[3-fluoro-4-(methylsulfonyl)phenyl]-2-(trifluoromethyl)-1H-imidazole;

5-(3-methyl-4-chlorophenyl)-4-[3-fluoro-4-methylsulfonyl)phenyl]-2-(trifluoromethyl)-1H-imidazole;

5-(4-methyl-3-chlorophenyl)-4-[3-fluoro-4-methylsulfonyl)phenyl]-2-(trifluoromethyl)-1H-imidazole;

5-(3-methyl-4-fluorophenyl)-4-[3-fluoro-4-(methylsulfonyl)phenyl]-2-(trifluoromethyl)-1H-imidazole;

5-(4-methyl-3-fluorophenyl)-4-[3-fluoro-4-(methylsulfonyl)phenyl]-2-(trifluoromethyl)-1H-imidazole;

5-(3-methyl-4-bromophenyl)-4-[3-fluoro-4-(methylsulfonyl)phenyl]-2-(trifluoromethyl)-1H-imidazole;

5-(4-methyl-3-bromophenyl)-4-[3-fluoro-4-(methylsulfonyl)phenyl]-2-(trifluoromethyl)-1H-imidazole;

5-(3-methyl-4-trifluoromethylphenyl)-4-[3-fluoro-4-(methylsulfonyl)phenyl]-2-(trifluoromethyl)-1H-imidazole;

5-(4-methyl-3-trifluoromethylphenyl)-4-[3-fluoro-4-(methylsulfonyl)phenyl]-2-(trifluoromethyl)-1H-imidazole;

5-(3-methyl-4-trifluoromethoxyphenyl)-4-[3-fluoro-4-(methylsulfonyl)phenyl]-2-(trifluoromethyl)-1H-imidazole;

5-(4-methyl-3-trifluoromethoxyphenyl)-4-[3-fluoro-4-(methylsulfonyl)phenyl]-2-(trifluoromethyl)-1H-imidazole;

5-(3-cyano-4-methylphenyl)-4-[3-fluoro-4-(methylsulfonyl)phenyl]-2-(trifluoromethyl)-1H-imidazole;

5-(4-cyano-3-methylphenyl)-4-[3-fluoro-4-(methylsulfonyl)phenyl]-2-(trifluoromethyl)-1H-imidazole;

5-(3-chloro-4-methoxyphenyl)-4-[3-fluoro-4-(methylsulfonyl)phenyl]-2-(trifluoromethyl)-1H-imidazole;

5-(4-chloro-3-methoxyphenyl)-4-[3-fluoro-4-(methylsulfonyl)phenyl]-2-(trifluoromethyl)-1H-imidazole;

5-(2-methylpyridin-6-yl)-4-[3-fluoro-4-(methylsulfonyl)phenyl]-2-(trifluoromethyl)-1H-imidazole;

5-(2-methylthiazol-4-yl)-4-[3-fluoro-4-(methylsulfonyl)phenyl]-2-(trifluoromethyl)-1H-imidazole;

5-(4-methylthiazol-2-yl)-4-[3-fluoro-4-(methylsulfonyl)phenyl]-2-(trifluoromethyl)-1H-imidazole;

5-(2-methylpyridin-3-yl)-4-[3-fluoro-4-(methylsulfonyl)phenyl]-2-(trifluoromethyl)-1H-imidazole;

5-(2-methylpyridin-3-yl)-4-[3-fluoro-4-(methylsulfonyl)phenyl]-2-(trifluoromethyl)-1H-imidazole;

5-(3-pyridinyl)-4-[3-fluoro-4-(methylsulfonyl)phenyl]-2-(trifluoromethyl)-1H-imidazole;

5-(5-methylpyridin-3-yl)-4-[3-fluoro-4-(methylsulfonyl)phenyl]-2-(trifluoromethyl)-1H-imidazole;

5-(2-methylpyridin-3-yl)-4-[3-fluoro-4-(methylsulfonyl)phenyl]-2-(trifluoromethyl)-1H-imidazole;

5-cyclohexyl-4-[3-fluoro-4-(methylsulfonyl)phenyl]-2-(trifluoromethyl)-1H-imidazole;

5-cyclopentyl-4-[3-fluoro-4-(methylsulfonyl)phenyl]-2-(trifluoromethyl)-1H-imidazole;

and the pharmaceutically-acceptable salts, tautomers and prodrugs thereof.

Definitions

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

Where the term “alkyl” is used, either alone or within other terms such as “haloalkyl”, “alkylsulfonyl” and “alkoxyalkyl”, it embraces linear or branched radicals having one to about twenty carbon atoms or, preferably, one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. Even more preferred are lower alkyl radicals having one to three carbon atoms.

Where the term “alkenyl” is used, either alone or within other terms such as “arylalkenyl”, it embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The terms “alkenyl” and “lower alkenyl”, embrace radicals having “cis” and “trans” orientations, or alternatively, “E” and “Z” orientations.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “cycloalkyl” embraces saturated carbocyclic radicals having three to about twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “cycloalkenyl” embraces partially saturated carbocyclic radicals having three to twelve carbon atoms. Cycloalkenyl radicals that are partially saturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “halo” and “halogen” means halogens such as fluorine, chlorine, bromine or iodine atoms. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl” means alkyl radicals having all hydrogen atoms replaced with fluoro atoms. Examples include trifluoromethyl and pentafluoroethyl.

The terms “hydroxyalkyl” and “hydroxylalkyl” embrace linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “cyanoalkyl” embraces linear or branched alkyl radicals having one to about ten carbon atoms any one of which may be substituted with one or more cyano radicals. More preferred cyanoalkyl radicals are “lower cyanoalkyl” radicals having one to six carbon atoms and one cyano radical. Even more preferred are lower cyanoalkyl radicals having one to three carbon atoms. Examples of such radicals include cyanomethyl.

The term “aryl”, alone or in combination, means a carbocyclic aromatic system containing one or two rings wherein such rings may be attached together in a pendent manner or may be fused. The term “aryl” embraces aromatic radicals such as phenyl, naphthyl, tetrahydronaphthyl, indane and biphenyl. More preferred aryl is phenyl. Said “aryl” group may have one to three substituents such as lower alkyl, hydroxy, halo, haloalkyl, nitro, cyano, alkoxy and lower alkylamino.

The term “heterocyclyl” or “heterocyclo” embraces 3-10 membered saturated, partially saturated and unsaturated heteroatom-containing ring-shaped radicals, where the heteroatoms may be selected from nitrogen, sulfur and oxygen. More preferred heterocyclyl are 5-8 membered ring heterocyclyl. Examples of saturated heterocyclic radicals include saturated 3 to 6-membered heteromonocyclic groups containing 1 to 4 nitrogen atoms [e.g. pyrrolidinyl, imidazolidinyl, piperidino, piperazinyl]; saturated 3 to 6-membered heteromonocyclic groups containing 1 to 2 oxygen atoms and 1 to 3 nitrogen atoms [e.g. morpholinyl]; saturated 3 to 6-membered heteromonocyclic groups containing 1 to 2 sulfur atoms and 1 to 3 nitrogen atoms [e.g., thiazolidinyl]. Examples of partially saturated heterocyclyl radicals include dihydrothiophene, dihydropyran, dihydrofuran and dihydrothiazole. Examples of unsaturated heterocyclic radicals, also termed “heteroaryl” radicals, include unsaturated 5 to 6 membered heteromonocyclyl groups containing 1 to 4 nitrogen atoms, for example, pyrrolinyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated condensed heterocyclic groups containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated 3 to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, isoxazolyl, oxadiazolyl [e.g., 1,2,4-oxadiazolyl, 1,3,4-oxadiazolyl, 1,2,5-oxadiazolyl]; unsaturated condensed heterocyclic groups containing 1 to 2 oxygen atoms and 1 to 3 nitrogen atoms [e.g. benzoxazolyl, benzoxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]; unsaturated condensed heterocyclic groups containing 1 to 2 sulfur atoms and 1 to 3 nitrogen atoms [e.g., benzothiazolyl, benzothiadiazolyl] and the like. The term also embraces radicals where heterocyclic radicals are fused with aryl radicals. Examples of such fused bicyclic radicals include benzofuran, benzothiophene, and the like. Said “heterocyclyl” group may have 1 to 3 substituents such as lower alkyl, hydroxy, oxo, amino and lower alkylamino.

Heterocyclic radicals can include 3-10 membered fused or unfused radicals. Preferred examples of heteroaryl radicals include benzofuryl, 2,3-dihydrobenzofuryl, benzothienyl, indolyl, dihydroindolyl, chromanyl, benzopyran, thiochromanyl, benzothiopyran, benzodioxolyl, benzodioxanyl, pyridyl, thienyl, thiazolyl, furyl, and pyrazinyl. More preferred heteroaryl radicals are 5- or 6-membered heteroaryl, containing one or two heteroatoms selected from sulfur nitrogen and oxygen, selected from thienyl, furanyl, thiazolyl, imidazolyl, pyrazolyl, isoxazolyl, isothiazolyl, pyridyl, piperidinyl and pyrazinyl.

The term “aralkyl” embraces aryl-substituted alkyl radicals. Preferable aralkyl radicals are “lower aralkyl” radicals having aryl radicals attached to alkyl radicals having one to six carbon atoms. Even more preferred are lower aralkyl radicals having phenyl attached to alkyl portions having one to three carbon atoms. Examples of such radicals include benzyl, diphenylmethyl and phenylethyl. The aryl in said aralkyl may be additionally substituted with halo, alkyl, alkoxy, halkoalkyl and haloalkoxy. The term “arylalkenyl” embraces aryl-substituted alkenyl radicals. Preferable arylalkenyl radicals are “lower arylalkenyl” radicals having aryl radicals attached to alkenyl radicals having two to six carbon atoms. Examples of such radicals include phenylethenyl. The aryl in said arylalkenyl may be additionally substituted with halo, alkyl, alkoxy, halkoalkyl and haloalkoxy. The aryl in said aralkyl and arylalkenyl may be additionally substituted with halo, alkyl, alkoxy, halkoalkyl and haloalkoxy.

The terms benzyl and phenylmethyl are interchangeable.

The term “heterocyclylalkyl” embraces heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkyl radicals having alkyl portions of one to three carbon atoms. Examples include such radicals as pyridylmethyl and thienylmethyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. The “alkoxy” radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloro-4-methoxy, trifluoromethoxy, trifluoroethoxy, fluoroethoxy and fluoropropoxy. The term “alkoxyalkyl” embraces alkyl radicals having one or more alkoxy radicals attached to the alkyl radical, that is, to form monoalkoxyalkyl and dialkoxyalkyl radicals.

The term “aryloxy” embraces aryl radicals attached through an oxygen atom to other radicals. The term “aralkoxy” embraces aralkyl radicals attached through an oxygen atom to other radicals. The term “aryloxyalkyl” embraces aryloxy radicals as described above attached through the oxygen atom to an alkyl radical. The term “heterocyclyloxy” embraces heterocyclyl radicals attached through an oxygen atom to other radicals.

The term “alkylsulfinyl” embraces radicals containing a linear or branched alkyl radical, of one to ten carbon atoms, attached to a divalent —S(═O)— atom. More preferred are lower alkylsulfinyl radicals having one to three carbon atoms.

The term “sulfonyl”, whether used alone or linked to other terms such as “alkylsulfonyl” and “arylsulfonyl”, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Even more preferred are lower alkylsulfonyl radicals having one to three carbon atoms. Examples of such lower alkylsulfonyl radicals include methylsulfonyl, ethylsulfonyl and propylsulfonyl. “Arylsulfonyl” embraces aryl radicals attached to a sulfonyl radical, where aryl is defined as above. A preferred arylsulfonyl radical is phenylsulfonyl.

The terms “sulfamyl,” “aminosulfonyl” and “sulfonamidyl,” whether alone or used with terms such as “N-alkylaminosulfonyl”, “N-arylaminosulfonyl”, “N,N-dialkylaminosulforiyl” and “N-alkyl-N-arylaminosulfonyl”, denotes a sulfonyl radical substituted with an amine radical, forming a sulfonamide (—SO₂NH₂). The term “alkylaminosulfonyl” includes “N-alkylarminosulfonyl” and “N,N-dialkylaminosulfonyl” where sulfamyl radicals are substituted, respectively, with one alkyl radical, or two alkyl radicals. More preferred alkylaminosulfonyl radicals are “lower alkylaminosulfonyl” radicals having one to six carbon atoms. Even more preferred are lower alkylaminosulfonyl radicals having one to three carbon atoms. Examples of such lower alkylaminosulfonyl radicals include N-methylaminosulfonyl, N-ethylaminosulfonyl and N-methyl-N-ethylaminosulfonyl. The terms “N-arylaminosulfonyl” and “N-alkyl-N-arylaminosulfonyl” denote sulfamyl radicals substituted, respectively, with one aryl radical, or one alkyl and one aryl radical. More preferred N-alkyl-N-arylaminosulfonyl radicals are “lower N-alkyl-N-arylsulfonyl” radicals having alkyl radicals of one to six carbon atoms. Even more preferred are lower N-alkyl-N-arylsulfonyl radicals having one to three carbon atoms. Examples of such lower N-alkyl-N-aryl-aminosulfonyl radicals include N-methyl-N-phenylaminosulfonyl and N-ethyl-N-phenylaminosulfonyl. Examples of such N-aryl-aminosulfonyl radicals include N-phenylaminosulfonyl.

The term “alkylthio” embraces radicals containing a linear or branched alkyl radical, of one to ten carbon atoms, attached to a divalent sulfur atom. Even more preferred are lower alkylthio radicals having one to three carbon atoms. An example of “alkylthio” is methylthio, (CH₃—S—). The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “arylthio” embraces aryl radicals of six to ten carbon atoms, attached to a divalent sulfur atom. An example of “arylthio” is phenylthio. The term “aralkylthio” embraces aralkyl radicals as described above, attached to a divalent sulfur atom. More preferred are phenyl-C₁-C₃-alkylthio radicals. An example of “aralkylthio” is benzylthio. The term “arylthioalkyl” embraces arylthio radicals as described above, through the sulfur atom to an alkyl radical.

The terms “carboxy” or “carboxyl”, whether used alone or with other terms, such as “carboxyalkyl”, denotes —CO₂H. The term “carboxyalkyl” embraces radicals having a carboxy radical as defined above, attached to an alkyl radical.

The term “carbonyl”, whether used alone or with other terms, such as “alkylcarbonyl”, denotes —(C═O)—.

The term “alkylcarbonyl” embraces radicals having a carbonyl radical substituted with an alkyl radical. More preferred alkylcarbonyl radicals are “lower alkylcarbonyl” radicals having one to six carbon atoms. Even more preferred are lower alkylcarbonyl radicals having one to three carbon atoms. The term “alkylcarbonyl” includes radicals having alkyl, hydroxylalkyl, radicals, as defined herein, attached to a carbonyl radical. Examples of such radicals include substituted or unsubstituted methylcarbonyl, ethylcarbonyl, propylcarbonyl, butylcarbonyl, pentylcarbonyl, hydroxymethylcarbonyl, hydroxyethylcarbonyl.

The term “arylcarbonyl” embraces radicals having a carbonyl radical substituted with an aryl radical. More preferred arylcarbonyl radicals include phenylcarbonyl. The term “arylalkylcarbonyl” embraces radicals having a carbonyl radical substituted with an arylalkyl radical. More preferred radicals are phenyl-C₁-C₃-alkylcarbonyl, including benzylcarbonyl.

The term “alkoxycarbonyl” means a radical containing an alkoxy radical, as defined above, attached via an oxygen atom to a carbonyl radical. Preferably, “lower alkoxycarbonyl” embraces alkoxy radicals having one to six carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl. Even more preferred are lower alkoxycarbonyl radicals having alkoxy portions of one to three carbon atoms. The term “alkoxycarbonylalkyl” embraces alkyl radicals substituted with an alkoxycarbonyl radical as defined above. More preferred are “lower alkoxycarbonylalkyl” radicals with alkyl portions having one to six carbons. Examples of such lower alkoxycarbonylalkyl radicals include substituted or unsubstituted methoxycarbonylmethyl, ethoxycarbonylmethyl, methoxycarbonylethyl and ethoxycarbonylethyl.

The term “aminocarbonyl” when used by itself or with other terms such as “aminocarbonylalkyl”, “N-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl” and “N-alkyl-N-hydroxyaminocarbonylalkyl”, denotes an amide group of the formula —C(═O)NH₂. The terms “N-alkylaminocarbonyl” and “N,N-dialkylaminocarbonyl” denote aminocarbonyl radicals which have been substituted with one alkyl radical and with two alkyl radicals, respectively. More preferred are “lower alkylaminocarbonyl” having lower alkyl radicals as described above attached to an aminocarbonyl radical. The terms “N-arylaminocarbonyl” and “N-alkyl-N-arylaminocarbonyl” denote aminocarbonyl radicals substituted, respectively, with one aryl radical, or one alkyl and one aryl radical.

The term “aminoalkyl” embraces alkyl radicals substituted with amino radicals. The term “alkylaminoalkyl” embraces aminoalkyl radicals having the nitrogen atom substituted with an alkyl radical. Even more preferred are lower alkylaminoalkyl radicals having one to three carbon atoms.

The terms “N-alkylamino” and “N,N-dialkylamino” denote amino groups which have been substituted with one alkyl radical and with two alkyl radicals, respectively. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable “alkylamino” may be mono or dialkylamino such as N-methylamino, N-ethylamino, N,N-dimethylamino, N,N-diethylamino or the like. The term “aryl amino” denotes amino groups which have been substituted with one or two aryl radicals, such as N-phenylamino. The “arylamino” radicals may be further substituted on the aryl ring portion of the radical. The term “aralkylamino” denotes amino groups that have been substituted with one or two aralkyl radicals. More preferred are phenyl-C₁-C₃-alkylamino radicals, such as N-benzylamino. The “aralkylamino” radicals may be further substituted on the aryl ring portion of the radical. The terms “N-alkyl-N-arylamino” and “N-aralkyl-N-alkylamino” denote amino groups which have been substituted with one aralkyl and one alkyl radical, or one aryl and one alkyl radical, respectively, to an amino group.

The term “aminocarbonyl” denotes an amide group of the formula —C(═O)NH₂. The term “alkylaminocarbonyl” denotes an aminocarbonyl group that has been substituted with one or two alkyl radicals on the amino nitrogen atom. Preferred are “N-alkylaminocarbonyl” and “N,N-dialkylaminocarbonyl” radicals. More preferred are “lower N-alkylaminocarbonyl” and “lower N,N-dialkylaminocarbonyl” radicals with lower alkyl portions as defined above. The term “alkylaminoalkyl” embraces radicals having one or more alkyl radicals attached to an aminoalkyl radical. The term “arylaminoalkyl” embraces radicals having one or more aryl radicals attached to an aminoalkyl radical.

The additional terms used to describe the substituents of the compounds of Formulae I-VII and not specifically defined herein are defined in a similar manner to that illustrated in the above definitions.

The terms “treatment” and “treating” refers to any process, action, application, therapy, or the like, wherein a subject, including a human being, is provided medical aid with the object of improving the subject's condition, directly or indirectly, or slowing the progression of a condition or disorder in the subject.

The term “prevention” or “prophylaxis” includes either preventing the onset of clinically evident inflammation or inflammation related disorders altogether or preventing the onset of a preclinically evident stage of inflammation or an inflammation related disorder in individuals.

The term “therapeutically-effective” is intended to qualify the amount of each agent that will achieve the goal of improvement in disease severity and the frequency of incidence while avoiding adverse side effects typically associated with alternative therapies.

The term “prodrug” refers to a compound that is a drug precursor that, following administration to a subject and subsequent absorption, is converted to an active species in vivo via some process, such as metabolic conversion. Other products from the conversion process are easily disposed of by the body. More preferred prodrugs produce products from the conversion process that are generally accepted as safe. By way of illustration and not limitation, U.S. Pat. No. 5,932,598 describes prodrug forms of compounds that are substituted sulfonamide compounds that selectively inhibit cyclooxygenase-2. For example, the prodrug may be an acylated form of the active compound such as an acylated sulfonamide.

The term “co-therapy” (or “combination-therapy”), in defining use of a cyclooxygenase-2 inhibitor agent and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent.

Stereoisomers, Tautomers, Protected Acids and Salts

Also included in the family of compounds of Formulae I through VII are the stereoisomers thereof including, but not limited to enantiomers, diastereomers, racemic mixtures and other mixtures thereof.

Also included in the family of compounds of Formulae I through VII are the tautomeric forms of those compounds.

Also included in the family of compounds of Formulae I through VII are the protected acids thereof, such as the esters, hydroxyamino derivatives, amides and sulfonamides. Thus, for example, primary and secondary amines can be reacted with carboxylic acid substituted forms of Formula I-VII to form amides which can be useful as prodrugs. Preferred amines are heterocyclicamines, including optionally substituted aminothiazoles, optionally substituted amino-isoxazoles, and optionally substituted aminopyridines; aniline derivatives; sulfonamides; aminocarboxylic acids; and the like. The esters, hydroxyamino derivatives and sulfonamides can be prepared from the acids by methods known to one skilled in the art.

Also included in the family of compounds of Formulae I-VII are the pharmaceutically-acceptable salts thereof. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formulae I-VII may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, N-hydroxybutyric, salicyclic, galactaric and galacturonic acid. Suitable pharmaceutically-acceptable base addition salts of compounds of Formulae I-VII include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. All of these salts may be prepared by conventional means from the corresponding compounds of the invention by reacting, for example, the appropriate acid or base with the compounds of Formulae I-VII.

Pharmaceutical Compositions

Also embraced within this invention is a class of pharmaceutical compositions comprising the one or more of the active compounds of Formulae I-VII in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended. The active compounds and compositions may, for example, be administered orally, pulmonary, mucosally, intravascularly, intraperitoneally, subcutaneously, intramuscularly or topically.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. The active ingredient may also be administered by injection as a composition wherein, for example, saline, dextrose or water may be used as a suitable carrier.

The amount of therapeutically active compounds which are administered and the dosage regimen for treating a disease condition with the compounds and/or compositions of this invention depends on a variety of factors, including the age, weight, sex and medical condition of the subject, the severity of the disease, the route and frequency of administration, and the particular compound employed, and thus may vary widely. The pharmaceutical compositions may contain active ingredients in the range of about 0.1 to 2000 mg, preferably in the range of about 0.5 to 500 mg and most preferably between about 1 and 100 mg. A daily dose of about 0.01 to 100 mg/kg body weight, preferably between about 0.5 and about 20 mg/kg body weight and most preferably between about 0.1 to 10 mg/kg body weight, may be appropriate. The daily dose can be administered in one to four doses per day.

In the case of psoriasis and other skin conditions, it may be preferable to apply a topical preparation of compounds of this invention to the affected area two to four times a day.

For inflammations of the eye or other external tissues, e.g., mouth and skin, the formulations are preferably applied as a topical ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base. Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% w/w.

For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

For pulmonary administration, the pharmaceutical composition may be administered in the form of an aerosol or with an inhaler including dry powder aerosol.

General Synthetic Procedures

The compounds of the invention can be synthesized according to the procedures set forth below. The substituents of the compounds shown in the following procedures generally have the same definition as the substituents at the corresponding position in the compounds of Formulae I-VII, except where further noted. For example, unless otherwise noted, R¹, R² and R³ as used in the procedures below correspond to R¹, R² and R³ as previously defined; R^{3A} and R^{3B} correspond to substituents independently selected from R³ as previously defined; and R^{S} corresponds to a functional group selected from the group consisting of hydrogen and the optional substituents previously defined for the R¹ cyclohexyl, pyridinyl and phenyl moieties. Unless otherwise noted, X as used in the procedures below corresponds to halogen.

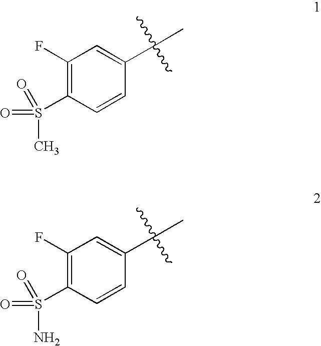

The 3-fluoro-4-methylsulfonylphenyl 1 and 3-fluoro-4-aminosulfonylphenyl 2 are specific regiochemically substituted aromatic rings present in the cyclooxygenase-2 inhibiting diaryl-heterocycles disclosed in this application. The described 3-fluoro-4-methyl-sulfonyphenyl 1 and 3-fluoro-4-amino-sulfonylphenyl 2 ring functionality can be prepared using the synthetic methodology outlined below for the various diaryl substituted heterocycles disclosed in this application.

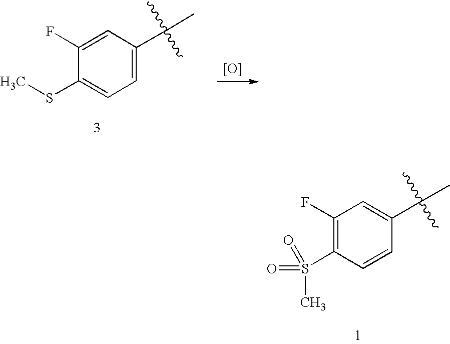

The methythio 3 group can be converted to the methylsulfone 1 through treatment with at least two molar oxidizing equivalents of reagents such as m-chloroperbenzoic acid, monoperoxyphthalic acid, peroxides, or OXONE®.

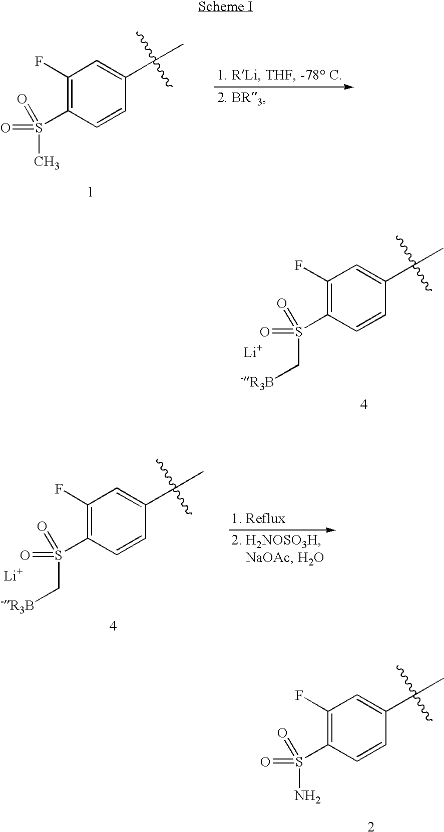

The sulfonamide 2 can be obtained from the sulfone 1 by treatment with an alkyl base such as butyllithium, methyllithium, and the like in ethereal solvents such as tetrahydrofuran at approximately −78° C. In a second step, a trialkyl-borane, such as triethylborane or tributylborane, is added affording the intermediate borate 4 which is warmed to room temperature prior to refluxing for 16 hours or an appropriate period of time. The solution is cooled to room temperature and water, sodium acetate and hydroxylamine-O-sulfonic acid are added to yield the sufonamide 2 [Huang; Tetrahedron Letters, 35, 7201-7204-(1994)].

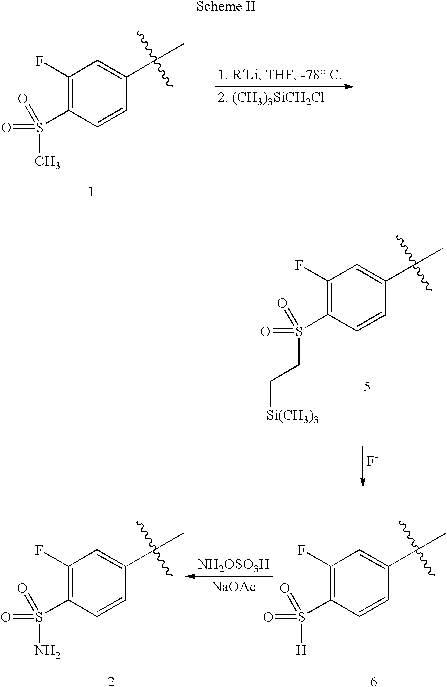

In Scheme II, the sulfonamide 2 can be obtained by treatment of the sulfone 1 with an alkyl base such as butyllithium. Addition of trimethylsilyl-methylchloride affords the trimethylsilylethylsulfone 5. Desilylation by the addition of a reagent such as tetra-n-butyl ammonium fluoride to a solution of the sulfone 5 followed by in situ ethylene extrusion affords the sulfinic acid 6 [Vhu; Steriods, 67, 543-545 (1997)]. The sulfinic acid 6 can be converted to the sulfonamide 2 by the addition of sodium acetate and hydroxylamine-O-sulfonic acid.

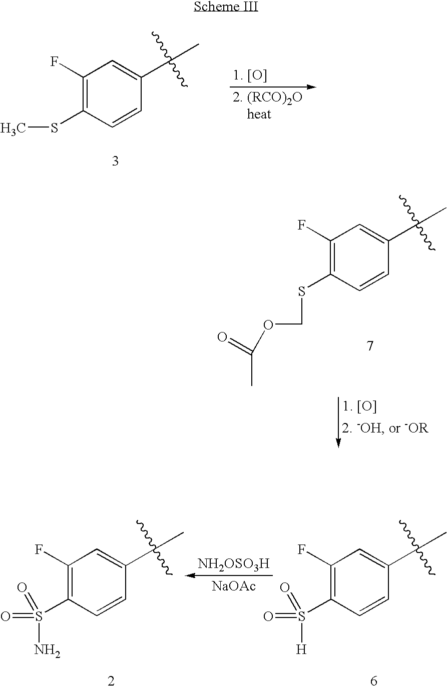

In Scheme III sulfonamides 2 can be prepared from the methylthio group 3 by conversion of the methythio moiety to the sulfoxide by careful addition of one equivalent of an oxidizing agent such as m-chloroperbenzoic acid, monoperoxyphthalic acid, a peroxide, or OXONE®. A Pummer rearrangement performed by mixing the resulting sulfoxide in anhydrides followed by heating provides the thioacetal 7. Oxidation to the sulfone as described earlier followed by treatment with either hydroxide or alkoxides provides sulfinic acid 6 [Vleeschauwer; Syn. Lett., 4, 375-377, (1997)] which can be converted to the sulfonamide 2 by the addition of a suitable base such as sodium acetate and hydroxylamine-O-sulfonic acid in aqueous alcoholic solvents such as methanol-water or ethanol-water.

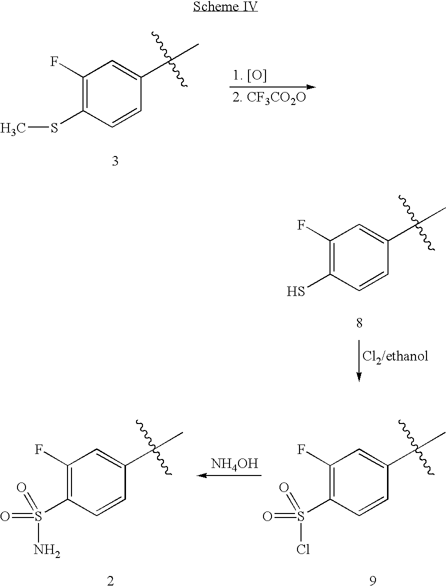

Alternatively, Scheme IV illustrates yet another procedure for the preparation of the sulfonamide 2 starting with the corresponding methylthio moiety. First, conversion of the methythio 3 moiety to the methylsulfoxide is accomplished by careful addition of one equivalent of an oxidizing agent such as m-chloro-perbenzoic acid, monoperoxyphthalic acid, a peroxide, or OXONE®. The methylsulfoxide is then mixed in a variety of inert solvents such as dichloromethane with trifluoroacetic anhydride which, after aqueous workup, provides the sulfide 8. The sulfide 8 is treated with chlorine providing the sulfonylchloride 9. Finally, addition of ammonia to the sulfonylchloride 9 affords the desired sulfonamide 2 [Kharash, J. Am. Chem. Soc., 73, 3240 (1951)].

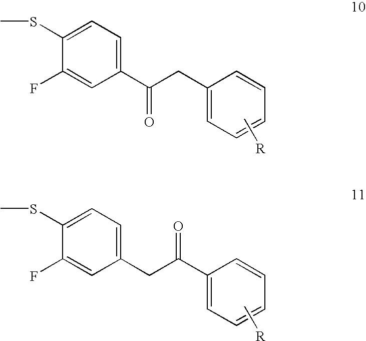

3-Fluoro-4-methylthiophenyl-substituted-phenyl-ethanones 10 and 11 are intermediates used in the preparation of many of the diaryl-heterocycles disclosed in this application. The following discussion illustrates representative methods for the preparation of these intermediates.

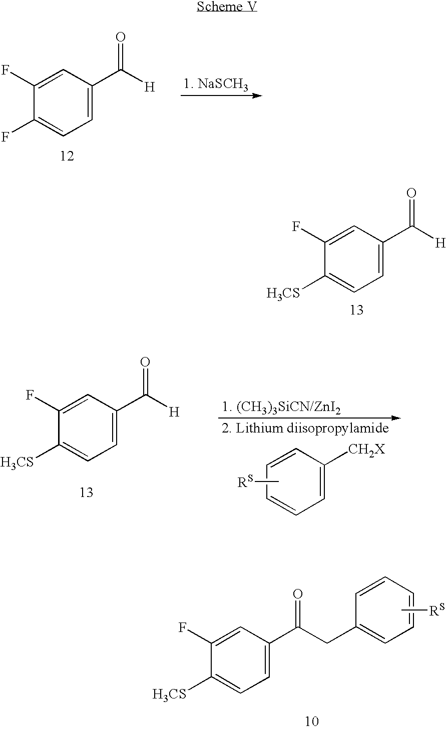

Scheme V outlines a preparation of a deoxybenzoin 10. Mixing commercially available 3,4-difluorobenzaldehyde 12 with sodium thiomethoxide in polar solvents such as acetonitrile or dimethylformamide produces the 3-fluoro-4-methylthio-benzldehyde 13. A solution of the aldehyde, zinc iodide, and trimethylsilyl cyanide in a halogenated solvent such as dichloromethane is mixed at room temperature to afford a trimethylsilylcyanohydrin. Deprotonation of the cyanohydrin with a base such as lithium diisopropylamide or lithium hexamethyl-disilylamide followed by addition of an appropriately substituted benzylhalide and acid-base workup of the reaction yields the 1-(3-fluoro-4-methylthiophenyl)-2-(substituted-phenyl)-ethanone 10.

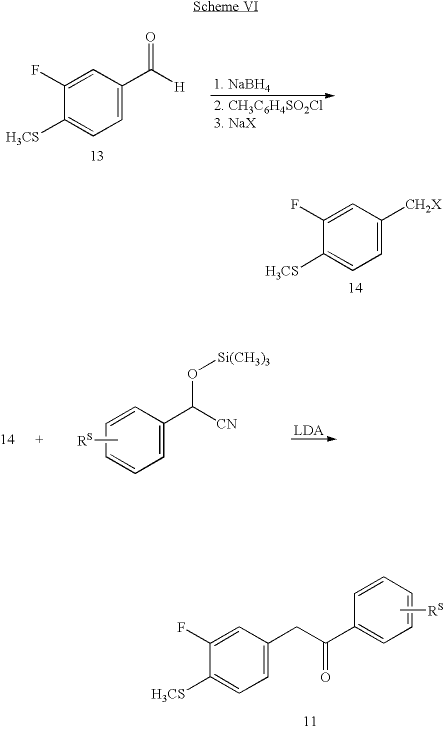

A synthetic scheme for the preparation the 3-fluoro-4-methylthiophenyl-substituted-phenyl-ethanone 11, which have the alternative regiochemistry, is outlined in Scheme VI. Reduction of the 3-fluoro-4-methylthiobenzaldehyde 13 with hydride reagents such as sodium borohydcide, lithium aluminum hydride, and the like affords the benzyl alcohol. Conversion of benzyl alcohol to a benzyl halide 14 can be accomplished through the preparation of the tosylate followed by displacement with chloride or bromide ion (represented by X in Scheme VI). These compounds can be utilized in the synthetic methodology described previously to afford 1-(substituted-phenyl)-2-(3-fluoro-4-methylthiophenyl)-ethanone 11.

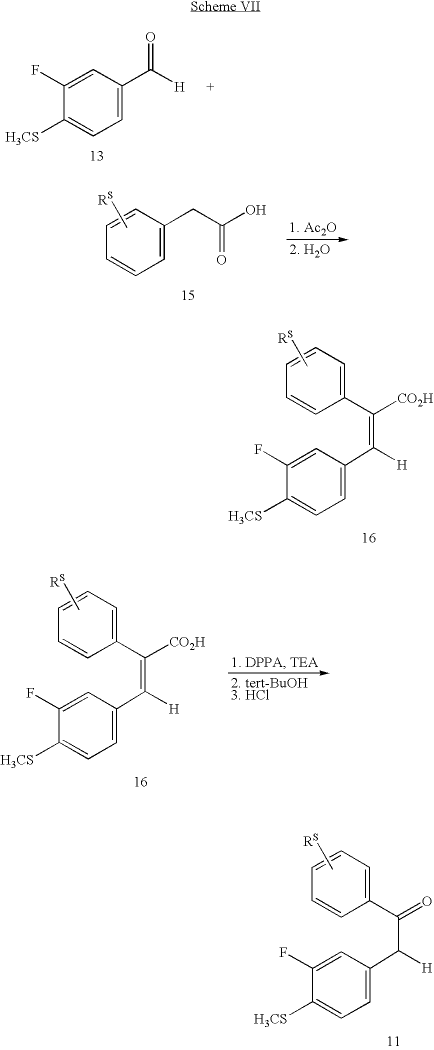

Scheme VII illustrates a procedure that can be used to prepare the 1-(substituted-phenyl)-2-(3-fluoro-4-methylthiophenyl)-ethanone 11 from the 3-fluoro-4-methylthiobenzaldehyde 13. In step one, the aldehyde 13 and substituted phenylacetic acid are heated in acetic anhydride and triethylamine which, upon aqueous quenching, affords the 2,3-disubstituted acrylic acid 16. Mixing of the acrylic acid 16 with diphenylphosphorylazide (DPPA) and triethylamine produces an acylazide. The acylazide undergoes a Curtius rearrangement to an isocyanate which is trapped with tert-butanol yielding an N-tert-butylcarboxy-carbamate. Treatment of the carbamate with concentrated aqueous hydrochloric acid provides the substituted ketone 11.

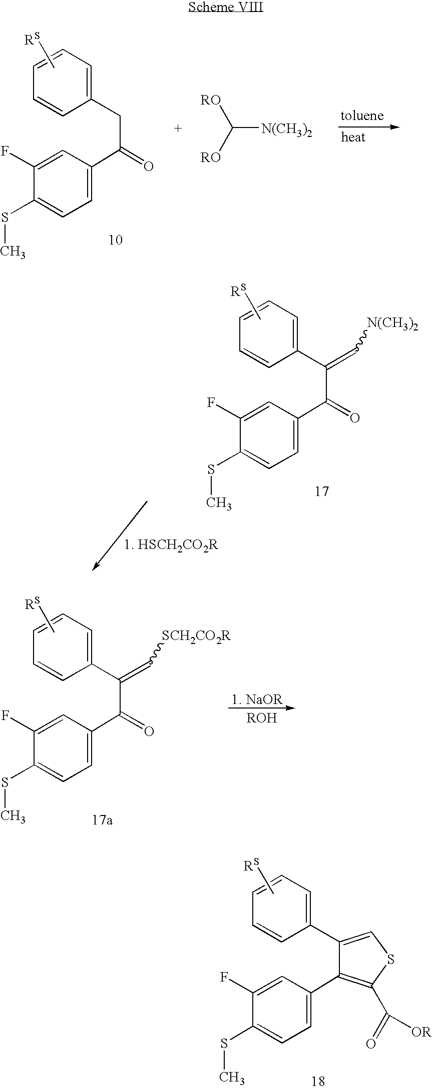

The preparation of 3,4-diarylthiophene 18 is illustrated in Scheme VIII. 3-Fluoro-4-methylthiophenyl-substituted-phenyl ethanone 10 and an acetal of dimethylformamide are refluxed together in toluene. Upon removal of the solvent and excess acetal, the en amine 17 is obtained. The enamine 17 is refluxed in 1,2 dichloroethane with esters of thioacetic acid affording the mixture of Michael addition products 17a. The 1,2-dichloroethane is removed at reduced pressure and the residue is taken up in an alcoholic solvent and the related sodium alkoxide added. Upon mixing at room temperature the desired trisubstituted thiophene 18 is obtained after purification.

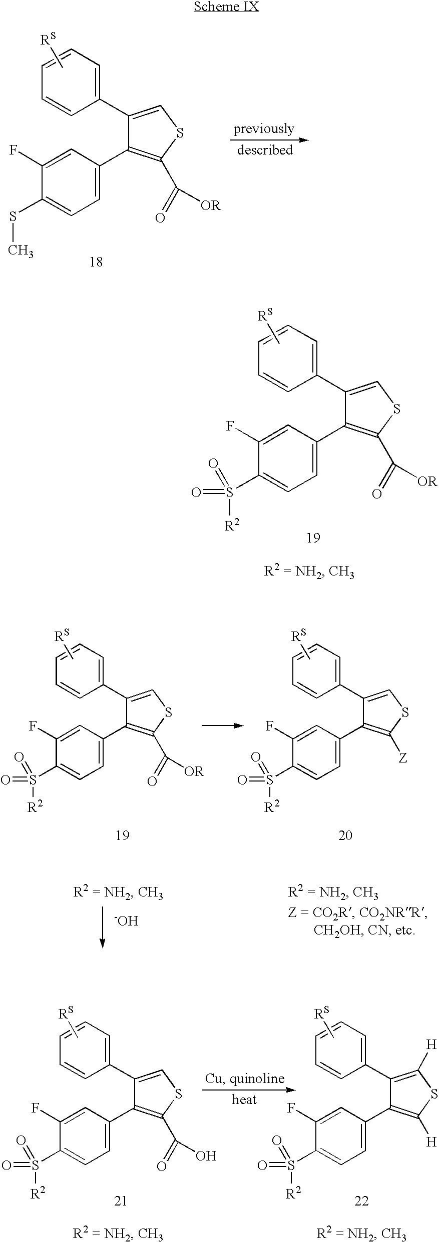

As illustrated in Scheme IX, the methylthiomoiety 18 can be converted to the methyl sulphone and sulfonamide 19 as described previously. The ester group of thiophene 19 can then be manipulated using conventional organic laboratory procedures into a number of functional groups such as alcohol, alkyl, alkenyl, alkynyl, amide, cyano, etc. The ester group also can be saponified and the resulting carboxylic acid 21 removed through a copper mediated decarboxylation affording the 3-4 substituted diphenylthiophenes 22.

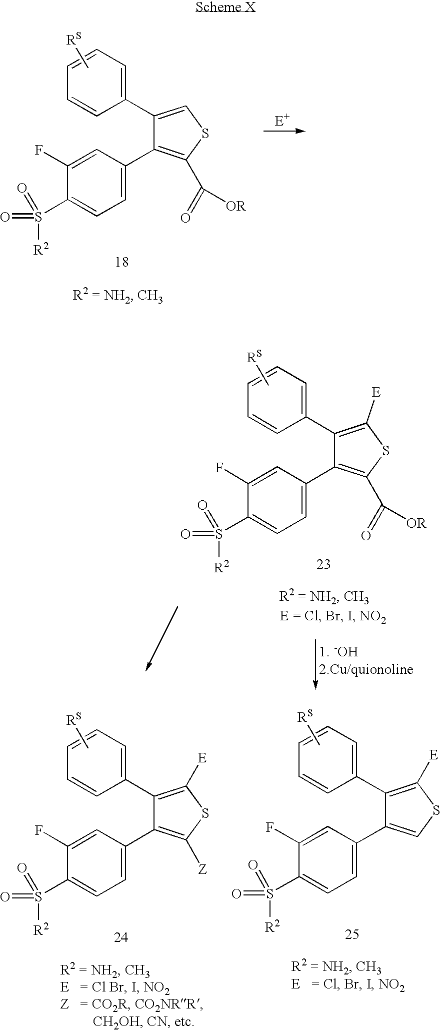

Alternatively, as illustrated in Scheme X, the remaining hydrogen of the thiophene ring 18 can be converted to an halogen or nitro group 23 and the ester once more manipulated as described to a variety of functional groups as in structures 24 and 25.

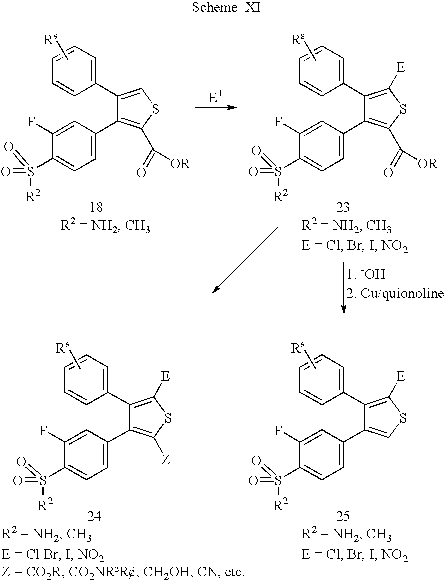

Initiation of the thiophene protocol with the second regioisomer of the 3-fluoro-4-methylthiophenyl-substituted phenyl ethanone 11 and implementation of the thiophene chemistry described in Scheme X affords thiophenes with the regiosubstitution shown in Scheme XI.

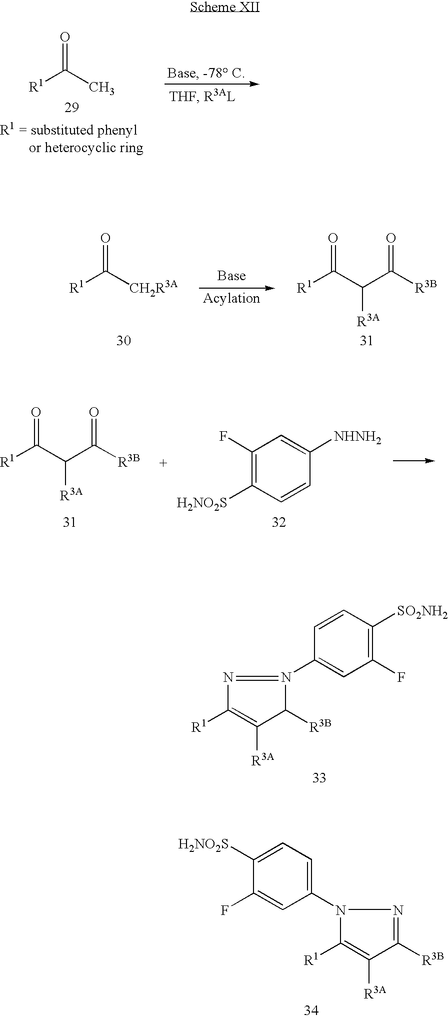

Synthetic Scheme XII illustrates the preparation of tetrasubstituted pyrazoles from acetophenone 29. In step 1 of synthetic Scheme XII, the phenyl-methyl ketone 29 is treated with a base and an alkylating reagent (R³L, where L represents a leaving group such as tosyl) to give the substituted ketone 30. In step 2, the substituted ketone 30 is treated with base, such as sodium methoxide, and an acylating reagent such as an ester (R^{3A}CO₂CH₃), or ester equivalent (R^{3A}CO-imidazole) to give the intermediate diketone 31 [Reid, Calvin; J. Amer. Chem. Soc., 72, 2948-2952 (1950)]. In step 3, the diketone 31 is reacted with a substituted hydrazine 32 in acetic acid or an alcoholic solvent to give a mixture of pyrazoles 33 and 34. Separation of the desired pyrazole 34 can be achieved by chromatography or recrystallization.

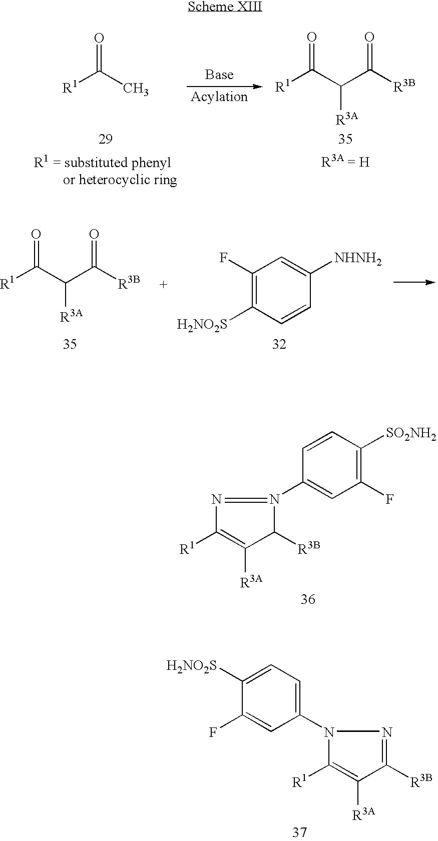

Synthetic Scheme XIII illustrates the preparation of 4-unsubstituted-pyrazoles (i.e., R^{3A} as used in Scheme XIII is hydrogen). In step 1, ketone 29 is treated with a base, preferably sodium methoxide or sodium hydride, and an ester, or ester equivalent, to form the intermediate diketone 35 which is used without further purification. In step 2, diketone 35 in an anhydrous protic solvent, such as absolute ethanol or acetic acid, is treated with the hydrochloride salt or the free base of a substituted hydrazine 32 at reflux for 10 to 24 hours to afford a mixture of pyrazoles 36 and 37. Recrystallization from diethyl ether/hexane or chromatography affords 37, usually as a light yellow or tan solid. Additional pyrazoles can be prepared by suitable modification of the methods described in U.S. Pat. Nos. 5,401,765, 5,434,178, 4,146,721, 5,051,518, 5,134,142 and 4,914,121 which are incorporated by reference.

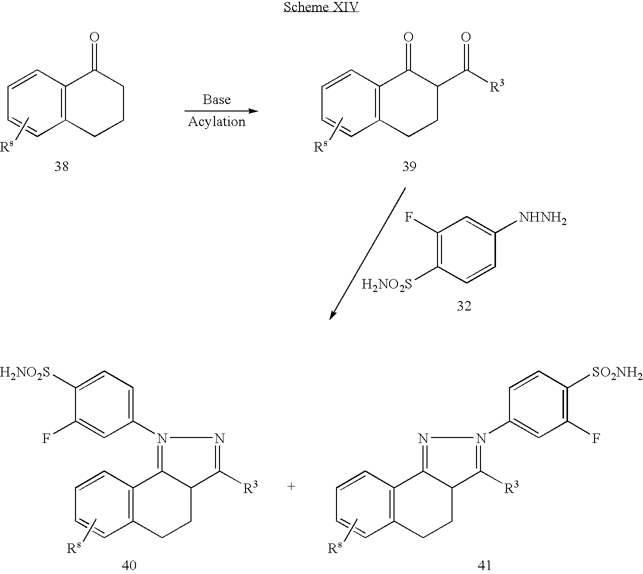

Synthetic Scheme XIV shows an illustrative procedure for the preparation of 4,5-dihydrobenz[g]indazole compounds. In step 1, ethyl esters of acetates are mixed with base, such as 25% sodium methoxide in a protic solvent, such as methanol, and a 1-tetralone derivative 38 to give the intermediate diketone 39. In step 2, the diketone 39 in an anhydrous protic solvent, such as absolute ethanol or acetic acid, is treated with the free base or hydrochloride salt of a substituted hydrazine 32 at reflux for 24 hours to afford a mixture of pyrazoles 40 and 41. Recrystallization gives the 4,5-dihydro benz[g]lindazolyl-benzenesulfonamide 40.

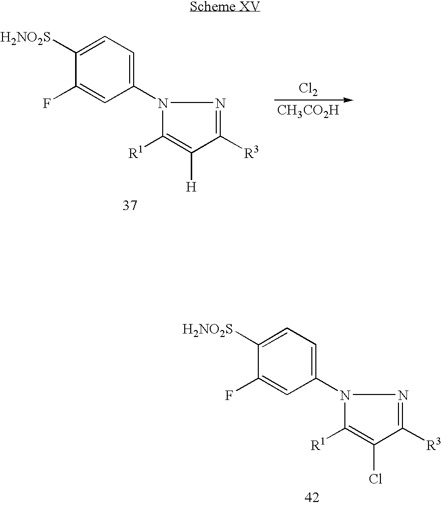

Synthetic Scheme XV illustrates the preparation of 4-chloro-pyrazole compound 42 from the pyrazole compound 37. Chlorination results from passing a stream of chlorine gas at room temperature through a solution containing pyrazole compound 37.

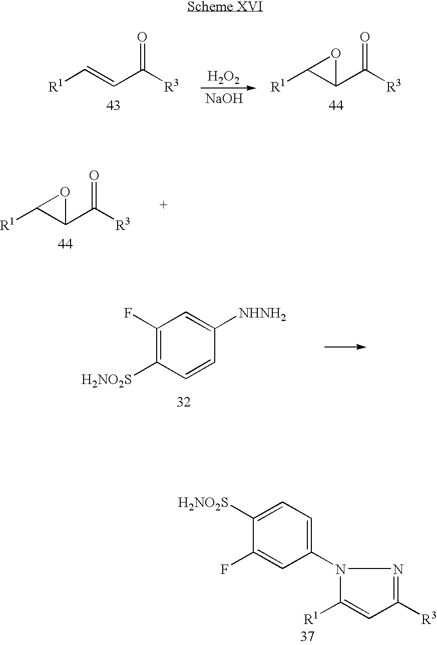

Synthetic Scheme XVI illustrates an alternative regioselective method of preparing the pyrazole 37. Commercially available enones 43 can be epoxidized to give epoxyketones 44, which are treated with 3-fluoro-4-sulfonamidophenylhydrazine hydrochloride to provide the pyrazole 37.

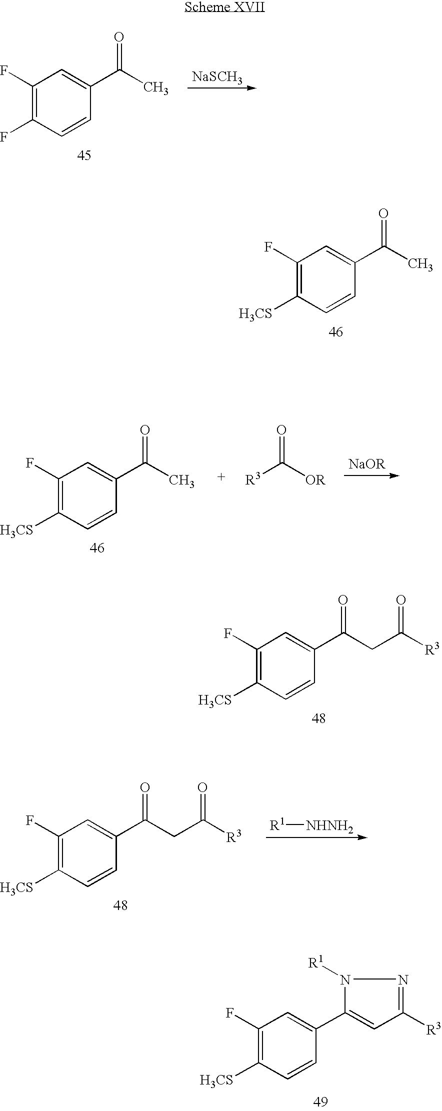

Scheme XVII illustrates a regioselective method of preparing methylthiopyrazole 49. The difluoro-acetophenone 45 is mixed with sodium thiomethoxide in a polar solvent such as acetonitrile of dimethylformamide to afford the 3-fluoro-4-methyl-sulfonylacetophenone 46. Mixing the acetophenone 46 with a base, such as sodium methoxide, and an acylating reagent 47 such as an ester (R¹CO₂CH₃), or activated ester equivalent (R¹CO-imidazole), gives the intermediate diketone 48 [J. Amer. Chem. Soc., 72, 2948-2952, (1950)]. This diketone 48 is refluxed with a substituted hydrazine in alcoholic solvents under acid conditons to afford the methylthiopyrazoles 49 after purification by chromatography or crystallization.

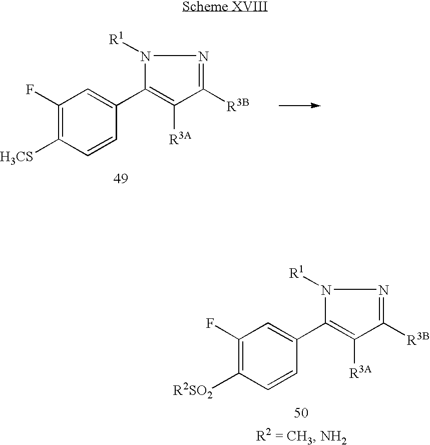

The methylthiopyrazole 49 can be converted to the desired 3-fluoro-4-methylsulfonylpyrazole or 3-fluoro-4-sulfonamidylpyrazole 50 using the procedures described earlier. Similar pyrazoles can be prepared by methods described in U.S. Pat. No. 5,486,534 which is incorporated by reference.

WO96/37476 describes methods for the preparation of 3-haloalkyl-1H-pyrazoles and is incorporated by reference.

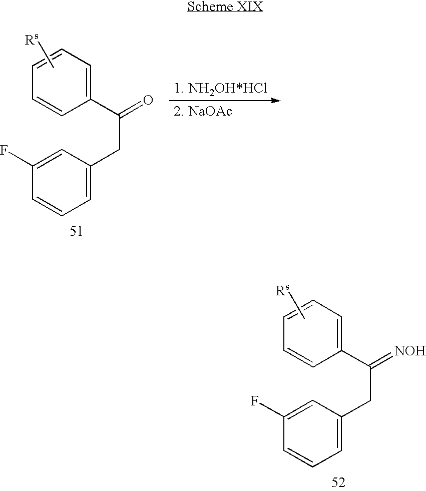

Synthetic Scheme XIX illustrates a procedure that can be used for the preparation of oxime intermediate 52. Treatment of ketone intermediate 51 with hydroxylamine, generally prepared from hydroxylamine hydrochloride by sodium acetate, provides the oxime intermediate 52. A wide variety of solvents can be used for this reaction including ethanol, toluene, and tetrahydrofuran.

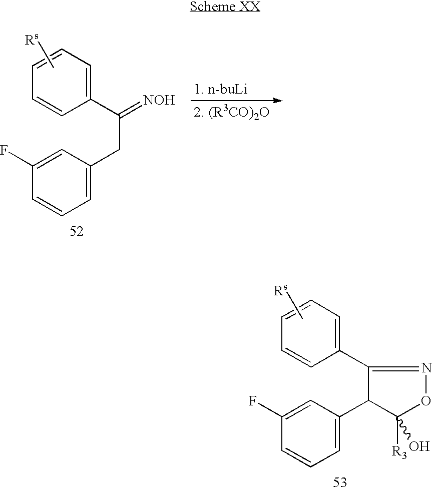

Synthetic Scheme XX illustrates a procedure that can be used for the preparation of hydrated isoxazole derivative 53. The substituted oxime 52 is treated with at least two equivalents of a base such as n-butyllithium in hexanes to produce a dianion that is subsequently acylated. Suitable acylating agents are anhydrides, acyl imidazoles, esters and the like. Upon quenching the reaction mixture with dilute aqueous acid, hydrated isoxazole derivative 53 can be isolated by crystallization or chromatography.

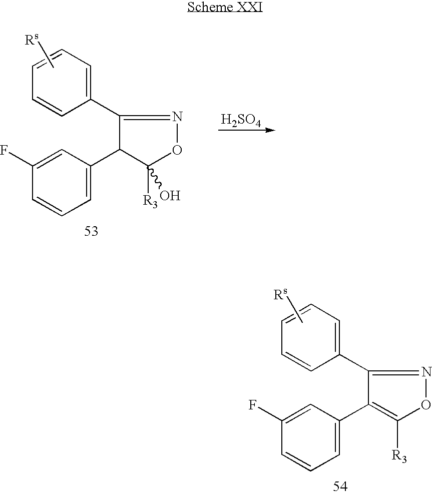

Synthetic Scheme XXI illustrates a procedure that can be used for the preparation of isoxazole analog 54 through dehydration of the hydrated isoxazole derivative 53. Substituted hydrated isoxazole 53 is dissolved in an appropriate solvent such as toluene and then treated with a catalytic to stochiometric amount of concentrated sulfuric acid to effect dehydration and thereby produce isoxazole derivative 54. Other acids can also be employed to effect this transformation such as concentrated HCl, concentrated HBr and many others.

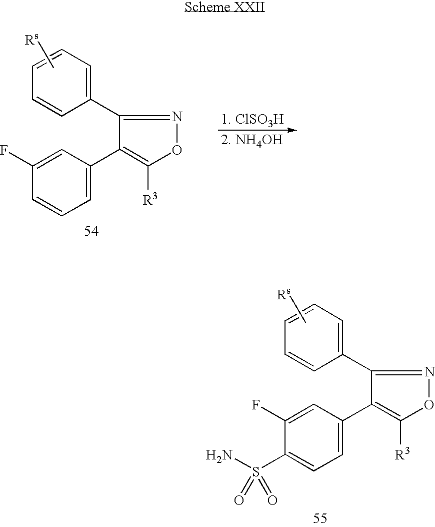

Synthetic Scheme XXII illustrates a procedure that can be used for the preparation of substituted 3-fluoro-(4-sulfonamidyl)phenylisoxazole analog 55 from the corresponding 3-fluorophenylisoxazole 54. The procedure is a two step process for the direct introduction of the sulfonamide moiety into 3-fluorophenylisoxazole 54 or hydrated isoxazole 53. In step one, isoxazole 54 or hydrated isoxazole 53 is treated at about 0° C. with two or three equivalents of chlorosulfonic acid to form the corresponding sulfonyl chloride. In step two, the sulfonyl chloride thus formed is treated with concentrated ammonia to provide the sulfonamide derivative 55.

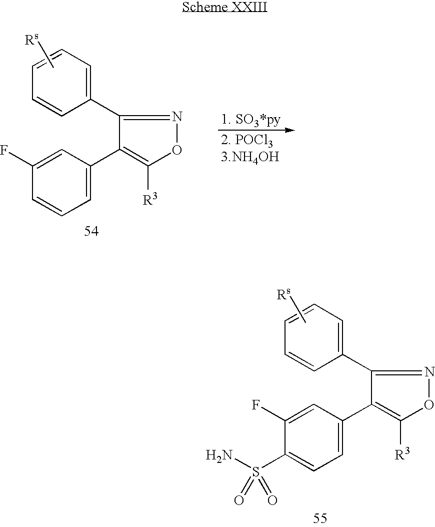

Synthetic Scheme XXIII illustrates a three step procedure used to prepare 3-fluoro-4-sulfonamidyl -phenylisoxazole 55 from 3-fluorophenyl isoxazole 54. In step one, the 3-fluorophenylisoxazole 54 is converted into the corresponding sulfonic acid by treatment with sulfur trioxide pyridine complex at about 100° C. In step two, the sulfonic acid is converted into the sulfonyl chloride by the action of phosphorus oxychloride. In step three, the sulfonyl chloride is treated with excess concentrated ammonia to provide the 3-fluoro-4-sulfonamidyl-phenylisoxazole 55.

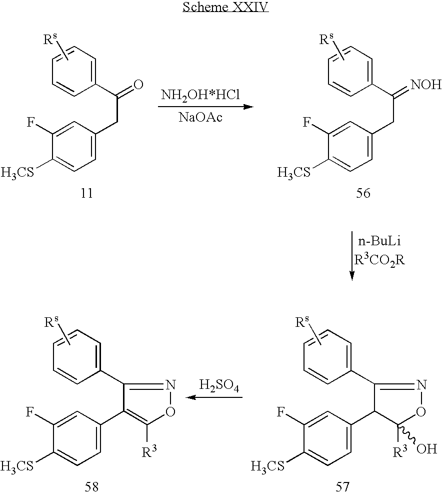

Scheme XXIV illustrates a five step procedure for the preparation of substituted isoxazole derivatives. In step one, substituted 1-substituted phenyl-2-(3-fluoro-4-methylthiophenyl)-ethanone 11 is converted to the oxime 56 by treatment with hydroxylamine hydrochloride in the presence of sodium acetate in aqueous ethanol. The oxime 56 is treated with slightly more than two equivalents of n-butyl lithium and then the resulting dianion is quenched by a suitable acylating agent such as an anhydride, acid chloride, ester, acyl imidazole and the like to afford hydrated isoxazole 57. In the last step, the hydrated isoxazole is dehydrated by an acid and the sulfonamide unmasked by treatment with aqueous sulfuric acid to form the isoxazole derivative 58.

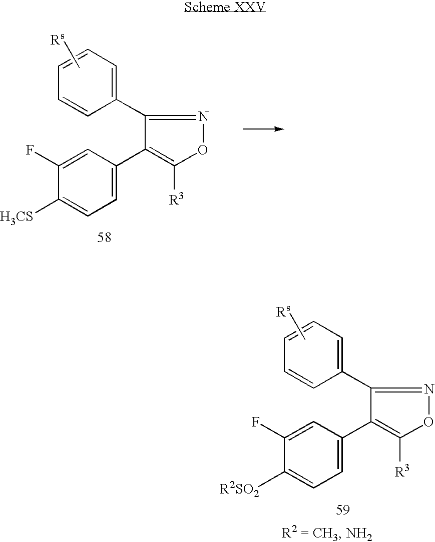

The methylsulfide moeity 58 prepared in Scheme XXIV can be converted to the sulfonamide or methylsulfone diarylisoxazole 59 as previously described.

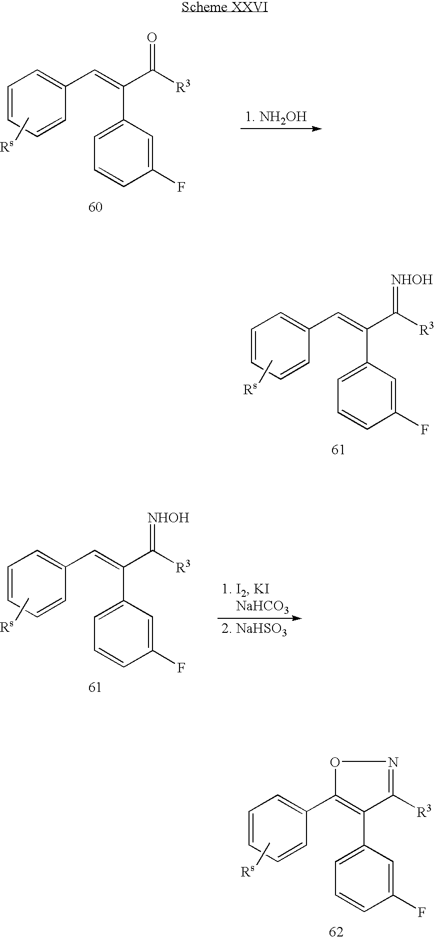

Scheme XXVI illustrates a three step procedure for the preparation of another isoxazole isomer. In step one, substituted 1,2-diphenylbutenone 60 is converted to the oxime 61 by treatment with hydroxylamine hydrochloride in the presence of sodium acetate in aqueous ethanol. The oxime 61 is then reacted with potassium iodide and iodine in the presence of base, such as sodium bicarbonate to afford a halo intermediate. Sodium bisulfite is added to form the isoxazole 62.

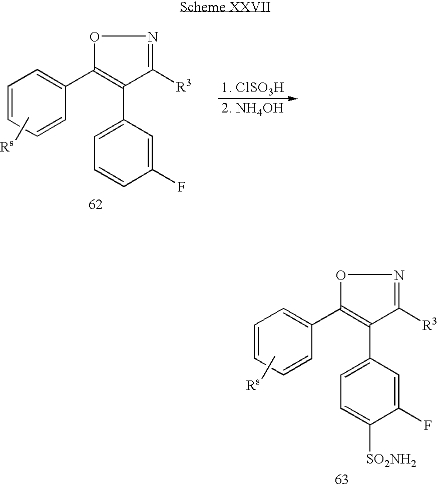

Treatment of the isoxazole 62 with chlorosulfonic acid provides, after workup, the intermediate sulfonyl chloride which can be converted to the sulfonamide 63 by mixing with ammonia in various solvents. In addition, diaryl/heteroaryl isoxazoles comprising the 3-fluoro-4-sulfonamidyl-phenyl moiety can be prepared in accordance with the methods described in PCT Application Serial No. US96/01869, PCT documents WO92/05162 and WO92/19604, and European Publication EP 26928, which are incorporated by reference.

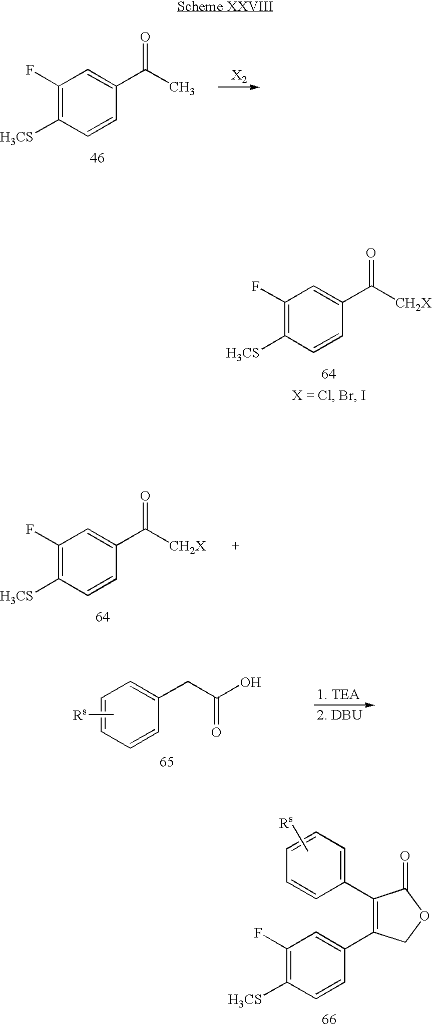

Scheme XXVIII illustrates a synthetic pathway for preparation of furanones. The acetophenone 46 is halogenated in acetic acid to afford the acylhalide 64. Displacement of the halide 64 with phenylacetic acid 65 in the presence of triethyl amine in acetonitrile followed by addition of diazobi-cyclo[5.4.0]undec-7-ene affords the furanone 66 [Ahluwalia, Synth. Commun. 19, 619-626 (1989)].

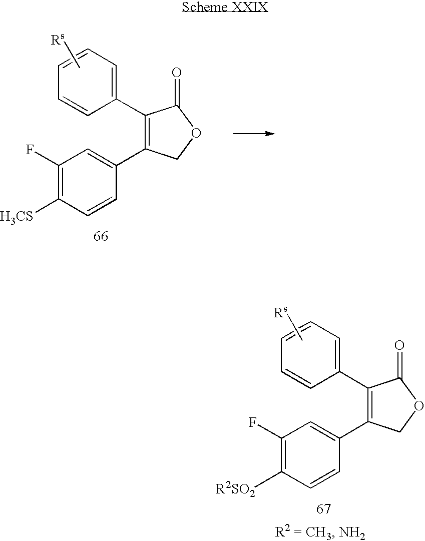

The methylthiofuranone 66 can be converted to the methylsulfonyl or sulfoamide furanone 67 as previously disclosed.

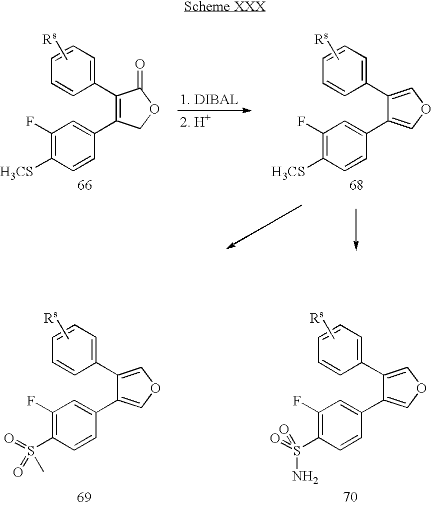

The furan 68 can be prepared from the furanone 66 by reduction and rearomatization though dehydration. Treatment of the furanone 66 in chlorinated solvents such as dichloromethane at reduced temperatures with hydride reagents such as diisobutylaluminum hydride (DIBAL-H) followed by treatment with mineral acids to catalyze the dehydration of the resulting hemiacetal affords the furan 68 [Singh, Indian J Chem, Sect B., 29, 954-960 (1990)]. The sulfide can be converted to the sulfone 69 and sulfonamide 70 as previously described.

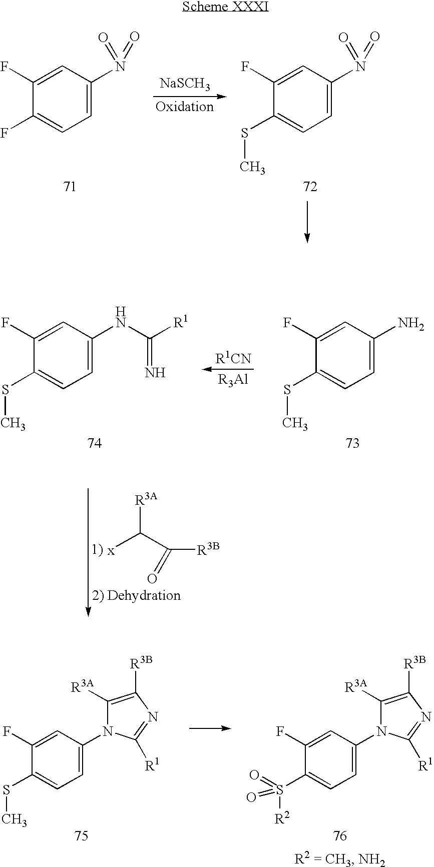

Scheme XXXI illustrates the preparation of the substituted diarylimidazole 76 which comprises a 3-fluoro-4-sulfonylphenyl moiety. Substitution of the 4-fluorine in 3,4-difluoro-nitrobenzene 71 with sodium thiomethoxide provides the 3-fluoro-4-methylthio-nitrobenzene 72. Reduction of the nitro group to an amine can be accomplished through hydrogenation over a metallic catalyst such as palladium or platinum on various supports. The resulting phenylamine 73 can be reacted with a substituted nitrile in the presence of alkylaluminum reagents such as trimethylaluminum in inert solvents such as toluene or benzene affording amidine 74. Mixing of the amidine 74 with a 2-haloketone in the presence of mild inorganic or organic bases such as triethylamine, diisopropylamine, or sodium bicarbonate in solvents such as acetone, acetonitrile, or dimethylformamide gives a 4,5-dihydroimidazole that can be dehydrated in the presence of catalytic mineral acids to afford the 1,2-disubstituted imidazole 75.

These types of diaryl/heteroaryl imidazoles also can be prepared in accordance with the methods described in U.S. Pat. No. 4,822,805 and PCT documents WO 93/14082 and WO96/03388, which are incorporated by reference. The methylsulfide then can be converted to the sulfone or sulfonamide affording substituted diarylimidazole 76 as previously described.

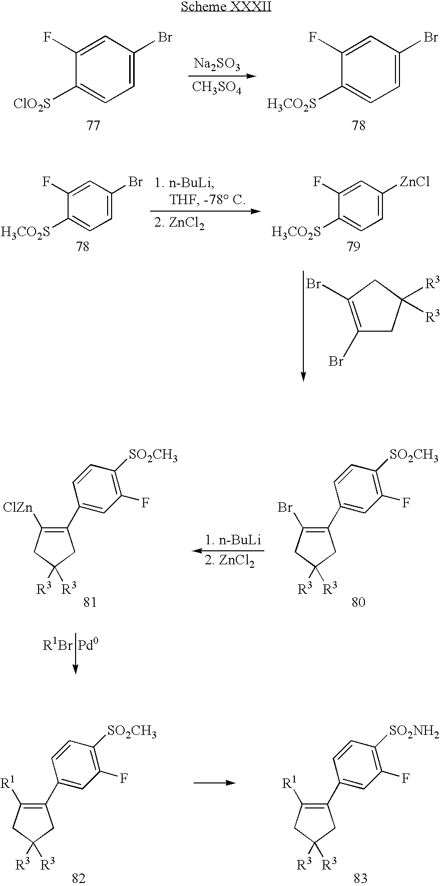

The commercially available 4-bromo-2-fluorobenzenesufonyl chloride 77 can be treated with sodium sulfite followed by treatment with dimethylsulfate to afford the 4-bromo-2-fluoromethylsulfonyl-benzene 78 [Organic Synthesis, Volume IV page 674]. Diaryl/heteroaryl cyclopentene 82 comprising the orthofluorosulfonyl functional group can be prepared from the 4-bromo-2-fluoromethylsulfonyl-benzene 78 as described in Scheme XXXII in accordance with the methods described in U.S. Pat. No. 5,344,991 and PCT document WO 95/00501, which are incorporated by reference. The sulfone can be converted to the cyclopentenphenylsufonamide 83 as described previously.

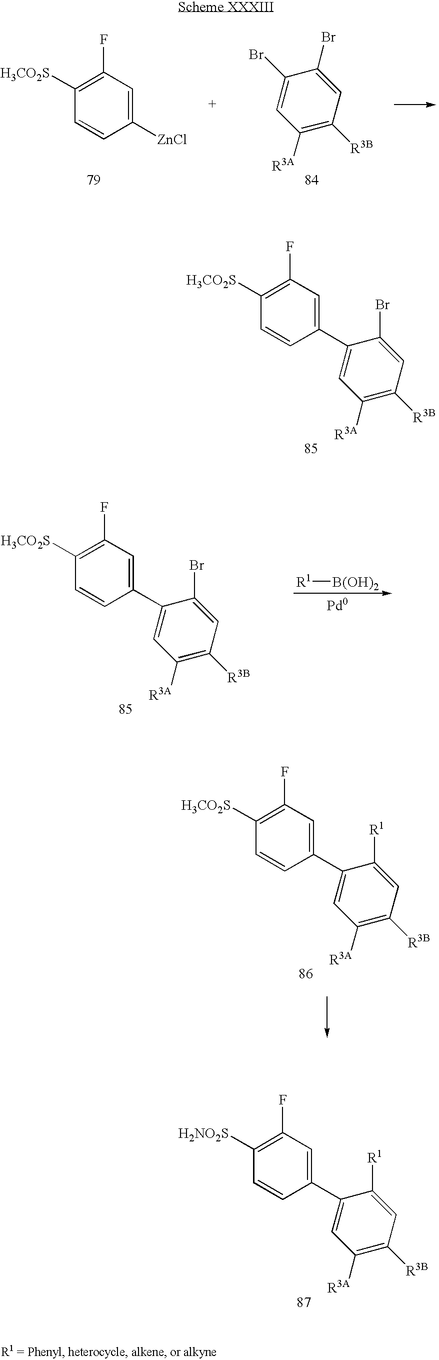

Scheme XXXIII similarly illustrates a procedure for the preparation of 1,2 diarylbenzene 87 comprising the orthofluorosulfonyl functional group from bromo-biphenyl intermediate 86 using the appropriate substituted phenyl boronic acid in a Suzuki coupling procedure [Synth. Commun., 11, 513 (1981)]. Bromo-biphenyl intermediate 86 can be prepared in a manner similar to the procedure described in Scheme XXXII. Sulfonamides can be prepared from the sulfones as described earlier in this application. U.S. application Ser. No. 08/346,533 generally describes the preparation of terphenyl compounds and is incorporated by reference.

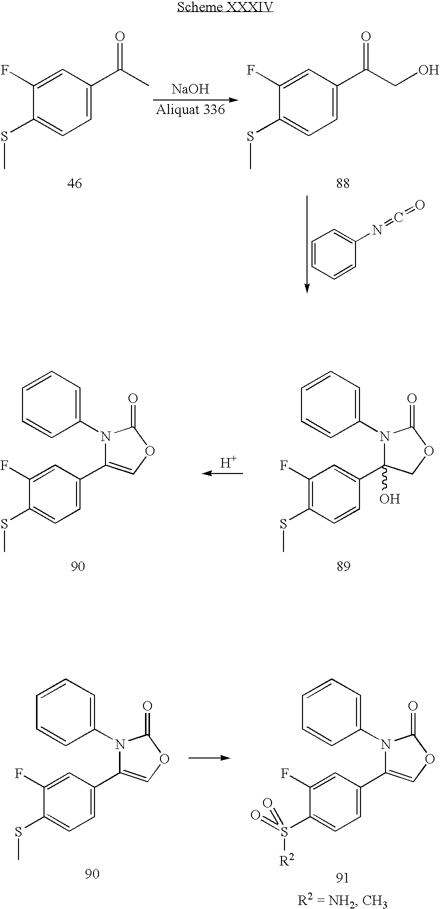

Scheme XXXIV illustrates the preparation of the 3,4-diaryloxazol-2-one 91 and its derivatives. Oxidation of the methyl group adjacent to the carbonyl carbon of the 3-fluoro-4-methyl-sulfonylacetophenone 46 to an alcohol can be accomplished using base, aliquat 336 and oxygen (see, e.g., EP 197704). Treatment of the resulting alcohol 88 with an isocynate followed by dehydration of the hydroxy intermediate 89 in the presence of acid affords the oxazolone 90. The methylsulfide of the oxazolone 90 can be converted to the sulfone or sulfonamide affording substituted 3,4 diaryloxazol-2-ones 91 as previously described. Other 3,4-diaryloxazol-2-one compounds with the basic structure of substituted 3,4 diaryloxazol-2-one 91 that comprise an orthofluorosulfonyl functional group can be prepared starting from the 2-hydroxy-1′-[3′-flouoro-4-methylsulifidophenyl]-ethanone 88 in accordance with the methods described in PCT documents WO 98/11080 and WO 99/14205, which are incorporated by reference.

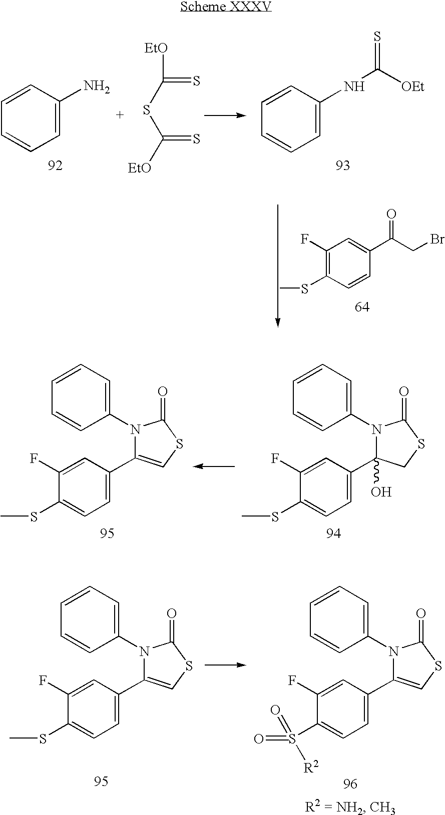

Scheme XXXV illustrates the preparation of 3,4-diarylthiazonlin-2-one. Mixing the analine 92 with bis(ethoxythiocarbonyl) in an alcoholic solvent affords the ethyl-thiocarbamate 93. Refluxing of ethyl-thiocarbamate 93 and the 2-bromoacetophenone 64 in an ethereal solvent provides the hydroxy intermediate 94. Heating of the hydroxy intermediate 94 with mineral acids in alcoholic solvents yields the desired 3,4 diarylthiazolin-2-one 95. U.S. Pat. No. 5,859,036 describes an alternative procedure that can be appropriately modified and used to prepare the compounds. U.S. Pat. No. 5,859,036 is incorporated by reference. The methyl-sulfide 95 can be converted to the corresponding sulfone or sulfonamide affording substituted 3,4 diarylthiazonlin-2-one 96 as previously described.

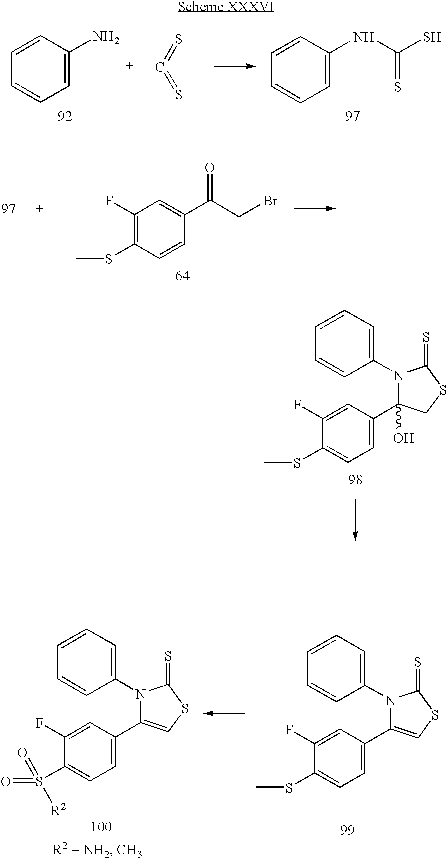

Scheme XXXVI illustrates the preparation of 3,4-diarylthiazonlin-2-thione 100. Mixing of analine 92 with carbon disulfide in an alcoholic solvent affords the dithiocarbamate 97. Refluxing of dithiocarbamate 97 and the 2-bromoacetophenone 64 in an ethereal solvent provides the hydroxy intermediate 98. Heating of the hydroxy intermediate 98 with mineral acids in alcoholic solvents yields the desired 3,4 diarylthiazonlin-2-thione 99. U.S. Pat. No. 5,859,036 illustrates a similar process and is incorporated by reference. The methylsulfide 99 can be converted to the corresponding sulfone or sulfonamide affording substituted 3,4 diarylthiazonlin-2-thione 100 as previously described.

### WORKING EXAMPLES

The following examples contain detailed descriptions of the methods of preparation of compounds of Formulae I-VII. These detailed descriptions fall within the scope, and serve to exemplify, the above-described General Synthetic Procedures which form part of the invention. These detailed descriptions are presented for illustrative purposes only and are not intended as a restriction on the scope of the invention. All parts are by weight and temperatures are in Degrees centigrade unless otherwise indicated. All compounds showed NMR spectra consistent with their assigned structures.

The following abbreviations are used:

HCl—hydrochloric acid

DMSO—dimethylsulfoxide

DMSOd₆—deuterated dimethylsulfoxide

CDCl₃—deuterated chloroform

MgSO₄—magnesium sulfate

NaHCO₃—sodium bicarbonate

KHSO₄—potassium hydrogen sulfate

DMF—dimethylformarmide

NaOH—sodium hydroxide

BOC—tert-butyloxycarbonyl

CD₃OD—deuterated methanol

EtOH—ethanol

LiOH—lithium hydroxide

CH₂Cl₂—methylene chloride

h—hour

hr—hour

min—minutes

TBF—tetrahydrofuran

TLC—thin layer chromatography

Et₃N—triethylamine

DBU—1,8-diazabicyclo[5,4.0]undec-7-ene

DMAP—4-dimethylaminopyridine

### Example 1

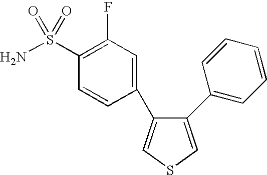

2-Fluoro-4-(4-phenyl-3-thienyl)benzenesulfonamide

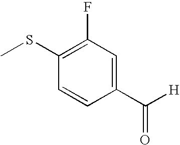

Step 1: Preparation of 3-Fluoro-4-(methylthio)-benzaldehyde

The 3,4-difluorobenzaldehyde (52.1 g, 0.36 mol) was dissolved in acetonitrile (500 mL). Sodium thiomethoxide (25.6 g, 0.36 mol) was added in four equal portions at 15 minute intervals. The slightly exothermic reaction was stirred at room temperature for 4 hours. The reaction mixture was poured into ethyl acetate (500 mL) and extracted with saturated sodium bicarbonate (2×200 mL) followed by saturated ammounium chloride (2×100 mL). The solution was dried over sodium sulfate and solvent removed at reduced pressure. The 3-fluoro-4-(methylthio)-benzaldehyde (38.5 g, 0.22 mol) was isolated by vacuum distillation (135-145° C. at 25 mm Hg) as clear liquid. (61% yield) ESHRMS m/z 171.0302 (calcd for M+H, 171.0280).

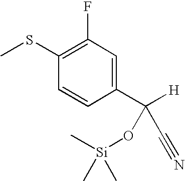

Step 2: Preparation of 3-fluoro-4-(methylthio)-□-[(trimethysilyl)oxy]-benzeneacetonitrile

The 3-fluoro-4-methylthiobenzaldehyde (33.0 g, 194.0 mmol), trimethylsilyl cyanide (19.8 g, 200 mmol) were mixed together in dichloromethane (350 mL) and zinc iodide (35 mg) was addded. The solution was heated to 35° C. The bath was removed and the solution stirred for 1 hour during which an exotherm was noted. After cooling to room temperature the solvent was removed at reduced pressure to afford the 3-fluoro-4-(methylthio)-□-[(trimethylsilyl)oxy]benzene-acetonitrile (51.6 g, 192 mmol) as a yellow oil. (98% yield): ¹H NMR (CHCl₃/300 MB) 7.24-7.29 (m, 3H), 5.48 (s, 1H), 2.51 (s, 3H), 0.27 (s, 9H). ESHRMS m/z 261.0769 (calcd for M+H, 261.0749).

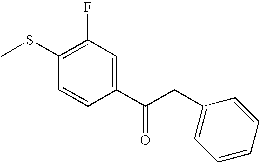

Step 3: Preparation of 1-[3-fluoro-4-(methylthio)-phenyl]-2-phenylethanone

The 3-fluoro-4-(methylthio)-a-[(trimethylsilyl)-oxy]-benzeneacetonitrile (40.1 g, 149 mmol) was cooled to −78° C. in tetrahydrofuran (400 mL). Lithium hexamethydisilazide (175 mL of 1.0 M in Hexanes, 175 mmol) was added drop-wise over 1 hour. The solution was stirred for 1 additional hour at −78° C. Benzylbromide (24.5 g 149 mmol) was added as a solution in tetrahydrofuran (100 mL) over 25 minutes. The solution was kept at −78° C. for 1 hour, warmed to room temperature, and kept at room temperature for six hours. The solution was poured into ethyl acetate (300 mL). Aqueous 1 N hydrochloric acid (200 mL) was added and the solution stirred for 48 hours at room temperature. The layers were separated and the organic layer collected. The organic layer was mixed with aqueous 15% sodium hydroxide and stirred for 30 minutes. The organic layer was collected washed with saturated ammounium chloride (200 mL) and solution was dried over sodium sulfate and solvent removed at reduced pressure to afford a yellow oil. The product was isolated by preparative silica chromatography followed by crystallization from, 2% ethyl acetate and hexanes (400 mL). 1-[3-fluoro-4-(methylthio)phenyl]-2-phenylethanone (21.5 grams, 82.0 mmol) was obtained as white crystals. (55% yield): Mp 77.1-77.2° C. ¹H NMR (CDCl₃/300 MHz) 7.77, (dd, 1H, J=8.0, 1.8 Hz), 7.64 (dd, 1H, J=10.7, 1.8 Hz), 7.20-7.40 (m, 6H), 4.22 (s, 3H), 2.51 (s, 1H).

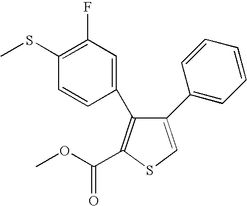

Step 4: Preparation of methyl 3-[3-fluoro-4-(methylthio)phenyl]-4-phenyl-2-thiophenecarboxylate

The 1-[3-fluoro-4-(methylthio)phenyl]-2-phenylethanone (21.9 g, 81.5 mmol) and dimethylacetal of dimethylformamide (41.3 g, 347 mmol) were refluxed in toluene (200 mL) for 16 hours. The yellow solution was cooled to room temperature and solvent removed ad reduced pressure. The resulting yellow oil was dissolved in 50% ethyl acetate/50% hexanes (200 mL) and vacuum filtered through silica gel. The silica was washed with 50% ethyl acetate/50% hexanes (150 mL). The filtrates were combined and solvent removed at reduced pressure to afford 25.19 g the enamine (shown below) as a yellow oil.

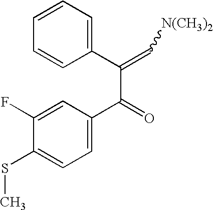

The enamine (25.09 g, 79.3 mmol) and methyl thio-glycolate (35.5 g, 326 mmol) were refluxed for 16 hours. The solvent was removed at reduced pressure and the resulting oil taken up in methanol (300 mL). 25% sodium methoxide in methanol (75.0 mL, 326 mmol) was added and the reaction stirred. After 20 minutes of mixing a precipitate formed and the mixing ceased. The solution was kept at room temperature for 6 hours and the crystals collected. Methyl 3-[3-fluoro-4-(methylthio)phenyl]-4-phenyl-2-thiophenecarboxylate (18.3 grams, 51.7 mmol) was isolated as white crystals. 200 mg of the lot was recrystalized from ethyl acetate and hexanes for analytical data and the remainder used without further purification. (64% Yield): Mp 140.6-141.0° C. ¹H NMR (CDCl₃/300 MHz) 7.52 (s, 1H), 7.52 (s, 1H), 7.21-7.80 (m, 3H), 7.17 (t, 1H, J=8.0 Hz), 7.06-7.12 (m, 2H), 6.86-6.97 (m, 2H), 3.81(s, 3H), 2.50(s, 3H). ESHRMS m/z 359.0598 (calcd for M+H⁺, 359.0576)

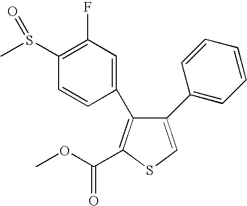

Step 5: Preparation of methyl 3-[3-fluoro-4-(methylsulfinyl)phenyl]-4-phenyl-2-thiophenecarboxylate.

The methyl 3-[3-fluoro-4-(methylthio)phenyl]-4-phenyl-2-thiophenecarboxylate (5.00 g, 13.9 mmol) was dissolved in dichloromethane (100 mL) and methanol (30 mL). Magnesium monoperoxyphathalate hexahydrate (MMP)(3.78 g of 80%, 7.6 mmol) was added in five equal portions at 1 minute intervals. The resulting heterogeneous solution was stirred for 1 hour. Additional MMPP (600 mg, 1.6 mmol) was added and the solution stirred for 15 min. The reaction was complete and solution extracted with saturated sodium bicarbonate (2×100 mL). The organic layer was dried over sodium sulfate and solvent removed at reduced pressure; Methyl 3-[3-fluoro-4-(methylsulfinyl)phenyl]-4-phenyl-2-thiophenecarboxylate was isolated by crystallization from dichloromethane and hexanes. (81% yield): Mp 164.0-164.1° C. ¹H NMR (CDCl₃/300 MHz) 7.75 (t, 1H, J=7.7 Hz), 7.53 (s, 1H), 7.15-7.26 (m, 3H), 6.94-7.40 (m, 3H), 3.90 (s, 3H), 2.85 (s, 3H). ESHRMS m/z 375.0536 (calcd for M+H, 375.0525).

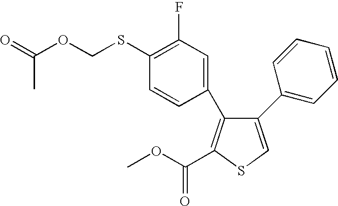

Step 6: Preparation of methyl 3-[4-[[(acetyloxy)-methyl]thio]-3-fluorophenyl]-4-phenyl-2-thiophenecarboxylate

The methyl 3-[3-fluoro-4-(methylsulfinyl)phenyl]-4-phenyl-2-thiophenecarboxylate (4.13 g, 11.04 mmol) was dissolved in acetic anhydride (45.0 mL). Powdered sodium acetate (4.0 g, 48.7 mmol) was added and the solution was refluxed for 8 hours. The solution was poured into a 500 mL round bottom flask and solvent removed at reduced pressure. The residue was take up in ethyl acetate (200 ml) and dichloromethane (20 mL). The solution was extracted with saturated sodium bicarbonate (3×100 mL) followed by saturated ammonium chloride (2×100 mL). The solvent was removed at reduce pressure and residue taken up in ether, dried over sodium sulfate and solvent removed at reduced pressure. Methyl 3-[4-[[(acetyloxy)methyl]thio]-3-fluorophenyl]-4-phenyl-2-thiophenecarboxylate (2.9 g, 6.90 mmol) was isolated by crystallization from diethyl ether and hexanes as white crystals. (62% yield). Mp 102.9-103.7° C. ¹H NMR (CDCl₃/300 MHz) 7.53 (s, 1H), 7.43 (t, 1H, J=7.8 Hz), 7.22-7.28 (m, 3H), 7.04-7.08 (m, 2H), 6.92-6.98 (m, 2H), 5.42 (s, 2H), 3.81 (s, 3H), 2.11 (s, 3H). ESHRMS m/z 434.0898 (calcd for M+NH₄⁺, 434.0896)

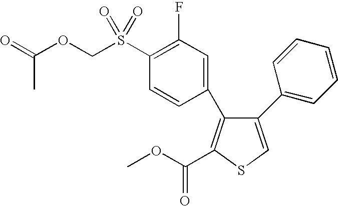

Step 7: Preparation of methyl 3-[4-[[(acetyloxy)methyl]-sulfonyl]3-fluorophenyl]-4-phenyl-2-thiophenecarboxylate

The methyl 3-[4-[[(acetyloxy)methyl]thio]-3-fluorophenyl]4-phenyl-2-thiophenecarboxylate (3.56 g, 8.55 mmol), MMPP (5.80 g of 80%, 9.45 mmol) were mixed in methanol (30 mL) and dichloromethane (100 mL). The solution was mixed at room temperature for 16 h and additional MMPP (2.00 g) was added. The solution was heated to reflux for 8 hours. The solution was poured into ethyl acetate (200 mL) and extracted with saturated aqueous sodium bicarbonate (2×100 mL) followed by brine (100 mL). The organic layer was collected and solvent removed at reduced pressure. The resulting white semi-solid was triturated with ethyl acetate and hexanes. Methyl 3-[4-[[(acetyloxy)methyl]-sulfonyl]3-fluorophenyl]-4-phenyl-2-thiophene-carboxylate (3.15 g, 7.03 mmol) was isolated as an off white solid. (82% yield). Mp 164.7-164.8° C. ¹H NMR (CDCl₃/300 MHz) 7.82 (t, 1H, J=7.5 Hz), 7.56 (s, 1H), 7.22-7.30 (m, 2H), 7.13 (d, 2H, J=9.2 Hz), 7.00-7.67 (m, 2H), 5,35 (s, 2H), 3.82 (s, 3H), 2.09 (s, 3H). ESHRMS m/z 466.0796 (calcd for M+NH₄⁺, 466.0794)

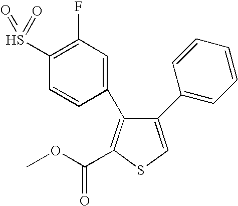

Step 8: Preparation of 2-methyl 3-(3-fluoro-4-sulfinophenyl)-4-phenyl-2-thiophenecarboxylate

The methyl 3-[4-[[(acetyloxy)methyl]sulfonyl]3-fluorophenyl]4-phenyl-2-thiophenecarboxylate (2.75 g, 6.13 mmol) was dissolved in tetrahydrofuran (100 mL) and stirred at room temperature. 25% Sodium methoxide in methanol (3.0 mL) was added and the solution stirred for 5 min. The resulting slurry was poured into ethyl acetate (200 mL) and aqueous 1N hydrochloric acid (100 mL) was added. The solution was stirred and the layers allowed to separate. The organic layer was collected and the aqueous layer back extracted with ethyl acetate (100 mL). The organic layers were combined, extracted with brine, dried over sodium sulfate, and solvent removed at reduced pressure. The 2-methyl 3-(3-fluoro-4-sulfinophenyl)-4-phenyl-2-thiophenecarboxylate (1.87 g, 4.59 mmol) was isolated as a white solid. (77% yield). Mp 132.6-133.1° C. ¹H NMR (CDCl₃/300 MHz) 7.74-7.80 (1H, m), 7.55 (s, 1H), 6.80-7.28 (m, 6H), 3.74 (s, 3H). ESHRMS m/z 394.0586 (calcd for M+NH₄⁺, 394.0583)

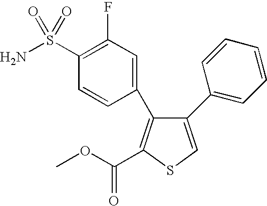

Step 9: Preparation of methyl 3-[3-fluoro-4-(sulfonamido)phenyl]-4-phenyl-2-thiophenecarboxylate

The 2-methyl 3-(3-fluoro-4-sulfinophenyl)-4-phenyl-2-thiophenecarboxylate (1.51 g, 4.01 mmol) was dissolved in methanol (25 mL). Water (10 mL) was added and the solution became slightly clouldy. Sodium acetate (2.62 g, 32.0 mmol) and hydroxyamine-O-sulfonic acid (1.80 g, 16.6 mmol) were mixed toeghter at room temperature for 4 Hours. The solution was poured into ethyl acetate (100 mL) and extracted with 1N aqueous hydrochloric acid (2×50 mL), water (2×50 mL), saturated sodium bicarbonate (2×50 mL) and brine (50 mL). The solution was dried over anhydrous sodium sulfate and solvent removed at reduced pressure. The methyl 3-[3-fluoro-4-(sulfonamido)phenyl]-4-phenyl-2-thiophene-carboxylate (1.04 g, 2.65 mmol) was isolated as a white solid by crystallizations from ethyl acetate and hexanes. (66% yield). Mp 168.3-168.4° C. ¹H NMR (CDCl₃/300 MHz) 7.78 (t, 1H, J=7.6 Hz), 7.51 (s, 1H), 7.20-7.61 (m, 3H), 6.80-7.10 (m, 4H), 5.01 (bs, 2H), 3.77 (s, 3H). ESHRMS m/z 409.0684 (calcd for M+NH₄⁺, 409.0692)

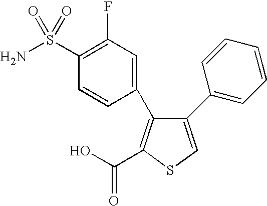

Step 10: Preparation of 3-[3-fluoro-4-(sulfonamido)-phenyl]-4-phenyl-2-thiophenecarboxylic Acid.

The methyl 3-[3-fluoro-4-(sulfonamido)phenyl]-4-phenyl-2-thiophene-carboxylate (922 mg, 2.35 mmol) and lithium hydroxide (356 mg, 8.00 mmol) were mixed in tetrahydrofuran (14.0 mL), methanol (4.0 mL), and water (2.0 mL) at room temperature for 16 hours. The solution was poured into ethyl acetate (100 mL) and extracted with 1 N hydrochloric acid (2×50 mL) followed by brine (50 mL). The solution was dried over sodium sulfate and solvent removed at reduced pressure. The 3-[3-fluoro-4-(sulfonamido)-phenyl]-4-phenyl-2-thiophenecarboxylic acid (723 mg, 2.14 mmol) was isolated as a white solid by crystallization from ethyl acetate and hexanes. (91% yield). Mp 214.6-214.7° C. ¹H NMR (DMSOd₆/400 MHz) 13.2 (bs, 1H), 7.92 (s, 1H), 7.66 (bs, 2H), 7.60 (t, 1H, J=7.9 Hz), 7.20-7.28 (m, 4H), 7.00-7.60 (m, 3H). ESHRMS m/z 395.0522 (calcd for M+NH₄⁺, 395.0536)

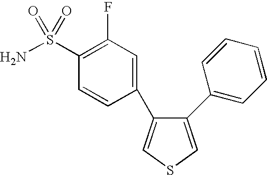

Step 11: Preparation of 2-fluoro-4-(4-phenyl-3-thienyl)benzenesulfonamide

The 3-[3-fluoro-4-(sulfonamido)-phenyl]-4-phenyl-2-thiophenecarboxylic acid (331 mg, 0.87 mmol) and copper powder (150 mg) were heated in freshly distilled quinoline (15 mL) to 160° C. for 1 hour. The reaction was cooled to room temperature and poured into ethyl acetate (100 mL). The solution was extracted to with 1N hydrochroic acid (2×100 mL), saturated sodium bicarbonate (2×100 mL) and brine (100 mL). The solution was dried over sodium sulfate and solvent removed at reduced pressure. The thiophene (113 mg, 0.34 mmol) was isolated as a white solid by flash chromatography (ethyl acetate/hexanes) followed by crystallization from hot diethyl ether and hexanes. (38% yield). Mp 170.9-171.0° C. ¹H NMR (CDCl₃/300 MHz) 7.79 (t, 1H, J=8.0 Hz), 7.45 (d, 1H, J=3.22 Hz), 7.38 (d, 1H, J=3.22 Hz), 7.32-7.60 (m, 3H), 7.18-7.22 (m 2H), 7.40-7.12 (m, 2H), 5.03 (bs, 2H). ESHRMS m/z 351.0647 (calcd for M+NH₄⁺, 351.0367).

### Example 2

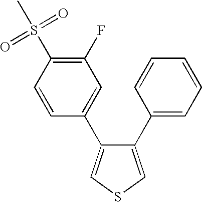

3-[3-Fluoro-4-(methylsulfonyl)phenyl]-4-phenylthiophene

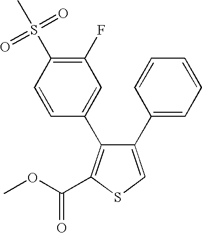

Step 1: Preparation of methyl 3-[3-fluoro-4-(methylsulfonyl)phenyl]-4-phenyl-2-thiophenecarboxylate

Methyl 3-[3-fluoro-4-(methylthio)phenyl]-4-phenyl-2-thiophenecarboxylate (5.15 g, 14 mmol, from example 1 step 4) was dissolved in dichlormethane (100 mL) and methanol (30 mL). Magnesium monoperoxyphathalate hexahydrate (MMPP)(10.25 g of 80%, 17 mmol) was added in five equal portions at 1 minute intervals. The resulting heterogeneous solution was stirred overnight at ambient temperature. Ethyl acetate (200 mL) was added and the solution extracted with saturated sodium bicarbonate (2×100 mL). The organic layer was dried over sodium sulfate and solvent removed in vacuo. Methyl 3-[3-fluoro-4-(methylsulfonyl)phenyl]-4-phenyl-2-thiophenecarboxylate (5.27 g, 12.9 mmol) was isolated by crystallization from ethyl acetate and hexanes (92% Yield): mp 181-182° C. ¹H NMR (CDCl₃/300 MHz) 7.87 (t, 1H, J=7.7 Hz), 7.56 (s, 1H), 7.26-7.24 (m, 4H), 7.15 (s, 1H), 7.12-7.10 (m, 1H), 7.04-7.01 (m, 2H), 3.83 (s, 3H), 3.26 (s, 3H). ESHRMS m/z 408.0738 (M+H⁺, Calcd 408.0740). Anal. Calcd for C₁₉H₁₅FO₄S₂: C, 58.45; H, 3.87; Found: C, 58.22; H, 3.78.

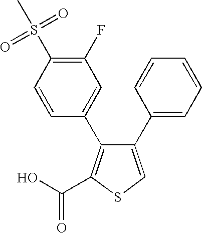

Step 2: Preparation of 3-[3-fluoro-4-(methylsulfonyl-phenyl-4-phenyl-2-thiophenecarboxylic Acid

Methyl 3-[3-fluoro-4-(methylsulfonyl)phenyl]-4-phenyl-2-thiophenecarboxylate (2.46 g, 63 mmol) was dissolved in tetrahydrofuran (25 mL), methanol (175 mL) and water (50 mL). Lithium hydroxide (0.66 g, 157 mmol) was added and the mixture was stirred overnight at ambient temperature. The organic solvents were removed in vacuo and the aqueous suspension was poured into ethyl acetate (100 mL). The solution was extracted with 10% aqueous hydrochloric acid (2×100 mL), water (2×100 mL), saturated ammonium chloride (2×100 mL) and dried over sodium sulfate. The solvent was removed in vacuo and 3-[3-fluoro-4-(methylsulfonylphenyl-4-phenyl-2-thiophenecarboxylic acid (2.12 g, 56.0 mmol) was obtained by crystallization from ethyl acetate and hexanes (90% yield): mp 216-218° C. ¹H NMR (CDCl₃/300 MHz) 7.86 (t, 1H, J=7.7 Hz), 7.65 (s, 1H), 7.29-7.26 (m, 3H), 7.14-7.11 (m, 2H), 7.04-7.00 (m, 2H), 3.28 (s, 3H). ESHRMS m/z 394.0539 (M+NH₄⁺, Calcd 394.0583). Anal. Calcd for C₁₈H₁₃FO₄S₂: C, 57.43; H, 3.48; Found: C, 56.94; H, 3.41.

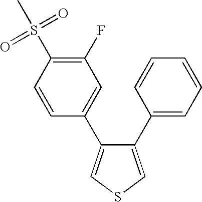

Step 3: Preparation of 3-[3-fluoro-4-(methylsulfonyl)-phenyl]-4-phenylthiophene

3-[3-Fluoro-4-(methylsulfonylphenyl-4-phenyl-2-thiophenecarboxylic acid (1.02 g, 3 mmol) was dissolved in freshly distilled quinoline (10 mL) and powdered copper (0.09 g, 1.3 mmol) was added. The reaction was heated to 130° C. for 3 hours. The reaction was cooled to ambient temperature and poured into aqueous 10% hydrochloric acid (50 mL) and ethyl acetate (50 mL). The solution was extracted with 10% aqueous hydrochloric acid (2×50 mL), water (2×50 mL), saturated ammonium chloride (2×50 mL) and dried over sodium sulfate. 3-[3-Fluoro-4-(methylsulfonyl)phenyl]-4-phenylthiophene (0.55 g, 1.60 mmol) was isolated by preparative silica chromatography followed by crystallization from ethyl acetate and hexanes (54% yield): mp 140-141° C. ¹H NMR (CDCl₃/300 MHz) 7.84 (t, 1H, J=7.9 Hz), 7.43 (ab, 2H, J=13.79 Hz, Δν=27.28), 7.35-7.33 (m, 3H), 7.21-7.07 (m, 4H), 3.25 (s, 3H). ESHRMS m/z 350.0686 (M+NH₄⁺, Calcd 350.0685). Anal. Calcd for C₁₇H₁₃FO₂S₂: C, 61.42; H, 3.94; Found: C, 61.44; H, 3.94.

### Example 3

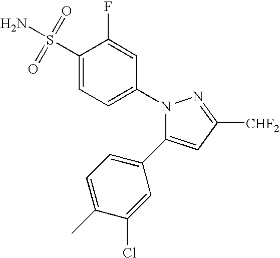

4-[5-(3-chloro-4-methylphenyl)-3-(difluoromethyl)-1H-pyrazol-1-yl]-2-fluorobenzenesulfonamide

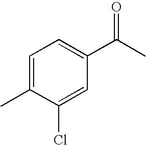

Step 1: Preparation of 1-(3-chloro-4-methlphenyl)-ethanone

3′-Chloro-4′-methylacetophenone (26.8 g, 200 mmol) and aluminum chloride (80.0 g, 600 mmol) were stirred in chloroform (200 mL) at room temperature while chlorine gas (14.2 g, 200 mmol) was added over a half hour period. The reaction was stirred for 2 hours at ambient temperature. The mixture was poured into ice and extracted with ethyl acetate (3×100 mL). The combined organic extracts were washed with 10% aqueous hydrochloric acid (2×100 mL) and water (2×100 mL). The resulting solution was dried over magnesium sulfate and concentrated. 1-(3-chloro-4-methylphenyl)ethanone was purified by distillation from 78-95° C. at 0.1 T and formed a light yellow solid upon standing. This material was used in the next step without further purification or characterization.

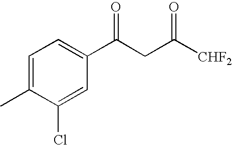

Step 2: Preparation of 1-(3-chloro-4-methylphenyl)-4,4-difluoro-1,3-butanedione

1-(3-chloro-4-methylphenyl)ethanone (7.60 g, 45 mmol), ethyl difluoroacetate (6.20 g, 50 mmol) and sodium methoxide (14 mL) were stirred in diethyl ether (50 mL) at ambient temperature for 5 hours. Ethyl acetate (50 mL) was added to the reaction and it was extracted with 10% aqueous hydrochloric acid (2×50 mL), water (2×50 mL), saturated ammonium chloride (2×50 mL) and dried over sodium sulfate. Solvent was removed in vacuo to yield a yellow solid, 1-(3-chloro-4-methylphenyl)-4,4-difluoro-1,3-butanedione (4.95 g, 20.2 mmol, 45% yield): mp 4041° C. ¹H NMR (CDCl₃/300 MHz) 7.93 (d, 1H, J=1.7 Hz), 7.56 (dd, 1H, J=7.9, 1.7 Hz), 7.37 (d, 1H, J=7.9 Hz), 6.54 (s, 1H), 6.04 (t, 1H, J=54.2), 2.48 (s, 3H). ESHRMS m/z 246.0286 (M+H⁺, Calcd 246.0259). Anal. Calcd for C₁₁H₉F₂ClO₂: C, 53.57; H, 3.68; Found: C, 53.10; H, 3.51.

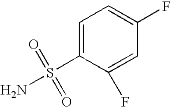

Step 3: Preparation of 2,4-difluorobenzenesulfonamide

2,4-Difluorobenzenesulphonyl chloride (50.26 g, 237 mmol) was combined with concentrated ammonium hydroxide (100 mL) in methylene chloride (800 mL) and stirred at ambient temperature for 2 hours. Methylene chloride was removed in vacuo and solids were dissolved in ethyl acetate (800 mL). The solution was extracted with water (2×500 mL), saturated ammonium chloride (2×500 mL) and dried over sodium sulfate. The solvent was removed in vauo and 2,4-difluorobenzenesulfonamide was obtained by crystallization from ethyl acetate and hexanes (43.27 g, 225 mmol, 95% yield): mp 155-156° C. ¹H NMR (CDCl₃/300 MHz) 7.46-7.38 (m, 1H), 6.87 (brs, 2H), 6.59-6.51 (m, 2H). ESHRMS m/z 192.9990 (M+H⁺, Calcd 193.0009). Anal. Calcd for C₆H₅F₂NO₂S: C, 37.31; H, 2.61; N, 7.25; Found: C, 37.38; H, 2.51, N 7.26.

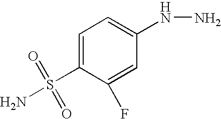

Step 4: Preparation of 2-fluoro-4-hydrazinobenzene-sulfonamide

2,4-difluorobenzenesulfonamide (23.43 g, 121 mmol) and anhydrous hydrazine (17.00 g, 364 mmol) were refluxed in acetonitrile (300 mL) for two hours. The solvent was removed in vacuo and the solid stirred vigorously in ethyl acetate (200 ml) overnight. The solids removed by filtration and discarded and the ethyl acetate was removed in vacuo to afford a white solid. 2-Fluoro-4-hydrazinobenzenesulfonamide (6.05 g, 29.5 mmol) was isolated by preparative silica chromatography followed by crystallization from ethyl acetate and hexanes (24% yield): mp 136-137° C. ¹H NMR (DMSOd₆/300 MHz) 7.73 (s, 1H), 7.43 (t, 1H, J=8.7 Hz), 7.17 (s, 2H), 6.62-6.51 (m, 2H), 4.28 (s, 2H). ESHRMS m/z 206.0451 (M+H⁺, Calcd 206.0400). Anal. Calcd for C₆H₈FN₃O₂S: C, 35.13; H, 3.93; N, 20.48; Found: C, 35.18; H, 3.85, N 20.29.

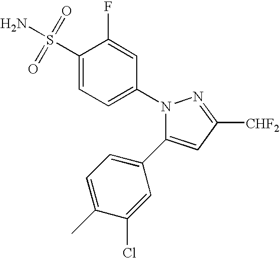

Step 5: Preparation of 4-[5-(3-chloro-4-methylphenyl)-3-(difluoromethyl)-1H-pyrazol-1-yl]-2-fluorobenzenesulfonamide

2-Fluoro-4-hydrazinobenzenesulfonamide (0.73 g, 4.0 mmol) was dissolved in ethanol (10 mL) and concentrated hydrochloric acid (0.5 mL) was added. After stirring for 5 minutes at ambient temperature, 1-(3-chloro-4-methylphenyl)-4,4-difluoro-1,3-butanedione (1.08 g, 4 mmol) was added and the reaction was refluxed for 1 hour. The solution was cooled to ambient temperature and water was added until the reaction became cloudy. The reaction mixture was stirred overnight at ambient temperature. The 4-[5-(3-chloro-4-methylphenyl)-3-(difluoromethyl)-1H-pyrazol-1-yl]-2-fluorobenzene-sulfonamide (0.88 g, 2.12 mmol) was isolated as light orange crystals by vacuum filtration directly from the reaction mixture. (0.88 g, (53% yield): mp 160-161° C. ¹H NMR (CD₃OD/300 MHz) 7.91 (t, 1H, J=8.1 Hz), 7.42-7.06 (m, 5H), 6.88 (s, 1H), 2.42 (s, 3H). ESHRMS m/z 416.0465 (M+H⁺, Calcd 416.0447). Anal. Calcd for C₁₇H₁₃ClF₃N₃O₂S: C, 49.10; H, 3.15; N, 10.11; Found: C, 49.09; H, 2.97, N 9.95.

### Example 4

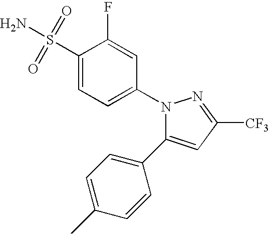

4-[5-(4-Methylphenyl)-3-(trifluoromethyl)-1H-pyrazol-1-yl]-2-fluorobenzenesulfonamide

The title compound was obtained by substituting 1-(4-chlorophenyl)-ethanone for 1-(3-chloro-4-methylphenyl)-ethanone and ethyl trifluoroacetate for ethyl difluoroacetate using the method described in example 3: mp 126-128° C. ¹H NMR (DMSOd₆/300 MHz) 7.80 (m, 2H,), 7.49 (dd, 1H, J=10.74, 1.8 Hz), 7.29 (dd, 1H, J=8.6, 1.88 Hz) (7.16-7.24 m, 5H), 2.29 (s, 3H). ESHRMS m/z 400.0753 (M+H⁺, Calcd 400.0742).

### Example 5

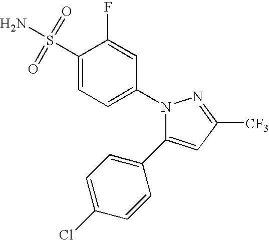

4-[5-(4-Chlorophenyl)-3-trifluoromethyl-1H-pyrazol-1-yl]-2-fluorobenizenesulfonamide

The title compound was obtained by substituting 1-(4-chlorophenyl)-ethanone for 1-(3-chloro-4-methylphenyl)-ethanone and ethyl trifluoroacetate for ethyl difluoroacetate using the method described in example 3: mp 140-141° C. ¹H NMR (CD₃OD/300 MHz) 7.82 (t, 1H, J=8.0 Hz), 7.36-7.14 (m, 6H), 6.91 (s, 1H). ESHRMS m/z 420.0224 (M+H⁺, Calcd 420.0197).

### Example 6

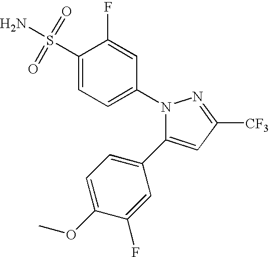

4-[5-(3-Fluoro-4-methoxy)-3-trifluoromethyl-1H-pyrazol-1-yl]-2 fluorobenzenesulfonamide

The title compound was obtained by substituting 1-(4-fluoro-3-methoxyphenyl)-ethanone for 1-(3-chloro-4-methylphenyl)-ethanone and ethyl trifluoroacetate for ethyl difluoroacetate using the method described in example 3: mp 124-126° C. ¹H NMR (CD₃OD/300 MHz) 7.94 (t, 1H, J=8.3 Hz), 7.29 (dd, 1H, J=10.5, 2.0 Hz), 7.29 (dd, 1H, J=8.3, 2.0 Hz), 7.16-7.06 (m, 3H), 6.96 (s, 1H), 3.92 (s, 3H). ESHRMS m/z 434.0598 (M+H⁺, Calcd 434.0617).

### Example 7

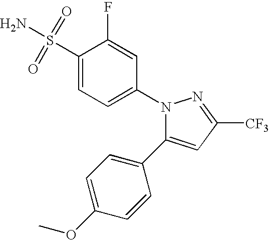

2-Fluoro-4-[5-(4-methoxyphenyl)-3-(trifluoromethyl)-1H-pyrazol-1-yl]benzenesulfonamide

The title compound was obtained by substituting 1-(4-methoxyphenyl)-ethanone for 1-(3-chloro-4-methylphenyl)-ethanone and ethyl trifluoroacetate for ethyl difluoroacetate using the method described in example 3: mp 130-131° C. ¹H NMR (CD₃OD/300 MHz) 7.88 (t, 1H, J=8.1 Hz), 7.34 (dd, 1H, J=10.7, 1.9 Hz), 7.25-7.22 (m, 3H), 6.95 (m, 2H), 6.87 (s, 1H), 3.80 (s, 3H). ESHRMS m/z 416.0692 (M+H⁺, Calcd 416.0680). Anal. Calcd for C₁₇H₁₃F₄N₃O₃S: C, 49.16; H, 3.15; N, 10.12; Found: C, 49.20; H, 3.01; N, 9.75.

### Example 8

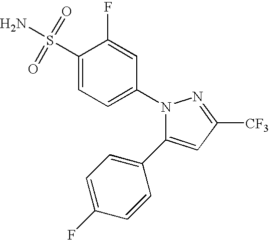

2-Fluoro-4-[5-(4-fluorophenyl)-3-(trifluoromethyl)-1H-pyrazol-1-yl]benzenesulfonamide

The title compound was obtained by substituting 1-(4-fluorophenyl)-ethanone for 1-(3-chloro-4-methylphenyl)-ethanone and ethyl trifluoroacetate for ethyl difluoroacetate using the method described in example 3: mp 127-130° C. ¹H NMR (CD₃OD/300 MHz) 7.92 (t, 1H, J=8.3 Hz). 7.42-7.17 (m, 6H), 7.00 (s, 1H). ESHRMS m/z 404.0497 (+H⁺, Calcd 404.0492). Anal. Calcd for C₁₆H₁₀F₅N₃O₂S: C, 47.65; H, 2.50; N, 10.42; Found: C, 47.43; H, 2.37; N, 10.24.

### Example 9

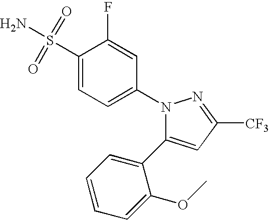

2-Fluoro-4-[5-(2-methoxyphenyl)-3-(trifluoromethyl)-1H-pyrazol-1-yl]benzenesulfonamide

The title compound was obtained by substituting 1-(2-methoxyphenyl)-ethanone for 1-(3-chloro-4-methylphenyl)-ethanone and ethyl trifluoroacetate for ethyl difluoroacetate using the method described in example 3: mp 159-160° C. ¹H NMR (CD₃OD/300 MHz) 7.61 (t, 1H, J=8.3 Hz), 7.41 (t, 1H, J=7.5 Hz), 7.34 (d, 1H, J=7.5), 7.17-7.13 (m, 2H), 7.01 (t, 1H, J=7.5), 6.91 (d, 1H, J=8.3), 6.76 (s, 1H), 3.37 (s, 3H). ESHRMS m/z 416.0675 (M+H⁺, Calcd 416.0692). Anal. Calcd for C₁₇H₁₁F₄N₃O₃S: C, 49.16; H, 3.15; N, 10.12; Found: C, 49.10; H, 3.12; N, 10.09.

### Example 10

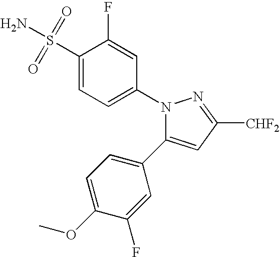

4-[3-(Difluoromethyl)-5-(3-fluoro-4-methoxyphenyl)-1H-pyrazol-1-yl]-2-fluorobenzenesulfonamide

The title compound was obtained by substituting 1-(4-fluoro-3-methoxyphenyl)-ethanone for 1-(3-chloro-4-methylphenyl)-ethanone: mp 167-169° C. ¹H NMR (CD₃OD/300 MHz) 7.91 (t, 1H, J=8.1 Hz), 7.39 (dd, 1H, J=10.9, 2.0 Hz), 7.25 (dd, 1H, J=8.5, 1.4), 7.17-7.05 (m, 3H), 6.86 (t, 1H, J=54.5), 6.83 (s, 1H), 3.92 (s, 3H). ESHRMS m/z 416.0721 (M+H⁺, Calcd 416.0692). Anal. Calcd for C₁₇H₁₃F₄N₃O₃S: C, 49.16; H, 3.15; N, 10.12; Found: C, 49.20; H, 3.24; N, 9.95

### Example 11

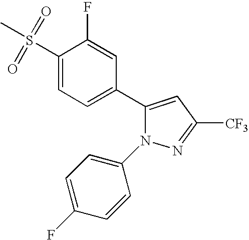

5-[3-Fluoro-4-(methylsulfonyl)phenyl]-1-(4-fluorophenyl)-3-(trifluoromethyl)-1H-pyrazole

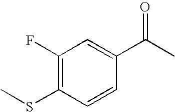

Step 1: Preparation of 1-[3-fluoro-4-methylthio)-phenyl]-ethanone

3′,4′-Difluoroacetophenone (100 g, 640 mmol) was dissolved in acetonitrile (700 mL). Sodium thiomethoxide was slowly added to the mixture keeping the temperature below 30° C. The reaction was stirred at ambient temperature overnight. The solids were removed by vacuum filtration and recrystallized from ethyl acetate and hexanes to produce a white crystalline solid, 1-[3-fluoro-4-(methylthio)-phenyl]ethanone (90.5 g, 494 mmol, 77% yield) mp 72-78° C. ¹H NMR (CDCl₃/300 MHz) 7.74 (dd, 1H, J=8.3, J=1.8 Hz). 7.61 (dd, 1H, J=10.7, J=1.8), 7.27 (t, 1H, J=7.8), 2.59 (s, 3H), 2.54 (s, 3H). ESHRMS m/z 185.0412 (M+H⁺, Calcd 184.0436). Anal. Calcd for C₉H₉FOS: C, 58.67; H, 4.92; Found: C, 58.76; H, 4.85.

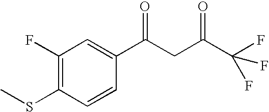

Step 2: Preparation of 4,4,4-trifluoro-1-[3-fluoro-4-(methylthio)phenyl]-1,3-butanedione

1-[3-fluoro-4-(methylthio)phenyl]ethanone (41.50 g, 225 mmol) and ethyl trifluoroacetate was dissolved in diethyl ether (1000 mL). The reaction was stirred at ambient temperature for one hour, aqueous hydrochloric acid was added to adjust the to pH<1.0. The organics were separated and extracted with water (2×250 mL), saturated ammonium chloride (2×250 mL) and dried over sodium sulfate. Solvent was removed in vacuo and 4,4,4-trifluoro-1-[3-fluoro-4-(methylthio)-phenyl]-1,3-butanedione was recrystallized from ethyl acetate and hexanes (48.00 g, 172 mmol, 76% yield): mp 66-68° C. ¹H NMR (CDCl₃/300 MHz) 15.13 (bs 1H), 7.74 (dd, 1H, J=8.3, J=1.8 Hz). 7.61 (dd, 1H, J=10.7, J=1.8), 7.28 (t, 1H, J=7.8), 6.53 (s, 1H), 2.59 (s, 3H). Anal. Calcd for C₉H₉FOS: C, 47.14; H, 2.88; Found: C, 47.50; H, 2.71.

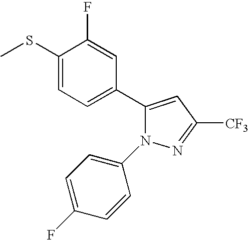

Step 3: Preparation of 5-[3-fluoro-4-(methyl-thio)phenyl]-1-(4-fluorophenyl)-3-(trifluoromethyl)-1H-pyrazole

4,4,4-trifluoro-1-[3-fluoro-4-(methylthio)phenyl]-1,3-butanedione (0.77 g, 2.8 mmol) was dissolved in ethanol (20 mL) and concentrated hydrochloric acid was added to adjust the ph<1.0. 4-Fluorophenylhydrazine hydrochloride (0.48 g, 2.8 mmol) was added and the mixture was heated to reflux. After one hour, the solvent was removed in vacuo and the solid dissolved in ethyl acetate (50 mL). The solution was extracted with water (2×50 mL), saturated ammonium chloride (2×50 mL), dried over sodium sulfate and solvent was removed in vacuo. The resulting yellow oil, 5-[3-fluoro-4-(methylthio)phenyl]-1-(4-fluorophenyl)-3(trifluoro-methyl)-1H-pyrazole was used without further purification. ¹H NMR (CDCl₃/300 MHz) 7.36-7.27 (m, 2H), 7.22-7.10 (m, 3H), 7.6.98-6.89 (m, 2H), 6.77 (s, 1H), 2.50 (s, 3H). ESHRMS m/z 371.0620 (M+H⁺, Calcd 371.0641).

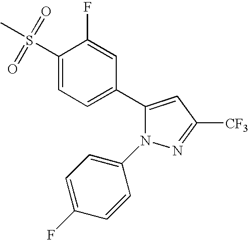

Step 4: Preparation of 5-[3-fluoro-4-(methyl-sulfonyl)phenyl]-1-(4-fluorophenyl)-3-(trifluoromethyl)-1H-pyrazole

5-[3-fluoro-4-(methylthio)phenyl]-1-(4-fluorophenyl)-3-(trifluoromethyl)-1H-pyrazole (1.00 g, 2.75 mmol) and monoperoxyphthalic acid, magnesium salt hexahydrate (2.30 g, 4.69 mmol) were mixed in methylene chloride:methanol (3:1, 20 mL). The mixture was stirred overnight at ambient temperatures. Solids were by vacuum filtration, solvent was removed in vacuo and the resulting slurry was dissolved in ethyl acetate (20 mL). The solution was extracted with water (2×25 mL), saturated ammonium chloride (2×25 mL), dried over sodium sulfate and solvent was removed in vacuo. Crystallization from ethyl acetate and hexanes yielded 5-[3-fluoro-4-(methylsulfonyl)phenyl]-1-(4-fluorophenyl)-3-(trifluoromethyl)-1H-pyrazole (0.66 g, 1.64 mmol, 60% yield): mp 228-229° C. ¹H NMR (CDCl₃/300 MHz) 7.85 (t, 1H, J=7.8 Hz). 7.59-7.28 (m, 7H), 3.37 (s, 3H). ESHRMS m/z 402.0474 (M+H⁺, Calcd 402.0461). Anal. Calcd for C₁₇H₁₁F₅O₂S: C, 50.75; H, 2.76; N, 6.96; Found: C, 50.63; H, 2.57; N, 6.76.

### Example 12

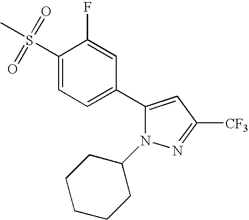

1-Cyclohexyl-5-[3-fluoro-4-(methylsulfonyl)phenyl]-3-(trifluoromethyl)-1H-pyrazole

The title compound was obtained by substituting cyclohexylhydrazine for 4-fluorophenylhydrazine using the method described in example 11: mp 228-229° C. ¹H NMR (DMSOd₆/300 MHz) 8.02 (t, 1H, J=7.6 Hz), 7.78 (d, 1H, J=10.7), 7.62 (d, 1H, J=8.1), 7.02 (s, 1H), 4.24 (m, 1H), 3.42 (s, 3H), 1.98-1.26 (m, 10H). ESHRMS m/z 390.1022 (M, Calcd 390.1025). Anal. Calcd for C₁₇H₁₈F₄N₂O₂S: C, 52.30; H, 4.65; N, 7.18; Found: C, 52.47; H, 4.51; N, 7.11.

### Example 13

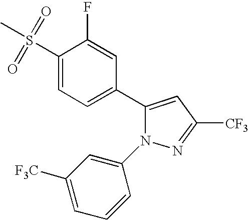

5-[3-Fluoro-4-(methylsulfonyl)phenyl]-3-(trifluoromethyl)-1-[3 (trifluoromethyl)phenyl]-1H-pyrazole

The title compound was obtained by substituting 3-trifluoromethylphenyhydrazine for 4-fluorophenyl-hydrazine using the method described in example 11: 162-168° C. ¹H NMR (DMSOd₆/300 MHz) 7.92-7.84 (m, 3H), 7.76 (m, 3H), 7.49 (s, 1H), 7.34 (dd, 1H, J=8.3, J=1.4), 3.36 (s, 3H). ESHRMS m/z 452.0418 (M, Calcd 452.0429). Anal. Calcd for C₁₈H₁₁F₇N₂O₂S: C, 47.79; H, 2.45; N, 6.19; Found: C, 47.87; H, 2.31; N, 6.12.

### Example 14

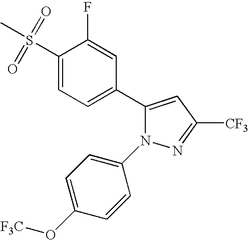

5-[3-Fluoro-4-(methylsulfonyl)phenyl]-3-(trifluoromethyl)-1-[4-(trifluoromethoxy)phenyl]-1H-pyrazole

The title compound was obtained by substituting 4-trifluoromethoxyphenyl-hydrazine for 4-fluorophenyl-hydrazine using the method described in example 11: mp 110-112° C. ¹H NMR (DMSOd₆/300 MHz) 7.86 (t, 1H, J=8.0), 7.62-7.52 (m, 5H), 7.47 (s, 1H), 7.34 (dd, 1H, J=8.0, J=1.4), 3.37 (s, 3H). ESHRMS m/z 468.0338 (M, Calcd 468.0379). Anal. Calcd for C₁₈H₁₁F₇N₂O₃S: C, 46.16; H, 2.37; N, 5.98; Found: C, 46.22; H, 2.49; N, 5.91.

### Example 15

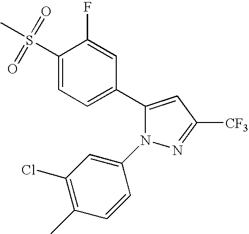

1-(3-Chloro-4-methylphenyl)-5-[3-fluoro-4-(methylsulfonyl)phenyl]-3-(trifluoromethyl)-1H-pyrazole

The title compound was obtained by substituting 3-chloro-4-methylphenylhydrazine for 4-fluorophenyl-hydrazine using the method described in example 11: mp 144-145° C. ¹H NMR (DMSOd₆/300 MHz) 7.86 (t, 1H, J=7.7), 7.64-7.60 (m, 2H), 7.48-7.45 (m, 2H), 7.39 (dd, 1H, J=8.3, J=2.2), 7.27 (dd, 1H, J=8.3, J=2.2), 3.37 (s, 3H), 2.39 (s, 3H). ESHRMS m/z 433.0395 (M+H⁺, Calcd 433.0401). Anal. Calcd for C₁₈H₁₃ClF₄N₂O₂S: C, 49.95; H, 3.03; N, 6.47; Found: C, 49.90; H, 2.84; N, 6.29.

### Example 16

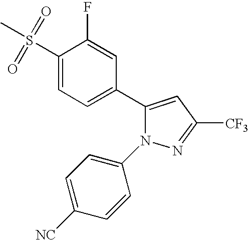

4-[5-[3-Fluoro-4-(methylsulfonyl)phenyl]-3-(trifluoromethyl)-1H-pyrazol-1-yl]benzonitrile

The title compound was obtained by substituting 4-cyanophenylhydrazine for 4-fluorophenylhydrazine using the method described in example 11: mp 152-153° C. ¹H NMR (DMSOd₆/300 MHz) 8.00 (d, 2H, J=8.7), 7.87 (t, 1H, J=7.9), 7.67-7.62 (m, 3H), 7.49 (s, 1H), 7.31 (dd, 1H, J=8.1, J=1.4), 3.38 (s, 3H). ESHRMS m/z 427.0830 (M+NH₄⁺, Calcd 427.0852). Anal. Calcd for C₁₉H₁₁F₄N₃O₂S: C, 52.81; H, 2.71; N, 10.26; Found: C, 52.49; H, 2.43; N, 9.87.

### Example 17

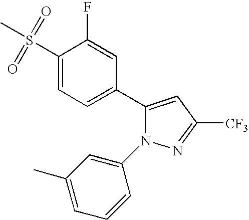

5-[3-Fluoro-4-(methylsulfonyl)phenyl]-1-(3-methylphenyl)-3-(trifluoromethyl)-1H-pyrazole

The title compound was obtained by substituting 3-methylphenylhydrazine for 4-fluorophenylhydrazine using the method described in example 11: mp 129-130° C. ¹H NMR (DMSOd₆/300 MHz) 7.84 (t, 1H, J=7.9), 7.55 (d, 1H, J=11.1), 7.44 (s, 1H), 7.38-7.30 (m, 4H), 7.12 (m, 1H), 3.36 (s, 3H), 2.36 (s, 3H). ESHRMS m/z 399.0838 (M+H⁺, Calcd 399.0790). Anal. Calcd for C₁₈H₁₄F₄N₃O₂S: C, 54.27; H, 3.54; N, 7.03; Found: C, 54.11; H, 3.36; N, 7.23.

### Example 18

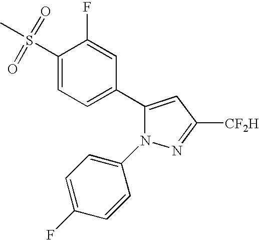

3-(Difluoromethyl)-5-[3-fluoro-4-(methylsulfonyl)phenyl]-1-(4-fluorophenyl)-1H-pyrazole

The title compound was obtained by substituting ethyl difluoroacetate for ethyl trifluoroacetate using the method described in example 11: mp 201-202° C. ¹H NMR (DMSOd₆/300 MHz) 7.79 (t, 1H, J=7.8), 7.65-7.23 (m, 6H), 7.17 (s, 1H), 7.13 (t, 1H, J=54.2), 3.36 (s, 3H). ESHRMS m/z 385.0643 (M+H⁺, Calcd 385.0634). Anal. 3.15; N, 7.29; Found: C, 53.01; H, 3.01; N, 7.23.

### Example 19

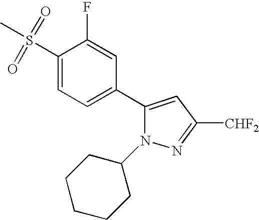

1-Cyclohexyl-3-(difluoromethyl)-5-[3-fluoro-4-(methylsulfonyl)phenyl]-1H-pyrazole

The title compound was obtained by substituting cyclohexylhydrazine for 4-fluorophenylhydrazine and ethyl difluoroacetate for ethyl trifluoroacetate using the method described in example 11: mp 138-139° C. ¹H NMR (DMSOd₆/300 MHz) 8.00 (t, 1H, J=7.9), 7.74 (d, 1H, J=10.9), 7.59 (d, 1H, J=8.1), 7.06 (t, 1H, J=54.6), 6.79 (s, 1H), 4.20 (m, 1H), 3.42 (s, 3H), 1.96-1.21 (m, 10H). ESHRMS m/z 373.1203 (M+H⁺, Calcd 373.1198). Anal. Calcd for C₁₇H₁₉F₃N₂O₂S: C, 54.83; H, 5.14; N, 7.52; Found: C, 54.84; H, 5.06; N, 7.59.

### Example 20

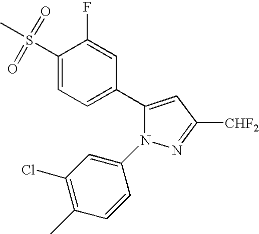

1-(3-Chloro-4-methylphenyl)-3-(difluoromethyl)-5-[3-fluoro-4-(methylsulfonyl)phenyl]-1H-pyrazole

The title compound was obtained by substituting 3-chloro-4-methylphenylhydrazine for 4-fluorophenyl-hydrazine and ethyl difluoroacetate for ethyl trifluoroacetate using the method described in example 11: mp 146-147° C. ¹H NMR (DMSOd₆/300 MHz) 7.85 (t, 1H, J=7.9), 7.62-7.57 (m, 2H), 7.45 (d, 1H, J=8.3), 7.35 (m, 1H), 7.21 (m, 2H), 7.17 (t, 1H, J=54.2), 3.37 (s, 3H), 2.39 (s, 3H). ESHRMS m/z 415.0494 (M+H⁺, Calcd 415.0495). Anal. Calcd for C₁₈H₁₄ClF₃N₂O₂S: C, 52.12; H, 3.40; N, 6.75; Found: C, 52.04; H, 3.29; N, 6.75.

### Example 21

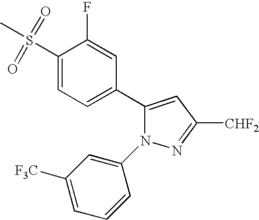

3-(Difluoromethyl)-5-[3-fluoro-4-(methylsulfonyl)-phenyl]-1-[3-(trifluoromethyl)-phenyl]-1H-pyrazole

The title compound was obtained by substituting 3-trifluoromethylphenylhydrazine for 4-fluorophenyl-hydrazine and ethyl difluoroacetate for ethyl trifluoroacetate using the method described in example 11: mp 110-111° C. ¹H NMR (DMSOd₆/300 MHz) 7.88-7.83 (m, 3H), 7.71 (t, 1H, J=8.1), 7.63-7.59 (m, 2H), 7.33 (dd, 1H, J=8.3, J=1.4), 7.25 (s, 1H), 7.21 (t, 1H, J=54.2), 3.36 (s, 3H). ESHRMS m/z 435.0599 (M+H⁺, Calcd 435.0602). Anal. Calcd for C₁₈H₁₂F₆N₂O₂S: C, 49.77; H, 2.78; N, 6.45; Found: C, 49.77; H, 2.68; N, 6.30.

### Example 22

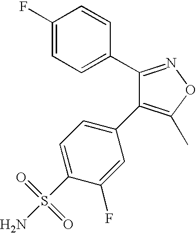

2-Fluoro-4-[3-(4-fluorophenyl)-5-methyl-4-isoxazolyl]benzenesulfonamide

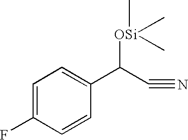

Step 1: Preparation of 4-fluoro-α-[(trimethylsilyl)-oxy]benzeneacetonitrile

4-Fluorobenzaldehyde (21.0 g, 170 mmol) and zinc iodide (30 mg) were mixed together in methylene chloride (100 mL). The solution was cooled to −70° C. and trimethylsilyl cyanide was slowly added (16.8 g, 170 mmol). The cooling bath was removed and the solution stirred for 1 hour while warming to ambient temperature. The solution was extracted with water (2×100 mL), saturated ammonium chloride (2×100 mL) and dried over sodium sulfate. Solvent was removed in vacuo to yield 4-fluoro-α-[(trimethylsilyl)oxy]benzene-acetonitrile as a yellow oil (23.0 g, 95.8 mmol, 61% yield): ¹H NMR (CDCl₃/300 MHz) 7.46-7.43 (m, 2H), 7.11-7.07 (m, 2H), 5.46 (s, 1H), 0.22 (s, 9H).

ESHRMS m/z 241.1172 (M+NH₄⁺, Calcd 241.1172).

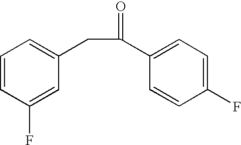

Step 2: Preparation of 2-(3-fluorophenyl)-1-(4-(fluorophenyl)ethanone.

4-Fluoro-α-[(trimethylsilyl)oxy]benzene-acetonitrile (23.0 g, 104 mmol) was cooled to −78° C. in tetrahydrofuran (300 mL). 1.0 M Lithium diisopropylamide in tetrahydrofuran (115 mL, 115 mmol) was added dropwise over 1 hour. The solution was stirred for 1 hour at −78° C. 3-Fluorobenzyl bromide (19.6 g, 104 mmol) was added as a solution in tetrahydrofuran (100 mL) over 25 minutes. The solution was held at −78° C. for 1 hour then warmed to 0° C. for two hours. Aqueous hydrochloric acid (10%×200 mL) was added and the solution and stirred for 24 hours at ambient temperature. The layers were separated and the organic layer was mixed with aqueous 15% sodium hydroxide (100 mL) and stirred for 24 hours at ambient temperature. The organic layer was collected, washed with saturated ammonium chloride (200 mL) and dried over sodium sulfate. The solvent was removed in vacuo to afford a yellow oil. 2-(3-fluorophenyl)-1-(4-(fluorophenyl)ethanone (10.15 g, 43.0 mmol) was isolated by preparative silica chromatography followed by crystallization from ethyl acetate and hexanes (42% yield): mp 47-48° C. ¹H NMR (CDCl₃/300 MHz) 8.08-8.03 (m, 2H), 7.33-7.29 (m, 1H), 7.14 (t, 2H, f=8.7), 7.07-6.96 (m, 3H), 4.28 (s, 2H). ESHRMS m/z 233.0791 (M+H⁺, Calcd 233.0778). Anal. Calcd for C₁₄H₁₁F₂O: C, 72.41; H, 4.34; Found: C, 72.32; H, 4.30.

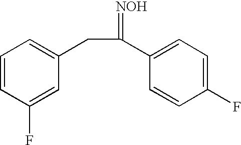

Step 3: Preparation of 2-(3-fluorophenyl)-[(4-fluorophenyl)ethanone Oxime

2-(3-fluorophenyl)-1-(4-(fluorophenyl)ethanone (8.82 g, 38 mmol), hydroxylamine hydrochloride (5.55 g, 38 mmol) and ammonium acetate (6.50 g, 38 mmol) was refluxed in ethanol (150 mL) and water (50 mL) for 1 hour. Ethanol was removed in vacuo and the aqueous suspension poured into ethyl acetate (200 mL), extracted with water (2×200 mL), saturated ammonium chloride (2×250 mL) and dried over sodium sulfate. The solvent was removed in vacuo and 2-(3-Fluorophenyl)-1(4-fluorophenyl)ethanone oxime (6.19 g, 25.0 mmol) was obtained by crystallization form boiling hexanes (65% yield): mp 60-67° C. ¹H NMR (CDCl₃/300 MHz) 7.64-7.59 (m, 2H), 7.28-7.23 (m, 1H), 7.10-6.93 (m, 5H), 4.23 (s, 2H). ESHRMS m/z 248.0899 (M+H⁺, Calcd 248.0887). Anal. Calcd for C₁₄H₁₁F₂NO: C, 68.01; H, 4.48; N, 5.67; Found: C, 67.86; H, 4.44; N, 5.63.

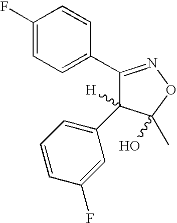

Step 4: Preparation of 4-(3-fluorophenyl)-3-(4-fluoro-phenyl-4,5-dihydro-5-methyl-5-isoxazolol (Mixture of Diastereoomers)

2-(3-Fluorophenyl)-1-(4-fluorophenyl)ethanone oxime (7.39 g, 30 mmol) was dissolved in tetrahydrofuran (500 mL) and cooled to −78° C. 2.5 M n-butyl lithium (50 mL) was added over 1 hour and held at −78° C. for an additional hour. Acetyl imidazole (6.51 g, 60 mmol) was added and the suspension was warmed to ambient temperature. 10% Aqueous hydrochloric acid was added to adjust the solution to pH to <1.0. The solution was extracted with water (2×250 mL), ammonium chloride (2×250 mL) and dried over sodium sulfate. A mixture of 4-(3-Fluorophenyl)-3-(4-fluorophenyl-4,5-dihydro-5-methyl-5-isoxazolol diastereomers was obtained by preparative silica chromatography followed by crystallization from ethyl acetate and hexanes (4.07 g, 14.08 mmol, 47% yield): mp 130-132° C. ¹H NMR (CD₃OD/300 M]) 7.65-7.60 (m, 2H), 7.39-7.30 (m, 1H), 7.05-6.99 (m, 5H), 4.50 (s, 1H), 3.00 (br s, 1H), 1.33 (s, 3H). ESHRMS m/z 290.0993 (M+H⁺, Calcd 290.0993). Anal. Calcd for C₁₆H₁₃F₂NO₂: C, 66.43; H, 4.53; N, 4.84; Found: C, 66.48; H, 4.54; N, 4.76.

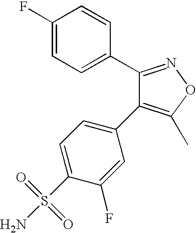

Step 5: Preparation of 2-fluoro-4-[3-(4-fluorophenyl)-5-methyl-4-isoxazolyl]benzenesulfonamide

Chlorosulfonic acid (8.0 mL) was cooled to 0° C. and 4-(3-fluorophenyl)-3-(4-fluorophenyl-4,5-dihydro-5-methyl-5-isoxazolol (0.53 g, 18 mmol) was added. After stirring at 0° C. for 1 hour, the solution was added drop-wise onto ice (200 mL) with vigorous stirring. The solution was extracted with ethyl acetate (2×100 mL). The ethyl acetate layers were combined, washed with water (2×100 mL), saturated ammonium chloride (2×100 mL). Ammonium hydroxide (50 mL) was added to the ethyl acetate solution and the mixture was stirred overnight at ambient temperature. Saturated ammonium chloride was added (50 mL) and the ethyl acetate layer isolated, extracted with saturated ammonium chloride (2×100 mL) and dried over sodium sulfate. 2-Fluoro-4-[3-(4-fluorophenyl)-5-methyl-4-isoxazolyl]benzene-sulfonamide (0.10 g, 0.28 mmol) was obtained by preparative silica chromatography followed by crystallization from ethyl acetate and hexanes (0.10 g, 15% yield): mp 176-177° C. ¹HNMR (CD₃OD/300 MHz) 6.37 (t, 1H, J=7.7 Hz), 5.92-5.88 (m, 2H), 5.67˜5.60 (m, 4H), 0.98 (s, 3H). ESHRMS m/z 351.0598 (M+H⁺, Calcd 351.0615). Anal. Calcd for C₁₆H₁₂F₂N₂O₃S: C, 54.85; H, 3.45; N, 8.00; Found: C, 54.99; H, 3.33; N, 7.82. Example 23

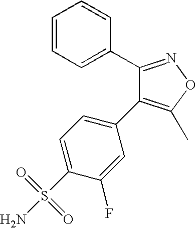

2-Fluoro-4-[3-(4-fluorophenyl)-5-methyl-4-isoxazolyl]-benzenesulfonamide

The title compound was obtained by substituting benzaldehyde for 4-fluorobenzaldehyde using the method described in example 11: mp 180-182° C. ¹H NMR (CD₃OD/300 MHz) 7.86 (m, 1H), 7.38-7.48 (m, 5H), 7.18-7.23 (m, 2H), 6.90 (bs, 1H), 2.52 (s, 3H), ESHRMS m/z 333.0727 (M+H⁺, Calcd 333.0709). Anal. Calcd for C₁₆H₁₃FN₂O₃S: C, 57.82; H, 3.94; N, 8.43; Found: C, 57.43; H, 4.00; N, 8.31.

### Example 24

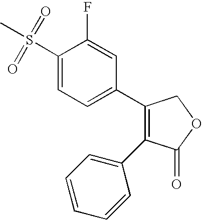

4-[3-Fluoro-4-(methylsulfonyl)phenyl]-3-phenyl-2(5H)-furanone

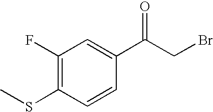

Step 1: Preparation of 2-bromo-1-[3-fluoro-4-(methylthio)phenyl]ethanone

1-[3-Fluoro-4-(methylthio)phenyl]ethanone (10.30 g, 56.0 mmol) was suspended in glacial acetic acid (50 mL) and hydrogen bromide (20 mL, 35% in acetic acid). Bromine (8.95 g, 56 mmol) was added and the reaction was stirred at ambient temperature for 30 minutes. Ethyl acetate (50 mL) was added to the reaction and it was extracted with water (2×50 mL), saturated ammonium chloride (2×50 mL) and dried over sodium sulfate. Solvent was removed in vacuo to yield 2-bromo-1-[3-fluoro-4-(methylthio)phenyl]ethanone as a yellow solid, (10.45 g, 39.8 mmol, 71% yield): mp 70-71° C. ¹H NMR (CDCl₃/300 MHz) 7.76 (dd, 1H, J=8.3, 1.8 Hz), 7.65 (dd, 1H, J=10.7, 1.8 Hz), 7.29 (t, 1H, J=7.7 Hz), 4.40 (s, 2H), 2.56 (s, 3H). ESHRMS m/z 262.9544 (M+H⁺, Calcd 262.9542). Anal. Calcd for C₉H₈FBrOS: C, 41.08; H, 3.06; Pound: C, 41.00; H, 2.88.

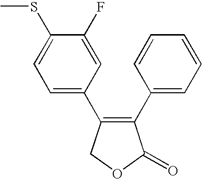

Step 2: Preparation of 4-[3-fluoro-4-(methylthio)-phenyl]-3-phenyl-2(5H)-furanone

Phenylacetic acid (3.61 g, 26.5 mmol) and sodium hydroxide (2.6 g, 50% aqueous) were dissolved in dimethylformamide and stirred vigorously for 15 min. 2-bromo-1-[3-fluoro-4-(methylthio)phenyl]ethanone (5.17 g, 19.6 mmol) was added and the reaction was warmed to 45° C. for one hour. Ethyl acetate (50 mL) was added to the reaction and the organics were extracted with 10% aqueous hydrochloric acid (2×50 mL), water (2×50 mL), saturated ammonium chloride (2×50 mL) and dried over sodium sulfate. Solvent was removed in vacuo to yield a yellow solid. The solid was dissolved in methylene chloride and concentrated sulfuric acid (1 mL) was added. After 15 min, the solution was extracted with water (2×50 mL), saturated ammonium chloride (2×50 mL) and dried over sodium sulfate. The solvent was removed in vauo and 4-[3-fluoro-4-(methylthio)phenyl]-3-phenyl-2(5H)-furanone (1.85 g, 6.16 mmol) was obtained by crystallization from ethyl acetate and (31% yield): mp 159-160° C. ¹H NMR (CDCl₃/300 MHz) 7.46-7.44 (m, 5H), 7.20-7.09 (m, 2H), 7.00 (dd, 1H, J=11.1, 1.8 Hz), 5.17 (s, 2H), 2.50 (s, 3H). ESHRMS iii/z 301.0680 (M+H⁺, Calcd 301.0699). Anal. Calcd for C₁₇H₁₃F O₂S: C, 67.98; H, 4.36; Found: C, 67.55; H, 4.10.

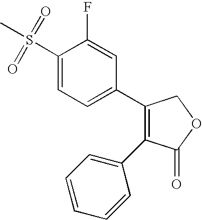

Step 3: Preparation of 4-[3-fluoro-4-(methylsulfonyl)-phenyl]-3-phenyl-2(5H)-furanone

4-[3-Fluoro-4-(methylthio)phenyl]-3-phenyl-2(5H)-furanone (1.80 g, 6.00 mmol) and monoperoxyphthalic acid magnesium salt hexahydrate (3.70 g, 6.00 mmol) were mixed in methylene chloride:methanol (3:1, 40 mL). The mixture was stirred overnight at ambient temperatures. Solids were removed by vacuum filtration and discarded, solvent was removed in vacuo and the resulting slurry was dissolved in ethyl acetate (20 mL). The solution was extracted with water (2×25 mL), saturated ammonium chloride (2×25 mL), dried over sodium sulfate and solvent was removed in vacuo. Crystallization from ethyl acetate and hexanes yielded 4-[3-fluoro-4-(methylsulfonyl)phenyl]-3-phenyl-2(5H)-furanone (0.63 g, 1.89 mmol, 32% yield): mp 161-162° C. ¹H NMR (CDCl₃/300 MHz) 7.98 (t, 1H, J=8.5 Hz), 7.47-7.40 (m, 5H), 7.30 (dd, 1H, J=8.3, 1.6), 7.21 (dd, 1H, J=10.5, 1.4), 5.20 (s, 2H), 3.26 (s, 1H). ESHRMS m/z 333.0606 (M+H⁺, Calcd 333.0597). Anal. Calcd for C₁₇H₁₃FO₄S: C, 61.44; H, 3.94; Found: C, 61.65; H, 3.93.

### Biological Evaluation

Evaluation of COX-1 and COX-2 Activity In Vitro

The compounds of this invention exhibited inhibition in vitro of COX-2. The COX-2 inhibition activity of the compounds of this invention illustrated in the Examples was determined by the following methods.

A. Preparation of Recombinant COX Baculoviruses

Recombinant COX-1 and COX-2 were prepared as described by Gierse et al, [J. Biochem., 305, 479-84 (1995)]. A 2.0 kb fragment containing the coding region of either human or murine COX-1 or human or murine COX-2 was cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses were isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses were purified by three rounds of plaque purification and high titer (10⁷-10⁸ pfu/mL) stocks of virus were prepared. For large scale production, SF9 insect cells were infected in 10 liter fermentors (0.5×10⁶/mL) with the recombinant baculovirus stock such that the multiplicity of infection was 0.1. After 72 hours the cells were centrifuged and the cell pellet homogenized in Tris/Sucrose (50 mM: 25%, pH 8.0) containing 1% 3-[(3-cholamidopropyl)dimethylammonio]-1-propanesulfonate (CHAPS). The homogenate was centrifuged at 10,000×G for 30 minutes, and the resultant supernatant was stored at −80° C. before being assayed for COX activity.

B. Assay for COX-1 and COX-2 Activity

COX activity was assayed as PGE₂ formed/μg protein/time using an ELISA to detect the prostaglandin released. CHAPS-solubilized insect cell membranes containing the appropriate COX enzyme were incubated in a potassium phosphate buffer (50 mM, pH 8.0) containing epinephrine, phenol, and heme with the addition of arachidonic acid (10 μM). Compounds were pre-incubated with the enzyme for 10-20 minutes prior to the addition of arachidonic acid. Any reaction between the arachidonic acid and the enzyme was stopped after ten minutes at 37° C./room temperature by transferring 40 μl of reaction mix into 160 μl ELISA buffer and 25 μM indomethacin. The PGE₂ formed was measured by standard ELISA technology (Cayman Chemical). Results are shown in Table I.

TABLE I

| | Human COX-2 | Human COX-1 |
|---|---|---|
| Example | IC₅₀ (μM) | IC₅₀ (μM) |
| 1 | 0.011 | 37.9 |
| 2 | 0.051 | >100 |
| 3 | 0.005 | 16.3 |
| 4 | 0.019 | 40.5 |
| 5 | 0.005 | 45.9 |
| 6 | 0.025 | 45.6 |
| 7 | 0.005 | 7.72 |
| 8 | 0.020 | 56.8 |
| 9 | 0.250 | >100 |
| 10 | 0.110 | >100 |
| 11 | 0.150 | >100 |
| 12 | 0.180 | >100 |
| 13 | 0.180 | >100 |
| 14 | 0.062 | >100 |
| 15 | 0.016 | >100 |
| 16 | 0.250 | >100 |
| 17 | 1.05 | >100 |
| 18 | 0.320 | >100 |
| 19 | 0.700 | >100 |
| 20 | 0.036 | >100 |
| 21 | 0.550 | >100 |
| 22 | 0.015 | >100 |
| 23 | 0.020 | >100 |
| 24 | 15.9 | >100 |

C. Fast assay for COX-1 and COX-2 Activity

COX activity was assayed as PGE₂ formed/μg protein/time using an ELISA to detect the prostaglandin released. CHAPS-solubilized insect cell membranes containing the appropriate COX enzyme were incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds were pre-incubated with the enzyme for 10 minutes at 25° C. prior to the addition of arachidonic acid. Any reaction between the arachidonic acid and the enzyme was stopped after two minutes at 37° C./room temperature by transferring 40 μl of reaction mix into 160 μl ELISA buffer and 25 μM indomethacin. The PGE₂ formed was measured by standard ELISA technology (Cayman Chemical). Results are shown in Table II.

TABLE II

| | Human COX-2 | Human COX-1 |
|---|---|---|
| Example | IC₅₀ (μM) | IC₅₀ (μM) |
| 1 | 0.013 | 29.4 |
| 2 | 0.043 | >100 |
| 3 | 0.004 | 26.9 |
| 4 | 0.006 | 20.3 |
| 5 | 0.004 | 19.9 |
| 6 | 0.018 | >100 |
| 7 | 0.004 | 5.25 |
| 8 | 0.021 | 41.5 |
| 9 | 0.180 | >100 |
| 10 | na | na |
| 11 | 0.130 | 22.6 |
| 12 | 0.140 | >100 |
| 13 | 0.150 | >100 |
| 14 | 0.064 | >100 |
| 15 | 0.015 | 2.36 |
| 16 | 0.870 | >100 |
| 17 | 0.027 | 4.36 |
| 18 | 0.310 | >100 |
| 19 | 0.340 | >100 |
| 20 | 0.053 | >100 |
| 21 | 0.290 | >100 |
| 22 | 0.005 | >100 |
| 23 | 0.019 | >100 |
| 24 | 3.73 | >100 |

Rat Carrageenan Foot Pad Edema Test

The carrageenan foot edema test is performed with materials, reagents and procedures essentially as described by Winter, et al., (Proc. Soc. Exp. Biol. Med., 111, 544 (1962)). Male Sprague-Dawley rats are selected in each group so that the average body weight is as close as possible. Rats are fasted with free access to water for over sixteen hours prior to the test. The rats are dosed orally (1 mL) with compounds suspended in vehicle containing 0.5% methylcellulose and 0.025% surfactant, or with vehicle alone. One hour later a subplantar injection of 0.1 mL of 1% solution of carrageenan/sterile 0.9% saline is administered and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDs, in Non-steroidal Anti-Inflammatory Drugs, (J. Lombardino, ed. 1985)).

Rat Carrageenan-Induced Analgesia Test

The rat carrageenan analgesia test is performed with materials, reagents and procedures essentially as described by Hargreaves, et al., (Pain, 32, 77 (1988)). Male Sprague-Dawley rats are treated as previously described for the Carrageenan Foot Pad Edema test. Three hours after the injection of the carrageenan, the rats are placed in a special plexiglass container with a transparent floor having a high intensity lamp as a radiant heat source, positionable under the floor. After an initial twenty minute period, thermal stimulation is begun on either the injected foot or on the contralateral uninjected foot. A photoelectric cell turns off the lamp and timer when light is interrupted by paw withdrawal. The time until the rat withdraws its foot is then measured. The withdrawal latency in seconds is determined for the control and drug-treated groups, and percent inhibition of the hyperalgesic foot withdrawal is determined.

As various changes could be made in the above methods and apparatus without departing from the scope of the invention, it is intended that all matter contained in the above description be interpreted as illustrative and not in a limiting sense. All documents mentioned in this application are expressly incorporated by reference as if fully set forth at length.

All mentioned references are incorporated by reference as if here written. When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A compound of Formula I: 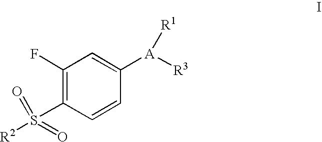
wherein:
A is a 5- or 6-member ring substituent selected from partially saturated or unsaturated-heterocyclic and carbocyclic rings;
R¹ is cyclohexyl, pyridinyl, or phenyl, wherein said cyclohexyl, pyridinyl, and phenyl may be may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R² is methyl or amino; and
R³ represents one or more radicals selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl;
a pharmaceutically-acceptable salt, tautomer or prodrug thereof;
provided that (a) A is not pyrrolyl, and (b) A is not oxazolyl other than oxazolonyl;
provided that when R¹ is 4-bromophenyl: (a) A is not pyrazolyl when R² is methyl and R³ is hydrogen, cyano, trifluoromethyl or ethoxycarbonyl; (b) A is not imidazolyl when R³ is trifluoromethyl; (c) A is not isoxazolyl when R³ is methyl; and (d) A is not 2-furanonyl when R³ is hydrogen; and
provided that when R¹ is 3-methyl-4-bromophenyl, R² is methyl and R³ is trifluoromethyl, A is not imidazolyl.

2. Compound of claim 1 wherein:
A is a 5- or 6-member ring substituent selected from partially saturated or unsaturated heterocyclic and carbocyclic rings;
R¹ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R² is methyl or amino; and
R³ represents one or more radicals selected from hydrido, halo, C₁₋₂-alkyl, C₂₋₃-alkenyl, C₂₋₃-alkynyl, oxo, cyano, carboxyl, cyano-C₁₋₃-alkyl, (5- or 6-member ring heterocyclyl)oxy, C₁₋₃-alkoxy, C₁₋₃-alkylthio, C₁₋₃-alkylcarbonyl, C₃₋₆-cycloalkyl, phenyl, C₁₋₃-haloalkyl, 5- or 6-member ring heterocyclyl, C₃₋₆-cycloalkenyl, phenyl-C₁₋₃-alkyl, (5- or 6-member ring heterocyclyl)-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxy-C₁₋₃-alkyl, C₁₋₃-alkoxyphenyl-C₁₋₃-alkoxy-C₁₋₃-alkyl, C₁₋₃-alkoxycarbonyl-C₁₋₃-alkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-phenylamino, N-(phenyl-C₁₋₃-alkyl)amino, N-(C₁₋₃-alkyl)-N-(phenyl-C₁₋₃-alkyl)amino, N-(C₁₋₃-alkyl)-N-phenylamino, amino-C₁₋₃-alkyl, C₁₋₃-alkylamino-C₁₋₃-alkyl, N-phenylamino-C₁₋₃-alkyl, N-phenyl-C₁₋₃-alkylamino-C₁₋₃-alkyl, N-(C₁₋₃-alkyl)-N-phenyl-C₁₋₃-alkylamino-C₁₋₃-alkyl, N-(C₁₋₃-alkyl)-N-phenylamino-C₁₋₃-alkyl, phenyloxy, phenyl-C₁₋₃-alkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl;
or a pharmaceutically-acceptable salt, tautomer or prodrug thereof.

3. Compound of claim 2 wherein A is a 5- or 6-member ring substituent selected from partially saturated or unsaturated heterocyclic rings.

4. Compound of claim 2 wherein A is a 5- or 6-member ring substituent selected from partially saturated or unsaturated carbocyclic rings.

5. Compound of claim 2 wherein A is a radical selected from thienyl, furyl, furanone, thiazolyl, oxothiazolyl, thioxothiazolyl, imidazolyl, benzofuryl, indenyl, benzothienyl, isoxazolyl, oxooxazolyl, pyrazolyl, cyclopentenyl, cyclopentadienyl, benzindazolyl, benzopyranopyrazolyl, phenyl, and pyridyl.

6. Compound of claim 2 wherein A is a radical selected from thienyl, furyl, furanone, thiazolyl, oxothiazolyl, thioxothiazolyl, imidazolyl, benzofuryl, indenyl, benzothienyl, isoxazolyl, pyrazolyl, cyclopentenyl, cyclopentadienyl, benzindazolyl, benzopyranopyrazolyl, phenyl, and pyridyl.

7. Compound of claim 2 wherein A is a radical selected from thienyl, furanone, isoxazolyl, pyrazolyl, cyclopentenyl and pyridinyl.

8. Compound of claim 2 wherein A is a radical selected from furanone, isoxazolyl, and pyrazolyl.

9. Compound of claim 6 wherein R¹ is optionally substituted cyclohexyl.

10. Compound of claim 6 wherein R¹ is optionally substituted pyridinyl.

11. Compound of claim 6 wherein R¹ is optionally substituted phenyl.

12. Compound of claim 6 wherein R¹ is cyclohexyl, pyridinyl, or phenyl, wherein said cyclohexyl, pyridinyl, and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio.

13. Compound of claim 6 wherein R³ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

14. Compound of claim 6 wherein
R¹ is cyclohexyl or phenyl, wherein said cyclohexyl and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio; and
R³ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

15. Compound of claim 6 wherein
R¹ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and
R³ is a radical selected from hydrido, C₁₋₂-alkyl, C₁₋₃-alkoxy, C₁₋₃-alkylcarbonyl, C₁₋₃-haloalkyl, C₁₋₃-hydroxyalkyl, and C₁₋₃-alkoxycarbonyl.

16. Compound of claim 15 wherein
R¹ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, and methoxy; and
R³ is a radical selected from hydrido, methyl, methoxy, methylcarbonyl, trifluoromethyl, difluoromethyl, hydroxymethyl, and methoxycarbonyl.

17. A compound of claim 1 having Formula II: 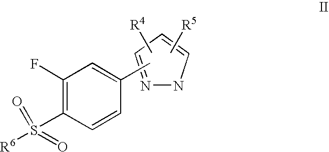
wherein:
R⁴ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R⁵ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy -C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R⁶ is methyl or amino;
or a pharmaceutically-acceptable salt, tautomer or prodrug thereof;
provided that when R⁴ is 4-bromophenyl and R⁶ is methyl, R⁵ is not hydrogen, cyano, trifluoromethyl or ethoxycarbonyl.

18. Compound of claim 17 wherein:
R⁴ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R⁵ is a radical selected from hydrido, halo, C₁₋₂-alkyl, C₂₋₃-alkenyl, C₂₋₃-alkynyl, oxo, cyano, carboxyl, cyano-C₁₋₃-alkyl, (5- or 6-member ring heterocyclyl)oxy, C₁₋₃-alkoxy, C₁₋₃-alkylthio, C₁₋₃-alkylcarbonyl, C₃₋₆-cycloalkyl, phenyl, C₁₋₃-haloalkyl, 5- or 6-member ring heterocyclyl, C₃₋₆-cycloalkenyl, phenyl-C₁₋₃-alkyl, (5- or 6-member ring heterocyclyl)-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxy-C₁₋₃-alkyl, C₁₋₃-alkoxyphenyl-C₁₋₃-alkoxy-C₁₋₃-alkyl, C₁₋₃-alkoxycarbonyl-C₁₋₃-alkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-phenylamino, N-(phenyl-C₁₋₃-alkyl)amino, N-(C₁₋₃-alkyl)-N-(phenyl-C₁₋₃-alkyl)amino, N-(C₁₋₃-alkyl)-N-phenylamino, amino-C₁₋₃-alkyl, C₁₋₃-alkylamino-C₁₋₃-alkyl, N-phenylamino-C₁₋₃-alkyl, N-phenyl-C₁₋₃-alkylamino -C₁₋₃-alkyl, N-(C₁₋₃-alkyl)-N-phenyl-C₁₋₃-alkylamino-C₁₋₃-alkyl, N-(C₁₋₃-alkyl) -N-phenylamino-C₁₋₃-alkyl, phenyloxy, phenyl-C₁₋₃-alkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R⁶ is methyl or amino; or
a pharmaceutically-acceptable salt, tautomer or prodrug thereof.

19. Compound of claim 18 wherein R⁴ is optionally substituted cyclohexyl.

20. Compound of claim 18 wherein R⁴ is optionally substituted pyridinyl.

21. Compound of claim 18 wherein R⁴ is optionally substituted phenyl.

22. Compound of claim 18 wherein R⁴ is cyclohexyl, pyridinyl, or phenyl, wherein said cyclohexyl, pyridinyl, and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio.

23. Compound of claim 18 wherein R⁵ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

24. Compound of claim 18 wherein:
R⁴ is cyclohexyl or phenyl, wherein said cyclohexyl and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio; and
R⁵ is a radical selected from hydrido, fluoro, chloro, bromo, methyl, oxo, cyano, carboxyl, cyanomethyl, methoxy, methylthio, methylcarbonyl, phenyl, trifluoromethyl,
difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-Ns phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

25. A compound of claim 24 having Formula IIA: 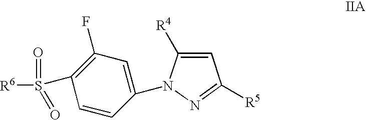
wherein R⁴, R⁵ and R⁶ are as defined in claim 24.

26. A compound of claim 24 having Formula IIB: 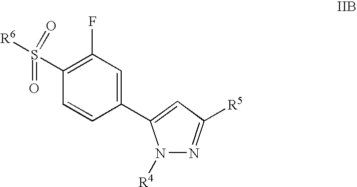
wherein R⁴, R⁵ and R⁶ are as defined in claim 24.

27. Compound of claim 18 wherein:
R⁴ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and
R⁵ is a radical selected from hydrido, C₁₋₂-alkyl, C₁₋₃-alkoxy, C₁₋₃-alkylcarbonyl, C₁₋₃-haloalkyl, C₁₋₃-hydroxyalkyl, and C₁₋₃-alkoxycarbonyl.

28. Compound of claim 18 wherein
R⁴ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, and methoxy; and
R⁵ is a radical selected from hydrido, methyl, methoxy, methylcarbonyl, trifluoromethyl, difluoromethyl, hydroxymethyl, and methoxycarbonyl.

29. Compound of claim 18 wherein the compound of Formula I is 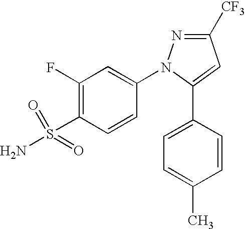
or a pharmaceutically-acceptable salt, tautomer or prodrug thereof.

30. Compound of claim 18 wherein the compound of Formula I is 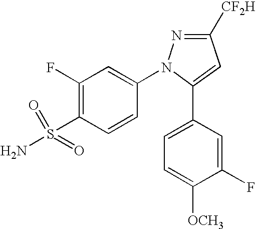
or a pharmaceutically-acceptable salt, tautomer or prodrug thereof.

31. A compound of claim 1 having Formula m: 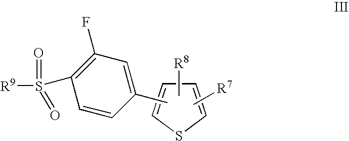
wherein:
R⁷ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R⁸ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-aryl amino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R⁹ is methyl or amino; or
a pharmaceutically-acceptable salt, tautomer or prodrug thereof.

32. Compound of claim 31 wherein:
R⁷ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R⁸ is a radical selected from hydrido, halo, C₁₋₂-alkyl, C₂₋₃-alkenyl, C₂₋₃-alkynyl, oxo, cyano, carboxyl, cyano-C₁₋₃-alkyl, (5- or 6-member ring heterocyclyl)oxy, C₁₋₃-alkoxy, C₁₋₃-alkylthio, C₁₋₃-alkylcarbonyl, C₃₋₆-cycloalkyl, phenyl, C₁₋₃-haloalkyl, 5- or 6-member ring heterocyclyl, C₃₋₆-cycloalkenyl, phenyl-C₁₋₃-alkyl, (5- or 6-member ring heterocyclyl)-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxy-C₁₋₃-alkyl, C₁₋₃-alkoxyphenyl-C₁₋₃-alkoxy-C₁₋₃-alkyl, C₁₋₃-alkoxycarbonyl-C₁₋₃-alkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-phenylamino, N-(phenyl-C₁₋₃-alkyl)amino, N-(C₁₋₃-alkyl)-N-(phenyl-C₁₋₃-alkyl)amino, N-(C₁₋₃-alkyl)-N-phenylamino, amino-C₁₋₃-alkyl, C₁₋₃-alkylamino-C₁₋₃-alkyl, N-phenylamino-C₁₋₃-alkyl, N-phenyl-C₁₋₃-alkylamino-C₁₋₃-alkyl, N-(C₁₋₃-alkyl)-N-phenyl-C₁₋₃-alkylamino-C₁₋₃-alkyl, N-(C₁₋₃-alkyl)-N-phenylamino-C₁₋₃-alkyl, phenyloxy, phenyl-C₁₋₃-alkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R⁹ is methyl or amino; or
a pharmaceutically-acceptable salt, tautomer or prodrug thereof.

33. Compound of claim 32 wherein R⁷ is optionally substituted cyclohexyl.

34. Compound of claim 32 wherein R⁷ is optionally substituted pyridinyl.

35. Compound of claim 32 wherein R⁷ is optionally substituted phenyl.

36. Compound of claim 32 wherein R⁷ is cyclohexyl, pyridinyl, or phenyl, wherein said cyclohexyl, pyridinyl, and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio.

37. Compound of claim 32 wherein R⁸ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

38. Compound of claim 32 wherein:
R⁷ is cyclohexyl or phenyl, wherein said cyclohexyl and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio; and
R⁸ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

39. A compound of claim 38 having Formula IIIA: 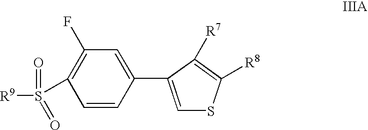
wherein R⁷, R⁸ and R⁹ are as defined in claim 38.

40. Compound of claim 32 wherein:
R⁷ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and
R⁸ is a radical selected from hydrido, halogen, C₁₋₂-alkyl, C₁₋₃-alkoxy, C₁₋₃-alkylcarbonyl, C₁₋₃-haloalkyl, C₁₋₃-hydroxyalkyl, and C₁₋₃-alkoxycarbonyl.

41. Compound of claim 32 wherein
R⁷ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, iodo and methoxy; and
R⁸ is a radical selected from hydrido, chloro, fluoro, bromo, cyano, methyl, methoxy, methylcarbonyl, trifluoromethyl, difluoromethyl, hydroxymethyl, and methoxycarbonyl.

42. A compound of claim 1 having Formula IV: 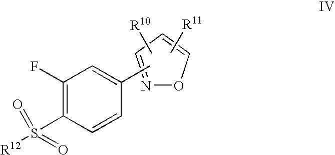
wherein:
R¹⁰ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹¹ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
wherein R¹² is methyl or amino; or
a pharmaceutically-acceptable salt, tautomer or prodrug thereof
provided that when R¹⁰ is 4-bromophenyl, R¹¹ not is methyl.

43. Compound of claim 42 wherein:
R¹⁰ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹¹ is a radical selected from hydrido, halo, C₁₋₂-alkyl, C₂₋₃-alkenyl, C₂₋₃-alkynyl, oxo, cyano, carboxyl, cyano-C₁₋₃-alkyl, (5- or 6-member ring heterocyclyl)oxy, C₁₋₃-alkoxy, C₁₋₃-alkylthio, C₁₋₃-alkylcarbonyl, C₁₋₃-cycloalkyl, phenyl, C₁₋₃-haloalkyl, 5- or 6-member ring heterocyclyl, C₃₋₆-Cycloalkenyl, phenyl-C₁₋₃-alkyl, (5- or 6-member ring heterocyclyl)-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxy-C₁₋₃-alkyl, C₁₋₃-alkoxyphenyl-C₁₋₃-alkoxy-C₁₋₃-alkyl, C₁₋₃-alkoxycarbonyl-C₁₋₃-alkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-phenylamino, N-(phenyl-C₁₋₃-alkyl)amino, N-(C₁₋₃-alkyl)-N-(phenyl-C₁₋₃-alkyl)amino, N-(C₁₋₃-alkyl)-N-phenylamino, amino-C₁₋₃-alkyl, C₁₋₃-alkylamino-C₁₋₃-alkyl, N-phenylamino-C₁₋₃-alkyl, N-phenyl-C₁₋₃-alkylamino-C₁₋₃-alkyl, N-(C₁₋₃-alkyl)-N-phenyl-C₁₋₃-alkylamino-C₁₋₃-alkyl, N-(C₁₋₃-alkyl)-N-phenylamino-C₁₋₃-alkyl, phenyloxy, phenyl-C₁₋₃-alkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N phenylaminosulfonyl; and
R¹² is methyl or amino; or
a pharmaceutically-acceptable salt, tautomer or prodrug thereof.

44. Compound of claim 43 wherein R¹⁰ is optionally substituted cyclohexyl.

45. Compound of claim 43 wherein R¹⁰ is optionally substituted pyridinyl.

46. Compound of claim 43 wherein R¹⁰ is optionally substituted phenyl.

47. Compound of claim 43 wherein R¹⁰ is cyclohexyl, pyridinyl, or phenyl, wherein said cyclohexyl, pyridinyl, and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio.

48. Compound of claim 43 wherein R¹¹ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

49. Compound of claim 43 wherein:
R¹⁰ is cyclohexyl or phenyl, wherein said cyclohexyl and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio; and
R¹¹ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

50. A compound of claim 49 having Formula IVA: 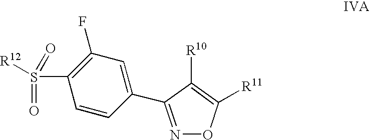
wherein R¹⁰, R¹¹ and R¹² are as defined in claim 49.

51. A compound of claim 49 having Formula IVB: 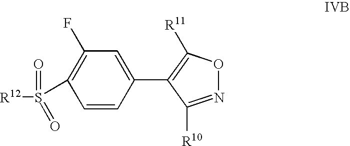
wherein R¹⁰, R¹¹ and R¹² are as defined in claim 49.

52. Compound of claim 43 wherein:
R¹⁰ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and
R¹¹ is a radical selected from hydrido, C₁₋₂-alkyl, C₁₋₃-alkoxy, C₁₋₃-alkylcarbonyl, C₁₋₃-haloalkyl, C₁₋₃-hydroxyalkyl, and C₁₋₃-alkoxycarbonyl.

53. Compound of claim 43 wherein
R¹⁰ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, and methoxy; and
R¹¹ is a radical selected from hydrido, methyl, methoxy, methylcarbonyl, trifluoromethyl, difluoromethyl, hydroxymethyl, and methoxycarbonyl.

54. Compound of claim 49 wherein the compound of Formula I is 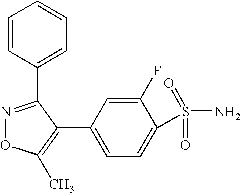
or a pharmaceutically-acceptable salt, tautomer or prodrug thereof.

55. A compound of claim 1 having Formula V: 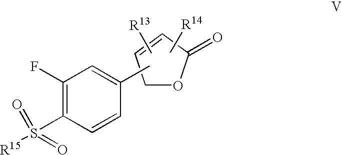
wherein:
R¹³ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹⁴ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R¹⁵ is methyl or amino; or
a pharmaceutically-acceptable salt, tautomer or prodrug thereof
provided that when R¹³ is 4-bromophenyl, R¹⁴ is not hydrogen.

56. Compound of claim 55 wherein:
R¹³ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹⁴ is a radical selected from hydrido, halo, C₁₋₂-alkyl, C₂₋₃-alkenyl, C₂₋₃-alkynyl, oxo, cyano, carboxyl, cyano-C₁₋₃-alkyl, (5- or 6-member ring heterocyclyl)oxy, C₁₋₃-alkoxy, C₁₋₃-alkylthio, C₁₋₃-alkylcarbonyl, C₃₋₆-cycloalkyl, phenyl, C₁₋₃-haloalkyl, 5- or 6-member ring heterocyclyl, C₃₋₆-cycloalkenyl, phenyl-C₁₋₃-alkyl, (5- or 6-member ring heterocyclyl)-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxy-C₁₋₃-alkyl, C₁₋₃-alkoxyphenyl-C₁₋₃-alkoxy-C₁₋₃-alkyl, C₁₋₃-alkoxycarbonyl-C₁₋₃-alkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-phenylamino, N-(phenyl-C₁₋₃-alkyl)amino, N-(C₁₋₃-alkyl)-N-(phenyl-C₁₋₃-alkyl)amino, N-(C₁₋₃-alkyl)-N-phenylamino, amino-C₁₋₃-alkyl, C₁₋₃-alkylamino-C₁₋₃-alkyl, N-phenylamino-C₁₋₃-alkyl, N-phenyl-C₁₋₃-alkylamino-C₁₋₃-alkyl, N-(C₁₋₃-alkyl)-N-phenyl-C₁₋₃-alkylamino-C₁₋₃-alkyl, N(C₁₋₃-alkyl)-N-phenylamino-C₁₋₃-alkyl, phenyloxy, phenyl-C₁₋₃-alkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R¹⁵ is methyl or amino; or
a pharmaceutically-acceptable salt, tautomer or prodrug thereof.

57. Compound of claim 56 wherein R¹³ is optionally substituted cyclohexyl.

58. Compound of claim 56 wherein R¹³ is optionally substituted pyridinyl.

59. Compound of claim 56 wherein R¹³ is optionally substituted phenyl.

60. Compound of claim 56 wherein R¹³ is cyclohexyl, pyridinyl, or phenyl, wherein said cyclohexyl, pyridinyl, and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio.

61. Compound of claim 56 wherein R¹⁴ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

62. Compound of claim 56 wherein:
R¹³ is cyclohexyl or phenyl, wherein said cyclohexyl and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio; and
R¹⁴ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

63. A compound of claim 62 having Formula VA: 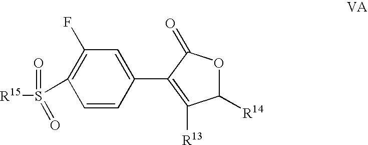
wherein R¹³, R¹⁴ and R¹⁵ are as defined in claim 62.

64. A compound of claim 62 having Formula VB: 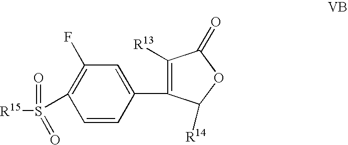
wherein R¹³, R¹⁴ and R¹⁵ are as defined in claim 62.

65. Compound of claim 56 wherein:
R¹³ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and
R¹⁴ is a radical selected from hydrido, Cl z-alkyl, C₁₋₃-alkoxy, C₁₋₃-alkylcarbonyl, C₁₋₃-haloalkyl, C₁₋₃-hydroxyalkyl, and C₁₋₃-alkoxycarbonyl.

66. Compound of claim 56 wherein
R¹³ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, and methoxy; and
R¹⁴ is a radical selected from hydrido, methyl, methoxy, methylcarbonyl, trifluoromethyl, difluoromethyl, hydroxymethyl, and methoxycarbonyl.

67. Compound of claim 62 wherein the compound of Formula I is 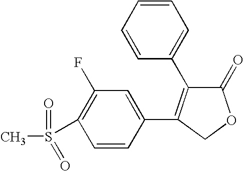
or a pharmaceutically-acceptable salt, tautomer or prodrug thereof.

68. A compound of claim 1 having Formula VI: 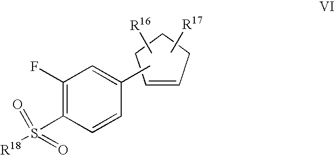
wherein:
R¹⁶ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹⁷ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenyl amino-C₁₋₃-alkyl, N-phenyl-C₁₋₃-alkylaminoalkyl, N-(C₁₋₃-alkyl)-N-(phenyl-C₁₋₃-alkylamino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R¹⁸ is methyl or amino; or
a pharmaceutically-acceptable salt, tautomer or prodrug thereof.

69. Compound of claim 68 wherein:
R¹⁶ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹⁷ is a radical selected from hydrido, halo, C₁₋₂-alkyl, C₂₋₃-alkenyl, C₂₋₃-alkynyl, oxo, cyano, carboxyl, cyano-C₁₋₃-alkyl, (5- or 6-member ring heterocyclyl)oxy, C₁₋₃-alkoxy, C₁₋₃-alkylthio, C₁₋₃-alkylcarbonyl, C₃₋₆-cycloalkyl, phenyl, C₁₋₃-haloalkyl, 5- or 6-member ring heterocyclyl, C₃₋₆-cycloalkenyl, phenyl-C₁₋₃-alkyl, (5- or 6-member ring heterocyclyl)-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxy-C₁₋₃-alkyl, C₁₋₃-alkoxyphenyl-C₁₋₃-alkoxy-C₁₋₃-alkyl, C₁₋₃-alkoxycarbonyl-C₁₋₃-alkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-phenylamino, N-(phenyl-C₁₋₃-alkyl)amino, N-(C₁₋₃-alkyl)-N-(phenyl-C₁₋₃-alkyl)amino, N-(C₁₋₃-alkyl)-N-phenylamino, amino-C₁₋₃-alkyl, C₁₋₃-alkylamino-C₁₋₃-alkyl, N-phenylamino-C₁₋₃-alkyl, N-phenyl-C₁₋₃-alkylamino-C₁₋₃-alkyl, N-(C₁₋₃-alkyl)-N-phenyl-C₁₋₃-alkylamino-C₁₋₃-alkyl, N-(C₁₋₃-alkyl)-N-phenylamino-C₁₋₃-alkyl, phenyloxy, phenyl-C₁₋₃-alkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R¹⁸ is methyl or amino; or
a pharmaceutically-acceptable salt, tautomer or prodrug thereof.

70. Compound of claim 69 wherein R¹⁶ is optionally substituted cyclohexyl.

71. Compound of claim 69 wherein R³⁶ is optionally substituted pyridinyl.

72. Compound of claim 69 wherein R¹⁶ is optionally substituted phenyl.

73. Compound of claim 69 wherein R¹⁶ is cyclohexyl, pyridinyl, or phenyl, wherein said cyclohexyl, pyridinyl, and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio.

74. Compound of claim 69 wherein R¹⁷ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

75. Compound of claim 69 wherein:
R¹⁶ is cyclohexyl or phenyl, wherein said cyclohexyl and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio; and
R¹⁷ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

76. A compound of claim 75 having Formula VIA: 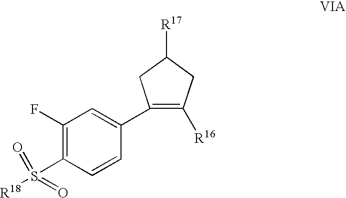
wherein R¹⁶, R¹⁷ and R¹⁸ are as defined in claim 75.

77. Compound of claim 69 wherein:
R¹⁶ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and
R¹⁷ is a radical selected from hydrido, C₁₋₂-alkyl, C₁₋₃-alkoxy, C₁₋₃-alkylcarbonyl, C₁₋₃-haloalkyl, C₁₋₃-hydroxyalkyl, and C₁₋₃-alkoxycarbonyl.

78. Compound of claim 69 wherein
R¹⁶ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, and methoxy; and
R¹⁷ is a radical selected from hydrido, methyl, methoxy, methylcarbonyl, trifluoromethyl, difluoromethyl, hydroxymethyl, and methoxycarbonyl.

79. Compound of claim 75 wherein the compound of Formula I is 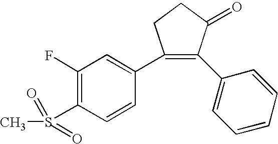
or a pharmaceutically-acceptable salt, tautomer or prodrug thereof.

80. A compound of claim 1 having Formula VII: 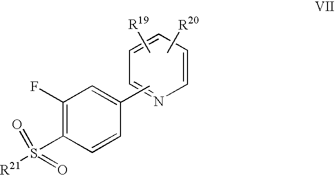
wherein:
R¹⁹ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R²⁰ is represents one or more radicals selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R²¹ is methyl or amino; or
a pharmaceutically-acceptable salt, tautomer or prodrug thereof.

81. Compound of claim 80 wherein:
R¹⁹ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R²⁰ is a radical selected from hydrido, halo, C₁₋₂-alkyl, C₂₋₃-alkenyl, C₂₋₃-alkynyl, oxo, cyano, carboxyl, cyano-C₁₋₃-alkyl, (5- or 6-member ring heterocyclyl)oxy, C₁₋₃-alkoxy, C₁₋₃-alkylthio, C₁₋₃-alkylcarbonyl, C₃₋₆-cycloalkyl, phenyl, C₁₋₃-haloalkyl, 5- or 6-member ring heterocyclyl, C₃₋₆-cycloalkenyl, phenyl-C₁₋₃-alkyl, (5- or 6-member ring heterocyclyl)-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxy-C₁₋₃-alkyl, C₁₋₃-alkoxyphenyl-C₁₋₃-alkoxy-C₁₋₃-alkyl, C₁₋₃-alkoxycarbonyl-C₁₋₃-alkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-phenylamino, N-(phenyl-C₁₋₃-alkyl)amino, N-(C₁₋₃-alkyl)-N-(phenyl-C₁₋₃-alkyl)amino, N-(C₁₋₃-alkyl)-N-phenylamino, amino-C₁₋₃-alkyl, C₁₋₃-alkylamino-C₁₋₃-alkyl, N-phenylamino-C₁₋₃-alkyl, N-phenyl-C₁₋₃-alkylamino-C₁₋₃-alkyl, N-(C₁₋₃-alkyl)-N-phenyl-C₁₋₃-alkylamino-C₁₋₃-alkyl, N-(C₁₋₃-alkyl)-N-phenylamino-C₁₋₃-alkyl, phenyloxy, phenyl-C₁₋₃-alkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R²¹ is methyl or amino; or
a pharmaceutically-acceptable salt, tautomer or prodrug thereof.

82. Compound of claim 81 wherein R¹⁹ is optionally substituted cyclohexyl.

83. Compound of claim 81 wherein R¹⁹ is optionally substituted pyridinyl.

84. Compound of claim 81 wherein R¹⁹ is optionally substituted phenyl.

85. Compound of claim 81 wherein R¹⁹ is cyclohexyl, pyridinyl, or phenyl, wherein said cyclohexyl, pyridinyl, and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio.

86. Compound of claim 81 wherein R²⁰ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

87. Compound of claim 81 wherein:
R¹⁹ is cyclohexyl or phenyl, wherein said cyclohexyl and phenyl may be optionally substituted with one, two or three radicals selected from methyl, difluoromethyl, trifluoromethyl, cyano, carboxyl, methoxycarbonyl, hydroxyl, hydroxymethyl, trifluoromethoxy, amino, methylamino, phenylamino, nitro, methoxymethyl, methylsulfinyl, fluoro, chloro, bromo, methoxy and methylthio; and
R²⁰ is a radical selected from hydrido, fluoro, chloro, bromo, methyl, oxo, cyano, carboxyl, cyanomethyl, methoxy, methylthio, methylcarbonyl, phenyl, trifluoromethyl, difluoromethyl, phenylmethyl, methylthiomethyl, hydroxymethyl, methoxycarbonyl, ethoxycarbonyl, phenylcarbonyl, phenylmethylcarbonyl, methoxymethyl, phenylthiomethyl, phenyloxymethyl, methoxyphenylmethoxymethyl, methoxycarbonylmethyl, aminocarbonyl, aminocarbonylmethyl, methylaminocarbonyl, N-phenylaminocarbonyl, N-methyl-N-phenylaminocarbonyl, methylaminocarbonylmethyl, carboxymethyl, methylamino, N-phenylamino, N-(phenylmethyl)amino, N-methyl-N-(phenylmethyl)amino, N-methyl-N-phenylamino, aminomethyl, methylaminomethyl, N-phenylaminomethyl, N-phenylmethylaminomethyl, N-methyl-N-phenylmethylaminomethyl, N-methyl-N-phenylaminomethyl, phenyloxy, phenylmethoxy, phenylthio, phenylmethylthio, methylsulfinyl, methylsulfonyl, aminosulfonyl, methylaminosulfonyl, N-phenylaminosulfonyl, phenylsulfonyl, and N-methyl-N-phenylaminosulfonyl.

88. A compound of claim 87 having Formula VIIA: 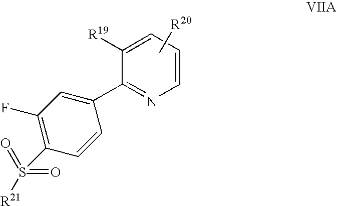
wherein R¹⁹, R²⁰ and R²¹ are as defined in claim 87.

89. Compound of claim 81 wherein:
R¹⁹ is cyclohexyl or phenyl, wherein said cyclohexyl and phenyl may be optionally substituted with one, two or three radicals selected from halo, cyano, C₁₋₂-alkyl, C₁₋₂-haloalkyl, C₁₋₂-alkoxy, and C₁₋₂-haloalkoxy; and
R²⁰ is a radical selected from hydrido, C₁₋₂-alkyl, C₁₋₃-alkoxy, C₁₋₃-alkylcarbonyl, C₁₋₃-haloalkyl, C₁₋₃-hydroxyalkyl, and C₁₋₃-alkoxycarbonyl.

90. Compound of claim 81 wherein
R¹⁹ is cyclohexyl or phenyl, wherein said cyclohexyl and phenyl may be optionally substituted with one, two or three radicals selected from methyl, difluoromethyl, trifluoromethyl, trifluoromethoxy, cyano, fluoro, chloro, bromo, and methoxy; and
R²⁰ is a radical selected from hydrido, methyl, methoxy, methylcarbonyl, trifluoromethyl, difluoromethyl, hydroxymethyl, and methoxycarbonyl.

91. Compound of claim 87 wherein the compound of Formula I is 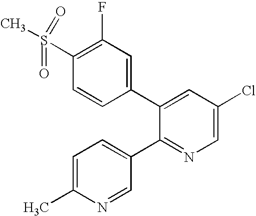
or a pharmaceutically-acceptable salt, tautomer or prodrug thereof.

92. A pharmaceutical composition comprising a therapeutically-effective amount of a compound of claim 1.

93. A pharmaceutical composition comprising a therapeutically-effective amount of a compound of claim 17.

94. A pharmaceutical composition comprising a therapeutically-effective amount of a compound of claim 31.

95. A pharmaceutical composition comprising a therapeutically-effective amount of a compound of claim 42.

96. A pharmaceutical composition comprising a therapeutically-effective amount of a compound of claim 55.

97. A pharmaceutical composition comprising a therapeutically-effective amount of a compound of claim 68.

98. A pharmaceutical composition comprising a therapeutically-effective amount of a compound of claim 80.

99. A method of treating inflammation, said method comprising administering to the subject having or susceptible to such inflammation or inflammation-associated disorder, a therapeutically-effective amount of a compound of Formula I 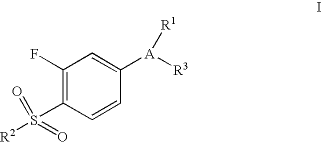
wherein:
A is a 5- or 6-member ring substituent selected from partially saturated or unsaturated heterocyclic and carbocyclic rings;
R¹ is cyclohexyl, pyridinyl, or phenyl, wherein said cyclohexyl, pyridinyl, and phenyl may be may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R² is methyl or amino; and
R³ represents one or more radicals selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkyl amino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl;
or a pharmaceutically-acceptable salt, tautomer or prodrug thereof.

100. The method of claim 99 wherein the compound corresponds to Formula II: 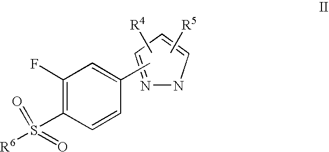
wherein:
R⁴ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R⁵ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R⁶ is methyl or amino;
or a pharmaceutically-acceptable salt, tautomer or prodrug thereof;
provided that when R¹ is 4-bromophenyl and R² is methyl, R³ is not hydrogen, cyano, trifluoromethyl or ethoxycarbonyl.

101. The method of claim 99 wherein the compound corresponds to Formula III: 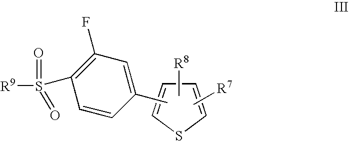
wherein:
R⁷ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R⁸ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R⁹ is methyl or amino; or
a pharmaceutically-acceptable salt, tautomer or prodrug thereof.

102. The method of claim 99 wherein the compound corresponds to Formula IV: 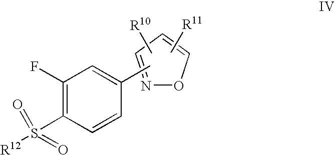
wherein:
R¹⁰ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹¹ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
wherein R¹² is methyl or amino; or
a pharmaceutically-acceptable salt, tautomer or prodrug thereof.

103. The method of claim 99 wherein the compound corresponds to Formula V: 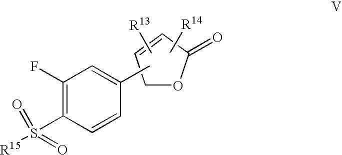
wherein:
R¹³ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹⁴ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R¹⁵ is methyl or amino; or
a pharmaceutically-acceptable salt, tautomer or prodrug thereof.

104. The method of claim 99 wherein the compound corresponds to Formula 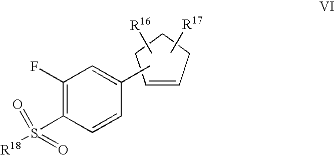
wherein:
R¹⁶ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R¹⁷ is a radical selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R¹⁸ is methyl or amino; or
a pharmaceutically-acceptable salt, tautomer or prodrug thereof.

105. The method of claim 99 wherein the compound corresponds to Formula VII: 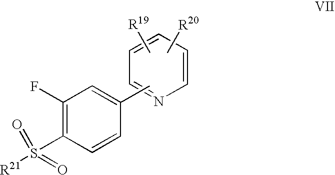
wherein:
R¹⁹ is cyclohexyl, pyridinyl, or phenyl, wherein said cyclohexyl, pyridinyl, and phenyl may be optionally substituted with one, two or three radicals selected from C₁₋₂-alkyl, C₁₋₂-haloalkyl, cyano, carboxyl, C₁₋₂-alkoxycarbonyl, hydroxyl, C₁₋₂-hydroxyalkyl, C₁₋₂-haloalkoxy, amino, C₁₋₂-alkylamino, phenylamino, nitro, C₁₋₂-alkoxy-C₁₋₂-alkyl, C₁₋₂-alkylsulfinyl, halo, C₁₋₂-alkoxy and C₁₋₃-alkylthio;
R²⁰ is represents one or more radicals selected from hydrido, halo, C₁₋₂-alkyl, C₂₋₃-alkenyl, C₂₋₃-alkynyl, oxo, cyano, carboxyl, cyano-C₁₋₃-alkyl, heterocyclyloxy, C₁₋₃-alkoxy, C₁₋₃-alkylthio, alkylcarbonyl, cycloalkyl, phenyl, C₁₋₃-haloalkyl, heterocyclyl, cycloalkenyl, phenyl-C₁₋₃-alkyl, heterocyclyl-C₁₋₃-alkyl, C₁₋₃-alkylthio-C₁₋₃-alkyl, C₁₋₃-hydroxyalkyl, C₁₋₃-alkoxycarbonyl, phenylcarbonyl, phenyl-C₁₋₃-alkylcarbonyl, phenyl-C₂₋₃-alkenyl, C₁₋₃-alkoxy-C₁₋₃-alkyl, phenylthio-C₁₋₃-alkyl, phenyloxyalkyl, alkoxyphenylalkoxyalkyl, alkoxycarbonylalkyl, aminocarbonyl, aminocarbonyl-C₁₋₃-alkyl, C₁₋₃-alkylaminocarbonyl, N-phenylaminocarbonyl, N-(C₁₋₃-alkyl)-N-phenylaminocarbonyl, C₁₋₃-alkylaminocarbonyl-C₁₋₃-alkyl, carboxy-C₁₋₃-alkyl, C₁₋₃-alkylamino, N-arylamino, N-aralkylamino, N-(C₁₋₃-alkyl)-N-aralkylamino, N-(C₁₋₃-alkyl)-N-arylamino, amino-C₁₋₃-alkyl, C₁₋₃-alkylaminoalkyl, N-phenylamino-C₁₋₃-alkyl, N-phenyl-C₁₋₃-alkylaminoalkyl, N-(C₁₋₃-alkyl)-N-(phenyl-C₁₋₃-alkyl)amino-C₁₋₃-alkyl, N-(C₁₋₃-alkyl)-N-phenylamino-C₁₋₃-alkyl, phenyloxy, phenylalkoxy, phenylthio, phenyl-C₁₋₃-alkylthio, C₁₋₃-alkylsulfinyl, C₁₋₃-alkylsulfonyl, aminosulfonyl, C₁₋₃-alkylaminosulfonyl, N-phenylaminosulfonyl, phenylsulfonyl, and N-(C₁₋₃-alkyl)-N-phenylaminosulfonyl; and
R²¹ is methyl or amino; or
a pharmaceutically-acceptable salt, tautomer or prodrug thereof.

106. The method of claim 99 for use in the treatment of inflammation.

107. The method of claim 99 for use in the treatment of an inflammation-associated disorder.

108. The method of claim 107 wherein the inflammation-associated disorder is arthritis.

109. The method of claim 107 wherein the inflammation-associated disorder is pain.

110. The method of claim 107 wherein the inflammation-associated disorder is fever.

111. A method of treating cancer, said method comprising administering to the subject having or susceptible to such cancer, a therapeutically-effective amount of a compound of claim 99.

112. The method of claim 111 wherein the compound is administered intravenously.

113. The method of claim 111 wherein the compound is administered intramuscularly.
